SEQUENCE LISTING (356 sequences)

SEQ ID NO: 1 - RNA, 49 nt, synthetic construct
  misc_feature 1..49: note = SERPINA1 editing sequence
  modified_base 37: mod_base = i
  source 1..49: mol_type = other RNA
taaaaacatggccccagcagcttcagtccctttctcntcgatggtcagc

SEQ ID NO: 2 - RNA, 61 nt, synthetic construct
  misc_feature 1..61: note = SERPINA1 editing sequence
  source 1..61: mol_type = other RNA
acatggccccagcagcttcagtccctttctcgtcgatggtcagcacagccttatgcacggc

SEQ ID NO: 3 - RNA, 61 nt, synthetic construct
  misc_feature 1..61: note = SERPINA1 editing sequences
  modified_base 32: mod_base = i
  source 1..61: mol_type = other RNA
acatggccccagcagcttcagtccctttctcntcgatggtcagcacagccttatgcacggc

SEQ ID NO: 4 - RNA, 55 nt, synthetic construct
  modified_base 27: mod_base = OTHER; note = thymine
  misc_feature 1..55: note = SERPINA1 editing sequences
  modified_base 29: mod_base = i
  source 1..55: mol_type = other RNA
tggccccagcagcttcagtccctttctcntcgatggtcagcacagccttatgcac

SEQ ID NO: 5 - RNA, 51 nt, synthetic construct
  modified_base 25: mod_base = OTHER; note = thymine
  misc_feature 1..51: note = SERPINA1 editing sequences
  modified_base 27: mod_base = i
  source 1..51: mol_type = other RNA
gccccagcagcttcagtccctttctcntcgatggtcagcacagccttatgc

SEQ ID NO: 6 - RNA, 36 nt, synthetic construct
  modified_base 25: mod_base = OTHER; note = thymine
  misc_feature 1..36: note = SERPINA1 editing sequence
  modified_base 27: mod_base = i
  source 1..36: mol_type = other RNA
gccccagcagcttcagtccctttctcntcgatggtc

SEQ ID NO: 7 - RNA, 46 nt, synthetic construct
  modified_base 30: mod_base = OTHER; note = thymine
  misc_feature 1..46: note = SERPINA1 editing sequence
  modified_base 32: mod_base = i
  source 1..46: mol_type = other RNA
acatggccccagcagcttcagtccctttctcntcgatggtcagcac

SEQ ID NO: 8 - RNA, 45 nt, synthetic construct
  modified_base 30: mod_base = OTHER; note = thymine
  misc_feature 1..45: note = SERPINA1 editing sequence
  modified_base 32: mod_base = i
  source 1..45: mol_type = other RNA
acatggccccagcagcttcagtccctttctcntcgatggtcagca

SEQ ID NO: 9 - RNA, 44 nt, synthetic construct
  modified_base 30: mod_base = OTHER; note = thymine
  misc_feature 1..44: note = SERPINA1 editing sequence
  modified_base 32: mod_base = i
  source 1..44: mol_type = other RNA
acatggccccagcagcttcagtccctttctcntcgatggtcagc

SEQ ID NO: 10 - RNA, 43 nt, synthetic construct
  modified_base 30: mod_base = OTHER; note = thymine
  misc_feature 1..43: note = SERPINA1 editing sequence
  modified_base 32: mod_base = i
  source 1..43: mol_type = other RNA
acatggccccagcagcttcagtccctttctcntcgatggtcag

SEQ ID NO: 11 - RNA, 42 nt, synthetic construct
  modified_base 30: mod_base = OTHER; note = thymine
  misc_feature 1..42: note = SERPINA1 editing sequence
  modified_base 32: mod_base = i
  source 1..42: mol_type = other RNA
acatggccccagcagcttcagtccctttctcntcgatggtca

SEQ ID NO: 12 - RNA, 41 nt, synthetic construct
  modified_base 30: mod_base = OTHER; note = thymine
  misc_feature 1..41: note = SERPINA1 editing sequence
  modified_base 32: mod_base = i
  source 1..41: mol_type = other RNA
acatggccccagcagcttcagtccctttctcntcgatggtc

SEQ ID NO: 13 - RNA, 40 nt, synthetic construct
  modified_base 30: mod_base = OTHER; note = thymine
  misc_feature 1..40: note = SERPINA1 editing sequence
  modified_base 32: mod_base = i
  source 1..40: mol_type = other RNA
acatggccccagcagcttcagtccctttctcntcgatggt

SEQ ID NO: 14 - RNA, 39 nt, synthetic construct
  modified_base 30: mod_base = OTHER; note = thymine
  misc_feature 1..39: note = SERPINA1 editing sequence
  modified_base 32: mod_base = i
  source 1..39: mol_type = other RNA
acatggccccagcagcttcagtccctttctcntcgatgg

SEQ ID NO: 15 - RNA, 38 nt, synthetic construct
  modified_base 30: mod_base = OTHER; note = thymine
  misc_feature 1..38: note = SERPINA1 editing sequence
  modified_base 32: mod_base = i
  source 1..38: mol_type = other RNA
acatggccccagcagcttcagtccctttctcntcgatg

SEQ ID NO: 16 - RNA, 37 nt, synthetic construct
  modified_base 30: mod_base = OTHER; note = thymine
  misc_feature 1..37: note = SERPINA1 editing sequence
  modified_base 32: mod_base = i
  source 1..37: mol_type = other RNA
acatggccccagcagcttcagtccctttctcntcgat

SEQ ID NO: 17 - RNA, 36 nt, synthetic construct
  modified_base 30: mod_base = OTHER; note = thymine
  misc_feature 1..36: note = SERPINA1 editing sequence
  modified_base 32: mod_base = i
  source 1..36: mol_type = other RNA
acatggccccagcagcttcagtccctttctcntcga

SEQ ID NO: 18 - RNA, 35 nt, synthetic construct
  modified_base 30: mod_base = OTHER; note = thymine
  misc_feature 1..35: note = SERPINA1 editing sequence
  modified_base 32: mod_base = i
  source 1..35: mol_type = other RNA
acatggccccagcagcttcagtccctttctcntcg

SEQ ID NO: 19 - RNA, 41 nt, synthetic construct
  modified_base 25: mod_base = OTHER; note = thymine
  misc_feature 1..41: note = SERPINA1 editing sequence
  modified_base 27: mod_base = i
  source 1..41: mol_type = other RNA
gccccagcagcttcagtccctttctcntcgatggtcagcac

SEQ ID NO: 20 - RNA, 40 nt, synthetic construct
  modified_base 25: mod_base = OTHER; note = thymine
  misc_feature 1..40: note = SERPINA1 editing sequence
  modified_base 27: mod_base = i
  source 1..40: mol_type = other RNA
gccccagcagcttcagtccctttctcntcgatggtcagca

SEQ ID NO: 21 - RNA, 39 nt, synthetic construct
  modified_base 25: mod_base = OTHER; note = thymine
  misc_feature 1..39: note = SERPINA1 editing sequence
  modified_base 27: mod_base = i
  source 1..39: mol_type = other RNA
gccccagcagcttcagtccctttctcntcgatggtcagc

SEQ ID NO: 22 - RNA, 38 nt, synthetic construct
  modified_base 25: mod_base = OTHER; note = thymine
  misc_feature 1..38: note = SERPINA1 editing sequence
  modified_base 27: mod_base = i
  source 1..38: mol_type = other RNA
gccccagcagcttcagtccctttctcntcgatggtcag

SEQ ID NO: 23 - RNA, 35 nt, synthetic construct
  modified_base 25: mod_base = OTHER; note = thymine
  misc_feature 1..35: note = SERPINA1 editing sequence
  modified_base 27: mod_base = i
  source 1..35: mol_type = other RNA
gccccagcagcttcagtccctttctcntcgatggt

SEQ ID NO: 24 - RNA, 34 nt, synthetic construct
  modified_base 25: mod_base = OTHER; note = thymine
  misc_feature 1..34: note = SERPINA1 editing sequence
  modified_base 27: mod_base = i
  source 1..34: mol_type = other RNA
gccccagcagcttcagtccctttctcntcgatgg

SEQ ID NO: 25 - RNA, 33 nt, synthetic construct
  modified_base 25: mod_base = OTHER; note = thymine
  misc_feature 1..33: note = SERPINA1 editing sequence
  modified_base 27: mod_base = i
  source 1..33: mol_type = other RNA
gccccagcagcttcagtccctttctcntcgatg

SEQ ID NO: 26 - RNA, 32 nt, synthetic construct
  modified_base 25: mod_base = OTHER; note = thymine
  misc_feature 1..32: note = SERPINA1 editing sequence
  modified_base 27: mod_base = i
  source 1..32: mol_type = other RNA
gccccagcagcttcagtccctttctcntcgat

SEQ ID NO: 27 - RNA, 31 nt, synthetic construct
  modified_base 25: mod_base = OTHER; note = thymine
  misc_feature 1..31: note = SERPINA1 editing sequence
  modified_base 27: mod_base = i
  source 1..31: mol_type = other RNA
gccccagcagcttcagtccctttctcntcga

SEQ ID NO: 28 - RNA, 30 nt, synthetic construct
  modified_base 25: mod_base = OTHER; note = thymine
  misc_feature 1..30: note = SERPINA1 editing sequence
  modified_base 27: mod_base = i
  source 1..30: mol_type = other RNA
gccccagcagcttcagtccctttctcntcg

SEQ ID NO: 29 - RNA, 45 nt, synthetic construct
  modified_base 29: mod_base = OTHER; note = thymine
  misc_feature 1..45: note = SERPINA1 editing sequence
  modified_base 31: mod_base = i
  source 1..45: mol_type = other RNA
catggccccagcagcttcagtccctttctcntcgatggtcagcac

SEQ ID NO: 30 - RNA, 44 nt, synthetic construct
  modified_base 28: mod_base = OTHER; note = thymine
  misc_feature 1..44: note = SERPINA1 editing sequence
  modified_base 30: mod_base = i
  source 1..44: mol_type = other RNA
atggccccagcagcttcagtccctttctcntcgatggtcagcac

SEQ ID NO: 31 - RNA, 43 nt, synthetic construct
  modified_base 27: mod_base = OTHER; note = thymine
  misc_feature 1..43: note = SERPINA1 editing sequence
  modified_base 29: mod_base = i
  source 1..43: mol_type = other RNA
tggccccagcagcttcagtccctttctcntcgatggtcagcac

SEQ ID NO: 32 - RNA, 40 nt, synthetic construct
  modified_base 24: mod_base = OTHER; note = thymine
  misc_feature 1..40: note = SERPINA1 editing sequence
  modified_base 26: mod_base = i
  source 1..40: mol_type = other RNA
ccccagcagcttcagtccctttctcntcgatggtcagcac

SEQ ID NO: 33 - RNA, 39 nt, synthetic construct
  modified_base 23: mod_base = OTHER; note = thymine
  misc_feature 1..39: note = SERPINA1 editing sequence
  modified_base 25: mod_base = i
  source 1..39: mol_type = other RNA
cccagcagcttcagtccctttctcntcgatggtcagcac

SEQ ID NO: 34 - RNA, 38 nt, synthetic construct
  modified_base 22: mod_base = OTHER; note = thymine
  misc_feature 1..38: note = SERPINA1 editing sequence
  modified_base 24: mod_base = i
  source 1..38: mol_type = other RNA
ccagcagcttcagtccctttctcntcgatggtcagcac

SEQ ID NO: 35 - RNA, 37 nt, synthetic construct
  modified_base 21: mod_base = OTHER; note = thymine
  misc_feature 1..37: note = SERPINA1 editing sequence
  modified_base 23: mod_base = i
  source 1..37: mol_type = other RNA
cagcagcttcagtccctttctcntcgatggtcagcac

SEQ ID NO: 36 - RNA, 36 nt, synthetic construct
  modified_base 20: mod_base = OTHER; note = thymine
  misc_feature 1..36: note = SERPINA1 editing sequence
  modified_base 22: mod_base = i
  source 1..36: mol_type = other RNA
agcagcttcagtccctttctcntcgatggtcagcac

SEQ ID NO: 37 - RNA, 41 nt, synthetic construct
  modified_base 15: mod_base = OTHER; note = thymine
  misc_feature 1..41: note = SERPINA1 editing sequence
  modified_base 17: mod_base = i
  source 1..41: mol_type = other RNA
cttcagtccctttctcntcgatggtcagcacagccttatgc

SEQ ID NO: 38 - RNA, 46 nt, synthetic construct
  modified_base 15: mod_base = OTHER; note = thymine
  misc_feature 1..46: note = SERPINA1 editing sequence
  modified_base 17: mod_base = i
  source 1..46: mol_type = other RNA
cttcagtccctttctcntcgatggtcagcacagccttatgcacggc

SEQ ID NO: 39 - RNA, 32 nt, synthetic construct
  modified_base 25: mod_base = OTHER; note = thymine
  misc_feature 1..32: note = SERPINA1 editing sequence
  modified_base 27: mod_base = i
  source 1..32: mol_type = other RNA
gccccagcagcatcactccctttctcntcgat

SEQ ID NO: 40 - RNA, 40 nt, synthetic construct
  modified_base 24: mod_base = OTHER; note = thymine
  misc_feature 1..40: note = SERPINA1 editing sequence
  modified_base 26: mod_base = i
  source 1..40: mol_type = other RNA
ccccagcagcttcagtccctttctantcgatggtcagcac

SEQ ID NO: 41 - RNA, 40 nt, synthetic construct
  modified_base 24: mod_base = OTHER; note = thymine
  misc_feature 1..40: note = SERPINA1 editing sequence
  modified_base 26: mod_base = i
  source 1..40: mol_type = other RNA
ccccagcagcttcagtccctttctgntcgatggtcagcac

SEQ ID NO: 42 - RNA, 40 nt, synthetic construct
  modified_base 24: mod_base = OTHER; note = thymine
  misc_feature 1..40: note = SERPINA1 editing sequence
  modified_base 25: mod_base = OTHER; note = 5-methyl cytosine
  modified_base 26: mod_base = i
  source 1..40: mol_type = other RNA
ccccagcagcttcagtccctttctnntcgatggtcagcac

SEQ ID NO: 43 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25..26: mod_base = i
  source 1..40: mol_type = other RNA

SEQ ID NO: 44 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = Benner's base
  modified_base 26: mod_base = i
  source 1..40: mol_type = other RNA

SEQ ID NO: 45 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = 7-deaza-8-aza guanine
  modified_base 26: mod_base = i
  source 1..40: mol_type = other RNA

SEQ ID NO: 46 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = pyrrolo cytosine
  modified_base 26: mod_base = i
  source 1..40: mol_type = other RNA

SEQ ID NO: 47 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = abasic
  modified_base 26: mod_base = i
  source 1..40: mol_type = other RNA

SEQ ID NO: 48 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = 5-propynyl cytosine
  modified_base 26: mod_base = i
  source 1..40: mol_type = other RNA

SEQ ID NO: 49 - RNA, 40 nt, synthetic construct; identical to SEQ ID NO: 42
  modified_base 24: mod_base = OTHER; note = thymine
  modified_base 25: mod_base = OTHER; note = 2-amino purine
  modified_base 26: mod_base = i
  source 1..40: mol_type = other RNA

SEQ ID NO: 50 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = aminoethyl phenoxazine
  modified_base 26: mod_base = i
  source 1..40: mol_type = other RNA

SEQ ID NO: 51 - RNA, 40 nt, synthetic construct; identical to SEQ ID NO: 42
  modified_base 24: mod_base = OTHER; note = thymine
  misc_feature 1..40: note = SERPINA1 editing sequence
  modified_base 25: mod_base = OTHER; note = 5-bromo cytosine
  modified_base 26: mod_base = i
  source 1..40: mol_type = other RNA

SEQ ID NO: 52 - RNA, 40 nt, synthetic construct
  modified_base 38: mod_base = OTHER; note = thymine
  misc_feature 1..40: note = SERPINA1 editing sequence
  modified_base 40: mod_base = i
  source 1..40: mol_type = other RNA
ctctaaaaacatggccccagcagcttcagtccctttctcn

SEQ ID NO: 53 - RNA, 40 nt, synthetic construct
  modified_base 37: mod_base = OTHER; note = thymine
  misc_feature 1..40: note = SERPINA1 editing sequence
  modified_base 39: mod_base = i
  source 1..40: mol_type = other RNA
tctaaaaacatggccccagcagcttcagtccctttctcnt

SEQ ID NO: 54 - RNA, 40 nt, synthetic construct
  modified_base 36: mod_base = OTHER; note = thymine
  misc_feature 1..40: note = SERPINA1 editing sequence
  modified_base 38: mod_base = i
  source 1..40: mol_type = other RNA
ctaaaaacatggccccagcagcttcagtccctttctcntc

SEQ ID NO: 55 - RNA, 40 nt, synthetic construct
  modified_base 35: mod_base = OTHER; note = thymine
  misc_feature 1..40: note = SERPINA1 editing sequence
  modified_base 37: mod_base = i
  source 1..40: mol_type = other RNA
taaaaacatggccccagcagcttcagtccctttctcntcg

SEQ ID NO: 56 - RNA, 40 nt, synthetic construct
  modified_base 34: mod_base = OTHER; note = thymine
  misc_feature 1..40: note = SERPINA1 editing sequence
  modified_base 36: mod_base = i
  source 1..40: mol_type = other RNA
aaaaacatggccccagcagcttcagtccctttctcntcga

SEQ ID NO: 57 - RNA, 40 nt, synthetic construct
  modified_base 33: mod_base = OTHER; note = thymine
  misc_feature 1..40: note = SERPINA1 editing sequence
  modified_base 35: mod_base = i
  source 1..40: mol_type = other RNA
aaaacatggccccagcagcttcagtccctttctcntcgat

SEQ ID NO: 58 - RNA, 40 nt, synthetic construct
  modified_base 32: mod_base = OTHER; note = thymine
  misc_feature 1..40: note = SERPINA1 editing sequence
  modified_base 34: mod_base = i
  source 1..40: mol_type = other RNA
aaacatggccccagcagcttcagtccctttctcntcgatg

SEQ ID NO: 59 - RNA, 40 nt, synthetic construct
  modified_base 31: mod_base = OTHER; note = thymine
  misc_feature 1..40: note = SERPINA1 editing sequence
  modified_base 33: mod_base = i
  source 1..40: mol_type = other RNA
aacatggccccagcagcttcagtccctttctcntcgatgg

SEQ ID NO: 60 - RNA, 40 nt, synthetic construct
  modified_base 29: mod_base = OTHER; note = thymine
  misc_feature 1..40: note = SERPINA1 editing sequence
  modified_base 31: mod_base = i
  source 1..40: mol_type = other RNA
catggccccagcagcttcagtccctttctcntcgatggtc

SEQ ID NO: 61 - RNA, 40 nt, synthetic construct
  modified_base 28: mod_base = OTHER; note = thymine
  misc_feature 1..40: note = SERPINA1 editing sequence
  modified_base 30: mod_base = i
  source 1..40: mol_type = other RNA
atggccccagcagcttcagtccctttctcntcgatggtca

SEQ ID NO: 62 - RNA, 40 nt, synthetic construct
  modified_base 27: mod_base = OTHER; note = thymine
  misc_feature 1..40: note = SERPINA1 editing sequence
  modified_base 29: mod_base = i
  source 1..40: mol_type = other RNA
tggccccagcagcttcagtccctttctcntcgatggtcag

SEQ ID NO: 63 - RNA, 40 nt, synthetic construct
  modified_base 26: mod_base = OTHER; note = thymine
  misc_feature 1..40: note = SERPINA1 editing sequence
  modified_base 28: mod_base = i
  source 1..40: mol_type = other RNA
ggccccagcagcttcagtccctttctcntcgatggtcagc

SEQ ID NO: 64 - RNA, 40 nt, synthetic construct
  modified_base 23: mod_base = OTHER; note = thymine
  misc_feature 1..40: note = SERPINA1 editing sequence
  modified_base 25: mod_base = i
  source 1..40: mol_type = other RNA
cccagcagcttcagtccctttctcntcgatggtcagcaca

SEQ ID NO: 65 - RNA, 40 nt, synthetic construct
  modified_base 22: mod_base = OTHER; note = thymine
  misc_feature 1..40: note = SERPINA1 editing sequence
  modified_base 24: mod_base = i
  source 1..40: mol_type = other RNA
ccagcagcttcagtccctttctcntcgatggtcagcacag

SEQ ID NO: 66 - RNA, 40 nt, synthetic construct
  modified_base 21: mod_base = OTHER; note = thymine
  misc_feature 1..40: note = SERPINA1 editing sequence
  modified_base 23: mod_base = i
  source 1..40: mol_type = other RNA
cagcagcttcagtccctttctcntcgatggtcagcacagc

SEQ ID NO: 67 - RNA, 40 nt, synthetic construct
  modified_base 20: mod_base = OTHER; note = thymine
  misc_feature 1..40: note = SERPINA1 editing sequence
  modified_base 22: mod_base = i
  source 1..40: mol_type = other RNA
agcagcttcagtccctttctcntcgatggtcagcacagcc

SEQ ID NO: 68 - RNA, 40 nt, synthetic construct
  modified_base 19: mod_base = OTHER; note = thymine
  misc_feature 1..40: note = SERPINA1 editing sequence
  modified_base 21: mod_base = i
  source 1..40: mol_type = other RNA
gcagcttcagtccctttctcntcgatggtcagcacagcct

SEQ ID NO: 69 - RNA, 40 nt, synthetic construct
  modified_base 18: mod_base = OTHER; note = thymine
  misc_feature 1..40: note = SERPINA1 editing sequence
  modified_base 20: mod_base = i
  source 1..40: mol_type = other RNA
cagcttcagtccctttctcntcgatggtcagcacagcctt

SEQ ID NO: 70 - RNA, 40 nt, synthetic construct
  modified_base 17: mod_base = OTHER; note = thymine
  misc_feature 1..40: note = SERPINA1 editing sequence
  modified_base 19: mod_base = i
  source 1..40: mol_type = other RNA
agcttcagtccctttctcntcgatggtcagcacagcctta

SEQ ID NO: 71 - RNA, 40 nt, synthetic construct
  modified_base 16: mod_base = OTHER; note = thymine
  misc_feature 1..40: note = SERPINA1 editing sequence
  modified_base 18: mod_base = i
  source 1..40: mol_type = other RNA
gcttcagtccctttctcntcgatggtcagcacagccttat

SEQ ID NO: 72 - RNA, 40 nt, synthetic construct
  modified_base 15: mod_base = OTHER; note = thymine
  misc_feature 1..40: note = SERPINA1 editing sequence
  modified_base 17: mod_base = i
  source 1..40: mol_type = other RNA
cttcagtccctttctcntcgatggtcagcacagccttatg

SEQ ID NO: 73 - RNA, 40 nt, synthetic construct
  modified_base 14: mod_base = OTHER; note = thymine
  misc_feature 1..40: note = SERPINA1 editing sequence
  modified_base 16: mod_base = i
  source 1..40: mol_type = other RNA
ttcagtccctttctcntcgatggtcagcacagccttatgc

SEQ ID NO: 74 - RNA, 40 nt, synthetic construct
  modified_base 13: mod_base = OTHER; note = thymine
  misc_feature 1..40: note = SERPINA1 editing sequence
  modified_base 15: mod_base = i
  source 1..40: mol_type = other RNA
tcagtccctttctcntcgatggtcagcacagccttatgca

SEQ ID NO: 75 - RNA, 40 nt, synthetic construct
  modified_base 12: mod_base = OTHER; note = thymine
  misc_feature 1..40: note = SERPINA1 editing sequence
  modified_base 14: mod_base = i
  source 1..40: mol_type = other RNA
cagtccctttctcntcgatggtcagcacagccttatgcac

SEQ ID NO: 76 - RNA, 40 nt, synthetic construct
  modified_base 11: mod_base = OTHER; note = thymine
  misc_feature 1..40: note = SERPINA1 editing sequence
  modified_base 13: mod_base = i
  source 1..40: mol_type = other RNA
agtccctttctcntcgatggtcagcacagccttatgcacg

SEQ ID NO: 77 - RNA, 40 nt, synthetic construct
  modified_base 10: mod_base = OTHER; note = thymine
  misc_feature 1..40: note = SERPINA1 editing sequence
  modified_base 12: mod_base = i
  source 1..40: mol_type = other RNA
gtccctttctcntcgatggtcagcacagccttatgcacgg

SEQ ID NO: 78 - RNA, 40 nt, synthetic construct
  modified_base 9: mod_base = OTHER; note = thymine
  misc_feature 1..40: note = SERPINA1 editing sequence
  modified_base 11: mod_base = i
  source 1..40: mol_type = other RNA
tccctttctcntcgatggtcagcacagccttatgcacggc

SEQ ID NO: 79 - RNA, 40 nt, synthetic construct
  modified_base 8: mod_base = OTHER; note = thymine
  misc_feature 1..40: note = SERPINA1 editing sequence
  modified_base 10: mod_base = i
  source 1..40: mol_type = other RNA
ccctttctcntcgatggtcagcacagccttatgcacggcc

SEQ ID NO: 80 - RNA, 40 nt, synthetic construct
  modified_base 7: mod_base = OTHER; note = thymine
  misc_feature 1..40: note = SERPINA1 editing sequence
  modified_base 9: mod_base = i
  source 1..40: mol_type = other RNA
cctttctcntcgatggtcagcacagccttatgcacggcct

SEQ ID NO: 81 - RNA, 40 nt, synthetic construct
  modified_base 6: mod_base = OTHER; note = thymine
  misc_feature 1..40: note = SERPINA1 editing sequence
  modified_base 8: mod_base = i
  source 1..40: mol_type = other RNA
ctttctcntcgatggtcagcacagccttatgcacggcctt

SEQ ID NO: 82 - RNA, 40 nt, synthetic construct
  modified_base 5: mod_base = OTHER; note = thymine
  misc_feature 1..40: note = SERPINA1 editing sequence
  modified_base 7: mod_base = i
  source 1..40: mol_type = other RNA
tttctcntcgatggtcagcacagccttatgcacggccttg

SEQ ID NO: 83 - RNA, 40 nt, synthetic construct
  modified_base 4: mod_base = OTHER; note = thymine
  misc_feature 1..40: note = SERPINA1 editing sequence
  modified_base 6: mod_base = i
  source 1..40: mol_type = other RNA
ttctcntcgatggtcagcacagccttatgcacggccttgg

SEQ ID NO: 84 - RNA, 40 nt, synthetic construct
  modified_base 3: mod_base = OTHER; note = thymine
  misc_feature 1..40: note = SERPINA1 editing sequence
  modified_base 5: mod_base = i
  source 1..40: mol_type = other RNA
tctcntcgatggtcagcacagccttatgcacggccttggt

SEQ ID NO: 85 - RNA, 40 nt, synthetic construct
  modified_base 2: mod_base = OTHER; note = thymine
  misc_feature 1..40: note = SERPINA1 editing sequence
  modified_base 4: mod_base = i
  source 1..40: mol_type = other RNA
ctcntcgatggtcagcacagccttatgcacggccttggtg

SEQ ID NO: 86 - RNA, 40 nt, synthetic construct
  modified_base 1: mod_base = OTHER; note = thymine
  misc_feature 1..40: note = SERPINA1 editing sequence
  modified_base 3: mod_base = i
  source 1..40: mol_type = other RNA
tcntcgatggtcagcacagccttatgcacggccttggtgt

SEQ ID NO: 87 - RNA, 77 nt, synthetic construct
  modified_base 38: mod_base = OTHER; note = thymine
  misc_feature 1..77: note = SERPINA1 editing sequence
  modified_base 40: mod_base = i
  source 1..77: mol_type = other RNA
ctctaaaaacatggccccagcagcttcagtccctttctcntcgatggtcagcacagccttatgcacggccttggtgt

SEQ ID NO: 88 - RNA, 52 nt, synthetic construct
  misc_feature 1..52: note = GAPDH editing sequences
  source 1..52: mol_type = other RNA
accttcttgatgtcatcatatttggcaggtttttccagacggcaggtcaggt

SEQ ID NO: 89 - RNA, 52 nt, synthetic construct; identical to SEQ ID NO: 88
  modified_base 8: mod_base = OTHER; note = thymine
  modified_base 16: mod_base = OTHER; note = thymine
  modified_base 22: mod_base = OTHER; note = thymine
  modified_base 30: mod_base = OTHER; note = thymine
  modified_base 32: mod_base = OTHER; note = thymine
  modified_base 34: mod_base = OTHER; note = thymine
  misc_feature 1..52: note = GAPDH editing sequences
  source 1..52: mol_type = other RNA

SEQ ID NO: 90 - RNA, 52 nt, synthetic construct; identical to SEQ ID NO: 88
  modified_base 8: mod_base = OTHER; note = thymine
  modified_base 11: mod_base = OTHER; note = thymine
  modified_base 23: mod_base = OTHER; note = thymine
  modified_base 32: mod_base = OTHER; note = thymine
  misc_feature 1..52: note = GAPDH editing sequence
  source 1..52: mol_type = other RNA

SEQ ID NO: 91 - RNA, 61 nt, synthetic construct
  misc_feature 1..61: note = GAPDH editing sequences
  source 1..61: mol_type = other RNA
cttgatgtcatcatatttggcaggtttttccagacggcaggtcaggtccaccactgacacg

SEQ ID NO: 92 - RNA, 40 nt, synthetic construct
  misc_feature 1..40: note = GAPDH editing sequence
  source 1..40: mol_type = other RNA
gtcatcatatttggcaggtttttccagacggcaggtcagg

SEQ ID NO: 93 - RNA, 61 nt, synthetic construct
  misc_feature 1..61: note = GAPDH editing sequence
  modified_base 31: mod_base = OTHER; note = Benner's base
  source 1..61: mol_type = other RNA
cttgatgtcatcatatttggcaggtttttcnagacggcaggtcaggtccaccactgacacg

SEQ ID NO: 94 - RNA, 37 nt, synthetic construct
  misc_feature 1..37: note = GAPDH editing sequence
  source 1..37: mol_type = other RNA
gtcatcatatttggcaggtttttccagacggcaggtc

SEQ ID NO: 95 - RNA, 35 nt, synthetic construct
  misc_feature 1..35: note = GAPDH editing sequence
  source 1..35: mol_type = other RNA
gtcatcatatttggcaggtttttccagacggcagg

SEQ ID NO: 96 - RNA, 33 nt, synthetic construct
  misc_feature 1..33: note = GAPDH editing sequence
  source 1..33: mol_type = other RNA
gtcatcatatttggcaggtttttccagacggca

SEQ ID NO: 97 - RNA, 31 nt, synthetic construct
  misc_feature 1..31: note = GAPDH editing sequence
  source 1..31: mol_type = other RNA
gtcatcatatttggcaggtttttccagacgg

SEQ ID NO: 98 - RNA, 29 nt, synthetic construct
  misc_feature 1..29: note = GAPDH editing sequence
  source 1..29: mol_type = other RNA
gtcatcatatttggcaggtttttccagac

SEQ ID NO: 99 - RNA, 35 nt, synthetic construct
  misc_feature 1..35: note = GAPDH editing sequence
  source 1..35: mol_type = other RNA
catcatatttggcaggtttttccagacggcaggtc

SEQ ID NO: 100 - RNA, 33 nt, synthetic construct
  misc_feature 1..33: note = GAPDH editing sequence
  source 1..33: mol_type = other RNA
catcatatttggcaggtttttccagacggcagg

SEQ ID NO: 101 - RNA, 31 nt, synthetic construct
  misc_feature 1..31: note = GAPDH editing sequence
  source 1..31: mol_type = other RNA
catcatatttggcaggtttttccagacggca

SEQ ID NO: 102 - RNA, 29 nt, synthetic construct
  misc_feature 1..29: note = GAPDH editing sequence
  source 1..29: mol_type = other RNA
catcatatttggcaggtttttccagacgg

SEQ ID NO: 103 - RNA, 27 nt, synthetic construct
  misc_feature 1..27: note = GAPDH editing sequence
  source 1..27: mol_type = other RNA
catcatatttggcaggtttttccagac

SEQ ID NO: 104 - RNA, 33 nt, synthetic construct
  misc_feature 1..33: note = GAPDH editing sequence
  source 1..33: mol_type = other RNA
tcatatttggcaggtttttccagacggcaggtc

SEQ ID NO: 105 - RNA, 31 nt, synthetic construct
  misc_feature 1..31: note = GAPDH editing sequence
  source 1..31: mol_type = other RNA
tcatatttggcaggtttttccagacggcagg

SEQ ID NO: 106 - RNA, 29 nt, synthetic construct
  misc_feature 1..29: note = GAPDH editing sequence
  source 1..29: mol_type = other RNA
tcatatttggcaggtttttccagacggca

SEQ ID NO: 107 - RNA, 27 nt, synthetic construct
  misc_feature 1..27: note = GAPDH editing sequence
  source 1..27: mol_type = other RNA
tcatatttggcaggtttttccagacgg

SEQ ID NO: 108 - RNA, 25 nt, synthetic construct
  misc_feature 1..25: note = GAPDH editing sequence
  source 1..25: mol_type = other RNA
tcatatttggcaggtttttccagac

SEQ ID NO: 109 - RNA, 40 nt, synthetic construct
  misc_feature 1..40: note = GAPDH editing sequence
  source 1..40: mol_type = other RNA
gtcatcatatttggcaggtttttcgagacggcaggtcagg

SEQ ID NO: 110 - RNA, 40 nt, synthetic construct
  misc_feature 1..40: note = GAPDH editing sequence
  source 1..40: mol_type = other RNA
gtcatcatatttggcaggtttttcaagacggcaggtcagg

SEQ ID NO: 111 - RNA, 40 nt, synthetic construct
  modified_base 25: mod_base = OTHER; note = thymine
  misc_feature 1..40: note = GAPDH editing sequence
  source 1..40: mol_type = other RNA
gtcatcatatttggcaggtttttctagacggcaggtcagg

SEQ ID NO: 112 - RNA, 40 nt, synthetic construct
  misc_feature 1..40: note = GAPDH editing sequence
  modified_base 25: mod_base = i
  source 1..40: mol_type = other RNA
gtcatcatatttggcaggtttttcnagacggcaggtcagg

SEQ ID NO: 113 - RNA, 40 nt, synthetic construct
  misc_feature 1..40: note = GAPDH editing sequence
  source 1..40: mol_type = other RNA
gtcatcatatttggcaggtttttgcagacggcaggtcagg

SEQ ID NO: 114 - RNA, 40 nt, synthetic construct
  misc_feature 1..40: note = GAPDH editing sequence
  source 1..40: mol_type = other RNA
gtcatcatatttggcaggtttttacagacggcaggtcagg

SEQ ID NO: 115 - RNA, 40 nt, synthetic construct
  modified_base 24: mod_base = OTHER; note = thymine
  misc_feature 1..40: note = GAPDH editing sequence
  source 1..40: mol_type = other RNA
gtcatcatatttggcaggttttttcagacggcaggtcagg

SEQ ID NO: 116 - RNA, 40 nt, synthetic construct
  misc_feature 1..40: note = GAPDH editing sequence
  modified_base 24: mod_base = i
  source 1..40: mol_type = other RNA
gtcatcatatttggcaggtttttncagacggcaggtcagg

SEQ ID NO: 117 - RNA, 40 nt, synthetic construct
  misc_feature 1..40: note = GAPDH editing sequence
  source 1..40: mol_type = other RNA
gtcatcatatttggcaggtttttccggacggcaggtcagg

SEQ ID NO: 118 - RNA, 40 nt, synthetic construct
  misc_feature 1..40: note = GAPDH editing sequence
  source 1..40: mol_type = other RNA
gtcatcatatttggcaggtttttcccgacggcaggtcagg

SEQ ID NO: 119 - RNA, 40 nt, synthetic construct
  modified_base 26: mod_base = OTHER; note = thymine
  misc_feature 1..40: note = GAPDH editing sequence
  source 1..40: mol_type = other RNA
gtcatcatatttggcaggtttttcctgacggcaggtcagg

SEQ ID NO: 120 - RNA, 40 nt, synthetic construct
  misc_feature 1..40: note = GAPDH editing sequence
  modified_base 26: mod_base = i
  source 1..40: mol_type = other RNA
gtcatcatatttggcaggtttttccngacggcaggtcagg

SEQ ID NO: 121 - RNA, 40 nt, synthetic construct; identical to SEQ ID NO: 112
  misc_feature 1..40: note = GAPDH editing sequence
  modified_base 25: mod_base = OTHER; note = 5-methyl cytosine
  source 1..40: mol_type = other RNA

SEQ ID NO: 122 - RNA, 40 nt, synthetic construct; identical to SEQ ID NO: 112
  misc_feature 1..40: note = GAPDH editing sequence
  modified_base 25: mod_base = OTHER; note = Benner's base
  source 1..40: mol_type = other RNA

SEQ ID NO: 123 - RNA, 40 nt, synthetic construct; identical to SEQ ID NO: 112
  misc_feature 1..40: note = GAPDH editing sequence
  modified_base 25: mod_base = OTHER; note = pyrrolo cytosine
  source 1..40: mol_type = other RNA

SEQ ID NO: 124 - RNA, 40 nt, synthetic construct; identical to SEQ ID NO: 112
  misc_feature 1..40: note = GAPDH editing sequence
  modified_base 25: mod_base = OTHER; note = abasic
  source 1..40: mol_type = other RNA

SEQ ID NO: 125 - RNA, 40 nt, synthetic construct; identical to SEQ ID NO: 112
  misc_feature 1..40: note = GAPDH editing sequence
  modified_base 25: mod_base = OTHER; note = 5-bromo cytosine
  source 1..40: mol_type = other RNA

SEQ ID NO: 126 - RNA, 61 nt, synthetic construct
  misc_feature 1..61: note = mHprt editing sequence
  source 1..61: mol_type = other RNA
ggcagatggccacacgactagaacacctgccaattttactggcaacataaacagcactcct

SEQ ID NO: 127 - RNA, 51 nt, synthetic construct
  misc_feature 1..51: note = mHprt editing sequence
  source 1..51: mol_type = other RNA
atggccacacgactagaacacctgccaattttactggcaacataaacagca

SEQ ID NO: 128 - RNA, 50 nt, synthetic construct
  misc_feature 1..50: note = mHprt editing sequence
  source 1..50: mol_type = other RNA
atggccacacgactagaacacctgccaattttactggcaacataaacagc

SEQ ID NO: 129 - RNA, 49 nt, synthetic construct
  misc_feature 1..49: note = mHprt editing sequence
  source 1..49: mol_type = other RNA
atggccacacgactagaacacctgccaattttactggcaacataaacag

SEQ ID NO: 130 - RNA, 48 nt, synthetic construct
  misc_feature 1..48: note = mHprt editing sequence
  source 1..48: mol_type = other RNA
atggccacacgactagaacacctgccaattttactggcaacataaaca

SEQ ID NO: 131 - RNA, 47 nt, synthetic construct
  misc_feature 1..47: note = mHprt editing sequence
  source 1..47: mol_type = other RNA
atggccacacgactagaacacctgccaattttactggcaacataaac

SEQ ID NO: 132 - RNA, 46 nt, synthetic construct
  misc_feature 1..46: note = mHprt editing sequence
  source 1..46: mol_type = other RNA
atggccacacgactagaacacctgccaattttactggcaacataaa

SEQ ID NO: 133 - RNA, 45 nt, synthetic construct
  source 1..45: mol_type = other RNA
  misc_feature 1..45: note = mHprt editing sequence
atggccacacgactagaacacctgccaattttactggcaacataa

SEQ ID NO: 134 - RNA, 44 nt, synthetic construct
  misc_feature 1..44: note = mHprt editing sequence
  source 1..44: mol_type = other RNA
atggccacacgactagaacacctgccaattttactggcaacata

SEQ ID NO: 135 - RNA, 43 nt, synthetic construct
  misc_feature 1..43: note = mHprt editing sequence
  source 1..43: mol_type = other RNA
atggccacacgactagaacacctgccaattttactggcaacat

SEQ ID NO: 136 - RNA, 42 nt, synthetic construct
  misc_feature 1..42: note = mHprt editing sequence
  source 1..42: mol_type = other RNA
atggccacacgactagaacacctgccaattttactggcaaca

SEQ ID NO: 137 - RNA, 41 nt, synthetic construct
  misc_feature 1..41: note = mHprt editing sequence
  source 1..41: mol_type = other RNA
atggccacacgactagaacacctgccaattttactggcaac

SEQ ID NO: 138 - RNA, 40 nt, synthetic construct
  misc_feature 1..40: note = mHprt editing sequence
  source 1..40: mol_type = other RNA
atggccacacgactagaacacctgccaattttactggcaa

SEQ ID NO: 139 - RNA, 39 nt, synthetic construct
  misc_feature 1..39: note = mHprt editing sequence
  source 1..39: mol_type = other RNA
atggccacacgactagaacacctgccaattttactggca

SEQ ID NO: 140 - RNA, 38 nt, synthetic construct
  misc_feature 1..38: note = mHprt editing sequence
  source 1..38: mol_type = other RNA
atggccacacgactagaacacctgccaattttactggc

SEQ ID NO: 141 - RNA, 37 nt, synthetic construct
  misc_feature 1..37: note = mHprt editing sequence
  source 1..37: mol_type = other RNA
atggccacacgactagaacacctgccaattttactgg

SEQ ID NO: 142 - RNA, 36 nt, synthetic construct
  misc_feature 1..36: note = mHprt editing sequence
  source 1..36: mol_type = other RNA
atggccacacgactagaacacctgccaattttactg

SEQ ID NO: 143 - RNA, 35 nt, synthetic construct
  misc_feature 1..35: note = mHprt editing sequence
  source 1..35: mol_type = other RNA
atggccacacgactagaacacctgccaattttact

SEQ ID NO: 144 - RNA, 34 nt, synthetic construct
  misc_feature 1..34: note = mHprt editing sequence
  source 1..34: mol_type = other RNA
atggccacacgactagaacacctgccaattttac

SEQ ID NO: 145 - RNA, 33 nt, synthetic construct
  misc_feature 1..33: note = mHprt editing sequence
  source 1..33: mol_type = other RNA
atggccacacgactagaacacctgccaatttta

SEQ ID NO: 146 - RNA, 32 nt, synthetic construct
  misc_feature 1..32: note = mHprt editing sequence
  source 1..32: mol_type = other RNA
atggccacacgactagaacacctgccaatttt

SEQ ID NO: 147 - RNA, 31 nt, synthetic construct
  misc_feature 1..31: note = mHprt editing sequence
  source 1..31: mol_type = other RNA
atggccacacgactagaacacctgccaattt

SEQ ID NO: 148 - RNA, 30 nt, synthetic construct
  misc_feature 1..30: note = mHprt editing sequence
  source 1..30: mol_type = other RNA
atggccacacgactagaacacctgccaatt

SEQ ID NO: 149 - RNA, 46 nt, synthetic construct
  misc_feature 1..46: note = mHprt editing sequence
  source 1..46: mol_type = other RNA
ggcagatggccacacgactagaacacctgccaattttactggcaac

SEQ ID NO: 150 - RNA, 45 nt, synthetic construct
  misc_feature 1..45: note = mHprt editing sequence
  source 1..45: mol_type = other RNA
gcagatggccacacgactagaacacctgccaattttactggcaac

SEQ ID NO: 151 - RNA, 44 nt, synthetic construct
  misc_feature 1..44: note = mHprt editing sequence
  source 1..44: mol_type = other RNA
cagatggccacacgactagaacacctgccaattttactggcaac

SEQ ID NO: 152 - RNA, 43 nt, synthetic construct
  misc_feature 1..43: note = mHprt editing sequence
  source 1..43: mol_type = other RNA
agatggccacacgactagaacacctgccaattttactggcaac

SEQ ID NO: 153 - RNA, 42 nt, synthetic construct
  misc_feature 1..42: note = mHprt editing sequence
  source 1..42: mol_type = other RNA
gatggccacacgactagaacacctgccaattttactggcaac

SEQ ID NO: 154 - RNA, 40 nt, synthetic construct
  misc_feature 1..40: note = mHprt editing sequence
  source 1..40: mol_type = other RNA
tggccacacgactagaacacctgccaattttactggcaac

SEQ ID NO: 155 - RNA, 39 nt, synthetic construct
  misc_feature 1..39: note = mHprt editing sequence
  source 1..39: mol_type = other RNA
ggccacacgactagaacacctgccaattttactggcaac

SEQ ID NO: 156 - RNA, 38 nt, synthetic construct
  misc_feature 1..38: note = mHprt editing sequence
  source 1..38: mol_type = other RNA
gccacacgactagaacacctgccaattttactggcaac

SEQ ID NO: 157 - RNA, 37 nt, synthetic construct
  misc_feature 1..37: note = mHprt editing sequence
  source 1..37: mol_type = other RNA
ccacacgactagaacacctgccaattttactggcaac

SEQ ID NO: 158 - RNA, 36 nt, synthetic construct
  misc_feature 1..36: note = mHprt editing sequence
  source 1..36: mol_type = other RNA
cacacgactagaacacctgccaattttactggcaac

SEQ ID NO: 159 - RNA, 37 nt, synthetic construct
  misc_feature 1..37: note = mHprt editing sequence
  source 1..37: mol_type = other RNA
tggccacacgactagaacacctgccaattttactggc

SEQ ID NO: 160 - RNA, 35 nt, synthetic construct
  misc_feature 1..35: note = mHprt editing sequence
  source 1..35: mol_type = other RNA
tggccacacgactagaacacctgccaattttactg

SEQ ID NO: 161 - RNA, 33 nt, synthetic construct
  misc_feature 1..33: note = mHprt editing sequence
  source 1..33: mol_type = other RNA
tggccacacgactagaacacctgccaattttac

SEQ ID NO: 162 - RNA, 31 nt, synthetic construct
  misc_feature 1..31: note = mHprt editing sequence
  source 1..31: mol_type = other RNA
tggccacacgactagaacacctgccaatttt

SEQ ID NO: 163 - RNA, 29 nt, synthetic construct
  misc_feature 1..29: note = mHprt editing sequence
  source 1..29: mol_type = other RNA
tggccacacgactagaacacctgccaatt

SEQ ID NO: 164 - RNA, 35 nt, synthetic construct
  misc_feature 1..35: note = mHprt editing sequence
  source 1..35: mol_type = other RNA
gccacacgactagaacacctgccaattttactggc

SEQ ID NO: 165 - RNA, 33 nt, synthetic construct
  misc_feature 1..33: note = mHprt editing sequence
  source 1..33: mol_type = other RNA
gccacacgactagaacacctgccaattttactg

SEQ ID NO: 166 - RNA, 31 nt, synthetic construct
  misc_feature 1..31: note = mHprt editing sequence
  source 1..31: mol_type = other RNA
gccacacgactagaacacctgccaattttac

SEQ ID NO: 167 - RNA, 29 nt, synthetic construct
  misc_feature 1..29: note = mHprt editing sequence
  source 1..29: mol_type = other RNA
gccacacgactagaacacctgccaatttt

SEQ ID NO: 168 - RNA, 27 nt, synthetic construct
  misc_feature 1..27: note = mHprt editing sequence
  source 1..27: mol_type = other RNA
gccacacgactagaacacctgccaatt

SEQ ID NO: 169 - RNA, 33 nt, synthetic construct
  misc_feature 1..33: note = mHprt editing sequence
  source 1..33: mol_type = other RNA
cacacgactagaacacctgccaattttactggc

SEQ ID NO: 170 - RNA, 31 nt, synthetic construct
  misc_feature 1..31: note = mHprt editing sequence
  source 1..31: mol_type = other RNA
cacacgactagaacacctgccaattttactg

SEQ ID NO: 171 - RNA, 29 nt, synthetic construct
  misc_feature 1..29: note = mHprt editing sequence
  source 1..29: mol_type = other RNA
cacacgactagaacacctgccaattttac

SEQ ID NO: 172 - RNA, 27 nt, synthetic construct
  misc_feature 1..27: note = mHprt editing sequence
  source 1..27: mol_type = other RNA
cacacgactagaacacctgccaatttt

SEQ ID NO: 173 - RNA, 25 nt, synthetic construct
  misc_feature 1..25: note = mHprt editing sequence
  source 1..25: mol_type = other RNA
cacacgactagaacacctgccaatt

SEQ ID NO: 174 - RNA, 40 nt, synthetic construct
  misc_feature 1..40: note = mHprt editing sequence
  source 1..40: mol_type = other RNA
tggccacacgactagaacacctgcgaattttactggcaac

SEQ ID NO: 175 - RNA, 40 nt, synthetic construct
  misc_feature 1..40: note = mHprt editing sequence
  source 1..40: mol_type = other RNA
tggccacacgactagaacacctgcaaattttactggcaac

SEQ ID NO: 176 - RNA, 40 nt, synthetic construct
  misc_feature 1..40: note = mHprt editing sequence
  source 1..40: mol_type = other RNA
tggccacacgactagaacacctgctaattttactggcaac

SEQ ID NO: 177 - RNA, 40 nt, synthetic construct
  misc_feature 1..40: note = mHprt editing sequence
  modified_base 25: mod_base = i
  source 1..40: mol_type = other RNA
tggccacacgactagaacacctgcnaattttactggcaac

SEQ ID NO: 178 - RNA, 40 nt, synthetic construct
  misc_feature 1..40: note = mHprt editing sequence
  source 1..40: mol_type = other RNA
tggccacacgactagaacacctggcaattttactggcaac

SEQ ID NO: 179 - RNA, 40 nt, synthetic construct
  misc_feature 1..40: note = mHprt editing sequence
  source 1..40: mol_type = other RNA
tggccacacgactagaacacctgacaattttactggcaac

SEQ ID NO: 180 - RNA, 40 nt, synthetic construct
  misc_feature 1..40: note = mHprt editing sequence
  source 1..40: mol_type = other RNA
tggccacacgactagaacacctgtcaattttactggcaac

SEQ ID NO: 181 - RNA, 40 nt, synthetic construct
  misc_feature 1..40: note = mHprt editing sequence
  modified_base 24: mod_base = i
  source 1..40: mol_type = other RNA
tggccacacgactagaacacctgncaattttactggcaac

SEQ ID NO: 182 - RNA, 40 nt, synthetic construct
  misc_feature 1..40: note = mHprt editing sequence
  source 1..40: mol_type = other RNA
tggccacacgactagaacacctgccgattttactggcaac

SEQ ID NO: 183 - RNA, 40 nt, synthetic construct
  misc_feature 1..40: note = mHprt editing sequence
  source 1..40: mol_type = other RNA
tggccacacgactagaacacctgcccattttactggcaac

SEQ ID NO: 184 - RNA, 40 nt, synthetic construct
  misc_feature 1..40: note = mHprt editing sequence
  source 1..40: mol_type = other RNA
tggccacacgactagaacacctgcctattttactggcaac

SEQ ID NO: 185 - RNA, 40 nt, synthetic construct
  misc_feature 1..40: note = mHprt editing sequence
  modified_base 26: mod_base = i
  source 1..40: mol_type = other RNA
tggccacacgactagaacacctgccnattttactggcaac

SEQ ID NO: 186 - DNA, 1185 bp, synthetic construct
  misc_feature 1..1185: note = SERPINA1 mRNA E342K mutant coding sequence
  source 1..1185: mol_type = other DNA
gaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacaagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 187 - DNA, 77 bp, synthetic construct
  misc_feature 1..77: note = Positions 986 to 1062 of SEQ ID NO 186
  source 1..77: mol_type = other DNA
tctccaaggccgtgcataaggctgtgctgaccatcgacaagaaagggactgaagctgctggggccatgtttttagag

SEQ ID NO: 188 - DNA, 37 bp, synthetic construct
  misc_feature 1..37: note = primer
  source 1..37: mol_type = other DNA
ccacttctagaccgaacggcacccctgaagctctcca

SEQ ID NO: 189 - DNA, 39 bp, synthetic construct
  misc_feature 1..39: note = primer
  source 1..39: mol_type = other DNA
ccataaccgttagccagcgatagacatgggtatggcctc

SEQ ID NO: 190 - DNA, 63 bp, synthetic construct
  misc_feature 1..63: note = primer
  modified_base 63: mod_base = OTHER; note = 3' Inverted dT
  source 1..63: mol_type = other DNA
gccgtgcataaggctgtgctgaccatcgactcgaaagggactgaagctgctggaaacatgtan

SEQ ID NO: 191 - DNA, 42 bp, synthetic construct
  misc_feature 1..42: note = primer
  source 1..42: mol_type = other DNA
ccacttctagaccgaacggaaaagccaaatacaaagcctaag

SEQ ID NO: 192 - DNA, 42 bp, synthetic construct
  misc_feature 1..42: note = primer
  source 1..42: mol_type = other DNA
ccataaccgttagccagcgtaacattcatagaaggttcatgc

SEQ ID NO: 193 - DNA, 72 bp, synthetic construct
  misc_feature 1..72: note = primer
  modified_base 72: mod_base = OTHER; note = 3' Inverted dT
  source 1..72: mol_type = other DNA
atacgaggagtcctgttgatgttgccagtaaaattcgcaggtgttctagtcctgtggccatctgcctagtcn

SEQ ID NO: 194 - DNA, 36 bp, synthetic construct
  misc_feature 1..36: note = primer
  source 1..36: mol_type = other DNA
ccacttctagaccgaacgcgtgtccccactgccaac

SEQ ID NO: 195 - DNA, 36 bp, synthetic construct
  misc_feature 1..36: note = primer
  source 1..36: mol_type = other DNA
ccataaccgttagccagcgcctgcttcaccaccttc

SEQ ID NO: 196 - DNA, 52 bp, synthetic construct
  misc_feature 1..52: note = primer
  modified_base 52: mod_base = OTHER; note = 3' Inverted dT
  source 1..52: mol_type = other DNA
cagtggtggacctgacctgccgtctcgaaaaacctgccaaatatgatgacan

SEQ ID NO: 197 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaggctataacactctttccctacacgacgctcttccgatctnnnnccataaccgttagccagc

SEQ ID NO: 198 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgcctctatacactctttccctacacgacgctcttccgatctnnnnccataaccgttagccagc

SEQ ID NO: 199 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaggataggacactctttccctacacgacgctcttccgatctnnnnccataaccgttagccagc

SEQ ID NO: 200 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactcagagccacactctttccctacacgacgctcttccgatctnnnnccataaccgttagccagc

SEQ ID NO: 201 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccttcgcctacactctttccctacacgacgctcttccgatctnnnnccataaccgttagccagc

SEQ ID NO: 202 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactaagattaacactctttccctacacgacgctcttccgatctnnnnccataaccgttagccagc

SEQ ID NO: 203 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacacgtcctgacactctttccctacacgacgctcttccgatctnnnnccataaccgttagccagc

SEQ ID NO: 204 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtcagtacacactctttccctacacgacgctcttccgatctnnnnccataaccgttagccagc

SEQ ID NO: 205 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatcgagtaatgtgactggagttcagacgtgtgctcttccgatctnnnnccacttctagaccgaacg

SEQ ID NO: 206 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattctccggagtgactggagttcagacgtgtgctcttccgatctnnnnccacttctagaccgaacg

SEQ ID NO: 207 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatctatcgctgtgactggagttcagacgtgtgctcttccgatctnnnnccacttctagaccgaacg

SEQ ID NO: 208 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatatgcgacggtgactggagttcagacgtgtgctcttccgatctnnnnccacttctagaccgaacg

SEQ ID NO: 209 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatttcgcggagtgactggagttcagacgtgtgctcttccgatctnnnnccacttctagaccgaacg

SEQ ID NO: 210 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatcatagccggtgactggagttcagacgtgtgctcttccgatctnnnnccacttctagaccgaacg

SEQ ID NO: 211 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatgcgcattagtgactggagttcagacgtgtgctcttccgatctnnnnccacttctagaccgaacg

SEQ ID NO: 212 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatagcttcaggtgactggagttcagacgtgtgctcttccgatctnnnnccacttctagaccgaacg

SEQ ID NO: 213 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatacgaattcgtgactggagttcagacgtgtgctcttccgatctnnnnccacttctagaccgaacg

SEQ ID NO: 214 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatttctgaatgtgactggagttcagacgtgtgctcttccgatctnnnnccacttctagaccgaacg

SEQ ID NO: 215 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagatggaatctcgtgactggagttcagacgtgtgctcttccgatctnnnnccacttctagaccgaacg

SEQ ID NO: 216 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagataatgagcggtgactggagttcagacgtgtgctcttccgatctnnnnccacttctagaccgaacg

SEQ ID NO: 217 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgctttataacactctttccctacacgacgctcttccgatctnnnnccataaccgttagccagc

SEQ ID NO: 218 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccgtccataacactctttccctacacgacgctcttccgatctnnnnccataaccgttagccagc

SEQ ID NO: 219 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacacatgataacactctttccctacacgacgctcttccgatctnnnnccataaccgttagccagc

SEQ ID NO: 220 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacacattctaacactctttccctacacgacgctcttccgatctnnnnccataaccgttagccagc

SEQ ID NO: 221 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactacctataacactctttccctacacgacgctcttccgatctnnnnccataaccgttagccagc

SEQ ID NO: 222 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaacagttaacactctttccctacacgacgctcttccgatctnnnnccataaccgttagccagc

SEQ ID NO: 223 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccgtaggtaacactctttccctacacgacgctcttccgatctnnnnccataaccgttagccagc

SEQ ID NO: 224 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaggcgataacactctttccctacacgacgctcttccgatctnnnnccataaccgttagccagc

SEQ ID NO: 225 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatggagcgtgactggagttcagacgtgtgctcttccgatctnnnnccacttctagaccgaacg

SEQ ID NO: 226 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattataagacgtgactggagttcagacgtgtgctcttccgatctnnnnccacttctagaccgaacg

SEQ ID NO: 227 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagggcttgtgactggagttcagacgtgtgctcttccgatctnnnnccacttctagaccgaacg

SEQ ID NO: 228 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagcgctgtgactggagttcagacgtgtgctcttccgatctnnnnccacttctagaccgaacg

SEQ ID NO: 229 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacagatagtgactggagttcagacgtgtgctcttccgatctnnnnccacttctagaccgaacg

SEQ ID NO: 230 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatcactagtgactggagttcagacgtgtgctcttccgatctnnnnccacttctagaccgaacg

SEQ ID NO: 231 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaaggctggtgactggagttcagacgtgtgctcttccgatctnnnnccacttctagaccgaacg

SEQ ID NO: 232 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaaatcctgtgactggagttcagacgtgtgctcttccgatctnnnnccacttctagaccgaacg

SEQ ID NO: 233 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaccctgagtgactggagttcagacgtgtgctcttccgatctnnnnccacttctagaccgaacg

SEQ ID NO: 234 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtcagggtgactggagttcagacgtgtgctcttccgatctnnnnccacttctagaccgaacg

SEQ ID NO: 235 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagatcgggtgactggagttcagacgtgtgctcttccgatctnnnnccacttctagaccgaacg

SEQ ID NO: 236 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtgatagtgactggagttcagacgtgtgctcttccgatctnnnnccacttctagaccgaacg

SEQ ID NO: 237 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacccacgttaacactctttccctacacgacgctcttccgatctnnnnccataaccgttagccagc

SEQ ID NO: 238 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacatgaactaacactctttccctacacgacgctcttccgatctnnnnccataaccgttagccagc

SEQ ID NO: 239 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgttccgtaacactctttccctacacgacgctcttccgatctnnnnccataaccgttagccagc

SEQ ID NO: 240 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactatgtctaacactctttccctacacgacgctcttccgatctnnnnccataaccgttagccagc

SEQ ID NO: 241 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccggatgtaacactctttccctacacgacgctcttccgatctnnnnccataaccgttagccagc

SEQ ID NO: 242 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacccagagtaacactctttccctacacgacgctcttccgatctnnnnccataaccgttagccagc

SEQ ID NO: 243 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactaggtataacactctttccctacacgacgctcttccgatctnnnnccataaccgttagccagc

SEQ ID NO: 244 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccggctataacactctttccctacacgacgctcttccgatctnnnnccataaccgttagccagc

SEQ ID NO: 245 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagcttgcgtgactggagttcagacgtgtgctcttccgatctnnnnccacttctagaccgaacg

SEQ ID NO: 246 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagaatcgtgactggagttcagacgtgtgctcttccgatctnnnnccacttctagaccgaacg

SEQ ID NO: 247 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagagccgtgactggagttcagacgtgtgctcttccgatctnnnnccacttctagaccgaacg

SEQ ID NO: 248 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatgctatgtgactggagttcagacgtgtgctcttccgatctnnnnccacttctagaccgaacg

SEQ ID NO: 249 - DNA, 88 bp, synthetic construct; identical to SEQ ID NO: 248
  misc_feature 1..88: note = primer
  misc_feature 67..70: note = n is a, c, g, t or u
  source 1..88: mol_type = other DNA

SEQ ID NO: 250 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagccaaggtgactggagttcagacgtgtgctcttccgatctnnnnccacttctagaccgaacg

SEQ ID NO: 251 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaccgtcagtgactggagttcagacgtgtgctcttccgatctnnnnccacttctagaccgaacg

SEQ ID NO: 252 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtattcgtgactggagttcagacgtgtgctcttccgatctnnnnccacttctagaccgaacg

SEQ ID NO: 253 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatgctgcgtgactggagttcagacgtgtgctcttccgatctnnnnccacttctagaccgaacg

SEQ ID NO: 254 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaactatcgtgactggagttcagacgtgtgctcttccgatctnnnnccacttctagaccgaacg

SEQ ID NO: 255 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaaggcctgtgactggagttcagacgtgtgctcttccgatctnnnnccacttctagaccgaacg

SEQ ID NO: 256 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacccgaagtgactggagttcagacgtgtgctcttccgatctnnnnccacttctagaccgaacg

SEQ ID NO: 257 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacatctactaacactctttccctacacgacgctcttccgatctnnnnccataaccgttagccagc

SEQ ID NO: 258 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactagttctaacactctttccctacacgacgctcttccgatctnnnnccataaccgttagccagc

SEQ ID NO: 259 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacatgtgataacactctttccctacacgacgctcttccgatctnnnnccataaccgttagccagc

SEQ ID NO: 260 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactgcgtctaacactctttccctacacgacgctcttccgatctnnnnccataaccgttagccagc

SEQ ID NO: 261 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtacaataacactctttccctacacgacgctcttccgatctnnnnccataaccgttagccagc

SEQ ID NO: 262 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactggtcctaacactctttccctacacgacgctcttccgatctnnnnccataaccgttagccagc

SEQ ID NO: 263 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacttagcttaacactctttccctacacgacgctcttccgatctnnnnccataaccgttagccagc

SEQ ID NO: 264 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacactgaataacactctttccctacacgacgctcttccgatctnnnnccataaccgttagccagc

SEQ ID NO: 265 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaccgcttgtgactggagttcagacgtgtgctcttccgatctnnnnccacttctagaccgaacg

SEQ ID NO: 266 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagcagcgtgactggagttcagacgtgtgctcttccgatctnnnnccacttctagaccgaacg

SEQ ID NO: 267 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgtgctgtgactggagttcagacgtgtgctcttccgatctnnnnccacttctagaccgaacg

SEQ ID NO: 268 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaaacttcgtgactggagttcagacgtgtgctcttccgatctnnnnccacttctagaccgaacg

SEQ ID NO: 269 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaaatgcagtgactggagttcagacgtgtgctcttccgatctnnnnccacttctagaccgaacg

SEQ ID NO: 270 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatagcttgtgactggagttcagacgtgtgctcttccgatctnnnnccacttctagaccgaacg

SEQ ID NO: 271 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagctattgtgactggagttcagacgtgtgctcttccgatctnnnnccacttctagaccgaacg

SEQ ID NO: 272 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagccatcgtgactggagttcagacgtgtgctcttccgatctnnnnccacttctagaccgaacg

SEQ ID NO: 273 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattataacaggtgactggagttcagacgtgtgctcttccgatctnnnnccacttctagaccgaacg

SEQ ID NO: 274 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagaccggtgactggagttcagacgtgtgctcttccgatctnnnnccacttctagaccgaacg

SEQ ID NO: 275 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgctgtgtgactggagttcagacgtgtgctcttccgatctnnnnccacttctagaccgaacg

SEQ ID NO: 276 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactttaggtgactggagttcagacgtgtgctcttccgatctnnnnccacttctagaccgaacg

SEQ ID NO: 277 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacccgttgtaacactctttccctacacgacgctcttccgatctnnnnccataaccgttagccagc

SEQ ID NO: 278 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactatacctaacactctttccctacacgacgctcttccgatctnnnnccataaccgttagccagc

SEQ ID NO: 279 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacacaagttaacactctttccctacacgacgctcttccgatctnnnnccataaccgttagccagc

SEQ ID NO: 280 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacttggaataacactctttccctacacgacgctcttccgatctnnnnccataaccgttagccagc

SEQ ID NO: 281 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgccctttaacactctttccctacacgacgctcttccgatctnnnnccataaccgttagccagc

SEQ ID NO: 282 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactgatcttaacactctttccctacacgacgctcttccgatctnnnnccataaccgttagccagc

SEQ ID NO: 283 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacggaatttaacactctttccctacacgacgctcttccgatctnnnnccataaccgttagccagc

SEQ ID NO: 284 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactgccgataacactctttccctacacgacgctcttccgatctnnnnccataaccgttagccagc

SEQ ID NO: 285 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattattgtcagtgactggagttcagacgtgtgctcttccgatctnnnnccacttctagaccgaacg

SEQ ID NO: 286 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaatttgcgtgactggagttcagacgtgtgctcttccgatctnnnnccacttctagaccgaacg

SEQ ID NO: 287 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgaatagtgactggagttcagacgtgtgctcttccgatctnnnnccacttctagaccgaacg

SEQ ID NO: 288 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaacgcgtgtgactggagttcagacgtgtgctcttccgatctnnnnccacttctagaccgaacg

SEQ ID NO: 289 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatcttaggtgactggagttcagacgtgtgctcttccgatctnnnnccacttctagaccgaacg

SEQ ID NO: 290 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactgcgtgtgactggagttcagacgtgtgctcttccgatctnnnnccacttctagaccgaacg

SEQ ID NO: 291 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaggtgcagtgactggagttcagacgtgtgctcttccgatctnnnnccacttctagaccgaacg

SEQ ID NO: 292 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacagtttgtgactggagttcagacgtgtgctcttccgatctnnnnccacttctagaccgaacg

SEQ ID NO: 293 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtgcgagtgactggagttcagacgtgtgctcttccgatctnnnnccacttctagaccgaacg

SEQ ID NO: 294 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagttagagtgactggagttcagacgtgtgctcttccgatctnnnnccacttctagaccgaacg

SEQ ID NO: 295 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaacccgagtgactggagttcagacgtgtgctcttccgatctnnnnccacttctagaccgaacg

SEQ ID NO: 296 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacccagtgtgactggagttcagacgtgtgctcttccgatctnnnnccacttctagaccgaacg

SEQ ID NO: 297 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtgaattaacactctttccctacacgacgctcttccgatctnnnnccataaccgttagccagc

SEQ ID NO: 298 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactcacattaacactctttccctacacgacgctcttccgatctnnnnccataaccgttagccagc

SEQ ID NO: 299 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgacacgtaacactctttccctacacgacgctcttccgatctnnnnccataaccgttagccagc

SEQ ID NO: 300 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtaagttaacactctttccctacacgacgctcttccgatctnnnnccataaccgttagccagc

SEQ ID NO: 301 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacggctgataacactctttccctacacgacgctcttccgatctnnnnccataaccgttagccagc

SEQ ID NO: 302 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaattcctaacactctttccctacacgacgctcttccgatctnnnnccataaccgttagccagc

SEQ ID NO: 303 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgcaggttaacactctttccctacacgacgctcttccgatctnnnnccataaccgttagccagc

SEQ ID NO: 304 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacttgctataacactctttccctacacgacgctcttccgatctnnnnccataaccgttagccagc

SEQ ID NO: 305 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacagcctgtgactggagttcagacgtgtgctcttccgatctnnnnccacttctagaccgaacg

SEQ ID NO: 306 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaccgttggtgactggagttcagacgtgtgctcttccgatctnnnnccacttctagaccgaacg

SEQ ID NO: 307 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtgaatgtgactggagttcagacgtgtgctcttccgatctnnnnccacttctagaccgaacg

SEQ ID NO: 308 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagtggcgtgactggagttcagacgtgtgctcttccgatctnnnnccacttctagaccgaacg

SEQ ID NO: 309 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactactagtgactggagttcagacgtgtgctcttccgatctnnnnccacttctagaccgaacg

SEQ ID NO: 310 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagacatgtgactggagttcagacgtgtgctcttccgatctnnnnccacttctagaccgaacg

SEQ ID NO: 311 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacaataggtgactggagttcagacgtgtgctcttccgatctnnnnccacttctagaccgaacg

SEQ ID NO: 312 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatggatagtgactggagttcagacgtgtgctcttccgatctnnnnccacttctagaccgaacg

SEQ ID NO: 313 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgcactgtgactggagttcagacgtgtgctcttccgatctnnnnccacttctagaccgaacg

SEQ ID NO: 314 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaggtagcgtgactggagttcagacgtgtgctcttccgatctnnnnccacttctagaccgaacg

SEQ ID NO: 315 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagccttggtgactggagttcagacgtgtgctcttccgatctnnnnccacttctagaccgaacg

SEQ ID NO: 316 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaagtcttgtgactggagttcagacgtgtgctcttccgatctnnnnccacttctagaccgaacg

SEQ ID NO: 317 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccgcatctaacactctttccctacacgacgctcttccgatctnnnnccataaccgttagccagc

SEQ ID NO: 318 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtcccataacactctttccctacacgacgctcttccgatctnnnnccataaccgttagccagc

SEQ ID NO: 319 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactgtgcctaacactctttccctacacgacgctcttccgatctnnnnccataaccgttagccagc

SEQ ID NO: 320 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactaacagtaacactctttccctacacgacgctcttccgatctnnnnccataaccgttagccagc

SEQ ID NO: 321 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccgagcataacactctttccctacacgacgctcttccgatctnnnnccataaccgttagccagc

SEQ ID NO: 322 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccctagctaacactctttccctacacgacgctcttccgatctnnnnccataaccgttagccagc

SEQ ID NO: 323 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactacgtttaacactctttccctacacgacgctcttccgatctnnnnccataaccgttagccagc

SEQ ID NO: 324 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccgactctaacactctttccctacacgacgctcttccgatctnnnnccataaccgttagccagc

SEQ ID NO: 325 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagctaaagtgactggagttcagacgtgtgctcttccgatctnnnnccacttctagaccgaacg

SEQ ID NO: 326 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaggccgcgtgactggagttcagacgtgtgctcttccgatctnnnnccacttctagaccgaacg

SEQ ID NO: 327 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactgcacgtgactggagttcagacgtgtgctcttccgatctnnnnccacttctagaccgaacg

SEQ ID NO: 328 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacatctagtgactggagttcagacgtgtgctcttccgatctnnnnccacttctagaccgaacg

SEQ ID NO: 329 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaggactggtgactggagttcagacgtgtgctcttccgatctnnnnccacttctagaccgaacg

SEQ ID NO: 330 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactatcagtgactggagttcagacgtgtgctcttccgatctnnnnccacttctagaccgaacg

SEQ ID NO: 331 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaacggtggtgactggagttcagacgtgtgctcttccgatctnnnnccacttctagaccgaacg

SEQ ID NO: 332 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatcaaaggtgactggagttcagacgtgtgctcttccgatctnnnnccacttctagaccgaacg

SEQ ID NO: 333 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactgaccgtgactggagttcagacgtgtgctcttccgatctnnnnccacttctagaccgaacg

SEQ ID NO: 334 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacaagtagtgactggagttcagacgtgtgctcttccgatctnnnnccacttctagaccgaacg

SEQ ID NO: 335 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattactacatgtgactggagttcagacgtgtgctcttccgatctnnnnccacttctagaccgaacg

SEQ ID NO: 336 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaatcttggtgactggagttcagacgtgtgctcttccgatctnnnnccacttctagaccgaacg

SEQ ID NO: 337 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgaacattaacactctttccctacacgacgctcttccgatctnnnnccataaccgttagccagc

SEQ ID NO: 338 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacaacgcctaacactctttccctacacgacgctcttccgatctnnnnccataaccgttagccagc

SEQ ID NO: 339 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacactgcggttaacactctttccctacacgacgctcttccgatctnnnnccataaccgttagccagc

SEQ ID NO: 340 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtcatttaacactctttccctacacgacgctcttccgatctnnnnccataaccgttagccagc

SEQ ID NO: 341 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgggacttaacactctttccctacacgacgctcttccgatctnnnnccataaccgttagccagc

SEQ ID NO: 342 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacacgtagattaacactctttccctacacgacgctcttccgatctnnnnccataaccgttagccagc

SEQ ID NO: 343 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccatgggtaacactctttccctacacgacgctcttccgatctnnnnccataaccgttagccagc

SEQ ID NO: 344 - DNA, 92 bp, synthetic construct
  misc_feature 1..92: note = primer
  misc_feature 71..74: note = n is a, c, g, t or u
  source 1..92: mol_type = other DNA
aatgatacggcgaccaccgagatctacaccagttttaacactctttccctacacgacgctcttccgatctnnnnccataaccgttagccagc

SEQ ID NO: 345 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatgagtagtgactggagttcagacgtgtgctcttccgatctnnnnccacttctagaccgaacg

SEQ ID NO: 346 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgagtggtgactggagttcagacgtgtgctcttccgatctnnnnccacttctagaccgaacg

SEQ ID NO: 347 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacaaaccgtgactggagttcagacgtgtgctcttccgatctnnnnccacttctagaccgaacg

SEQ ID NO: 348 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatgacttgtgactggagttcagacgtgtgctcttccgatctnnnnccacttctagaccgaacg

SEQ ID NO: 349 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattaattccagtgactggagttcagacgtgtgctcttccgatctnnnnccacttctagaccgaacg

SEQ ID NO: 350 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgactcgtgactggagttcagacgtgtgctcttccgatctnnnnccacttctagaccgaacg

SEQ ID NO: 351 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattagtccgtgtgactggagttcagacgtgtgctcttccgatctnnnnccacttctagaccgaacg

SEQ ID NO: 352 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattattgcatgtgactggagttcagacgtgtgctcttccgatctnnnnccacttctagaccgaacg

SEQ ID NO: 353 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatgttcagtgactggagttcagacgtgtgctcttccgatctnnnnccacttctagaccgaacg

SEQ ID NO: 354 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacgccaagtgactggagttcagacgtgtgctcttccgatctnnnnccacttctagaccgaacg

SEQ ID NO: 355 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattatctacagtgactggagttcagacgtgtgctcttccgatctnnnnccacttctagaccgaacg

SEQ ID NO: 356 - DNA, 88 bp, synthetic construct
  misc_feature 1..88: note = primer
  misc_feature 67..70: note = n is a, c, g, t or u
  source 1..88: mol_type = other DNA
caagcagaagacggcatacgagattacggctagtgactggagttcagacgtgtgctcttccgatctnnnnccacttctagaccgaacg

(19) United States
(12) Patent Application Publication
(10) Pub. No.: US 2025/0304961 A1
(43) Pub. Date: Oct. 2, 2025

(54) EDITING OLIGONUCLEOTIDE

(71) Applicant: Hoffmann-La Roche Inc., Little Falls, NJ (US)

(72) Inventors: MOELHART HOEG Anja, Hillerød (DK); Tobias MERKLE, Copenhagen (DK)

(73) Assignee: Hoffmann-La Roche Inc., Little Falls, NJ (US)

(21) Appl. No.: 19/024,757

(22) Filed: Jan. 16, 2025

Related U.S. Application Data
(63) Continuation of application No. PCT/EP2023/069757, filed on Jul. 17, 2023.

(30) Foreign Application Priority Data
Jul. 18, 2022 (EP) 22185561.2

(51) Int. Cl. C12N 15/113; A61P 3/00; C12N 15/11

(52) U.S. Cl. CPC C12N 15/113; A61P 3/00; C12N 15/111; C12N 2310/315; C12N 2310/321; C12N 2310/322; C12N 2310/3231; C12N 2310/351

(57) ABSTRACT

The present invention relates to oligonucleotides for editing a target nucleic acid, as well as conjugates, salts and pharmaceutical compositions thereof. The invention also relates to uses of such oligonucleotides, conjugates, salts and pharmaceutical compositions in methods for editing target nucleic acids and in medical uses and methods of treatment of disease.

### CROSS REFERENCE TO RELATED APPLICATIONS

This application is a continuation of PCT Application No. PCT/EP2023/069757 filed Jul. 17, 2023, which claims priority to European Application No. 22185561.2 filed Jul. 18, 2022, each of which is incorporated by reference in its entirety.

### SEQUENCE LISTING

This application contains a Sequence Listing which has been submitted electronically in XML format and is hereby incorporated by reference in its entirety. Said XML copy, created Jan. 15, 2025, is named P37335-US-sequence-listing.xml and is 623,480 bytes in size.

### FIELD OF THE INVENTION

The present invention relates to oligonucleotides for editing a target nucleic acid, as well as conjugates, salts and pharmaceutical compositions thereof. The invention also relates to uses of such oligonucleotides, conjugates, salts and pharmaceutical compositions in methods for editing target nucleic acids and in medical uses and methods of treatment of disease.

### BACKGROUND

Editing of nucleic acids holds the promise to treat or prevent disease by altering or removing genetic sequences which produce dysfunctional gene products that lead to disease phenotypes.

RNA sequences, especially mRNA sequences encoding protein, may be edited using adenosine deaminase acting on RNA (ADAR) enzymes. An ADAR binds to double-stranded RNA and deaminates an adenine nucleobase to form a hypoxanthine nucleobase, thereby converting an adenosine (A) nucleoside to an inosine (1) nucleoside. An inosine nucleoside is structurally similar to guanosine (G) and forms complementary base pairs with cytidine (C). Inosine nucleosides are therefore “read” as guanosine (G) nucleosides by cellular translation machinery. Thus, adenosine-to-inosine editing of a codon in an mRNA sequence can after the amino acid sequence, and thereby the function, of a protein encoded by the mRNA. ADARs may also be used to edit non-coding sequences. Three ADAR genes (ADAR1, ADAR2 and ADAR3) have been identified in mammals (Nishikura 2016 Nat Rev Mol Cell Bio 17 83-96).

ADARs can be recruited to single-stranded nucleic acids, such as mRNA, by a guide oligonucleotide forming complementary base pairs with the target nucleic acid, thereby forming a double-stranded molecule to which an ADAR can bind. The sequence of the guide oligonucleotide therefore determines the site(s) at which the ADAR acts, i.e. the adenosines which the ADAR may convert to inosines. Endogenous ADARs may thus be directed to convert specific adenosines to inosines by introducing into a cell a guide oligonucleotide of a specific sequence. In a therapeutic context, use of endogenous ADARs advantageously avoids ectopic expression of exogenous protein (Merkle et al. 2019 Nat Biotech 37 133-138; Qu et al. Nat Biotech 37 1059-1069).

Alpha-1 antitrypsin (A1AT) deficiency (A1AD) is a disease that may be treatable using RNA editing techniques. A1AD is associated with mutations in the SERPINA1 gene. SERPINA1 encodes A1AT, a serine protease inhibitor synthesized in the liver that is released to other tissues to protect them from endogenous inflammatory serine proteases, such as neutrophil elastase. Subjects suffering from A1AD express reduced levels of A1AT, which can lead to excessive breakdown of elastin in the lungs, and thereby reduced lung elasticity and associated health problems such as emphysema. Build-up of misfolded A1AT in the liver may also lead to liver-associated problems such as cirrhosis and jaundice. The most severe form of A1AD is associated with a single base pair substitution leading to mutation in A1AT of E342 to K (E342K mutation). Less severe A1AD is associated with a single base pair substitution leading to mutation in A1AT of E264 to V (E264V mutation). SERPINA1 mRNA in cultured cells has been targeted by RNA editing techniques to attempt to correct the E342K mutation (see WO 2021/071858 A1 and WO 2021/243023 A1).

### SUMMARY OF THE INVENTION

The invention relates to oligonucleotides which provide ADAR-mediated editing of target nucleic acids, such as RNA. The oligonucleotides of the invention comprise a mixmer structure.

The invention provides an oligonucleotide comprising an editing region that comprises an editing nucleoside, a 5′ mixmer region positioned 5′ to the editing region, and a 3′ mixmer region positioned 3′ to the editing region.

The invention also provides an oligonucleotide conjugate comprising an oligonucleotide of the invention covalently attached to at least one conjugate moiety.

The invention also provides a pharmaceutical composition comprising the oligonucleotide or the oligonucleotide conjugate of the invention, and a pharmaceutically acceptable diluent, solvent, carrier, salt and/or adjuvant.

The invention also provides in vitro and in vivo method for editing a target nucleic acid in a target cell, the method comprising administering an effective amount of the oligonucleotide, oligonucleotide conjugate or pharmaceutical composition of the invention to the target cell.

The invention also provides a method for treating or preventing a disease comprising administering a therapeutically or prophylactically effective amount of the oligonucleotide, oligonucleotide conjugate or pharmaceutical composition of the invention to a subject suffering from or susceptible to a disease. The invention also provides the oligonucleotide, oligonucleotide conjugate or pharmaceutical composition of the invention for use in the treatment or prevention of a disease in a subject. The invention also provides use of the oligonucleotide, oligonucleotide conjugate or pharmaceutical composition of the invention for the preparation of a medicament for treatment or prevention of a disease in a subject. In preferred embodiments, the disease is A1AD.

### BRIEF DESCRIPTION OF FIGURES

FIG. 1 shows the degradation over time of oligonucleotides comprising a mixmer region of alternating 2′-O-methyl-RNA and 2′-fluoro-RNA nucleosides (CMP ID NOs 3_3 and 91_1) and an oligonucleotide comprising a region of RNA nucleosides (CMP ID NO 91_2), wherein the oligonucleotides are incubated in foetal bovine serum (FBS), cerebrospinal fluid (CSF) or lysosomes isolated from rat liver (rat liver tritosome—Trit.).

FIG. 2A and FIG. 2B show the editing efficiency of oligonucleotides comprising mixmer or non-mixmer regions on GAPDH mRNA (FIG. 2A) and SERPINA1 mRNA (FIG. 2B).

FIG. 3A and FIG. 3B show the editing efficiency of oligonucleotides comprising mixmer or non-mixmer regions SERPINA1 mRNA (FIG. 3A and FIG. 3B).

FIG. 4 compares the editing efficiency on SERPINA1 mRNA of an oligonucleotide comprising a guanosine (G) nucleoside at position −1 (CMP ID NO 2_1) with an oligonucleotide comprising an inosine (1) nucleoside at position −1 (CMP ID NO 3_1).

FIG. 5 compares the editing efficiency on GAPDH mRNA of an oligonucleotide comprising a DNA cytidine (C) nucleoside at position 0 (CMP ID NO 91_3) with an oligonucleotide comprising DNA nucleoside with Benner's base at position 0 (CMP ID NO 93_1).

FIG. 6A, FIG. 66, FIG. 6C, and FIG. 6D show the editing efficiency on SERPINA1 mRNA of oligonucleotides with different symmetries, in particular oligonucleotides with different numbers of nucleosides 3′ to the editing nucleoside (FIG. 6A and FIG. 6B) and 5′ to the editing nucleoside (FIG. 6C). The effect of inverting the symmetry of the oligonucleotide was also tested (FIG. 6D).

FIG. 7 shows the editing efficiency on SERPINA1 mRNA of oligonucleotides with different symmetries.

FIG. 8 shows the editing efficiency on GAPDH mRNA of oligonucleotides with different symmetries (A and B).

FIG. 9 shows the editing efficiency on GAPDH mRNA of oligonucleotides with different symmetries.

FIG. 10A, FIG. 10B and FIG. 10C show the editing efficiency on mHprt mRNA of oligonucleotides with different symmetries, in particular oligonucleotides with different numbers of nucleosides 3′ to the editing nucleoside (FIG. 10A, FIG. 10C) and 5′ to the editing nucleoside (FIG. 10B, FIG. 10C).

FIG. 11 shows the editing efficiency on SERPINA1 mRNA of oligonucleotides with different modifications to the editing nucleoside.

FIG. 12 shows the editing efficiency on SERPINA1 mRNA of oligonucleotides with different modifications to the editing nucleoside.

FIG. 13 shows the editing efficiency on GAPDH mRNA of oligonucleotides with different nucleobase and sugar moiety modifications to the editing nucleoside.

FIG. 14 shows the editing efficiency on GAPDH mRNA of oligonucleotides with different nucleobase and sugar moiety modifications to the editing nucleoside expressing exogenous human ADAR1 p110.

FIG. 15 shows the editing efficiency on GAPDH mRNA of oligonucleotides with different nucleobase and sugar moiety modifications to the editing nucleoside in HEK293 cells expressing exogenous human ADAR1 p150.

FIG. 16 shows the editing efficiency on GAPDH mRNA of oligonucleotides with different nucleobase and sugar moiety modifications to the editing nucleoside in HEK293 cells expressing exogenous human ADAR2.

FIG. 17A and FIG. 17B show the editing efficiency on GAPDH mRNA of oligonucleotides with different nucleobase and sugar moiety modifications in the editing triplet in HEK293 cells expressing exogenous human ADAR2 (FIG. 17A) and exogenous human ADAR1 p110 (FIG. 17B).

FIG. 18 shows the editing efficiency on mHprt mRNA of oligonucleotides with different nucleobase modifications in the editing triplet.

FIG. 19 shows the editing efficiency on GAPDH mRNA of oligonucleotides comprising different numbers of phosphorothioate (PS) internucleoside linkages.

FIG. 20A and FIG. 20B show the editing efficiency on GAPDH mRNA of oligonucleotides wherein all internucleoside linkages are phosphorothioate (PS) internucleoside linkages except for phosphodiester (PO) linkages at the indicated positions, in cells expressing human ADAR2 (FIG. 20A) and human ADAR1 p110 (FIG. 20B).

FIG. 21A and FIG. 21B show the editing efficiency on GAPDH mRNA of oligonucleotides wherein all internucleoside linkages are phosphorothioate (PS) internucleoside linkages except for phosphodiester (PO) linkages at the indicated positions, in cells expressing human ADAR2 (FIG. 21A) and human ADAR1 p110 (FIG. 21B).

FIG. 22 shows the editing efficiency on GAPDH mRNA of oligonucleotides wherein all internucleoside linkages are phosphorothioate (PS) internucleoside linkages except for phosphodiester (PO) linkages at the indicated positions, in Huh7 cells.

FIG. 23 shows the editing efficiency on mHprt mRNA of oligonucleotides wherein all internucleoside linkages are phosphorothioate (PS) internucleoside linkages except for phosphodiester (PO) linkages at the indicated positions.

FIG. 24A and FIG. 24B show the editing efficiency on SERPINA1 mRNA of oligonucleotides comprising a mixmer region of 2′-fluoro RNA nucleosides and 2′-O-methyl RNA nucleosides in a first (FIG. 24A) or second (FIG. 24B) alternating pattern, with an additional 2′-O-methyl RNA nucleoside at particular positions.

FIG. 25A and FIG. 25B show the editing efficiency on SERPINA1 mRNA of oligonucleotides comprising a mixmer region of 2′-fluoro RNA nucleosides and 2′-O-methyl RNA nucleosides in a first (FIG. 25A) or second (FIG. 25B) alternating pattern, with an additional 2′-fluoro RNA nucleoside at particular positions.

FIG. 26A, FIG. 26B, and FIG. 26C show the editing efficiency on GAPDH mRNA of oligonucleotides with or without MOE flank regions in cells expressing human ADAR2 (FIG. 26A) or human ADAR1 p110 (FIG. 26B) and in HuH-7 cells (FIG. 26C).

FIG. 27 shows the editing efficiency on mHprt mRNA of oligonucleotides and a corresponding oligonucleotide conjugate (CNJ ID NO 126_2_1).

FIGS. 28A, 28B and 28C show the editing efficiency on mHprt mRNA of oligonucleotides delivered to cells by reverse gymnosis.

FIG. 29 shows the chemical structure of CMP ID NO 32_1. Due to the length of the oligonucleotide CMP ID NO 32_1, FIG. 29 is split across three pages to ensure that all atoms in the structure are legible. The structure on page 1 of FIG. 29 (page 32 of the Figures) is connected to the structure on page 2 of FIG. 29 (page 33 of the Figures) by a carbon-carbon bond split between the two pages. The end of this split carbon-carbon bond that is on page 1 of FIG. 29 is indicated by “1”, and the end of this split carbon-carbon bond that is on page 2 of FIG. 29 is indicated by “*2”. Likewise, the structure on page 2 of FIG. 29 is connected to the structure on page 3 of FIG. 29 (page 34 of the Figures) by a carbon-carbon bond split between the two pages. The end of this split carbon-carbon bond that is on page 2 of FIG. 29 is indicated by “*3”, and the end of this split carbon-carbon bond that is on page 3 of FIG. 29 is indicated by “*4”.

Chemical drawings of oligonucleotides herein show the protonated form of the oligonucleotide, and it will be understood that each hydrogen on sulphur atoms in phosphorothioate internucleoside linkages may independently be present or absent. It will be understood that the presence of protons will depend on the acidity of the environment of the molecule. In a salt form, one or more of the hydrogens may for example be replaced with a cation, such as a metal cation, such as a sodium cation or a potassium cation. Protonated phosphorothioates exist in tautomeric forms.

### DETAILED DESCRIPTION OF THE INVENTION

Unless otherwise stated, all ranges are inclusive of the start and end value.

The invention provides an oligonucleotide comprising an editing region that comprises an editing nucleoside, a 5′ mixmer region positioned 5′ to the editing region, and a 3′ mixmer region positioned 3′ to the editing region.

The invention also provides an oligonucleotide comprising an editing region that comprises an editing nucleoside.

### Oligonucleotide

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

Oligonucleotides are commonly made in a laboratory by solid-phase chemical synthesis followed by purification and isolation. When referring to the sequence of an oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides, comprise a ribose sugar moiety, a nucleobase moiety and one or more phosphate groups. The one or more phosphate groups are absent in nucleosides. Nucleosides and nucleotides may also interchangeably be referred to as “units” or “monomers”. The terms “nucleoside” and “nucleotide” may be used interchangeably herein when referring to these units in the context of the oligonucleotide of the invention.

The nucleosides of the oligonucleotides of the invention may be referred to by their position in the oligonucleotide relative to the editing nucleoside. Thus, in some embodiments of the oligonucleotide of the invention, the editing nucleoside is designated as position 0, each nucleoside 5′ to the editing nucleoside is designated as position +x, wherein x is the number of nucleosides 5′ to the editing nucleoside at that position including the nucleoside at that position, and each nucleoside 3′ to the editing nucleoside is designated as position −y, wherein y is the number of nucleosides 3′ to the editing nucleoside at that position including the nucleoside at that position. For example, the nucleoside that is two nucleosides 5′ to the editing nucleoside would be position +2, whilst the nucleoside that is three nucleosides 3′ to the editing nucleoside would be position −3. As a further example, SEQ ID NO 32 is presented below, with the editing region underlined and the editing nucleoside in bold:
CCCCAGCAGCUUCAGUCCCUUUCTCIUCGAUGGUCAGCAC (SEQ ID NO 32)

The editing nucleoside C is at position 0. The T of the editing region is at position +1, the C immediately 5′ to that is at position +2 and so on. The I of the editing region is at position −1, the U immediately 3′ to that is at position −2 and so on.

In some embodiments, the oligonucleotide of the invention comprises an inosine nucleoside (i.e. a nucleoside comprising the nucleobase hypoxanthine). In some embodiments, the oligonucleotide of the invention comprises one or more abasic nucleoside (i.e. a nucleoside without a nucleobase). In some embodiments, the oligonucleotide of the invention comprises one or more TNA nucleoside. In some embodiments, the oligonucleotide of the invention comprises one or more SNA nucleoside. In some embodiments, the oligonucleotide of the invention comprises one or more iDNA nucleoside. In some embodiments, the oligonucleotide of the invention comprises one or more ScEt nucleoside. Examples of such nucleosides are depicted below:

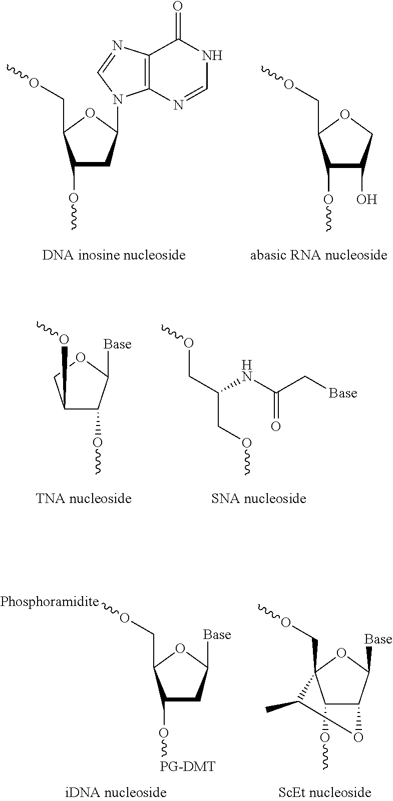

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., 2012, Accounts of Chemical Research, 45, 2055-2065 and Bergstrom, 2009, Curr. Protoc. Nucleic Acid Chem., 37, 1.4.1-1.4.32.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 7-deaza-8-azaguanine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

Structures of modified nucleobases that may be included in the oligonucleotides of the invention are depicted below, along with hypoxanthine:

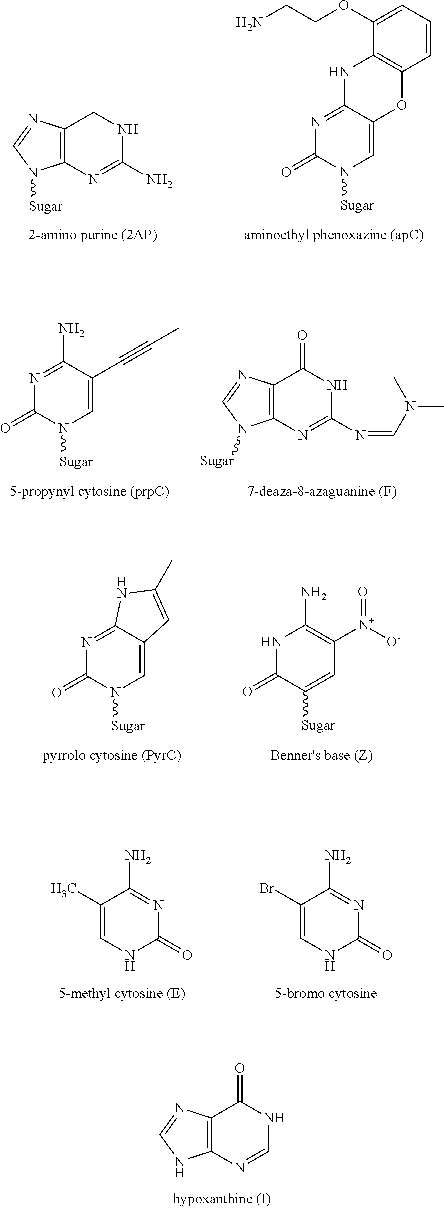

The nucleobase moieties may be indicated by the letter code for each corresponding nucleobase, e.g. A, T, G, C or U, wherein each letter may optionally include modified nucleobases of equivalent function. 5-methyl cytosine may be denoted as “E”. 7-deaza-8-azaguanine may be denoted as “F”. Hypoxanthine (such as in an inosine nucleoside) may be denoted as “1”.

### Editing a Target Nucleic Acid

The oligonucleotide of the invention is suitable for editing a target nucleic acid. The oligonucleotide of the invention is capable of editing a target nucleic acid. The term “editing” refers to altering the nucleobase sequence of the target nucleic acid. The oligonucleotide of the invention may be referred to as an “editing oligonucleotide”. The target nucleic acid is the nucleic acid which is intended to be edited.

The target nucleic acid comprises a target adenosine. The term “target adenosine” refers to an adenosine nucleoside of the target nucleic acid which is converted to an inosine nucleoside by deamination of the adenine nucleobase to form a hypoxanthine nucleobase. Accordingly, the oligonucleotide of the invention is capable of effecting conversion of the target adenosine (A) to inosine (1).

In some embodiments, the target nucleic acid is RNA. In some embodiments, the target nucleic acid encodes a protein. In some embodiments, the target nucleic acid is mRNA. In some embodiments, the target nucleic acid is a non-coding RNA.

### ADAR

The oligonucleotide of the invention is a guide oligonucleotide for an adenosine deaminase acting on RNA (ADAR). The oligonucleotide of the invention may thus be referred to as a “guide oligonucleotide” or an “editing guide oligonucleotide”.

ADARs are enzymes that bind to double-stranded RNA and deaminate an adenine nucleobase to form a hypoxanthine nucleobase, thereby converting an adenosine (A) nucleoside to an inosine (1) nucleoside. Three ADAR genes (ADAR1, ADAR2 and ADAR3) have been identified in mammals. ADAR1 and ADAR2 are expressed in many tissues, whereas ADAR3 is specifically expressed in the brain and may be catalytically inactive. In some embodiments, the ADAR for which the oligonucleotide of the invention is a guide oligonucleotide is ADAR1 or ADAR2. In some embodiments, the ADAR is endogenous human ADAR1 or ADAR2. Reference sequences in the UniProtKB and NCBI databases for human ADAR1 and ADAR2 are given in Table 1 below.

TABLE 1
Human ADAR1 and ADAR2 reference sequences

| | UniProtKB | NCBI | |
|---|---|---|---|
| | protein ref | gene ref | NCBI mRNA ref |
| ADAR1 | P55265 | NG_011844.2 | NM_001111.5 (transcript |
| | | | variant 1) or any other |
| | | | variant thereof in the |
| | | | NCBI databse |
| ADAR2 | P78563 | NG_052015.1 | NM_001112.4 (transcript |
| | | | variant 1) or any other |
| | | | variant thereof in the |
| | | | NCBI database |

The term “guide oligonucleotide” indicates that the oligonucleotide of the invention is capable of directing an ADAR to the target nucleic acid and to the specific target adenosine on the target nucleic acid, so that the ADAR deaminates the adenine of the target adenosine. The oligonucleotide achieves this by hybridizing with the target nucleic acid in the region of the target adenosine to forming a double-stranded molecule. The oligonucleotide of the invention is therefore capable of binding to the target nucleic acid by complementary base pairing. An ADAR can associate with the stretch of double-stranded nucleic acid. The oligonucleotide of the invention is thus capable of recruiting an ADAR to the target nucleic acid. The specific sequence of the oligonucleotide thereby determines to which adenosine the deaminating activity of the ADAR is directed, as the sequence of the oligonucleotide determines with which target nucleic acid the oligonucleotide is capable of hybridizing.

### Editing SERPINA1

In some embodiments of the invention, the target nucleic acid encodes alpha-1 antitrypsin (A1AT). In other words, in some embodiments, the oligonucleotide of the invention is for editing a target nucleic acid that encodes A1AT.

A1AT is a serine protease inhibitor synthesized in the liver that is released to other tissues to protects them from endogenous inflammatory serine proteases, such as neutrophil elastase. A1AT is encoded by the SERPINA1 (serpin family A member 1) gene. Thus, in some embodiments, the target nucleic acid is a SERPINA1 mRNA. In other words, in some embodiments, the oligonucleotide of the invention is for editing a SERPINA1 mRNA. The term “SERPINA1 mRNA” refers to any mRNA transcribed from the SERPINA1 gene. The UnitProtKB entry for A1AT is P01009 and the Reference Sequence number for the SERPINA1 gene in the NCBI database is NG_008290.1. Reference Sequence numbers for the eleven known mRNA transcript variants from the SERPINA1 gene are presented in Table 2 below.

TABLE 2
SERPINA1 mRNA reference sequences

| SERPINA1 | | Corresponding |
|---|---|---|
| mRNA transcript | mRNA Reference | protein Reference |
| variant | Sequence number | Sequence number |
| 1 | NM_000295.5 | NP_000286.3 |
| 2 | NM_001002236.3 | NP_001002236.1 |
| 3 | NM_001002235.3 | NP_001002235.1 |
| 4 | NM_001127700.2 | NP_001121172.1 |
| 5 | NM_001127701.2 | NP_001121173.1 |
| 6 | NM_001127702.2 | NP_001121174.1 |
| 7 | NM_001127703.2 | NP_001121175.1 |
| 8 | NM_001127704.2 | NP_001121176.1 |
| 9 | NM_001127705.2 | NP_001121177.1 |
| 10 | NM_001127706.2 | NP_001121178.1 |
| 11 | NM_001127707.2 | NP_001121179.1 |

In some embodiments, the SERPINA1 mRNA comprises or consists of the sequence of any one of the mRNA transcripts listed in Table 2.

Mutations in the SERPINA1 gene are associated with the disease termed A1AT deficiency (A1AD). The most severe form of A1AD is associated with a single base pair substitution leading to mutation in A1AT of glutamate (E) 342 to lysine (K) (E342K mutation).

As described herein, the oligonucleotide of the invention may be used to treat A1AD by editing mutated SERPINA1 mRNA. In particular, the mutated codon encoding the E342K mutation may be edited to treat A1AD. Glutamate (E) is encoded by the codons GAA and GAG. Lysine (K) is encoded by the codons AAA and AAG. In some embodiments, the oligonucleotide of the invention is capable of effecting conversion of an AAA codon encoding lysine to an IAA codon encoding glutamate on the target nucleic acid. In such embodiments, the oligonucleotide of the invention recruits an ADAR which converts the first adenosine (the target adenosine) of the AAA codon (encoding lysine) to inosine, producing an IAA codon which is read as GAA (encoding glutamate), thereby correcting the glutamate to lysine mutation. In some embodiments, the oligonucleotide of the invention is capable of effecting conversion of an AAG codon encoding lysine to an IAG codon encoding glutamate on the target nucleic acid. In such embodiments, the oligonucleotide of the invention recruits an ADAR which converts the first adenosine (the target adenosine) of the AAG codon (encoding lysine) to inosine, producing an IAG codon which is read as GAG (encoding glutamate), thereby correcting the glutamate to lysine mutation.

In some embodiments, the SERPINA1 mRNA comprises or consists of the sequence of any one of the mRNA transcripts listed in Table 2, and a GAG codon is mutated to AAG. In some embodiments, the SERPINA1 mRNA comprises or consists of the sequence of any one of the mRNA transcripts listed in Table 2, and a GAG codon encoding E342 in the A1AT protein is mutated to AAG.

The coding sequence from SERPINA1 mRNA transcript variant 11 (NM_001127707.2) comprising the E342K mutation is presented below as SEQ ID NO 186. The codon for K342 is AAG (shown underlined in the sequence below) and the first adenosine in the AAG codon is A1024 (shown in bold below).

SERPINA1 mRNA E342K mutant coding sequence -
SEQ ID NO 186:
GAGGATCCCCAGGGAGATGCTGCCCAGAAGACAGATACATCCCACCATGA
TCAGGATCACCCAACCTTCAACAAGATCACCCCCAACCTGGCTGAGTTCG
CCTTCAGCCTATACCGCCAGCTGGCACACCAGTCCAACAGCACCAATATC
TTCTTCTCCCCAGTGAGCATCGCTACAGCCTTTGCAATGCTCTCCCTGGG
GACCAAGGCTGACACTCACGATGAAATCCTGGAGGGCCTGAATTTCAACC
TCACGGAGATTCCGGAGGCTCAGATCCATGAAGGCTTCCAGGAACTCCTC
CGTACCCTCAACCAGCCAGACAGCCAGCTCCAGCTGACCACCGGCAATGG
CCTGTTCCTCAGCGAGGGCCTGAAGCTAGTGGATAAGTTTTTGGAGGATG
TTAAAAAGTTGTACCACTCAGAAGCCTTCACTGTCAACTTCGGGGACACC
GAAGAGGCCAAGAAACAGATCAACGATTACGTGGAGAAGGGTACTCAAGG
GAAAATTGTGGATTTGGTCAAGGAGCTTGACAGAGACACAGTTTTTGCTC
TGGTGAATTACATCTTCTTTAAAGGCAAATGGGAGAGACCCTTTGAAGTC
AAGGACACCGAGGAAGAGGACTTCCACGTGGACCAGGTGACCACCGTGAA
GGTGCCTATGATGAAGCGTTTAGGCATGTTTAACATCCAGCACTGTAAGA
AGCTGTCCAGCTGGGTGCTGCTGATGAAATACCTGGGCAATGCCACCGCC
ATCTTCTTCCTGCCTGATGAGGGGAAACTACAGCACCTGGAAAATGAACT
CACCCACGATATCATCACCAAGTTCCTGGAAAATGAAGACAGAAGGTCTG
CCAGCTTACATTTACCCAAACTGTCCATTACTGGAACCTATGATCTGAAG
AGCGTCCTGGGTCAACTGGGCATCACTAAGGTCTTCAGCAATGGGGCTGA
CCTCTCCGGGGTCACAGAGGAGGCACCCCTGAAGCTCTCCAAGGCCGTGC
ATAAGGCTGTGCTGACCATCGACAAGAAAGGGACTGAAGCTGCTGGGGCC
ATGTTTTTAGAGGCCATACCCATGTCTATCCCCCCCGAGGTCAAGTTCAA
CAAACCCTTTGTCTTCTTAATGATTGAACAAAATACCAAGTCTCCCCTCT
TCATGGGAAAAGTGGTGAATCCCACCCAAAAATAA

In some embodiments, the target adenosine corresponds to A1024 of SEQ ID NO 186. The term ‘corresponds to’ means that the target adenosine is not required to be the 1024^{th} nucleoside of the target nucleic acid and the sequence of the target nucleic acid is not otherwise limited to SEQ ID NO 186 but must be the first nucleoside in an AAG codon encoding a K342 within the coding sequence for A1AT, as per A1024. Thus, in some embodiments the target adenosine is the first nucleoside in an AAG codon encoding a K342 in a coding sequence for AAT.

In some embodiments, the target nucleic acid comprises a sequence having at least 80% identity to SEQ ID NO 186, such as at least 85%, at least 90%, at least 91%, at least 92%, at least 93%, at least 94%, at least 95%, at least 96%, at least 97%, at least 98%, at least 99% or 100% identity to SEQ ID NO 186, wherein the target nucleic acid comprises A1024 of SEQ ID NO 186. In some embodiments, the target nucleic acid comprises the sequence according to SEQ ID NO 186. In some embodiments, the target nucleic acid consists of a sequence having at least 80% identity to SEQ ID NO 186, such as at least 85%, at least 90%, at least 91%, at least 92%, at least 93%, at least 94%, at least 95%, at least 96%, at least 97%, at least 98%, at least 99% or 100% identity to SEQ ID NO 186, wherein the target nucleic acid comprises A1024 of SEQ ID NO 186. In some embodiments, the target nucleic acid consists of the sequence according to SEQ ID NO 186.

### Editing Nucleoside and Editing Region

The term “editing nucleoside” refers to the nucleoside in the oligonucleotide of the invention which is opposite the target adenosine when the oligonucleotide of the invention is hybridized with the target nucleic acid. In other words, the oligonucleotide of the invention forms a double-stranded molecule with the target nucleic acid through complementary base pairing between the nucleosides of the oligonucleotide and the target nucleic acid, wherein the sequences of the oligonucleotide and target nucleic acid are aligned such that the editing nucleoside aligns with the target adenosine. The editing nucleoside is not complementary to the target adenosine. In other words, the editing nucleoside forms a mismatch with the target adenosine. In some embodiments, the nucleobase of the editing nucleoside is selected from the group consisting of cytosine, 5-methyl cytosine, guanine and hypoxanthine. In some embodiments, the editing nucleoside is cytidine (C). In some embodiments, the nucleobase of the editing nucleoside is cytosine (C) or 5-methyl cytosine (m5C). In some embodiments, the nucleobase of the editing nucleoside is 5-methyl cytosine (m5C). In some embodiments, the editing nucleoside is guanosine (G). In some embodiments, the editing nucleoside is inosine (1).

The term “editing region” refers to one or more contiguous nucleosides (i.e. nucleosides linked by internucleoside linkages) which include the editing nucleoside. The editing region may be defined so as to differentiate nucleosides of the editing region from nucleosides of other regions of the oligonucleotide of the invention, such as mixmer regions and flank regions. The nucleosides of the editing region may have common features as described herein.

In some embodiments, the editing region consists of the editing nucleoside. In other words, there are no nucleosides in the editing region other than the editing nucleoside; in such embodiments, the editing nucleoside and editing region are synonymous.

In some embodiments, the editing regions comprises the nucleosides at positions +1, 0 and −1. In some embodiments, the editing regions consists of the nucleosides at positions +1, 0 and −1. In some embodiments, the editing region comprises an editing triplet consisting of 3 nucleosides, wherein the editing nucleoside is the central nucleoside of the editing triplet. In some embodiments, the editing region consists of an editing triplet consisting of 3 nucleosides, wherein the editing nucleoside is the central nucleoside of the editing triplet. In some embodiments, the editing triplet is 5′-thymidine-cytidine-inosine-3′ (TCI). Thus, in such embodiments, the central C is the editing nucleoside, with a T nucleoside immediately 5′ and an inosine nucleoside immediately 3′.

In some embodiments of the oligonucleotide of the invention, each nucleoside of the editing region comprises the same sugar moiety. In some embodiments, each nucleoside of the editing region is independently selected from the group consisting of DNA, RNA, 2′-O-methyl-RNA, 2′-fluoro-RNA, MOE-RNA, LNA, ANA, and FANA nucleosides. The different types of sugar moieties which may be comprised in the oligonucleotide of the invention are described elsewhere herein.

In some embodiments, the editing region comprises one or more DNA nucleosides. In some embodiments, the editing nucleoside is a DNA nucleoside. In some embodiments, all nucleosides of the editing region are DNA nucleosides. In some embodiments, the nucleoside at one or more of positions +1, 0 and −1 is a DNA nucleoside, such as at two or more of positions +1, 0 and −1. In some embodiments, the nucleoside at position +1 is a DNA nucleoside. In some embodiments, the nucleoside at position 0 is a DNA nucleoside. In some embodiments, the nucleoside at position −1 is a DNA nucleoside. In some embodiments the nucleoside at each of positions +1, 0 and −1 is a DNA nucleoside. In some embodiments, the editing region consists of the nucleosides at positions +1, 0 and −1 of the oligonucleotide, wherein the nucleoside at position +1 is a thymidine DNA nucleoside, the nucleoside at position 0 is a cytidine DNA nucleoside, and the nucleoside at position −1 is an inosine DNA nucleoside.

In some embodiments, the editing nucleoside is a FANA nucleoside. In some embodiments, the nucleoside at position 0 is a FANA nucleoside. In some embodiments, the editing nucleoside is a FANA nucleoside and all other nucleosides in the editing region are DNA nucleosides. In some embodiments, the nucleoside at position 0 is a FANA nucleoside, and (a) the nucleoside at position +1 is a DNA nucleoside, or (b) the nucleoside at position −1 is a DNA nucleoside, or (c) the nucleoside at each of positions +1 and −1 is a DNA nucleoside. In some embodiments, the editing region consists of the nucleosides at positions +1, 0 and −1 of the oligonucleotide, wherein the nucleoside at position +1 is a thymidine DNA nucleoside, the nucleoside at position 0 is a cytidine FANA nucleoside, and the nucleoside at position −1 is an inosine DNA nucleoside.

In some embodiments, the editing region comprises one or more nucleosides which comprise a different sugar moiety to the first type of 5′ mixmer nucleoside. In some embodiments, the editing region comprises one or more nucleosides which comprise a different sugar moiety to the second type of 5′ mixmer nucleoside. In some embodiments, the editing region comprises one or more nucleosides which comprise a different sugar moiety to the first type of 5′ mixmer nucleoside and to the second type of 5′ mixmer nucleoside. In some embodiments, the editing region comprises one or more nucleosides which comprise a different sugar moiety to the first type of 3′ mixmer nucleoside. In some embodiments, the editing region comprises one or more nucleosides which comprise a different sugar moiety to the second type of 3′ mixmer nucleoside. In some embodiments, the editing region comprises one or more nucleosides which comprise a different sugar moiety to the first type of 3′ mixmer nucleoside and to the second type of 3′ mixmer nucleoside.

In some embodiments, the editing region comprises one or more nucleosides which comprise a different sugar moiety to the sugar moiety of the first type of 5′ mixmer nucleoside, the second type of 5′ mixmer nucleoside, the first type of 3′ mixmer nucleoside, and the second type of 3′ mixmer nucleoside.

In some embodiments, each nucleoside of the editing region comprises a different sugar moiety to the first type of 5′ mixmer nucleoside. In some embodiments, each nucleoside of the editing region comprises a different sugar moiety to the second type of 5′ mixmer nucleoside. In some embodiments, each nucleoside of the editing region comprises a different sugar moiety to the first type of 3′ mixmer nucleoside. In some embodiments, each nucleoside of the editing region comprises a different sugar moiety to the second type of 3′ mixmer nucleoside. In some embodiments, each nucleoside of the editing region comprises a different sugar moiety to the sugar moiety of the first type of 5′ mixmer nucleoside, the second type of 5′ mixmer nucleoside, the first type of 3′ mixmer nucleoside, and the second type of 3′ mixmer nucleoside.

### Mixmer Regions

The oligonucleotide of the invention comprises one or more mixmer regions. The oligonucleotide of the invention comprises a 5′ mixmer region positioned 5′ to the editing region and a 3′ mixmer region positioned 3′ to the editing region.

The term “mixmer region” refers to two or more contiguous nucleosides (i.e. nucleosides linked by internucleoside linkages) which comprise more than one type of sugar moiety. In other words, a mixmer region comprises nucleosides with different types of sugar modifications. Nucleosides with different types of sugar moiety or sugar modifications may be referred to herein as “different types of nucleoside” or “different types of sugar-modified nucleosides”.

In some embodiments, the oligonucleotide comprises a mixmer region. In some embodiments, the oligonucleotide is a mixmer. In some embodiments, the oligonucleotide comprises a mixmer region split into two parts by the editing region. In other words, the mixmer region comprises the editing region. In some embodiments, the editing region splits the mixmer region into a 5′ mixmer region and a 3′ mixmer region.

In some embodiments, the 5′ mixmer region is positioned immediately 5′ to the editing region. In other words, there are no additional nucleosides between the 3′-most nucleoside of the 5′ mixmer region and the 5′-most nucleoside of the editing region; the 3′-most nucleoside of the 5′ mixmer region and the 5′-most nucleoside of the editing region are linked by an internucleoside linkage. In some embodiments, the 3′ mixmer region is positioned immediately 3′ to the editing region. In other words, there are no additional nucleosides between the 5′-most nucleoside of the 3′ mixmer region and the 3-most nucleoside of the editing region; the 5′-most nucleoside of the 3′ mixmer region and the 3′-most nucleoside of the editing region are linked by an internucleoside linkage. In some embodiments, the 5′ mixmer region is positioned immediately 5′ to the editing region and the 3′ mixmer region is positioned immediately 3′ to the editing region.

In some embodiments, the oligonucleotide comprises the structure:

X⁺¹—X⁰—X⁻¹
where in
X⁺¹ is the 5′ mixmer region,
X⁰ is the editing region, and
X⁻¹ is the 3′ mixmer region.

Thus, according to the above formula, the 5′ mixmer region, editing region and 3′ mixmer region are contiguous (i.e. linked by internucleoside linkages). Hyphens in the formula above represent internucleoside linkages.

A mixmer region comprises more than one type of sugar-modified nucleoside. The 5′ mixmer region comprises more than one type of sugar-modified nucleoside. The 3′ mixmer region comprises more than one type of sugar-modified nucleoside. In some embodiments, the mixmer region comprises more than one type of sugar-modified nucleoside. In some embodiments, the 5′ mixmer region comprises exactly two types of sugar-modified nucleoside. In some embodiments, the 3′ mixmer region comprises exactly two types of sugar-modified nucleoside.

In some embodiments, the mixmer region comprises a first type of mixmer nucleoside and a second type of mixmer nucleoside, wherein the sugar moiety of the first type of mixmer nucleoside is different to the sugar moiety of the second type of mixmer nucleoside. In other words, the first type of mixmer nucleoside and second type of mixmer nucleoside are differentiated by the type of sugar moiety they comprise.

In some embodiments, the 5′ mixmer region comprises a first type of 5′ mixmer nucleoside and a second type of 5′ mixmer nucleoside, wherein the sugar moiety of the first type of 5′ mixmer nucleoside is different to the sugar moiety of the second type of 5′ mixmer nucleoside. In other words, the first type of 5′ mixmer nucleoside and second type of 5′ mixmer nucleoside are differentiated by the type of sugar moiety they comprise.

In some embodiments, the 3′ mixmer region comprises a first type of 3′ mixmer nucleoside and a second type of 3′ mixmer nucleoside, wherein the sugar moiety of the first type of 3′ mixmer nucleoside is different to the sugar moiety of the second type of 3′ mixmer nucleoside. In other words, the first type of 3′ mixmer nucleoside and second type of 3′ mixmer nucleoside are differentiated by the type of sugar moiety they comprise.

Suitable sugar moieties (i.e. different types of sugar-modified nucleosides or sugar modifications) are described elsewhere herein.

In some embodiments, the mixmer region comprises a first type of mixmer nucleoside and a second type of mixmer nucleoside. In some embodiments, the first type of mixmer nucleoside is a 2′-O-methyl-RNA nucleoside and the second type of mixmer nucleoside is a 2′-fluoro-RNA nucleoside. In some embodiments, the first type of mixmer nucleoside is a 2′-fluoro-RNA nucleoside and the second type of mixmer nucleoside is a 2′-O-methyl-RNA nucleoside.

Notably, other features (e.g. the nucleobase) of a given nucleoside of a first type of mixmer nucleoside may differ from those of a given nucleoside of the second type, but it is the differing sugar moiety that determines to which type of mixmer nucleoside the nucleoside belongs. For example, two nucleosides may have different nucleobases but be the same type of mixmer nucleoside due to having the same type of sugar moiety; conversely two nucleosides may have the same nucleobase but be of different types of mixmer nucleoside due to their different sugar moieties.

In some embodiments, the first type of 5′ mixmer nucleoside and the first type of 3′ mixmer nucleoside are the same type of sugar-modified nucleoside (i.e. they have the same sugar moiety). In some embodiments, the first type of 5′ mixmer nucleoside and the first type of 3′ mixmer nucleoside are the same type of sugar-modified nucleoside and are referred to together as the “first type of mixmer nucleoside”. In some embodiments, the second type of 5′ mixmer nucleoside and the second type of 3′ mixmer nucleoside are the same type of sugar-modified nucleoside (i.e. they have the same sugar moiety). In some embodiments, the second type of 5′ mixmer nucleoside and the second type of 3′ mixmer nucleoside are the same type of sugar-modified nucleoside and are referred to together as the “second type of mixmer nucleoside”.

In some embodiments, the first type of 5′ mixmer nucleoside and second type of 5′ mixmer nucleoside are arranged in an alternating pattern within the 5′ mixmer region. In other words, a stretch of nucleosides of the first type of 5′ mixmer nucleoside is followed by a stretch of nucleosides of the second type of 5′ mixmer nucleoside, which in turn is followed by a stretch of nucleosides of the first type of 5′ mixmer nucleoside, and so on. In other words, in some embodiments, the 5′ mixmer region comprises an alternating pattern of the first type of 5′ mixmer nucleosides and the second type of 5′ mixmer nucleosides.

Likewise, in some embodiments, the first type of 3′ mixmer nucleoside and second type of 3′ mixmer nucleoside are arranged in an alternating pattern within the 3′ mixmer region. In other words, a stretch of nucleosides of the first type of 3′ mixmer nucleoside is followed by a stretch of nucleosides of the second type of 3′ mixmer nucleoside, which in turn is followed by a stretch of nucleosides of the first type of 3′ mixmer nucleoside, and so on. In other words, in some embodiments, the 3′ mixmer region comprises an alternating pattern of the first type of 3′ mixmer nucleosides and the second type of 3′ mixmer nucleosides.

There may be from 1 to about 4 nucleosides in each stretch of nucleosides of the first type or second type of mixmer nucleoside. In some embodiments, the number of nucleosides in each stretch of nucleosides of a given type varies independently. Thus, in some embodiments, the number of nucleosides in each stretch varies between the types of nucleoside (e.g. there may be a certain number of nucleosides of the first type of nucleoside in each stretch of the first type, and a different number of nucleosides of the second type of nucleoside in each stretch of the second type). In some embodiments, the number of nucleosides varies between stretches of nucleosides of the same type (e.g. there may be a certain number of nucleosides in a given stretch of nucleosides of the first type, but a different number of nucleosides in a different stretch of nucleosides of the first type).

In some embodiments, each stretch of nucleosides of the same type comprises the same number of nucleosides. For example, all stretches of nucleosides of the first type may be 2 nucleosides long and all stretches of nucleosides of the second type may be 1 nucleoside long. In some embodiments, each stretch of nucleosides comprises the same number of nucleosides. For example, all stretches of nucleosides of the first and second type may be 2 nucleosides long.

In some embodiments, each stretch of nucleosides of the first or second type is a single nucleoside. In other words, in some embodiments, the 5′ mixmer region comprises a single nucleoside (i.e. 1 nucleoside) of the first type of 5′ mixmer oligonucleoside, followed by a single nucleoside of the second type of 5′ mixmer oligonucleoside, followed by a single nucleoside of the first type of 5′ mixmer oligonucleoside, and so on. In some embodiments, the first type of 5′ mixmer nucleoside and second type of 5′ mixmer nucleoside are arranged in an alternating pattern of single nucleosides. In other words, in some embodiments, the 5′ mixmer region comprises an alternating pattern of single nucleosides of the first type of 5′ mixmer nucleoside and single nucleosides of the second type of 5′ mixmer nucleosides.

Likewise, in some embodiments, the 3′ mixmer region comprises a single nucleoside (i.e. 1 nucleoside) of the first type of 3′ mixmer oligonucleoside, followed by a single nucleoside of the second type of 3′ mixmer oligonucleoside, followed by a single nucleoside of the first type of 3′ mixmer oligonucleoside, and so on. In some embodiments, the first type of 3′ mixmer nucleoside and second type of 3′ mixmer nucleoside are arranged in an alternating pattern of single nucleosides. In other words, in some embodiments, the 3′ mixmer region comprises an alternating pattern of single nucleosides of the first type of 3′ mixmer nucleoside and single nucleosides of the second type of 3′ mixmer nucleosides.

In some embodiments, the 5′ mixmer region comprises
(a) one or more first 5′ mixmer sub-regions of one or more nucleosides of the first type of 5′ mixmer nucleoside, and
(b) one or more second 5′ mixmer sub-regions of one or more nucleosides of the second type of 5′ mixmer nucleoside,
   wherein the first 5′ mixmer sub-regions and second 5′ mixmer sub-regions are arranged in an alternating pattern. In other words, a first 5′ mixmer sub-region is followed by a second 5′ mixmer sub-region, which may be followed by another first 5′ mixmer sub-region, which is followed by a second 5′ mixmer sub-region, and so on.

In some embodiments, the 5′ mixmer region comprises
(a) one or more first 5′ mixmer sub-regions of 1, 2, 3 or 4 nucleosides of the first type of 5′ mixmer nucleoside, and
(b) one or more second 5′ mixmer sub-regions of 1, 2, 3 or 4 nucleosides of the second type of 5′ mixmer nucleoside,
   wherein the first 5′ mixmer sub-regions and second 5′ mixmer sub-regions are arranged in an alternating pattern.

In some embodiments, the 5′ mixmer region comprises
(a) one or more first 5′ mixmer sub-regions of 1 nucleoside of the first type of 5′ mixmer nucleoside, and
(b) one or more second 5′ mixmer sub-regions of 1 nucleoside of the second type of 5′ mixmer nucleoside,
   wherein the first 5′ mixmer sub-regions and second 5′ mixmer sub-regions are arranged in an alternating pattern.

Likewise, in some embodiments, the 3′ mixmer region comprises
(a) one or more first 3′ mixmer sub-regions of one or more nucleosides of the first type of 3′ mixmer nucleoside, and
(b) one or more second 3′ mixmer sub-regions of one or more nucleosides of the second type of 3′ mixmer nucleoside,
   wherein the first 3′ mixmer sub-regions and second 3′ mixmer sub-regions are arranged in an alternating pattern. In other words, a first 3′ mixmer sub-region is followed by a second 3′ mixmer sub-region, which may be followed by another first 3′ mixmer sub-region, which is followed by a second 3′ mixmer sub-region, and so on.

In some embodiments, the 3′ mixmer region comprises
(a) one or more first 3′ mixmer sub-regions of 1, 2, 3 or 4 nucleosides of the first type of 3′ mixmer nucleoside, and
(b) one or more second 3′ mixmer sub-regions of 1, 2, 3 or 4 nucleosides of the second type of 3′ mixmer nucleoside,
   wherein the first 3′ mixmer sub-regions and second 3′ mixmer sub-regions are arranged in an alternating pattern.

In some embodiments, the 3′ mixmer region comprises
(a) one or more first 3′ mixmer sub-regions of 1 nucleoside of the first type of 3′ mixmer nucleoside, and
(b) one or more second 3′ mixmer sub-regions of 1 nucleoside of the second type of 3′ mixmer nucleoside,
   wherein the first 3′ mixmer sub-regions and second 3′ mixmer sub-regions are arranged in an alternating pattern.

In some embodiments, the 5′ mixmer region comprises one or more sub-regions of one or more nucleosides of the first type of 5′ mixmer nucleoside, wherein each sub-region is separated by one or more nucleosides of the second type of 5′ mixmer nucleoside. In some embodiments, the 3′ mixmer region comprises one or more sub-regions of one or more nucleosides of the first type of 3′ mixmer nucleoside, wherein each sub-region is separated by one or more nucleosides of the second type of 3′ mixmer nucleoside.

In some embodiments, the 5′ mixmer region comprises one or more sub-regions of 1, 2, 3 or 4 nucleosides of the first type of 5′ mixmer nucleoside, wherein each sub-region is separated by 1, 2, 3 or 4 nucleosides of the second type of 5′ mixmer nucleoside. In some embodiments, the 3′ mixmer region comprises one or more sub-regions of 1, 2, 3 or 4 nucleosides of the first type of 3′ mixmer nucleoside, wherein each sub-region is separated by 1, 2, 3 or 4 nucleosides of the second type of 3′ mixmer nucleoside.

In some embodiments, the 5′ mixmer region comprises one or more sub-regions of 1 nucleoside of the first type of 5′ mixmer nucleoside, wherein each sub-region is separated by 1 nucleoside of the second type of 5′ mixmer nucleoside. In some embodiments, the 3′ mixmer region comprises one or more sub-regions of 1 nucleoside of the first type of 3′ mixmer nucleoside, wherein each sub-region is separated by 1 nucleoside of the second type of 3′ mixmer nucleoside.

The structure of the mixmer regions may be represented formulaically. Thus, in some embodiments, the 5′ mixmer region comprises the structure:

(Y⁵—Z⁵—)ₘ
wherein
Y⁵ is one or more nucleosides of the first type of 5′ mixmer nucleoside,
Z⁵ is one or more nucleosides of the second type of 5′ mixmer nucleoside, and
m is a number from 1 to 20.

In some embodiments, the 3′ mixmer region comprises the structure:

(Y³—Z³—)ₙ
wherein
Y³ is one or more nucleosides of the first type of 3′ mixmer nucleoside,
Z³ is one or more nucleosides of the second type of 3′ mixmer nucleoside, and
n is a number from 1 to 20.

The nucleosides in each mixmer region are contiguous (i.e. linked by internucleoside linkages). Hyphens in the formula above represent internucleoside linkages. In some embodiments, each Y⁵ is independently 1, 2, 3 or 4 nucleosides. In some embodiments, each Y⁵ is independently 1 nucleoside. In some embodiments, each Z⁵ is independently 1, 2, 3 or 4 nucleosides. In some embodiments, each Z⁵ is independently 1 nucleoside. In some embodiments, each Y³ is independently 1, 2, 3 or 4 nucleosides. In some embodiments, each Y³ is independently 1 nucleoside. In some embodiments, each Z³ is independently 1, 2, 3 or 4 nucleosides. In some embodiments, each Z³ is independently 1 nucleoside.

In some embodiments, each Y⁵ is independently 1 nucleoside, each Z⁵ is independently 1 nucleoside, each Y³ is independently 1 nucleoside, and each Z³ is independently 1 nucleoside.

In some embodiments, m is 1, 2, 3, 4, 5, 6, 7, 8, 9, 10, 11, 12, 13, 14, 15, 16, 17, 18, 19 or 20. In some embodiments, m is 6, 7, 8, 9, 10, 11, 12, 13, 14 or 15. In some embodiments, m is 9.

In some embodiments, n is 1, 2, 3, 4, 5, 6, 7, 8, 9, 10, 11, 12, 13, 14, 15, 16, 17, 18, 19 or 20. In some embodiments, n is 3, 4, 5, 6, 7, 8 or 9. In some embodiments, n is 5.

In other words, the 5′ mixmer region may be represented as:

Y⁵—Z⁵—Y⁵—Z⁵—Y⁵—Z⁵—Y⁵—Z⁵—Y⁵—Z⁵—Y⁵—Z⁵ . . .

wherein each Y⁵ is a stretch of nucleosides of the first type of 5′ mixmer nucleoside, and each Z⁵ is a stretch of nucleosides of the second type of 5′ mixmer nucleoside. The stretches of nucleosides are linked by internucleosides linkages and thus form a contiguous 5′ mixmer region. Each Y⁵ stretch of nucleosides is independently 1, 2, 3 or 4 nucleosides. Each Z⁵ stretch of nucleosides is independently 1, 2, 3 or 4 nucleosides. There are up to 20 pairs of Y and Z⁵.

Likewise, the 3′ mixmer region may be represented as:

Y³—Z³—Y³—Z³—Y³—Z³—Y³—Z³—Y³—Z³—Y³—Z³ . . .

wherein each Y³ is a stretch of nucleosides of the first type of 3′ mixmer nucleoside, and each Z³ is a stretch of nucleosides of the second type of 3′ mixmer nucleoside. The stretches of nucleosides are linked by internucleosides linkages and thus form a contiguous 3′ mixmer region. Each Y³ stretch of nucleosides is independently 1, 2, 3 or 4 nucleosides. Each Z³ stretch of nucleosides is independently 1, 2, 3 or 4 nucleosides. There are up to 20 pairs of Y³ and Z³.

### Mixmer Region Length

In some embodiments of the oligonucleotide, the 5′ mixmer region is from 15 to 25 nucleosides long. In some embodiments, the 5′ mixmer region is 15, 16, 17, 18, 19, 20, 21, 22, 23, 24 or 25 nucleosides long. In some embodiments, the 5′ mixmer region is 18 nucleosides long.

In some embodiments of the oligonucleotide, the 3′ mixmer region is from 4 to 12 nucleosides long. In some embodiments, the 3′ mixmer region is 4, 5, 6, 7, 8, 9, 10, 11, or 12 nucleosides long. In some embodiments, the 3′ mixmer region is 9 nucleosides long.

In some embodiments, the 5′ mixmer region is 24 nucleosides long and the 3′ mixmer region is 9 nucleosides long. In some embodiments, the 5′ mixmer region is 24 nucleosides long and the 3′ mixmer region is 8 nucleosides long. In some embodiments, the 5′ mixmer region is 24 nucleosides long and the 3′ mixmer region is 7 nucleosides long. In some embodiments, the 5′ mixmer region is 24 nucleosides long and the 3′ mixmer region is 6 nucleosides long. In some embodiments, the 5′ mixmer region is 23 nucleosides long and the 3′ mixmer region is 9 nucleosides long. In some embodiments, the 5′ mixmer region is 23 nucleosides long and the 3′ mixmer region is 4 nucleosides long. In some embodiments, the 5′ mixmer region is 22 nucleosides long and the 3′ mixmer region is 9 nucleosides long. In some embodiments, the 5′ mixmer region is 22 nucleosides long and the 3′ mixmer region is 5 nucleosides long. In some embodiments, the 5′ mixmer region is 21 nucleosides long and the 3′ mixmer region is 9 nucleosides long. In some embodiments, the 5′ mixmer region is 21 nucleosides long and the 3′ mixmer region is 6 nucleosides long. In some embodiments, the 5′ mixmer region is 20 nucleosides long and the 3′ mixmer region is 9 nucleosides long. In some embodiments, the 5′ mixmer region is 20 nucleosides long and the 3′ mixmer region is 7 nucleosides long. In some embodiments, the 5′ mixmer region is 19 nucleosides long and the 3′ mixmer region is 9 nucleosides long. In some embodiments, the 5′ mixmer region is 19 nucleosides long and the 3′ mixmer region is 8 nucleosides long. In some embodiments, the 5′ mixmer region is 19 nucleosides long and the 3′ mixmer region is 7 nucleosides long. In some embodiments, the 5′ mixmer region is 19 nucleosides long and the 3′ mixmer region is 6 nucleosides long. In some embodiments, the 5′ mixmer region is 18 nucleosides long and the 3′ mixmer region is 9 nucleosides long. In some embodiments, the 5′ mixmer region is 17 nucleosides long and the 3′ mixmer region is 9 nucleosides long.

In some embodiments, the oligonucleotide is 35, 36, 37, 38, 39, 40, 41, 42, 43, 44 or 45 nucleosides long, the 5′ mixmer region is 15, 16, 17, 18, 19, 20, 21, 22, 23, 24 or 25 nucleosides long and the 3′ mixmer region is 5, 6, 7, 8, 9, 10, 11, or 12 nucleosides long. In some embodiments, the oligonucleotide is 35, 36, 37, 38, 39, 40, 41, 42, 43, 44 or 45 nucleosides long, the 5′ mixmer region is 16, 17, 18, 19, 20, 21, 22 or 23 nucleosides long and the 3′ mixmer region is 8 or 9 nucleosides long. In some embodiments, the oligonucleotide is 40 nucleosides long, the 5′ mixmer region is 18 nucleosides long and the 3′ mixmer region is 9 nucleosides long.

In some embodiments of the oligonucleotide of the invention, the 5′ mixmer region comprises the nucleosides of from position +1 to position +10, +11, +12, +13, +14, +15, +16, +17, +18, +19, +20, +21, +22, +23, +24 or +25. In other words, the 5′ mixmer region comprises the nucleosides of from position +1 to position +10, or from position +1 to position +11, or from position +1 to position +12, and so on. In some embodiments, the 5′ mixmer region comprises the nucleosides of from position +2 to position +10, +11, +12, +13, +14, +15, +16, +17, +18, +19, +20, +21, +22, +23, +24 or +25. In some embodiments, the 5′ mixmer region comprises the nucleosides of from position +2 to position +15, +16, +17, +18, +19, +20, +21, +22, +23 or +24. In some embodiments, the 5′ mixmer region comprises the nucleosides of from position +2 to position +19, +20, +21, +22, +23 or +24.

In some embodiments, the 5′ mixmer region consists of the nucleosides of from position +1 to position +10, +11, +12, +13, +14, +15, +16, +17, +18, +19, +20, +21, +22, +23, +24 or +25. In some embodiments, the 5′ mixmer region consists of the nucleosides of from position +2 to position +10, +11, +12, +13, +14, +15, +16, +17, +18, +19, +20, +21, +22, +23, +24 or +25. In some embodiments, the 5′ mixmer region consists of the nucleosides of from position +2 to position +15, +16, +17, +18, +19, +20, +21, +22, +23 or +24. In some embodiments, the 5′ mixmer region consists of the nucleosides of from position +2 to position +19, +20, +21, +22, +23 or +24.

In some embodiments, the 5′ mixmer region comprises the nucleosides of positions +2 to +19. In some embodiments, the 5′ mixmer region consists of the nucleosides of positions +2 to +19.

In some embodiments of the oligonucleotide of the invention, the 3′ mixmer region comprises the nucleosides of from position −1 to position −3, −4, −5, −6, −7, −8, −9. −10, −11, −12, −13, −14, −15, −16 or −17. In other words, the 3′ mixmer region comprises the nucleosides of from position −1 to position −3, or from position −1 to position −4, or from position −1 to position −5, and so on. In some embodiments, the 3′ mixmer region comprises the nucleosides of from position −2 to position −3, −4, −5, −6, −7, −8, −9. −10, −11, −12, −13, −14, −15, −16 or −17. In some embodiments, the 3′ mixmer region comprises the nucleosides of from position −2 to position −5, −6, −7, −8, −9, −10, −11, −12. −13, −14 or −15. In some embodiments, the 3′ mixmer region comprises the nucleosides of from position −2 to position −5, −6, −7, −8, −9 or −10.

In some embodiments, the 3′ mixmer region comprises the nucleosides of positions −2 to −10. In some embodiments, the 3′ mixmer region consists of the nucleosides of positions −2 to −10.

In some embodiments of the oligonucleotide of the invention, the 5′ mixmer region consists of the nucleosides of from position +2 to position +15, +16, +17, +18, +19, +20, +21, +22, +23 or +24; and the 3′ mixmer region consists of the nucleosides of from position −2 to position −5, −6, −7, −8, −9, −10, −11, −12, −13, −14 or −15. In some embodiments, the 5′ mixmer region consists of the nucleosides of from position +2 to position +17, +18, +19, +20, +21, +22, +23 or +24; and the 3′ mixmer region consists of the nucleosides of from position −2 to position −7. −8, −9 or −10.

In some embodiments, the 5′ mixmer region consists of the nucleosides of from position +2 to position +17, +18, +19, +20, +21, +22, +23 or +24; and the 3′ mixmer region consists of the nucleosides of from position −2 to position −10. In some embodiments, the 5′ mixmer region consists of the nucleosides of from position +2 to position +17; and the 3′ mixmer region consists of the nucleosides of from position −2 to position −10. In some embodiments, the 5′ mixmer region consists of the nucleosides of from position +2 to position +18; and the 3′ mixmer region consists of the nucleosides of from position −2 to position −10. In some embodiments, the 5′ mixmer region consists of the nucleosides of from position +2 to position +19; and the 3′ mixmer region consists of the nucleosides of from position −2 to position −10. In some embodiments, the 5′ mixmer region consists of the nucleosides of from position +2 to position +20; and the 3′ mixmer region consists of the nucleosides of from position −2 to position −10. In some embodiments, the 5′ mixmer region consists of the nucleosides of from position +2 to position +21; and the 3′ mixmer region consists of the nucleosides of from position −2 to position −10. In some embodiments, the 5′ mixmer region consists of the nucleosides of from position +2 to position +22; and the 3′ mixmer region consists of the nucleosides of from position −2 to position −10. In some embodiments, the 5′ mixmer region consists of the nucleosides of from position +2 to position +23; and the 3′ mixmer region consists of the nucleosides of from position −2 to position −10. In some embodiments, the 5′ mixmer region consists of the nucleosides of from position +2 to position +24; and the 3′ mixmer region consists of the nucleosides of from position −2 to position −10. In some embodiments, the 5′ mixmer region consists of the nucleosides of from position +2 to position +25; and the 3′ mixmer region consists of the nucleosides of from position −2 to position −10.

In some embodiments, the 5′ mixmer region consists of the nucleosides of from position +2 to position +25; and the 3′ mixmer region consists of the nucleosides of from position −2 to position −9. In some embodiments, the 5′ mixmer region consists of the nucleosides of from position +2 to position +25; and the 3′ mixmer region consists of the nucleosides of from position −2 to position −8. In some embodiments, the 5′ mixmer region consists of the nucleosides of from position +2 to position +25; and the 3′ mixmer region consists of the nucleosides of from position −2 to position −7.

In some embodiments, the 5′ mixmer region consists of the nucleosides of from position +2 to position +20; and the 3′ mixmer region consists of the nucleosides of from position −2 to position −8. In some embodiments, the 5′ mixmer region consists of the nucleosides of from position +2 to position +20; and the 3′ mixmer region consists of the nucleosides of from position −2 to position −7.

In some embodiments, the 5′ mixmer region consists of the nucleosides of from position +2 to position +20; and the 3′ mixmer region consists of the nucleosides of from position −2 to position −9. In some embodiments, the 5′ mixmer region consists of the nucleosides of from position +2 to position +21; and the 3′ mixmer region consists of the nucleosides of from position −2 to position −8. In some embodiments, the 5′ mixmer region consists of the nucleosides of from position +2 to position +22; and the 3′ mixmer region consists of the nucleosides of from position −2 to position −7. In some embodiments, the 5′ mixmer region consists of the nucleosides of from position +2 to position +23; and the 3′ mixmer region consists of the nucleosides of from position −2 to position −6. In some embodiments, the 5′ mixmer region consists of the nucleosides of from position +2 to position +24; and the 3′ mixmer region consists of the nucleosides of from position −2 to position −5.

In some embodiments, the oligonucleotide is 35, 36, 37, 38, 39, 40, 41, 42, 43, 44 or 45 nucleosides long, the 5′ mixmer region consists of the nucleosides of from position +2 to position +15, +16, +17, +18, +19, +20, +21, +22, +23 or +24, and the 3′ mixmer region consists of the nucleosides of from position −2 to position −5, −6, −7, −8, −9, −10, −11, −12, −13, −14 or −15. In some embodiments, the oligonucleotide is 35, 36, 37, 38, 39, 40, 41, 42, 43, 44 or 45 nucleosides long, the 5′ mixmer region consists of the nucleosides of from position +2 to position +17, +18, +19, +20, +21, +22, +23 or +24, and the 3′ mixmer region consists of the nucleosides of from position −2 to position −7. −8. −9 or −10. In some embodiments, the oligonucleotide is 40 nucleosides long, the 5′ mixmer region consists of the nucleosides of from position +2 to position +17, +18, +19, +20, +21, +22, +23 or +24, and the 3′ mixmer region consists of the nucleosides of from position −2 to position −7, −8, −9 or −10. In some embodiments, the oligonucleotide is 40 nucleosides long, the 5′ mixmer region consists of the nucleosides of from position +2 to position +19, and the 3′ mixmer region consists of the nucleosides of from position −2 to position −10.

### Mixmer Region Patterns

In some embodiments, the nucleoside at one or more of positions +3, +5, +7, +9, +11, +13, +15, +17 and +19 is of the first type of mixmer nucleoside. In some embodiments, the nucleoside at one or more of positions +3, +5, +7, +9, +11, +13, +15, +17 and +19 is of the first type of 5′ mixmer nucleoside. In some embodiments, the nucleoside at position +3 is of the first type of 5′ mixmer nucleoside, and/or the nucleoside at position +5 is of the first type of 5′ mixmer nucleoside, and/or the nucleoside at position +7 is of the first type of 5′ mixmer nucleoside, and/or the nucleoside at position +9 is of the first type of 5′ mixmer nucleoside, and/or the nucleoside at position +11 is of the first type of 5′ mixmer nucleoside, and/or the nucleoside at position +13 is of the first type of 5′ mixmer nucleoside, and/or the nucleoside at position +15 is of the first type of 5′ mixmer nucleoside, and/or the nucleoside at position +17 is of the first type of 5′ mixmer nucleoside and/or the nucleoside at position +19 is of the first type of 5′ mixmer nucleoside. In some embodiments, the nucleoside at each of positions +3, +5, +7, +9, +11, +13, +15, +17 and +19 is of the first type of 5′ mixmer nucleoside.

In some embodiments, the nucleoside at one or more of positions −3, −5, −7 and −9 is of the first type of mixmer nucleoside. In some embodiments, the nucleoside at one or more of positions −3, −5, −7 and −9 is of the first type of 3′ mixmer nucleoside. In some embodiments, the nucleoside at position −3 is of the first type of 3′ mixmer nucleoside, and/or the nucleoside at position −5 is of the first type of 3′ mixmer nucleoside, and/or the nucleoside at position −7 is of the first type of 3′ mixmer nucleoside, and/or the nucleoside at position −9 is of the first type of 3′ mixmer nucleoside. In some embodiments, the nucleoside at each of positions −3, −5, −7 and −9 is of the first type of 3′ mixmer nucleoside.

In some embodiments, the nucleoside at one or more of positions +2, +4, +6, +8, +10, +12, +14 and +18 is of the second type of mixmer nucleoside. In some embodiments, the nucleoside at one or more of positions +2, +4, +6, +8, +10, +12, +14 and +18 is of the second type of 5′ mixmer nucleoside. In some embodiments, the nucleoside at position +2 is of the second type of 5′ mixmer nucleoside, and/or the nucleoside at position +4 is of the second type of 5′ mixmer nucleoside, and/or the nucleoside at position +6 is of the second type of 5′ mixmer nucleoside, and/or the nucleoside at position +8 is of the second type of 5′ mixmer nucleoside, and/or the nucleoside at position +10 is of the second type of 5′ mixmer nucleoside, and/or the nucleoside at position +12 is of the second type of 5′ mixmer nucleoside, and/or the nucleoside at position +14 is of the second type of 5′ mixmer nucleoside, and/or the nucleoside at position +18 is of the second type of 5′ mixmer nucleoside. In some embodiments, the nucleoside at each of positions +2, +4, +6, +8, +10, +12, +14 and +18 is of the second type of 5′ mixmer nucleoside.

In some embodiments, the nucleoside at one or more of positions −2, −4 and −6 is of the second type of mixmer nucleoside. In some embodiments, the nucleoside at one or more of positions −2, −4 and −6 is of the second type of 3′ mixmer nucleoside. In some embodiments, the nucleoside at position −2 is of the second type of 3′ mixmer nucleoside, and/or the nucleoside at position −4 is of the second type of 3′ mixmer nucleoside, and/or the nucleoside at position −6 is of the second type of 3′ mixmer nucleoside. In some embodiments, the nucleoside at each of positions −2, −4 and −6 is of the second type of 3′ mixmer nucleoside.

In some embodiments, the nucleoside at one or more of positions +3, +5, +7, +9, +11, +13, +15, +17, +19, +21 and +23 is of the first type of mixmer nucleoside. In some embodiments, the nucleoside at one or more of positions +3, +5, +7, +9, +11, +13, +15, +17, +19, +21 and +23 is of the first type of 5′ mixmer nucleoside. In some embodiments, the nucleoside at position +3 is of the first type of 5′ mixmer nucleoside, and/or the nucleoside at position +5 is of the first type of 5′ mixmer nucleoside, and/or the nucleoside at position +7 is of the first type of 5′ mixmer nucleoside, and/or the nucleoside at position +9 is of the first type of 5′ mixmer nucleoside, and/or the nucleoside at position +11 is of the first type of 5′ mixmer nucleoside, and/or the nucleoside at position +13 is of the first type of 5′ mixmer nucleoside, and/or the nucleoside at position +15 is of the first type of 5′ mixmer nucleoside, and/or the nucleoside at position +17 is of the first type of 5′ mixmer nucleoside, and/or the nucleoside at position +19 is of the first type of 5′ mixmer nucleoside, and/or the nucleoside at position +21 is of the first type of 5′ mixmer nucleoside, and/or the nucleoside at position +23 is of the first type of 5′ mixmer nucleoside. In some embodiments, the nucleoside at each of positions +3, +5, +7, +9, +11, +13, +15, +17, +19, +21 and +23 is of the first type of 5′ mixmer nucleoside.

In some embodiments, the nucleoside at one or more of positions −3, −5, −7, −9, −11, −13 and −15 is of the first type of mixmer nucleoside. In some embodiments, the nucleoside at one or more of positions −3, −5, −7, −9, −11, −13 and −15 is of the first type of 3′ mixmer nucleoside. In some embodiments, the nucleoside at position −3 is of the first type of 3′ mixmer nucleoside, and/or the nucleoside at position −5 is of the first type of 3′ mixmer nucleoside, and/or the nucleoside at position −7 is of the first type of 3′ mixmer nucleoside, and/or the nucleoside at position −9 is of the first type of 3′ mixmer nucleoside, and/or the nucleoside at position −11 is of the first type of 3′ mixmer nucleoside, and/or the nucleoside at position −13 is of the first type of 3′ mixmer nucleoside, and/or the nucleoside at position −15 is of the first type of 3′ mixmer nucleoside. In some embodiments, the nucleoside at each of positions −3, −5, −7, −9. −11, −13 and −15 is of the first type of 3′ mixmer nucleoside.

In some embodiments, the nucleoside at one or more of positions +2, +4, +6, +8, +10, +12, +14, +18, +20, +22 and +24 is of the second type of mixmer nucleoside. In some embodiments, the nucleoside at one or more of positions +2, +4, +6, +8, +10, +12, +14, +18, +20, +22 and +24 is of the second type of 5′ mixmer nucleoside. In some embodiments, the nucleoside at position +2 is of the second type of 5′ mixmer nucleoside, and/or the nucleoside at position +4 is of the second type of 5′ mixmer nucleoside, and/or the nucleoside at position +6 is of the second type of 5′ mixmer nucleoside, and/or the nucleoside at position +8 is of the second type of 5′ mixmer nucleoside, and/or the nucleoside at position +10 is of the second type of 5′ mixmer nucleoside, and/or the nucleoside at position +12 is of the second type of 5′ mixmer nucleoside, and/or the nucleoside at position +14 is of the second type of 5′ mixmer nucleoside, and/or the nucleoside at position +18 is of the second type of 5′ mixmer nucleoside, and/or the nucleoside at position +20 is of the second type of 5′ mixmer nucleoside, and/or the nucleoside at position +22 is of the second type of 5′ mixmer nucleoside, and/or the nucleoside at position +24 is of the second type of 5′ mixmer nucleoside. In some embodiments, the nucleoside at each of positions +2, +4, +6, +8, +10, +12, +14, +18, +20, +22 and +24 is of the second type of 5′ mixmer nucleoside.

In some embodiments, the nucleoside at one or more of positions −2, −4, −6, −10, −12 and −14 is of the second type of mixmer nucleoside. In some embodiments, the nucleoside at one or more of positions −2, −4, −6, −10, −12 and −14 is of the second type of 3′ mixmer nucleoside. In some embodiments, the nucleoside at position −2 is of the second type of 3′ mixmer nucleoside, and/or the nucleoside at position −4 is of the second type of 3′ mixmer nucleoside, and/or the nucleoside at position −6 is of the second type of 3′ mixmer nucleoside, and/or the nucleoside at position −10 is of the second type of 3′ mixmer nucleoside, and/or the nucleoside at position −12 is of the second type of 3′ mixmer nucleoside, and/or the nucleoside at position −14 is of the second type of 3′ mixmer nucleoside. In some embodiments, the nucleoside at each of positions −2, −4, −6, −10, −12 and −14 is of the second type of 3′ mixmer nucleoside.

In some embodiments of the oligonucleotide of the invention, the nucleosides of from position +2 to position +19 are the 5′ mixmer region, the nucleoside at each of positions +3, +5, +7, +9, +11, +13, +15, +17 and +19 is of the first type of 5′ mixmer nucleoside, and the nucleoside at each of positions +2, +4, +6, +8, +10, +12, +14, +16 and +18 is of the second type of 5′ mixmer nucleoside.

In some embodiments, the nucleoside at position +16 is of the second type of 3′ mixmer nucleoside. In some embodiments, the nucleoside at position −8 is of the second type of 3′ mixmer nucleoside.

In some embodiments of the oligonucleotide of the invention, the nucleosides of from position −2 to position −10 are the 3′ mixmer region, the nucleoside at each of positions −3, −5, −7 and −9 is of the first type of 3′ mixmer nucleoside, and the nucleoside at each of positions −2, −4, −6, −8 and −10 is of the second type of 3′ mixmer nucleoside.

In some embodiments of the oligonucleotide of the invention, the nucleosides of from position +2 to position +24 are the 5′ mixmer region, the nucleoside at each of positions +3, +5, +7, +9, +11, +13, +15, +17, +19, +21 and +23 is of the first type of 5′ mixmer nucleoside, and the nucleoside at each of positions +2, +4, +6, +8, +10, +12, +14, +16, +18, +20, +22 and +24 is of the second type of 5′ mixmer nucleoside.

In some embodiments of the oligonucleotide of the invention, the nucleosides of from position −2 to position −15 are the 3′ mixmer region, the nucleoside at each of positions −3, −5, −7, −9, −11, −13 and −15 is of the first type of 3′ mixmer nucleoside, and the nucleoside at each of positions −2, −4, −6, −8. −10, −12 and −14 is of the second type of 3′ mixmer nucleoside.

### Types of Mixmer Nucleosides

In some embodiments of the oligonucleotide of the invention, the mixmer regions comprise a first type of mixmer nucleoside and a second type of mixmer nucleoside, wherein the sugar moiety of the first type of mixmer nucleoside is different to the sugar moiety of the second type of mixmer nucleoside.

In some embodiments, the first type of mixmer nucleoside is selected from the group consisting of DNA, RNA, 2′-O-methyl-RNA, 2′-O-methoxyethyl-RNA (MOE-RNA), 2′-fluoro-RNA, linked nucleic acid (LNA), arabinonucleic acid (ANA) and 2′-fluoroarabinonucelic acid (FANA) nucleosides. In some embodiments, the first type of mixmer nucleoside is selected from the group consisting of RNA, 2′-O-methyl-RNA and 2′-fluoro-RNA nucleosides. In some embodiments, the first type of mixmer nucleoside is a 2′-fluoro-RNA nucleoside or a 2′-O-methyl-RNA nucleoside. In some embodiments, the first type of mixmer nucleoside is a 2′-fluoro-RNA nucleoside.

In some embodiments, the second type of mixmer nucleoside is selected from the group consisting of DNA, RNA, 2′-O-methyl-RNA, MOE-RNA, 2′-fluoro-RNA, LNA, ANA and FANA nucleosides. In some embodiments, the second type of mixmer nucleoside is selected from the group consisting of RNA, 2′-O-methyl-RNA and 2′-fluoro-RNA nucleosides. In some embodiments, the second type of mixmer nucleoside is a 2′-fluoro-RNA nucleoside or a 2′-O-methyl-RNA nucleoside. In some embodiments, the second type of mixmer nucleoside is a 2′-O-methyl-RNA nucleoside.

In some embodiments, the first type of 5′ mixmer nucleoside is selected from the group consisting of DNA, RNA, 2′-O-methyl-RNA, MOE-RNA, 2′-fluoro-RNA, LNA, ANA and FANA nucleosides. In some embodiments, the first type of 5′ mixmer nucleoside is selected from the group consisting of RNA, 2′-O-methyl-RNA and 2′-fluoro-RNA nucleosides. In some embodiments, the first type of 5′ mixmer nucleoside is a 2′-fluoro-RNA nucleoside or a 2′-O-methyl-RNA nucleoside. In some embodiments, the first type of 5′ mixmer nucleoside is a 2′-fluoro-RNA nucleoside.

In some embodiments, the second type of 5′ mixmer nucleoside is selected from the group consisting of DNA, RNA, 2′-O-methyl-RNA, MOE-RNA, 2′-fluoro-RNA, LNA, ANA and FANA nucleosides. In some embodiments, the second type of 5′ mixmer nucleoside is selected from the group consisting of RNA, 2′-O-methyl-RNA and 2′-fluoro-RNA nucleosides. In some embodiments, the second type of 5′ mixmer nucleoside is a 2′-fluoro-RNA nucleoside or a 2′-O-methyl-RNA nucleoside. In some embodiments, the second type of 5′ mixmer nucleoside is a 2′-O-methyl-RNA nucleoside.

In some embodiments, the first type of 3′ mixmer nucleoside is selected from the group consisting of DNA, RNA, 2′-O-methyl-RNA, MOE-RNA, 2′-fluoro-RNA, LNA, ANA and FANA nucleosides. In some embodiments, the first type of 3′ mixmer nucleoside is selected from the group consisting of RNA, 2′-O-methyl-RNA and 2′-fluoro-RNA nucleosides. In some embodiments, the first type of 3′ mixmer nucleoside is a 2′-fluoro-RNA nucleoside or a 2′-O-methyl-RNA nucleoside. In some embodiments, the first type of 3′ mixmer nucleoside is a 2′-fluoro-RNA nucleoside.

In some embodiments, the second type of 3′ mixmer nucleoside is selected from the group consisting of DNA, RNA, 2′-O-methyl-RNA, MOE-RNA, 2′-fluoro-RNA, LNA, ANA and FANA nucleosides. In some embodiments, the second type of 3′ mixmer nucleoside is selected from the group consisting of RNA, 2′-O-methyl-RNA and 2′-fluoro-RNA nucleosides. In some embodiments, the second type of 3′ mixmer nucleoside is a 2′-fluoro-RNA nucleoside or a 2′-O-methyl-RNA nucleoside. In some embodiments, the second type of 3′ mixmer nucleoside is a 2′-O-methyl-RNA nucleoside.

In some embodiments:
(a) the first type of 5′ mixmer nucleoside is selected from the group consisting of DNA, RNA, 2′-O-methyl-RNA, MOE-RNA, 2′-fluoro-RNA, LNA, ANA and FANA nucleosides; and/or
(b) the second type of 5′ mixmer nucleoside is selected from the group consisting of DNA, RNA, 2′-O-methyl-RNA, MOE-RNA, 2′-fluoro-RNA, LNA, ANA and FANA nucleosides; and/or
(c) the first type of 3′ mixmer nucleoside is selected from the group consisting of DNA, RNA, 2′-O-methyl-RNA, MOE-RNA, 2′-fluoro-RNA, LNA, ANA and FANA nucleosides; and/or
(d) the second type of 3′ mixmer nucleoside is selected from the group consisting of DNA, RNA, 2′-O-methyl-RNA, MOE-RNA, 2′-fluoro-RNA, LNA, ANA and FANA nucleosides.

In some embodiments, the first type of 5′ mixmer nucleoside is a 2′-fluoro-RNA nucleoside, and/or the second type of 5′ mixmer nucleoside is a 2′-O-methyl-RNA nucleoside, and/or the first type of 3′ mixmer nucleoside is a 2′-fluoro-RNA nucleoside, and/or the second type of 3′ mixmer nucleoside is a 2′-O-methyl-RNA nucleoside.

In some embodiments, the first type of mixmer nucleoside is a 2′-fluoro-RNA nucleoside and the second type of mixmer nucleoside is a 2′-O-methyl-RNA nucleoside. In some embodiments, the first type of mixmer nucleoside is a 2′-O-methyl-RNA nucleoside and the second type of mixmer nucleoside is a 2′-fluoro-RNA nucleoside. In some embodiments, the first type of 5′ mixmer nucleoside is a 2′-fluoro-RNA nucleoside, the second type of 5′ mixmer nucleoside is a 2′-O-methyl-RNA nucleoside, the first type of 3′ mixmer nucleoside is a 2′-fluoro-RNA nucleoside, and the second type of 3′ mixmer nucleoside is a 2′-O-methyl-RNA nucleoside.

The different types of sugar-modified nucleosides listed above are described in detail elsewhere herein.

### 2′-Fluoro-RNA and 2′-O-Methyl-RNA Mixmer Regions

In some embodiments, the mixmer region comprises 2′-fluoro-RNA nucleosides and 2′-O-methyl-RNA nucleosides. In some embodiments, the mixmer region consists of 2′-fluoro-RNA nucleosides and 2′-O-methyl-RNA nucleosides. In some embodiments, each nucleoside of the mixmer region is either a 2′-fluoro-RNA nucleoside or a 2′-O-methyl-RNA nucleoside. In some embodiments, the mixmer region comprises alternating 2′-fluoro-RNA nucleosides and 2′-O-methyl-RNA nucleosides. In some embodiments, the mixmer region consists of alternating 2′-fluoro-RNA nucleosides and 2′-O-methyl-RNA nucleosides.

In some embodiments, the 5′ mixmer region comprises 2′-fluoro-RNA nucleosides and 2′-O-methyl-RNA nucleosides. In some embodiments, the 5′ mixmer region consists of 2′-fluoro-RNA nucleosides and 2′-O-methyl-RNA nucleosides. In some embodiments, each nucleoside of the 5′ mixmer region is either a 2′-fluoro-RNA nucleoside or a 2′-O-methyl-RNA nucleoside. In some embodiments, the 5′ mixmer region comprises alternating 2′-fluoro-RNA nucleosides and 2′-O-methyl-RNA nucleosides. In some embodiments, the 5′ mixmer region consists of alternating 2′-fluoro-RNA nucleosides and 2′-O-methyl-RNA nucleosides.

In some embodiments, the 3′ mixmer region comprises 2′-fluoro-RNA nucleosides and 2′-O-methyl-RNA nucleosides. In some embodiments, each nucleoside of the 3′ mixmer region is either a 2′-fluoro-RNA nucleoside or a 2′-O-methyl-RNA nucleoside. In some embodiments, the 3′ mixmer region comprises alternating 2′-fluoro-RNA nucleosides and 2′-O-methyl-RNA nucleosides. In some embodiments, the 3′ mixmer region consists of alternating 2′-fluoro-RNA nucleosides and 2′-O-methyl-RNA nucleosides.

In some embodiments, each nucleoside at positions +2 to +19 is a 2′-fluoro-RNA nucleoside or a 2′-O-methyl-RNA nucleoside. In some embodiments, the nucleoside at position +2 is a 2′-fluoro-RNA nucleoside or a 2′-O-methyl-RNA nucleoside, and/or the nucleoside at position +3 is a 2′-fluoro-RNA nucleoside or a 2′-O-methyl-RNA nucleoside, and/or the nucleoside at position +4 is a 2′-fluoro-RNA nucleoside or a 2′-O-methyl-RNA nucleoside, and/or the nucleoside at position +5 is a 2′-fluoro-RNA nucleoside or a 2′-O-methyl-RNA nucleoside, and/or the nucleoside at position +6 is a 2′-fluoro-RNA nucleoside or a 2′-O-methyl-RNA nucleoside, and/or the nucleoside at position +7 is a 2′-fluoro-RNA nucleoside or a 2′-O-methyl-RNA nucleoside, and/or the nucleoside at position +8 is a 2′-fluoro-RNA nucleoside or a 2′-O-methyl-RNA nucleoside, and/or the nucleoside at position +9 is a 2′-fluoro-RNA nucleoside or a 2′-O-methyl-RNA nucleoside, and/or the nucleoside at position +10 is a 2′-fluoro-RNA nucleoside or a 2′-O-methyl-RNA nucleoside, and/or the nucleoside at position +11 is a 2′-fluoro-RNA nucleoside or a 2′-O-methyl-RNA nucleoside, and/or the nucleoside at position +12 is a 2′-fluoro-RNA nucleoside or a 2′-O-methyl-RNA nucleoside, and/or the nucleoside at position +13 is a 2′-fluoro-RNA nucleoside or a 2′-O-methyl-RNA nucleoside, and/or the nucleoside at position +14 is a 2′-fluoro-RNA nucleoside or a 2′-O-methyl-RNA nucleoside, and/or the nucleoside at position +15 is a 2′-fluoro-RNA nucleoside or a 2′-O-methyl-RNA nucleoside, and/or the nucleoside at position +16 is a 2′-fluoro-RNA nucleoside or a 2′-O-methyl-RNA nucleoside, and/or the nucleoside at position +17 is a 2′-fluoro-RNA nucleoside or a 2′-O-methyl-RNA nucleoside, and/or the nucleoside at position +18 is a 2′-fluoro-RNA nucleoside or a 2′-O-methyl-RNA nucleoside, and/or the nucleoside at position +19 is a 2′-fluoro-RNA nucleoside or a 2′-O-methyl-RNA nucleoside.

In some embodiments, each nucleoside at positions −2 to −9 is a 2′-fluoro-RNA nucleoside or a 2′-O-methyl-RNA nucleoside. In some embodiments, the nucleoside at position −2 is a 2′-fluoro-RNA nucleoside or a 2′-O-methyl-RNA nucleoside, and/or the nucleoside at position −3 is a 2′-fluoro-RNA nucleoside or a 2′-O-methyl-RNA nucleoside, and/or the nucleoside at position −4 is a 2′-fluoro-RNA nucleoside or a 2′-O-methyl-RNA nucleoside, and/or the nucleoside at position −5 is a 2′-fluoro-RNA nucleoside or a 2′-O-methyl-RNA nucleoside, and/or the nucleoside at position −6 is a 2′-fluoro-RNA nucleoside or a 2′-O-methyl-RNA nucleoside, and/or the nucleoside at position −7 is a 2′-fluoro-RNA nucleoside or a 2′-O-methyl-RNA nucleoside, and/or the nucleoside at position −8 is a 2′-fluoro-RNA nucleoside or a 2′-O-methyl-RNA nucleoside, and/or the nucleoside at position −9 is a 2′-fluoro-RNA nucleoside or a 2′-O-methyl-RNA nucleoside.

In some embodiments each nucleoside at positions +2 to +24 is a 2′-fluoro-RNA nucleoside or a 2′-O-methyl-RNA nucleoside. In some embodiments, the nucleoside at position +2 is a 2′-fluoro-RNA nucleoside or a 2′-O-methyl-RNA nucleoside, and/or the nucleoside at position +3 is a 2′-fluoro-RNA nucleoside or a 2′-O-methyl-RNA nucleoside, and/or the nucleoside at position +4 is a 2′-fluoro-RNA nucleoside or a 2′-O-methyl-RNA nucleoside, and/or the nucleoside at position +5 is a 2′-fluoro-RNA nucleoside or a 2′-O-methyl-RNA nucleoside, and/or the nucleoside at position +6 is a 2′-fluoro-RNA nucleoside or a 2′-O-methyl-RNA nucleoside, and/or the nucleoside at position +7 is a 2′-fluoro-RNA nucleoside or a 2′-O-methyl-RNA nucleoside, and/or the nucleoside at position +8 is a 2′-fluoro-RNA nucleoside or a 2′-O-methyl-RNA nucleoside, and/or the nucleoside at position +9 is a 2′-fluoro-RNA nucleoside or a 2′-O-methyl-RNA nucleoside, and/or the nucleoside at position +10 is a 2′-fluoro-RNA nucleoside or a 2′-O-methyl-RNA nucleoside, and/or the nucleoside at position +11 is a 2′-fluoro-RNA nucleoside or a 2′-O-methyl-RNA nucleoside, and/or the nucleoside at position +12 is a 2′-fluoro-RNA nucleoside or a 2′-O-methyl-RNA nucleoside, and/or the nucleoside at position +13 is a 2′-fluoro-RNA nucleoside or a 2′-O-methyl-RNA nucleoside, and/or the nucleoside at position +14 is a 2′-fluoro-RNA nucleoside or a 2′-O-methyl-RNA nucleoside, and/or the nucleoside at position +15 is a 2′-fluoro-RNA nucleoside or a 2′-O-methyl-RNA nucleoside, and/or the nucleoside at position +16 is a 2′-fluoro-RNA nucleoside or a 2′-O-methyl-RNA nucleoside, and/or the nucleoside at position +17 is a 2′-fluoro-RNA nucleoside or a 2′-O-methyl-RNA nucleoside, and/or the nucleoside at position +18 is a 2′-fluoro-RNA nucleoside or a 2′-O-methyl-RNA nucleoside, and/or the nucleoside at position +19 is a 2′-fluoro-RNA nucleoside or a 2′-O-methyl-RNA nucleoside, and/or the nucleoside at position +20 is a 2′-fluoro-RNA nucleoside or a 2′-O-methyl-RNA nucleoside, and/or the nucleoside at position +21 is a 2′-fluoro-RNA nucleoside or a 2′-O-methyl-RNA nucleoside, and/or the nucleoside at position +22 is a 2′-fluoro-RNA nucleoside or a 2′-O-methyl-RNA nucleoside, and/or the nucleoside at position +23 is a 2′-fluoro-RNA nucleoside or a 2′-O-methyl-RNA nucleoside, and/or the nucleoside at position +24 is a 2′-fluoro-RNA nucleoside or a 2′-O-methyl-RNA nucleoside.

In some embodiments, each nucleoside at positions −2 to −15 is a 2′-O-methyl-RNA nucleoside or a 2′-fluoro-RNA nucleoside. In some embodiments, the nucleoside at position −2 is a 2′-fluoro-RNA nucleoside or a 2′-O-methyl-RNA nucleoside, and/or the nucleoside at position −3 is a 2′-fluoro-RNA nucleoside or a 2′-O-methyl-RNA nucleoside, and/or the nucleoside at position −4 is a 2′-fluoro-RNA nucleoside or a 2′-O-methyl-RNA nucleoside, and/or the nucleoside at position −5 is a 2′-fluoro-RNA nucleoside or a 2′-O-methyl-RNA nucleoside, and/or the nucleoside at position −6 is a 2′-fluoro-RNA nucleoside or a 2′-O-methyl-RNA nucleoside, and/or the nucleoside at position −7 is a 2′-fluoro-RNA nucleoside or a 2′-O-methyl-RNA nucleoside, and/or the nucleoside at position −8 is a 2′-fluoro-RNA nucleoside or a 2′-O-methyl-RNA nucleoside, and/or the nucleoside at position −9 is a 2′-fluoro-RNA nucleoside or a 2′-O-methyl-RNA nucleoside, and/or the nucleoside at position −10 is a 2′-fluoro-RNA nucleoside or a 2′-O-methyl-RNA nucleoside, and/or the nucleoside at position −11 is a 2′-fluoro-RNA nucleoside or a 2′-O-methyl-RNA nucleoside, and/or the nucleoside at position −12 is a 2′-fluoro-RNA nucleoside or a 2′-O-methyl-RNA nucleoside, and/or the nucleoside at position −13 is a 2′-fluoro-RNA nucleoside or a 2′-O-methyl-RNA nucleoside, and/or the nucleoside at position −14 is a 2′-fluoro-RNA nucleoside or a 2′-O-methyl-RNA nucleoside, and/or the nucleoside at position −15 is a 2′-fluoro-RNA nucleoside or a 2′-O-methyl-RNA nucleoside.

A 2′-fluoro-RNA nucleoside or 2′-O-methyl-RNA nucleoside at particular positions provides enhanced editing efficiency. Thus, in some embodiments, the nucleoside at position −2 is a 2′-O-methyl-RNA nucleoside. In some embodiments, the nucleoside at position −3 is a 2′-fluoro-RNA nucleoside. In some embodiments, the nucleoside at position −8 is a 2′-fluoro-RNA nucleoside. In some embodiments, the nucleoside at position +16 is a 2′-fluoro-RNA nucleoside. In some embodiments, the nucleoside at position +17 is a 2′-fluoro-RNA nucleoside.

In some embodiments, the nucleoside at one or more of positions +3, +5, +7, +9, +11, +13, +15, +17 and +19 is a 2′-fluoro-RNA nucleoside. In some embodiments, the nucleoside at position +3 is a 2′-fluoro-RNA nucleoside, and/or the nucleoside at position +5 is a 2′-fluoro-RNA nucleoside, and/or the nucleoside at position +7 is a 2′-fluoro-RNA nucleoside, and/or the nucleoside at position +9 is a 2′-fluoro-RNA nucleoside, and/or the nucleoside at position +11 is a 2′-fluoro-RNA nucleoside, and/or the nucleoside at position +13 is a 2′-fluoro-RNA nucleoside, and/or the nucleoside at position +15 is a 2′-fluoro-RNA nucleoside, and/or the nucleoside at position +17 is a 2′-fluoro-RNA nucleoside, and/or the nucleoside at position +19 is a 2′-fluoro-RNA nucleoside. In some embodiments the nucleoside at each of positions +3, +5, +7, +9, +11, +13, +15, +17 and +19 is a 2′-fluoro-RNA nucleoside.

In some embodiments, the nucleoside at one or more of positions −3, −5, −7 and −9 is a 2′-fluoro-RNA nucleoside. In some embodiments, the nucleoside at position −3 is a 2′-fluoro-RNA nucleoside, and/or the nucleoside at position −5 is a 2′-fluoro-RNA nucleoside, and/or the nucleoside at position −7 is a 2′-fluoro-RNA nucleoside, and/or the nucleoside at position −9 is a 2′-fluoro-RNA nucleoside. In some embodiments, the nucleoside at each of positions −3, −5, −7 and −9 is a 2′-fluoro-RNA nucleoside.

In some embodiments, the nucleoside at one or more of positions +2, +4, +6, +8, +10, +12, +14 and +18 is a 2′-O-methyl-RNA nucleoside. In some embodiments, the nucleoside at position +2 is a 2′-O-methyl-RNA nucleoside, and/or the nucleoside at position +4 is a 2′-O-methyl-RNA nucleoside, and/or the nucleoside at position +6 is a 2′-O-methyl-RNA nucleoside, and/or the nucleoside at position +8 is a 2′-O-methyl-RNA nucleoside, and/or the nucleoside at position +10 is a 2′-O-methyl-RNA nucleoside, and/or the nucleoside at position +12 is a 2′-O-methyl-RNA nucleoside, and/or the nucleoside at position +14 is a 2′-O-methyl-RNA nucleoside, and/or the nucleoside at position +18 is a 2′-O-methyl-RNA nucleoside. In some embodiments, the nucleoside at each of positions +2, +4, +6, +8, +10, +12, +14 and +18 is a 2′-O-methyl-RNA nucleoside.

In some embodiments, the nucleoside at one or more of positions −2, −4 and −6 is a 2′-O-methyl-RNA nucleoside. In some embodiments, the nucleoside at position −2 is a 2′-O-methyl-RNA nucleoside, and/or the nucleoside at position −4 is a 2′-O-methyl-RNA nucleoside, and/or the nucleoside at position −6 is a 2′-O-methyl-RNA nucleoside. In some embodiments, the nucleoside at each of positions −2, −4 and −6 is a 2′-O-methyl-RNA nucleoside.

In some embodiments, the nucleoside at one or more of positions +3, +5, +7, +9, +11, +13, +15, +17, +19, +21 and +23 is a 2′-fluoro-RNA nucleoside. In some embodiments, the nucleoside at position +3 is a 2′-fluoro-RNA nucleoside, and/or the nucleoside at position +5 is a 2′-fluoro-RNA nucleoside, and/or the nucleoside at position +7 is a 2′-fluoro-RNA nucleoside, and/or the nucleoside at position +9 is a 2′-fluoro-RNA nucleoside, and/or the nucleoside at position +11 is a 2′-fluoro-RNA nucleoside, and/or the nucleoside at position +13 is a 2′-fluoro-RNA nucleoside, and/or the nucleoside at position +15 is a 2′-fluoro-RNA nucleoside, and/or the nucleoside at position +17 is a 2′-fluoro-RNA nucleoside, and/or the nucleoside at position +19 is a 2′-fluoro-RNA nucleoside, and/or the nucleoside at position +21 is a 2′-fluoro-RNA nucleoside, and/or the nucleoside at position +23 is a 2′-fluoro-RNA nucleoside. In some embodiments, the nucleoside at each of positions +3, +5, +7, +9, +11, +13, +15, +17, +19, +21 and +23 is a 2′-fluoro-RNA nucleoside.

In some embodiments, the nucleoside at one or more of positions −3, −5, −7, −9, −11, −13 and −15 is a 2′-fluoro-RNA nucleoside. In some embodiments, the nucleoside at position −3 is a 2′-fluoro-RNA nucleoside, and/or the nucleoside at position −5 is a 2′-fluoro-RNA nucleoside, and/or the nucleoside at position −7 is a 2′-fluoro-RNA nucleoside, and/or the nucleoside at position −9 is a 2′-fluoro-RNA nucleoside, and/or the nucleoside at position −11 is a 2′-fluoro-RNA nucleoside, and/or the nucleoside at position −13 is a 2′-fluoro-RNA nucleoside, and/or the nucleoside at position −15 is a 2′-fluoro-RNA nucleoside. In some embodiments, the nucleoside at each of positions −3, −5, −7, −9, −11, −13 and −15 is a 2′-fluoro-RNA nucleoside.

In some embodiments, the nucleoside at one or more of positions +2, +4, +6, +8, +10, +12, +14, +18, +20, +22 and +24 is a 2′-O-methyl-RNA nucleoside. In some embodiments, the nucleoside at position +2 is a 2′-O-methyl-RNA nucleoside, and/or the nucleoside at position +4 is a 2′-O-methyl-RNA nucleoside, and/or the nucleoside at position +6 is a 2′-O-methyl-RNA nucleoside, and/or the nucleoside at position +8 is a 2′-O-methyl-RNA nucleoside, and/or the nucleoside at position +10 is a 2′-O-methyl-RNA nucleoside, and/or the nucleoside at position +12 is a 2′-O-methyl-RNA nucleoside, and/or the nucleoside at position +14 is a 2′-O-methyl-RNA nucleoside, and/or the nucleoside at position +18 is a 2′-O-methyl-RNA nucleoside, and/or the nucleoside at position +20 is a 2′-O-methyl-RNA nucleoside, and/or the nucleoside at position +22 is a 2′-O-methyl-RNA nucleoside, and/or the nucleoside at position +24 is a 2′-O-methyl-RNA nucleoside. In some embodiments, the nucleoside at each of positions +2, +4, +6, +8, +10, +12, +14, +18, +20, +22 and +24 is a 2′-O-methyl-RNA nucleoside.

In some embodiments, the nucleoside at one or more of positions −2, −4, −6, −10, −12 and −14 is a 2′-O-methyl-RNA nucleoside. In some embodiments, the nucleoside at position −2 is a 2′-O-methyl-RNA nucleoside, and/or the nucleoside at position −4 is a 2′-O-methyl-RNA nucleoside, and/or the nucleoside at position −6 is a 2′-O-methyl-RNA nucleoside, and/or the nucleoside at position −10 is a 2′-O-methyl-RNA nucleoside, and/or the nucleoside at position −12 is a 2′-O-methyl-RNA nucleoside, and/or the nucleoside at position −14 is a 2′-O-methyl-RNA nucleoside. In some embodiments, the nucleoside at each of positions −2, −4, −6, −10, −12 and −14 is a 2′-O-methyl-RNA nucleoside.

In some embodiments, the nucleoside at position −8 is a 2′-O-methyl-RNA nucleoside. In some embodiments, the nucleoside at position +16 is a 2′-O-methyl-RNA nucleoside.

### Sugar-Modified Nucleosides

In some embodiments, the oligonucleotide of the invention comprises one or more sugar-modified nucleosides. In other words, the oligonucleotides of the invention may comprise one or more nucleosides which have a modified sugar moiety, i.e. a modification of the sugar moiety when compared to the ribose sugar moiety found in DNA and RNA. In particular, as described herein, the mixmer region of oligonucleotides of the invention comprises sugar-modified nucleosides.

Numerous nucleosides with modification of the ribose sugar moiety have been developed, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar-modified nucleosides include, for example, bicyclohexose nucleic acids (WO 2011/017521) or tricyclic nucleic acids (WO 2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar-Modified Nucleosides

In some embodiments, the oligonucleotide of the invention comprises one or more 2′ sugar-modified nucleosides. A 2′ sugar-modified nucleoside is a nucleoside which has a substituent other than —H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′OMe), 2′-alkoxy-RNA, 2′-O-methoxyethyl-RNA (MOE), 2′-amino-DNA, 2′-fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development 2000, 3(2), 203-213, and Deleavey and Damha, Chemistry and Biology 2012, 19, 937. Below are illustrations of some 2′ substituted modified nucleosides.

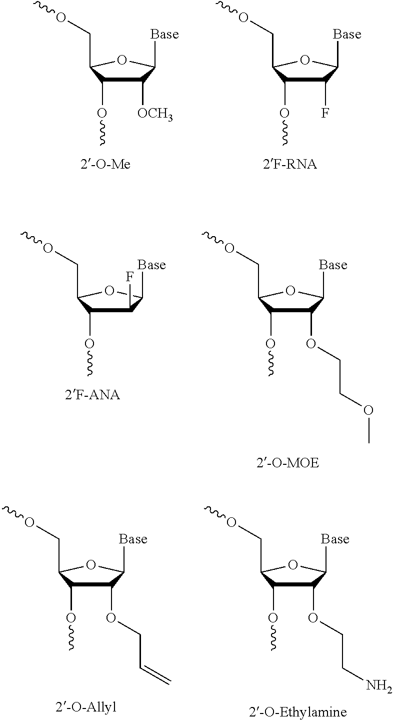

### Locked Nucleic Acid Nucleosides (LNA Nucleoside)

In some embodiments, the oligonucleotide comprises one or more LNA nucleosides.

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non-limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett., 12, 73-76, Seth et al., J. Org. Chem., 2010, Vol 75(5) pp. 1569-81, Mitsuoka et al., Nucleic Acids Research, 2009, 37(4), 1225-1238, and Wan and Seth, J. Medical Chemistry, 2016, 59, 9645-9667.

Further non limiting, exemplary LNA nucleosides are disclosed in Scheme 1.

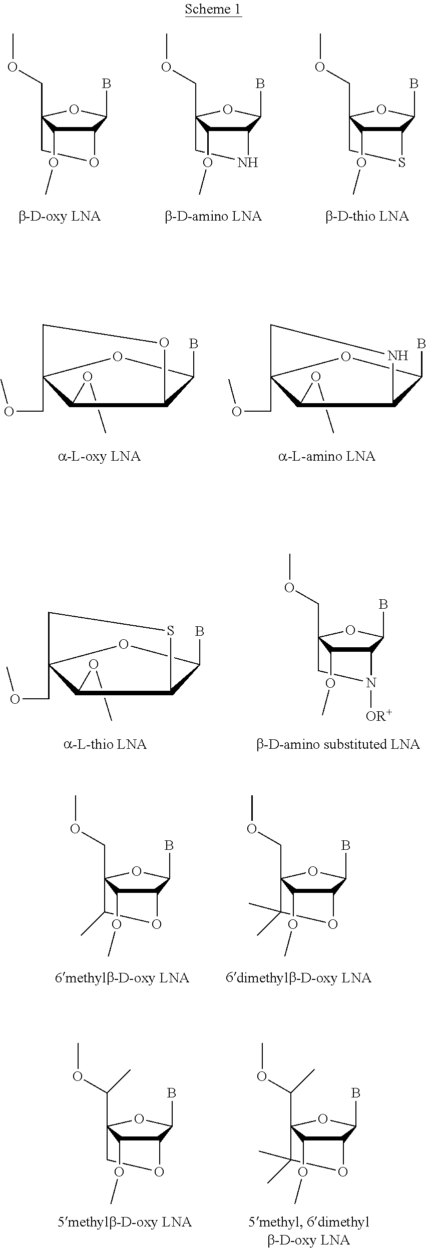

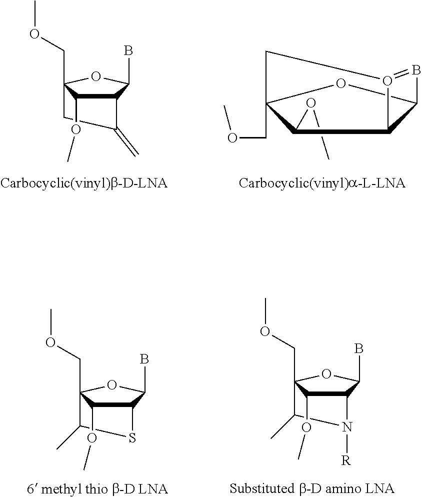

Particular LNA nucleosides are beta-D-oxy-LNA, 6′-methyl-beta-D-oxy LNA such as (S)-6′-methyl-beta-D-oxy-LNA (ScET) and ENA.

In some embodiments, the oligonucleotide comprises one or more LNA beta-D-oxy-LNA nucleosides.

### Oligonucleotide Length

In some embodiments, the oligonucleotide of the invention is from 30 to 61 nucleosides long. In some embodiments, the oligonucleotide is from 30 to 50 nucleosides long. In some embodiments, the oligonucleotide is from 35 to 45 nucleosides long.

In some embodiments, the oligonucleotide is 30, 31, 32, 33, 34, 35, 36, 37, 38, 39, 40, 41, 42, 43, 44, 45, 46, 47, 48, 49 or 50 nucleosides long. In some embodiments, the oligonucleotide is 35, 36, 37, 38, 39, 40, 41, 42, 43, 44 or 45 nucleosides long. In some embodiments, the oligonucleotide is 40 nucleosides long.

### Oligonucleotide Symmetry

The term “symmetry” refers to the number of nucleosides on either side of the editing nucleoside in the oligonucleotide. In other words, the symmetry of the oligonucleotide describes the number of nucleosides 5′ to the editing nucleoside and the number of nucleosides 3 to the editing nucleoside. The symmetry of an oligonucleotide of the invention may be described herein using the formula “X-1-Y”, wherein “X” is the number of nucleosides 5′ to the editing nucleoside, “1” represents the editing nucleoside, and “Y” is the number of nucleosides 3′ to the editing nucleoside. For example, an oligonucleotide with 24-1-15 symmetry is 40 nucleosides long, having exactly 24 nucleosides 5′ to the editing nucleoside and exactly 15 nucleosides 3′ to the editing nucleoside.

In some embodiments, the oligonucleotide comprises from 20 to 30 nucleosides 5′ to the editing nucleoside. In some embodiments, the oligonucleotide comprises from 22 to 29 nucleosides 5′ to the editing nucleoside. In some embodiments, the oligonucleotide comprises from 24 to 27 nucleosides 5′ to the editing nucleoside. In some embodiments, the oligonucleotide comprises 24 nucleosides 5′ to the editing nucleoside.

In some embodiments, the oligonucleotide comprises exactly 20, 21, 22, 23, 24, 25, 26, 27, 28, 29 or 30 nucleosides 5′ to the editing nucleoside. In some embodiments, the oligonucleotide comprises exactly 22, 23, 24, 25, 26, 27, 28 or 29 nucleosides 5′ to the editing nucleoside. In some embodiments, the oligonucleotide comprises exactly 24, 25, 26 or 27 nucleosides 5′ to the editing nucleoside. In some embodiments, the oligonucleotide comprises exactly 24 nucleosides 5′ to the editing nucleoside.

In some embodiments, the oligonucleotide comprises 4 to 20 nucleosides 3′ to the editing nucleoside. In some embodiments, the oligonucleotide comprises 12 to 15 nucleosides 3′ to the editing nucleoside. In some embodiments, the oligonucleotide comprises 15 nucleosides 3′ to the editing nucleoside.

In some embodiments, the oligonucleotide comprises exactly 4, 5, 6, 7, 8, 9, 10, 11, 12, 13, 14, 15, 16, 17, 18, 19 or 20 nucleosides 3′ to the editing nucleoside. In some embodiments, the oligonucleotide comprises exactly 12, 13, 14 or 15 nucleosides 3′ to the editing nucleoside. In some embodiments, the oligonucleotide comprises exactly 15 nucleosides 3′ to the editing nucleoside.

In some embodiments, the oligonucleotide comprises exactly 22 to 29 nucleosides 5′ to the editing nucleoside and 12 to 15 nucleosides 3′ to the editing nucleoside. In some embodiments, the oligonucleotide comprises 24 to 27 nucleosides 5′ to the editing nucleoside and 14 or 15 nucleosides 3′ to the editing nucleoside.

In some embodiments, the oligonucleotide comprises exactly 22, 23, 24, 25, 26, 27, 28 or 29 nucleosides 5′ to the editing nucleoside and exactly 12, 13, 14 or 15 nucleosides 3′ to the editing nucleoside. In some embodiments, the oligonucleotide comprises exactly 24, 25, 26 or 27 nucleosides 5′ to the editing nucleoside and exactly 14 or 15 nucleosides 3′ to the editing nucleoside.

In some embodiments, the oligonucleotide comprises 21 to 30 nucleosides 5′ to the editing nucleoside and 15 nucleosides 3′ to the editing nucleoside. In some embodiments, the oligonucleotide comprises exactly 22 to 27 nucleosides 5′ to the editing nucleoside and 15 nucleosides 3′ to the editing nucleoside. In some embodiments, the oligonucleotide comprises 24 nucleosides 5′ to the editing nucleoside and 15 nucleosides 3′ to the editing nucleoside.

In some embodiments, the oligonucleotide comprises exactly 21, 22, 23, 24, 25, 26, 27, 28, 29 or 30 nucleosides 5′ to the editing nucleoside and exactly 15 nucleosides 3′ to the editing nucleoside. In some embodiments, the oligonucleotide comprises exactly 22, 23, 24, 25, 26 or 27 nucleosides 5′ to the editing nucleoside and exactly 15 nucleosides 3′ to the editing nucleoside. In some embodiments, the oligonucleotide comprises exactly 24 nucleosides 5′ to the editing nucleoside and exactly 15 nucleosides 3′ to the editing nucleoside.

In some embodiments, the oligonucleotide comprises exactly 25 nucleosides 5′ to the editing nucleoside and exactly 14 nucleosides 3′ to the editing nucleoside. In some embodiments, the oligonucleotide comprises exactly 26 nucleosides 5′ to the editing nucleoside and exactly 13 nucleosides 3′ to the editing nucleoside. In some embodiments, the oligonucleotide comprises exactly 27 nucleosides 5′ to the editing nucleoside and exactly 12 nucleosides 3′ to the editing nucleoside. In some embodiments, the oligonucleotide comprises exactly 28 nucleosides 5′ to the editing nucleoside and exactly 11 nucleosides 3′ to the editing nucleoside. In some embodiments, the oligonucleotide comprises exactly 29 nucleosides 5′ to the editing nucleoside and exactly 10 nucleosides 3′ to the editing nucleoside.

### Flank Regions

In some embodiments, the oligonucleotide of the invention comprises one or more flank regions. The term “flank region” refers to a stretch of nucleosides in the oligonucleotide that is in a flanking position relative to the editing nucleoside and mixmer regions. In other words, a flank region is outside both the editing region and mixmer regions within the oligonucleotide. In other words, a flank region is either 5′ or 3′ to both the editing region and mixmer regions.

In some embodiments, the oligonucleotide comprises a 5′ flank region positioned 5′ to the 5′ mixmer region. In some embodiments, the oligonucleotide comprises a 3′ flank region positioned 3′ to the 3′ mixmer region. In some embodiments, comprises a 5′ flank region positioned 5′ to the 5′ mixmer region and a 3′ flank region positioned 3′ to the 3′ mixmer region.

In some embodiments, the 5′ flank region is positioned immediately 5′ to the 5′ mixmer region. In other words, there are no additional nucleosides between the 3′-most nucleoside of the 5′ flank region and the 5′-most nucleoside of the 5′ mixmer region; the 3′-most nucleoside of the 5′ flank region and the 5′-most nucleoside of the 5′ mixmer region are linked by an internucleoside linkage. In some embodiments, the 3′ flank region is positioned immediately 3′ to the 3′ mixmer region. In other words, there are no additional nucleosides between the 5′-most nucleoside of the 3′ flank region and the 3′-most nucleoside of the 3′ mixmer region; the 5′-most nucleoside of the 3′ flank region and the 3′-most nucleoside of the 3′ mixmer region are linked by an internucleoside linkage. In some embodiments, the 5′ flank region is positioned immediately 5′ to the 5′ mixmer region and the 3′ flank region is positioned immediately 3′ to the 3′ mixmer region.

In some embodiments, the oligonucleotide comprises the structure:

X⁺²—X⁺¹—X⁰—X⁻¹—X⁻²
wherein
X⁺² is the 5′ flank region,
X⁺¹ is the 5′ mixmer region,
X⁰ is the editing region,
X⁻¹ is the 3′ mixmer region, and
X⁻¹ is the 3′ flank region.

In other words, in some embodiments, the 5′ flank region is immediately 5′ to the 5′ mixmer region, which is immediately 5′ to the editing nucleoside, which is immediately 5′ to the 3′ mixmer region, which is immediately 5′ to the 3′ flank region. The 5′ flank region, 5′ mixmer region, editing region, 3′ mixmer region and 3′ flank region are thus contiguous (i.e. linked by internucleoside linkages).

In some embodiments, the 5′ flank region is 1, 2, 3, 4 or 5 nucleosides long. In some embodiments, the 5′ flank region is 5 nucleosides long. In some embodiments, the 3′ flank region is 1, 2, 3, 4 or 5 nucleosides long. In some embodiments, the 3′ flank region is 5 nucleosides long. In some embodiments, both the 5′ flank region and the 3′ flank region are each 5 nucleosides long.

In some embodiments, the flank regions are the ends of the oligonucleotide. In some embodiments, the 5′ flank region is the 5′ end of the oligonucleotide. In other words, in some embodiments, the 5′-most nucleosides of the oligonucleotide are the 5′ flank region. In some embodiments, the 3′ flank region is the 3′ end of the oligonucleotide. In other words, in some embodiments, the 3′-most nucleosides of the oligonucleotide are the 3′ flank region. In some embodiments, the 5′ flank region consists of the 1, 2, 3, 4 or 5 nucleosides at the 5′ end of the oligonucleotide. In some embodiments, the 5′ flank region consists of the 5 nucleosides at the 5′ end of the oligonucleotide. In some embodiments, the 3′ flank region consists of the 1, 2, 3, 4 or 5 nucleosides at the 3′ end of the oligonucleotide. In some embodiments, the 3′ flank region consists of the 5 nucleosides at the 3′ end of the oligonucleotide.

In some embodiments, the 5′ flank region comprises one or more sugar-modified nucleosides. In some embodiments, the 3′ flank region comprises one or more sugar-modified nucleosides. In some embodiments, each of the 5′ flank region and 3′ flank region comprises one or more sugar-modified nucleosides. In some embodiments, the one or more sugar-modified nucleosides in the 5′ flank region or the 3′ flank region are independently selected from the group consisting of 2′-O-methyl-RNA, 2′-fluoro-RNA, 2′-O-methoxyethyl-RNA (MOE-RNA) and LNA nucleosides.

In some embodiments, all nucleosides of the 5′ flank region are sugar-modified nucleosides. In some embodiments, all nucleosides of the 3′ flank region are sugar-modified nucleosides. In some embodiments, all nucleosides of both the 5′ flank region and the 3′ flank region are sugar-modified nucleosides. In some embodiments, all nucleosides of the 5′ flank region are 2′-O-methyl-RNA nucleosides. In some embodiments, all nucleosides of the 3′ flank region are 2′-O-methyl-RNA nucleosides. In some embodiments, all nucleosides of both the 5′ flank region and the 3′ flank region are 2′-O-methyl-RNA nucleosides.

In some embodiments, the 5′ flank region consists of five 2′-O-methyl-RNA nucleosides. In some embodiments, 3′ flank region consists of five 2′-O-methyl-RNA nucleosides. In some embodiments, the 5′ flank region consists of five 2′-O-methyl-RNA nucleosides and the 3′ flank region consists of five 2′-O-methyl-RNA nucleosides.

In some embodiments, the 5′ flank region consists of five 2′-O-methyl-RNA nucleosides at the 5′ end of the oligonucleotide. In some embodiments, 3′ flank region consists of five 2′-O-methyl-RNA nucleosides at the 3′ end of the oligonucleotide. In some embodiments, the 5′ flank region consists of five 2′-O-methyl-RNA nucleosides at the 5′ end of the oligonucleotide and the 3′ flank region consists of five 2′-O-methyl-RNA nucleosides at the 3′ end of the oligonucleotide.

In some embodiments, the 5′ flank region comprises the nucleosides at positions +20 to +24 (i.e. positions +20, +21, +22, +23 and +24). In some embodiments, the nucleosides at positions +20 to +24 are 2′-O-methyl-RNA nucleosides. In some embodiments, the 3′ flank region comprises the nucleosides at positions −11 to −15 (i.e. positions −1, −12, −13, −14 and −15). In some embodiments, the nucleosides at positions −11 to −15 are 2′-O-methyl-RNA nucleosides.

### Modified Internucleoside Linkages

In some embodiments, the oligonucleotide of the invention may comprise one or more modified internucleoside linkages.

The term “modified internucleoside linkage” is defined, as generally understood by the skilled person, as linkages other than phosphodiester (PO) linkages that covalently couple two nucleosides together.

In some embodiments, all internucleoside linkages in the oligonucleotide are modified internucleoside linkages.

In some embodiments, each modified internucleoside linkage is independently selected from the group consisting of phosphorothioate internucleoside linkages and phosphorodithioate internucleoside linkages. In some embodiments, the oligonucleotide comprises one or more phosphorothioate internucleoside linkages. In some embodiments, all internucleoside linkages of the oligonucleotide are phosphorothioate internucleoside linkages.

Phosphorothioate internucleoside linkages and phosphorodithioate internucleoside linkages are useful in that they may render the oligonucleotide more resistant to degradation by nucleases. In a phosphorothioate internucleoside linkage, relative to a naturally occurring phosphodiester internucleoside linkage, one of the oxygen atoms in the phosphate group that is not bonded to a carbon of a nucleoside sugar moiety is replaced with a sulphur atom. In a phosphorodithioate internucleoside linkage, each of the two oxygen atoms in the phosphate group that is not bonded to a carbon of a nucleoside sugar moiety is replaced with a sulphur atom. Thus, where a phosphodiester bond may be represented by the formula —O—P(O)₂—O—, a phosphorothioate internucleoside linkage may be represented by the formula —O—P(O,S)—O—, and a phosphorodithioate internucleoside linkage may be represented by the formula —O—P(S)₂—O—.

Phosphorothioate internucleoside linkages are chiral (see, for example, Jahns et al. 2022 Nucleic Acids Research Vol. 50, No. 3, 1221-1240), with right-handed (Rp) and left-handed (Sp) isomers. The Rp diastereomer may be referred to as an R-PS internucleoside linkage or an srP internucleoside linkage. The Sp diastereomer may be referred to as an S-PS internucleoside linkage or ssP internucleoside linkage. In some embodiments, the oligonucleotide comprises one or more srP internucleoside linkages. In some embodiments, the oligonucleotide comprises one or more ssP internucleoside linkages. Where the chirality of a phosphorothioate internucleoside linkage is not specified, that phosphorothioate internucleoside linkage may be either an srP linkage or an ssP linkage. The structures of srP and ssP linkages are shown below:

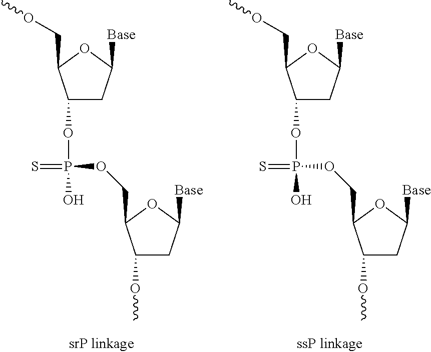

Particular types of internucleoside linkages between particular nucleosides may improve editing efficiency of the oligonucleotide.

In some embodiments, the internucleoside linkage between nucleosides at positions +1 and 0 is a phosphodiester internucleoside linkage. In some embodiments, the internucleoside linkage between nucleosides at positions +1 and 0 is a phosphodiester internucleoside linkage and all other internucleoside linkages are phosphorothioate internucleoside linkages.

In some embodiments, the internucleoside linkage between nucleosides at positions 0 and −1 is a phosphodiester internucleoside linkage. In some embodiments, the internucleoside linkage between nucleosides at positions 0 and −1 is a phosphodiester internucleoside linkage and all other internucleoside linkages are phosphorothioate internucleoside linkages.

In some embodiments, the internucleoside linkage between nucleosides at positions −1 and −2 is a phosphorothioate internucleoside linkage.

In some embodiments, the internucleoside linkage between nucleosides at positions −2 and −3 is a phosphodiester internucleoside linkage. In some embodiments, the internucleoside linkage between nucleosides at positions −2 and −3 is a phosphodiester internucleoside linkage and all other internucleoside linkages are phosphorothioate internucleoside linkages.

In some embodiments, all internucleoside linkages in the oligonucleotide are phosphorothioate internucleoside linkages except that:
the internucleoside linkage between nucleosides at positions +1 and 0 is a phosphodiester internucleoside linkage; and/or
the internucleoside linkage between nucleosides at positions 0 and −1 is a phosphodiester internucleoside linkage; and/or
the internucleoside linkage between nucleosides at positions −2 and −3 is a phosphodiester internucleoside linkage.

### Complementarity

In some embodiments, the oligonucleotide of the invention is complementary to a target nucleic acid. In some embodiments, oligonucleotide of the invention comprises or consists of a sequence that is complementary to a target nucleic acid. In some embodiments, the target nucleic acid is SERPINA1 mRNA. Thus, in some embodiments, the oligonucleotide of the invention is complementary to SERPINA1 mRNA. In some embodiments, oligonucleotide of the invention comprises or consists of a sequence that is complementary to a SERPINA1 mRNA.

The term “complementarity” describes the capacity for Watson-Crick base-pairing of nucleosides/nucleotides. Watson-Crick base pairs are cytosine-guanine (C-G) and adenine-thymine/uracil (A-T/U). It will be understood that oligonucleotides may comprise nucleosides with modified nucleobases. For example 5-methyl cytosine (E) may be used in place of cytosine and 7-deaza-8-azaguanine (F) may be used in place of guanine. The term complementarity encompasses Watson-Crick base-pairing between non-modified and modified nucleobases (see for example Hirao et al., 2012, Accounts of Chemical Research, 45, 2055 and Bergstrom, 2009, Curr. Protoc. Nucleic Acid Chem., 37, 1.4.1). In particular, Watson-Crick base-pairing encompasses E-G, C-F and E-F base pairs. Thus, an oligonucleotide that is complementary to a target nucleic acid forms base-pairs with the target nucleic acid, thereby binding to the target nucleic acid. An oligonucleotide that is complementary to SERPINA1 mRNA forms base-pairs with the SERPINA1 mRNA, thereby binding to the SERPINA1 mRNA.

“Complementarity” does not require that the oligonucleotide is complementary to a target nucleic acid across the entire length of the target nucleic acid. The oligonucleotide is typically shorter than the target nucleic acid. Rather, “complementarity” refers to the proportion of the nucleobases/nucleotides of the oligonucleotide which form base-pairs with the target nucleic acid. In some embodiments, the oligonucleotide is complementary to a portion of the target nucleic acid. In other words, the oligonucleotide is complementary to a sequence in a target nucleic acid. In some embodiments, the oligonucleotide is complementary to a portion of the target nucleic acid around a target adenosine. In some embodiments, the oligonucleotide is complementary to a portion of the SERPINA1 mRNA. In other words, the oligonucleotide is complementary to a sequence in a SERPINA1 mRNA. In some embodiments, the oligonucleotide is complementary to a portion of the SERPINA1 mRNA around a target adenosine.

As described herein, A1AD is associated with a single base pair substitution leading to mutation in A1AT of glutamate (E) 342 to lysine (K) (E342K mutation). Thus, in some embodiments, the oligonucleotide is complementary to a portion of SERPINA1 mRNA around a mutant AAG codon encoding lysine. In other words, in some embodiments, the oligonucleotide is complementary to a portion of SERPINA1 mRNA around a target adenosine that is the first nucleoside of a mutant AAG codon encoding lysine.

“Complementarity” does not require that the oligonucleotide is complementary to a contiguous sequence within the target nucleic acid. There may be mismatches between the oligonucleotide and the target nucleic acid. Therefore, complementarity between the oligonucleotide of the invention and the target nucleic acid may be expressed as a percentage. Furthermore, it will be understood that the target adenosine on the target nucleic acid and the editing nucleoside on the oligonucleotide do not form base-pairs. In other words, the target adenosine and editing nucleoside form a mismatch.

Thus, in some embodiments, the sequence of the oligonucleotide of the invention comprises or consists of a sequence having at least 80% complementarity to a sequence in a target nucleic acid, wherein any match or mismatch between the editing nucleoside and the target adenosine is not included when determining the percentage complementarity. In some embodiments, the sequence of the oligonucleotide of the invention comprises or consists of a sequence having at least 85%, at least 90%, at least 95%, at least 96%, at least 97%, at least 98%, at least 99% or 100% complementarity to a sequence in a target nucleic acid, wherein any match or mismatch between the editing nucleoside and the target adenosine is not included when determining the percentage complementarity.

In some embodiments, the sequence of the oligonucleotide of the invention comprises or consists of a sequence having at least 80% complementarity to a sequence in a SERPINA1 mRNA, wherein any match or mismatch between the editing nucleoside and the target adenosine is not included when determining the percentage complementarity. In some embodiments, the sequence of the oligonucleotide of the invention comprises or consists of a sequence having at least 85%, at least 90%, at least 95%, at least 96%, at least 97%, at least 98%, at least 99% or 100% complementarity to a sequence in a SERPINA1 mRNA, wherein any match or mismatch between the editing nucleoside and the target adenosine is not included when determining the percentage complementarity.

The term “% complementary” as used herein, refers to the proportion of nucleotides (in percent) of an oligonucleotide of the invention which are complementary to a sequence in a target nucleic acid, such as a SERPINA1 mRNA, excluding any base-pairing (or lack thereof) between the editing nucleoside and target adenosine. In other words, any match or mismatch between the editing nucleoside and the target adenosine is not included when determining the percentage complementarity. The percentage of complementarity is thus calculated by counting the number of aligned nucleobases that are complementary (i.e. form Watson Crick base pairs) between the two sequences except for the editing nucleoside and target adenosine, dividing that number by the total number of nucleotides in the oligonucleotide (except for the editing nucleoside) and multiplying by 100. In such a comparison, a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine and 7-deaza-8-azaguanine is considered identical to a guanine for the purpose of calculating % complementarity).

The term “complementary” (such as in the phrase “the oligonucleotide is complementary to a sequence in a SERPINA1 mRNA”) does not require 100% complementarity. Rather, within the present invention, the term “complementary” requires the oligonucleotide to be at least 75% to the target nucleic acid. In some embodiments, the term “complementary” requires the oligonucleotide to be at least 75% complementary to the SERPINA1 mRNA. In some embodiments the oligonucleotide is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary, or 100% complementary to the target nucleic acid. In some embodiments the oligonucleotide is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary, or 100% complementary to the SERPINA1 mRNA.

The term “fully complementary”, refers to 100% complementarity. In some embodiments, the oligonucleotide is fully complementary to a sequence in a target nucleic acid. In other words, every nucleobase in the oligonucleotide except for the nucleobase of the editing nucleoside forms a base-pair with a nucleobase of the target nucleic acid. In some embodiments, the oligonucleotide is fully complementary to a sequence in a SERPINA1 mRNA. In other words, every nucleobase in the oligonucleotide except for the nucleobase of the editing nucleoside forms a base-pair with a nucleobase of the SERPINA1 mRNA.

In some embodiments, the oligonucleotide of the invention is complementary to any one of the SERPINA1 mRNA transcripts listed in Table 2 herein. In some embodiments, the oligonucleotide of the invention is complementary to SEQ ID NO 186 (SERPINA1 mRNA transcript variant 11 (NM_001127707.2) comprising the E342K mutation).

The editing oligonucleotide compounds exemplified herein bind to SEQ ID NO 186 at positions 986 to 1062. Therefore, in some embodiments, the sequence of the oligonucleotide of the invention comprises or consists of a sequence having at least 80%, at least 85%, at least 90%, at least 95%, at least 96%, at least 97%, at least 98%, at least 99% or 100% complementarity to a sequence within positions 986 to 1062 of SEQ ID NO 186, wherein any match or mismatch between the editing nucleoside and the target adenosine is not included when determining the percentage complementarity. Positions 986 to 1062 of SEQ ID NO 186 are represented below as SEQ ID NO 187. The AAG codon encoding K342 is underlined and the target adenosine is in bold. The target adenosine is A39 of SEQ ID NO 187.

(SEQ ID NO 187)
TCTCCAAGGCCGTGCATAAGGCTGTGCTGACCATCGACAAGAAAGG
GACTGAAGCTGCTGGGGCCATGTTTTTAGAG

In some embodiments, the sequence of the oligonucleotide of the invention comprises or consists of a sequence having at least 80%, at least 85%, at least 90%, at least 95%, at least 96%, at least 97%, at least 98%, at least 99% or 100% complementarity to SEQ ID NO 187, wherein any match or mismatch between the editing nucleoside and the target adenosine is not included when determining the percentage complementarity. The oligonucleotide may form complementary base-pairs with any stretch of nucleosides within the sequence of SEQ ID NO 187.

### Sequences

The term “sequence” as used herein refers to the order of nucleobases in a nucleic acid, such as an oligonucleotide. When used to refer to an oligonucleotide or an oligonucleotide conjugate of the invention, the term “sequence” does not limit the form of the sugar moieties of the nucleic acid and does not limit the form of the internucleoside linkages of the nucleic acid. Thus, a given sequence of an oligonucleotide or an oligonucleotide conjugate may comprise any types of nucleoside sugar moieties (e.g. RNA, DNA, LNA, 2′-O-methyl-RNA, MOE-RNA, as described herein), in any combination, and may comprise any types of internucleoside linkages (e.g. phosphodiester, phosphorothioate, phosphorodithioate, as described herein). Each reference herein to a SEQ ID NO (sequence identifier number) refers to the sequence represented by that SEQ ID NO. Unless stated otherwise, all sequences herein are presented in the 5′ to 3′ direction, as is conventional in the art.

In some embodiments of the oligonucleotide of the invention, the sequence of the oligonucleotide comprises or consists of from 30 to 61 contiguous nucleosides from the following sequence:

(SEQ ID NO 87)
CUCUAAAAACAUGGCCCCAGCAGCUUCAGUCCCUUUCTCIUCGAUGGUC
AGCACAGCCUUAUGCACGGCCUUGGUGU,

or from a variant of SEQ ID NO 87 comprising exactly 1, exactly 2 or exactly 3 single nucleoside substitutions, wherein I is inosine, and wherein the oligonucleotide comprises positions 38, 39 and 40 of SEQ ID NO 87. Positions 38, 39 and 40 of SEQ ID NO 87 (underlined above) are the editing triplet. Position 39 of SEQ ID NO 87 is the editing triplet. That the sequence of the oligonucleotide “comprises positions 38, 39 and 40” means that the sequence must include nucleosides corresponding to these positions. It is not allowed that, for example, the sequence comprise only nucleosides corresponding to the nucleosides 5′ to position 38 (e.g. position 1 to 37) or 3′ to position 40 (e.g. positions 41 to 61). However, it is not required that the nucleosides at positions 38, 39 and 40 have the sequence TCI.

Nucleosides at these positions may be substituted for different types of nucleoside (within the limit of exactly 3 nucleoside substitutions across the whole sequence); but there must be nucleosides corresponding to these positions in the sequence.

In some embodiments, the sequence of the oligonucleotide comprises or consists of from 30 to 50 contiguous nucleosides from SEQ ID NO 87 or from the variant thereof. In some embodiments, the sequence comprises or consists of 30, 31, 32, 33, 34, 35, 36, 37, 38, 39, 40, 41, 42, 43, 44, 45, 46, 47, 48, 49 or 50 contiguous nucleosides from SEQ ID NO 87 or from the variant thereof. In some embodiments, the sequence comprises or consists of 35, 36, 37, 38, 39, 40, 41, 42, 43, 44 or 45 contiguous nucleosides from SEQ ID NO 87 or from the variant thereof. In some embodiments, the sequence comprises or consists of 40 contiguous nucleosides from SEQ ID NO 87 or from the variant thereof.

In some embodiments, the variant of SEQ ID NO 87 comprises exactly 1 single nucleoside substitution. In some embodiments, the variant of SEQ ID NO 87 comprises a nucleoside substitution at position 38, 39 and/or 40 of SEQ ID NO 87. In some embodiments, the variant of SEQ ID NO 87 comprises a nucleoside substitution at position 39 of SEQ ID NO 87.

### Sequences of Exemplified Compounds

In some embodiments, the sequence of the oligonucleotide of the invention comprises a sequence having at least 80% identity to any one of SEQ ID NOs 1 to 86 (as shown in Table 3 in Example 1 herein). In some embodiments, the sequence of the oligonucleotide comprises a sequence having at least 85% identity, at least 90% identity, at least 95% identity, at least 96% identity, at least 97% identity, at least 98% identity, at least 99% identity or 100% identity to any one of SEQ ID NOs 1 to 86. In some embodiments, the sequence of the oligonucleotide comprises the sequence of any one of SEQ ID NOs 1 to 86.

In some embodiments, the sequence of the oligonucleotide of the invention consists of a sequence having at least 80% identity to any one of SEQ ID NOs 1 to 86. In some embodiments, the sequence of the oligonucleotide consists of a sequence having at least 85% identity, at least 90% identity, at least 95% identity, at least 96% identity, at least 97% identity, at least 98% identity, at least 99% identity or 100% identity to any one of SEQ ID NOs 1 to 86. In some embodiments, the sequence of the oligonucleotide consists of the sequence of any one of SEQ ID NOs 1 to 86.

In some embodiments, the sequence of the oligonucleotide of the invention comprises a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86. In some embodiments, the sequence of the oligonucleotide comprises a sequence having at least 85% identity, at least 90% identity, at least 95% identity, at least 96% identity, at least 97% identity, at least 98% identity, at least 99% identity or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86. In some embodiments, the sequence of the oligonucleotide comprises the sequence of any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86.

In some embodiments, the sequence of the oligonucleotide of the invention consists of a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86. In some embodiments, the sequence of the oligonucleotide consists of a sequence having at least 85% identity, at least 90% identity, at least 95% identity, at least 96% identity, at least 97% identity, at least 98% identity, at least 99% identity or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86. In some embodiments, the sequence of the oligonucleotide consists of the sequence of any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86.

In some embodiments, the sequence of the oligonucleotide of the invention comprises a sequence having at least 80% identity to any one of SEQ ID NOs 3, 7, 8, 9, 10, 19, 20, 21, 22, 23, 26, 29, 30, 31, 32, 33, 34, 35, 36, 41, 42, 43, 45, 60, 61, 62 and 63. In some embodiments, the sequence of the oligonucleotide comprises a sequence having at least 85% identity, at least 90% identity, at least 95% identity, at least 96% identity, at least 97% identity, at least 98% identity, at least 99% identity or 100% identity to any one of SEQ ID NOs 3, 7, 8, 9, 10, 19, 20, 21, 22, 23, 26, 29, 30, 31, 32, 33, 34, 35, 36, 41, 42, 43, 45, 60, 61, 62 and 63. In some embodiments, the sequence of the oligonucleotide comprises the sequence of any one of SEQ ID NOs 2, 3, 7, 8, 9, 10, 19, 20, 21, 22, 23, 26, 29, 30, 31, 32, 33, 34, 35, 36, 41, 42, 43, 45, 60, 61, 62 and 63.

In some embodiments, the sequence of the oligonucleotide of the invention consists of a sequence having at least 80% identity to any one of SEQ ID NOs 3, 7, 8, 9, 10, 19, 20, 21, 22, 23, 26, 29, 30, 31, 32, 33, 34, 35, 36, 41, 42, 43, 45, 60, 61, 62 and 63. In some embodiments, the sequence of the oligonucleotide consists of a sequence having at least 85% identity, at least 90% identity, at least 95% identity, at least 96% identity, at least 97% identity, at least 98% identity, at least 99% identity or 100% identity to any one of SEQ ID NOs 3, 7, 8, 9, 10, 19, 20, 21, 22, 23, 26, 29, 30, 31, 32, 33, 34, 35, 36, 41, 42, 43, 45, 60, 61, 62 and 63. In some embodiments, the sequence of the oligonucleotide consists of the sequence of any one of SEQ ID NOs 3, 7, 8, 9, 10, 19, 20, 21, 22, 23, 26, 29, 30, 31, 32, 33, 34, 35, 36, 41, 42, 43, 45, 60, 61, 62 and 63.

In some embodiments, the sequence of the oligonucleotide of the invention comprises a sequence having at least 80% identity to any one of SEQ ID NOs 3, 7, 8, 9, 19, 20, 21, 22, 29, 30, 31, 32, 33, 34, 42, 43, 62 and 63. In some embodiments, the sequence of the oligonucleotide comprises a sequence having at least 85% identity, at least 90% identity, at least 95% identity, at least 96% identity, at least 97% identity, at least 98% identity, at least 99% identity or 100% identity to any one of SEQ ID NOs 3, 7, 8, 9, 19, 20, 21, 22, 29, 30, 31, 32, 33, 34, 42, 43, 62 and 63. In some embodiments, the sequence of the oligonucleotide comprises the sequence of any one of SEQ ID NOs 3, 7, 8, 9, 19, 20, 21, 22, 29, 30, 31, 32, 33, 34, 42, 43, 62 and 63.

In some embodiments, the sequence of the oligonucleotide of the invention consists of a sequence having at least 80% identity to any one of SEQ ID NOs 3, 7, 8, 9, 19, 20, 21, 22, 29, 30, 31, 32, 33, 34, 42, 43, 62 and 63. In some embodiments, the sequence of the oligonucleotide consists of a sequence having at least 85% identity, at least 90% identity, at least 95% identity, at least 96% identity, at least 97% identity, at least 98% identity, at least 99% identity or 100% identity to any one of SEQ ID NOs 3, 7, 8, 9, 19, 20, 21, 22, 29, 30, 31, 32, 33, 34, 42, 43, 62 and 63. In some embodiments, the sequence of the oligonucleotide consists of the sequence of any one of SEQ ID NOs 3, 7, 8, 9, 19, 20, 21, 22, 29, 30, 31, 32, 33, 34, 42, 43, 62 and 63.

In some embodiments, the sequence of the oligonucleotide of the invention comprises a sequence having at least 80% identity to SEQ ID NOs 32. In some embodiments, the sequence of the oligonucleotide comprises a sequence having at least 85% identity, at least 90% identity, at least 95% identity, at least 96% identity, at least 97% identity, at least 98% identity, at least 99% identity or 100% identity to SEQ ID NO 32. In some embodiments, the sequence of the oligonucleotide comprises the sequence of SEQ ID NO 32. In some embodiments, the sequence of the oligonucleotide consists of a sequence having at least 80% identity to SEQ ID NO 32. In some embodiments, the sequence of the oligonucleotide consists of a sequence having at least 85% identity, at least 90% identity, at least 95% identity, at least 96% identity, at least 97% identity, at least 98% identity, at least 99% identity or 100% identity to SEQ ID NO 32. In some embodiments, the sequence of the oligonucleotide consists of the sequence of SEQ ID NO 32.

### Identity

The term “identity” as used herein, refers to the proportion of nucleotides (expressed in percent) of a nucleotide sequence in a nucleic acid molecule (e.g. oligonucleotide, such as an oligonucleotide of the invention) which, across the nucleotide sequence, are identical when compared to a reference sequence. The nucleotide sequence that is compared to the reference sequence may be referred to as the ‘query sequence’ herein in the context of determination of sequence identity.

For determination of sequence identity, sequence comparison may be performed by any method known in the art. Suitably, sequence comparisons can be conducted by eye, or more usually, with the aid of readily available sequence comparison programs. These publicly and commercially available computer programs can calculate sequence identity between two or more sequences.

Sequence identity may be calculated over contiguous sequences, i.e. one sequence is aligned with the other sequence and each nucleotide in one sequence directly compared with the corresponding nucleotide in the other sequence, one nucleotide at a time. This is called an “ungapped” alignment. Typically, such ungapped alignments are performed only over a relatively short number of nucleotides (for example less than 50 contiguous nucleotides).

Although this is a very simple and consistent method, it fails to take into consideration that, for example, in an otherwise identical pair of sequences, one insertion or deletion will cause the following nucleotides to be put out of alignment, thus potentially resulting in a large reduction in percentage identity when a global alignment is performed. Consequently, most sequence comparison methods are designed to produce optimal alignments that take into consideration possible insertions and deletions without penalising unduly the overall identity. This is achieved by inserting “gaps” in the sequence alignment to try to maximise local identity.

However, these more complex methods assign “gap penalties” to each gap that occurs in the alignment so that, for the same number of identical nucleotides, a sequence alignment with as few gaps as possible (reflecting higher relatedness between the two compared sequences) will achieve a higher score than one with many gaps. “Affine gap costs” are typically used that charge a relatively high cost for the existence of a gap and a smaller penalty for each subsequent nucleotide in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum percentage sequence identity therefore firstly requires the production of an optimal alignment, taking into consideration gap penalties. A suitable computer program for carrying out such an alignment is the GCG Wisconsin Bestfit package (University of Wisconsin, U.S.A; Devereux et al. 1984 Nucleic Acids Research 12:387). Examples of other software than can perform sequence comparisons include, but are not limited to, the BLAST package (see Ausubel et al. 1999 ibid—Chapter 18), FASTA (Atschul et al. 1990, J. Mol. Biol., 403-410) and the GENEWORKS suite of comparison tools. Both BLAST and FASTA are available for offline and online searching (see Ausubel et al. 1999 ibid, pages 7-58 to 7-60). However it is preferred to use the GCG Bestfit program.

Suitably, the sequence identity may be determined across the entirety of the sequence. Suitably, the sequence identity may be determined across the entirety of the query sequence being compared to a reference sequence.

Although the final sequence identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see user manual for further details). Preferably, the public default values for the GCG package, or in the case of other software the default matrix, such as BLOSUM62, are used.

Once the software has produced an optimal alignment, it is possible to calculate percentage sequence identity. The software typically does this as part of the sequence comparison and generates a numerical result.

In some embodiments, the percentage of identity is calculated by counting the number of aligned nucleobases that are identical (a Match) between the query sequence and the reference sequence, dividing that number by the total number of nucleotides in the reference sequence and multiplying by 100. Therefore, Percentage of Identity=(Matches×100)/Length of the reference sequence).

Thus, in embodiments wherein the oligonucleotide of the invention or a sequence of the oligonucleotide of the invention is stated to have at least a certain identity to another sequence, the oligonucleotide of the invention or sequence of the oligonucleotide of the invention is the query sequence, and the other sequence is the reference sequence. In embodiments wherein the oligonucleotide of the invention or a sequence of the oligonucleotide of the invention is stated to have at least a certain identity to a SEQ ID NO (i.e. the sequence indicated by the SEQ ID NO), the oligonucleotide of the invention or sequence of the oligonucleotide of the invention is the query sequence, and the SEQ ID NO is the reference sequence.

For example, in the case of an oligonucleotide of the invention having at least 80% identity to SEQ ID NO 32, the oligonucleotide of the invention is the query sequence and SEQ ID NO 32 (i.e. the sequence indicated by the SEQ ID NO) is the reference sequence. In the case of a sequence of an oligonucleotide of the invention having at least 80% identity to SEQ ID NO 32, the sequence of the oligonucleotide of the invention is the query sequence and SEQ ID NO 32 is the reference sequence

Suitably, insertions and deletions may not be allowed in the calculation of the percentage of identity of a nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine and 7-deaza-8-azaguanine is considered identical to a guanine for the purpose of calculating % identity).

### Compounds

The term “compound” is used herein to refer to the combination of sequence (i.e. the order of nucleobases), sugar moieties and internucleoside linkages in an oligonucleotide of the invention. For a given compound, the nucleobase, the type of sugar moiety and the internucleoside linkages of each nucleotide are specified. Thus, the order of sugar moieties and internucleoside linkages is also specified in a given compound. However, unless otherwise indicated, a compound may comprise other elements in addition to the specified sequence, sugar moieties and internucleoside linkages.

In some embodiments, the term “oligonucleotide” is interchangeable with the term “compound”. In other words, in some embodiments, an oligonucleotide of the invention is a compound of the invention, and vice versa.

Specific compounds are referred to herein using a compound identifier number (CMP ID NO) of the form X_Y, wherein X and Y are each a number. For each CMP ID NO, X is the number of the SEQ ID NO that corresponds to the sequence of the compound. For example, the compound designated CMP ID NO 2_1 has the same nucleobase sequence as SEQ ID NO 2.

The structures of the compounds referred to here are presented in Table 4 of Example 1 herein using a HELM string. Another section herein explains how to read HELM strings. The HELM strings are not written out again here for sake of brevity, but it will be understood that each reference here to CMP ID NO refers to the compound represented by the HELM string for that CMP ID NO as shown in Table 4. The CMP ID NO and the HELM string in Table 4 corresponding to that CMP ID NO are interchangeable.

In some embodiments, the oligonucleotide of the invention comprises any one of CMP ID NOs 1_1, 2_1, 3_1, 3_2, 3_3, 4_1, 5_1, 6_1, 7_1, 8_1, 9_1, 10_1, 11_1, 12_1, 13_1, 14_1, 15_, 16_1, 17_1, 18_1, 19_1, 20_1, 21_1, 22_1, 23_1, 24_1, 25_1, 26_1, 26_2, 26_3, 26_4, 26_5, 26_6, 26_7, 26_8, 26_9, 27_1, 28_1, 29_1, 30_1, 31_1, 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82, 32_83, 33_1, 34_1, 35_1, 36_1, 37_1, 38_1, 39_1, 40_1, 40_2, 40_3, 40_4, 40_5, 40_6, 40_7, 40_8, 40_9, 40_10, 41_1, 41_2, 41_3, 41_4, 41_5, 41_6, 41_7, 41_8, 41_9, 41_10, 42_1, 42_2, 42_3, 42_4, 42_5, 42_6, 43_1, 43_2, 43_3, 44_1, 45_1, 46_1, 47_1, 47_2, 48_1, 49_1, 50_1, 51_1, 52_1, 53_1, 54_1, 55_1, 56_1, 57_1, 58_1, 59_1, 60_1, 61_1, 62_1, 63_1, 64_1, 65_1, 66_1, 67_1, 68_1, 69_1, 70_1, 71_1, 72_1, 73_1, 74_1, 75_1, 76_1, 77_1, 78_1, 79_1, 80_1, 81_1, 82_1, 83_1, 84_1, 85_1 and 86_1. In some embodiments, the antisense oligonucleotide consists of any one of CMP ID NOs 1_1, 2_1, 3_1, 3_2, 3_3, 4_1, 5_1, 6_1, 7_1, 8_1, 9_1, 10_1, 11_1, 12_1, 13_1, 14_1, 15_1, 16_1, 17_1, 18_1, 19_1, 20_1, 21_1, 22_1, 23_1.24_1, 25_1, 26_1, 26_2, 26_3, 26_4, 26_5, 26_6, 26_7, 26_8.26_9, 27_1, 28_1, 29_1, 30_1, 31_1, 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82, 32_83, 33_1, 34_1, 35_1, 36_1, 37_1, 38_1, 39_1, 40_1, 40_2, 40_3, 40_4, 40_5, 40_6, 40_7, 40_8, 40_9, 40_10, 41_1, 41_2, 41_3, 41_4, 41_5, 41_6, 41_7, 41_8, 41_9, 41_10, 42_1, 42_2, 42_3, 42_4, 42_5, 42_6, 43_1, 43_2, 43_3, 44_1, 45_1, 46_1, 47_1, 47_2, 48_1, 49_1, 50_1, 51_1, 52_1, 53_1.54_1, 55_1, 56_1, 57_1, 58_1, 59_1, 60_1, 61_1, 62_1, 63_1.64_1, 65_1, 66_1, 67_1, 68_1, 69_1, 70_1, 71_1.72_1, 73_1, 74_1, 75_1, 76_1.77_1, 78_1, 79_1, 80_1, 81_1, 82_1, 83_1, 84_1, 85_1 and 86_1.

In some embodiments, the oligonucleotide of the invention comprises any one of CMP ID NOs 3_3, 7_1, 8_1, 9_1, 10_1, 19_1, 20_1, 21_1.22_1, 23_1, 26_8, 29_1, 30_1, 31_1, 32_1, 32_2, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_29, 32_33, 32_34, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 33_1, 34_1, 35_1, 36_1, 41_8, 42_6, 43_1, 43_3, 45_1, 60_1, 61_1, 62_1 and 63_1. In some embodiments, the oligonucleotide of the invention consists of any one of CMP ID NOs 3_3, 7_1, 8_1, 9_1, 10_1, 19_1, 20_1, 21_1, 22_1, 23_1, 26_8, 29_1, 301, 31_1, 32_1, 32_2, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_29, 32_33, 32_34, 32_47, 32_48, 32_49, 32_50, 32_51, 32_52, 32_53, 32_54, 32_55, 32_56, 32_57, 32_58, 32_60, 32_61, 32_62, 32_63, 32_64, 32_65, 33_1, 34_1, 35_1, 36_1, 41_8, 42_6, 43_1, 433, 45_1, 60_1, 61_1, 62_1 and 63_1.

In some embodiments, the oligonucleotide of the invention comprises any one of CMP ID NOs 7_1, 8_1, 9_1, 19_1, 20_1, 21_1, 22_1, 29_1, 30_1, 31_1, 19_1, 32_1, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_33, 32_47, 32_48, 32_49, 32_50, 32_51, 32_52, 32_53, 32_54, 32_55, 32_56, 32_57, 32_58, 32_60, 32_61, 32_62, 32_63, 32_64, 32_65, 33_1, 34_1, 42_6, 43_1, 62_1 and 63_1. In some embodiments, the oligonucleotide of the invention consists of any one of CMP ID NOs 7_1, 8_1, 9_1, 19_1, 20_1, 21_1, 22_1, 29_1, 30_1, 31_1, 19_1, 32_1, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_33, 32_47, 32_48, 32_49, 32_50, 32_51, 32_52, 32_53, 32_54, 32_55, 32_56, 32_57, 32_58, 32_60, 32_61, 32_62, 32_63, 32_64, 32_65, 33_1, 34_1, 42_6, 43_1, 62_1 and 63_1.

In some embodiments, the oligonucleotide of the invention comprises any one of CMP ID NOs 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82 and 32_83. In some embodiments, the oligonucleotide of the invention consists of any one of CMP ID NOs 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82 and 32_83.

In some embodiments, the oligonucleotide of the invention comprises any one of CMP ID NOs 32_1, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_33, 32_47, 32_48, 32_49, 32_50, 32_51, 32_52, 32_53, 32_54, 32_55, 32_56, 32_57, 32_58, 32_60, 32_61, 32_62, 32_63, 32_64 and 32_65. In some embodiments, the oligonucleotide of the invention consists of any one of CMP ID NOs 32_1, 32_4, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_33, 32_47, 32_48, 32_49, 32_50, 32_51, 32_52, 32_53, 32_54, 32_55, 32_56, 32_57, 32_58, 32_60, 32_61, 32_62, 32_63, 32_64 and 32_65.

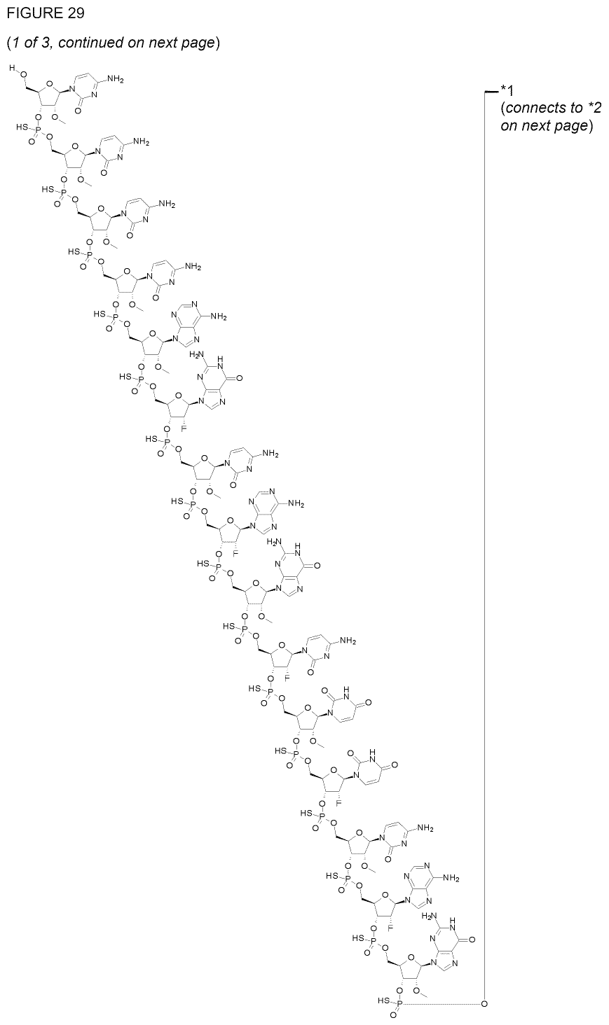

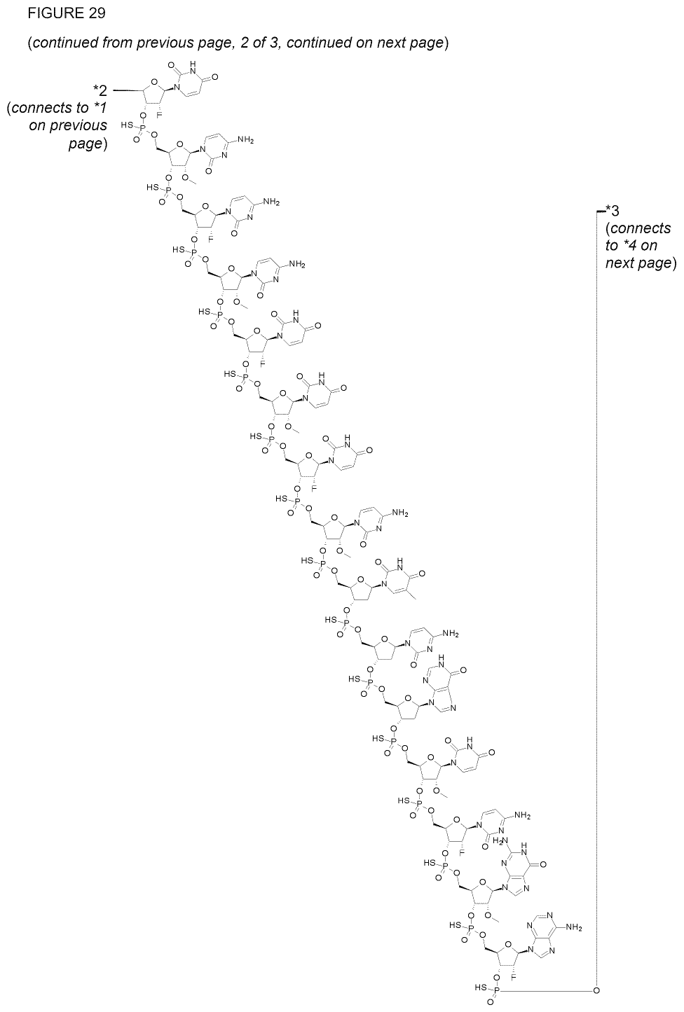

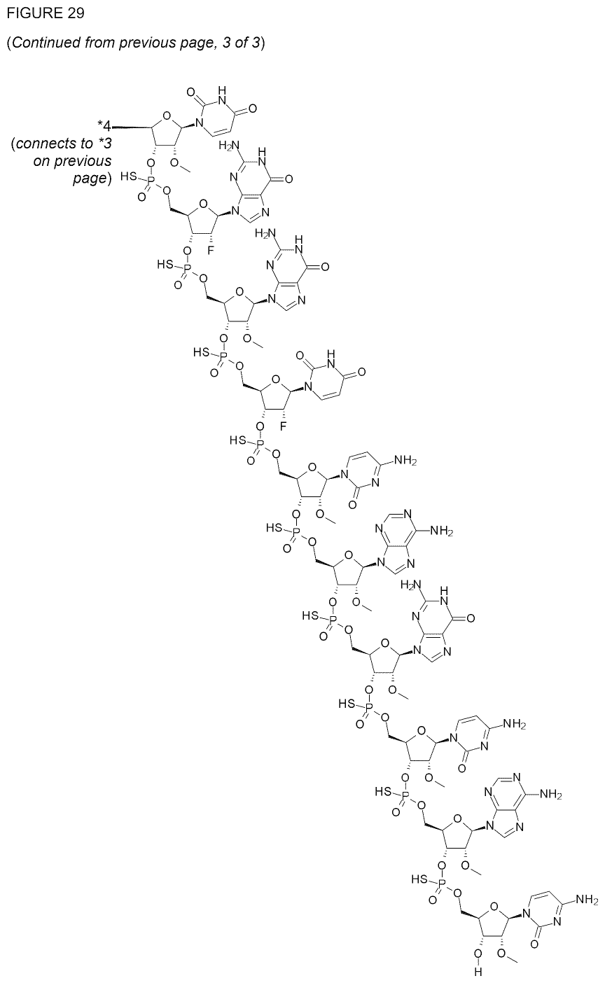

In some embodiments, the oligonucleotide of the invention comprises CMP ID NO 32_1. In some embodiments, the oligonucleotide of the invention consists of CMP ID NO 32_1. In some embodiments, the oligonucleotide of the invention is CMP ID NO 32_1. In some embodiments, the oligonucleotide of the invention is the compound depicted in FIG. 29.

### Conjugates

The invention provides an oligonucleotide conjugate comprising the oligonucleotide of the invention covalently attached to at least one conjugate moiety. In other words, the invention provides an oligonucleotide covalently attached to at least one conjugate moiety.

The term “oligonucleotide conjugate” is used interchangeably herein with the terms “conjugate” and “conjugate of the invention”. The term “conjugate moiety” refers to a non-nucleotide moiety which can be covalently attached to an oligonucleotide of the invention. Thus, the term “conjugate” as used herein refers to an oligonucleotide of the invention which is covalently attached to a non-nucleotide moiety (conjugate moiety).

Oligonucleotide conjugates and their synthesis has also been reported in comprehensive reviews by Manoharan in Antisense Drug Technology, Principles. Strategies, and Applications, S. T. Crooke, ed., Ch. 16, Marcel Dekker, Inc., 2001 and Manoharan, Antisense and Nucleic Acid Drug Development, 2002, 12, 103.

The term “conjugate” may be used herein to refer to the combination of sequence (i.e. the order of nucleobases), sugar moieties, internucleoside linkages and conjugate moiety in a given oligonucleotide conjugate of the invention. For a given conjugate, the nucleobase, the type of sugar moiety and the internucleoside linkages of each nucleotide in the oligonucleotide of the conjugate are specified. Thus, the order of sugar moieties and internucleoside linkages is also specified in a given conjugate. However, unless otherwise indicated, a conjugate may comprise other elements in addition to the specified sequence, sugar moieties, internucleoside linkages and conjugate moiety.

Specific conjugates are referred to herein using a conjugate identifier number (CNJ ID NO) of the form X_Y_Z, wherein X, Y and Z are each a number. For each CNJ ID NO, X_Y is the CMP ID NO of the oligonucleotide compound of the conjugate (recalling that X is the SEQ ID NO of the nucleobase sequence of that compound). For example, the conjugate designated CNJ ID NO 126_2_1 comprises the oligonucleotide compound CMP ID NO 126_2, which has the nucleobase sequence of SEQ ID NO 126.

In some embodiments, the at least one conjugate moiety is covalently attached to the 5′ end of the oligonucleotide. In some embodiments, the at least one conjugate moiety is covalently attached to the 3′ end of the oligonucleotide.

### Conjugate Moieties

In some embodiments, the conjugate moiety (i.e. non-nucleotide moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

In some embodiments, the conjugate moiety is capable of binding to the asialoglycoprotein receptor, such as the human asialoglycoprotein receptor (ASGPR). For example, the conjugate moiety may comprise at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.

In some embodiments, the asialoglycoprotein receptor-targeting moiety is N-acetylgalactosamine (GalNAc). In some embodiments, the conjugate moiety is an N-acetylgalactosamine (GalNAc) conjugate moiety. Thus, the oligonucleotide of the present invention may be conjugated to at least one conjugate moiety comprising at least one N-acetylgalactosamine (GalNAc) moiety, such as at least one conjugate moiety comprising at least one N-acetylgalactosamine (GalNAc) moiety as described below.

In some embodiments, the conjugate moiety is an at least divalent, such as a divalent, trivalent or tetravalent, GalNAc. In preferred embodiments, the conjugate moiety is a trivalent GalNAc. Tri-valent N-acetylgalactosamine conjugate moieties are suitable for binding to the ASGPR, see for example WO 2014/076196, WO 2014/207232 and WO 2014/179620. Such conjugate moieties serve to enhance uptake of the oligonucleotide to the liver. The term “trivalent GalNAc” as used herein refers to a residue comprising three N-acetylgalactosamine moieties, i.e. preferably three moieties of formula

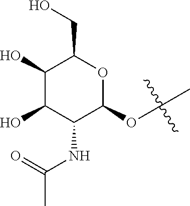 [SMILES: *O[C@@H]1O[C@H](CO)[C@H](O)[C@H](O)[C@H]1NC(C)=O]

In some embodiments, the GalNAc conjugate moiety is aminohexyl conjugated tri(N-acetyl-galactosamine) as depicted below.

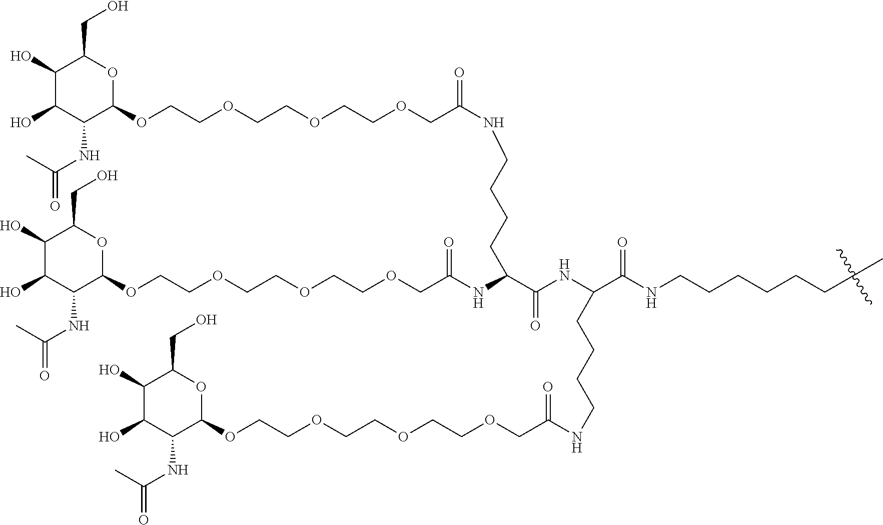 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

The trivalent N-acetylgalactosamine (GalNAc) shown above is also referred to herein as “5gn2c6”.

“Aminohexyl conjugated tri(N-acetyl-galactosamine)” may also be referred to as “-hexylene-NH-tri(N-acetyl-galactosamine)”.

In some embodiments, the conjugate moiety is covalently attached to the oligonucleotide via a phosphodiester bond. In some embodiments, the conjugate moiety is covalently attached to the linker via a phosphodiester bond.

### Linkers

In some embodiments of the oligonucleotide conjugate of the invention, the conjugate moiety is covalently attached to the oligonucleotide via a linker. Thus, in some embodiments, the conjugate comprises a linker. In some embodiments, the conjugate comprises a linker which is positioned between the oligonucleotide and the conjugate moiety.

In some embodiments of the oligonucleotide conjugate of the invention, the conjugate moiety is covalently attached to the oligonucleotide via a linker nucleoside sequence.

In some embodiments, the linker nucleoside sequence comprises or consists of 1 to 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides. In some embodiments, the linker nucleoside sequence is 2 nucleosides in length. In some embodiments, the linker comprises two linked nucleosides. In some embodiments, the linker consists of two linked nucleosides.

In some embodiments, the linker nucleoside sequence comprises DNA nucleosides. In some embodiments, the linker nucleoside sequence consists of DNA nucleosides. In other words, in some embodiments, all nucleosides in the linker nucleoside sequence are DNA nucleosides.

In some embodiments, the nucleosides of the linker nucleoside sequence are linked via phosphodiester internucleoside linkages. In some embodiments, the linker is linked to the oligonucleotide via a phosphodiester internucleoside linkage. In some embodiments, the linker is linked to the conjugate moiety via a phosphodiester bond.

The terms “phosphodiester internucleoside linkage” and “phosphodiester bond” as used herein refer to the same chemical structure, known in the art, wherein a first chemical entity is linked to a second chemical entity via an intermediate phosphate group. The term “phosphodiester internucleoside linkage” is used particularly wherein the first chemical entity and second chemical entity that are linked are nucleosides, such as nucleosides of the oligonucleotide of the invention. As is known in the art, the phosphodiester internucleoside linkage is the naturally occurring internucleoside in naturally occurring nucleic acids such as genomic DNA. The term “phosphodiester bond” is used particularly in the context of the present invention to refer to the covalent attachment of a conjugate moiety to the oligonucleotide of the invention or to a linker, such as a nucleoside linker sequence, because the conjugate moiety is not a nucleoside so the term “phosphodiester internucleoside linkage” is not appropriate.

In some embodiments, the linker comprises or consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides and at least one further phosphodiester at the 5′ or 3′ end of the dinucleotide linking either the oligonucleotide to the dinucleotide or the conjugate moiety to the dinucleotide. For example, the linker may by a CA dinucleotide. In some embodiments, the linker comprises or consists of a DNA trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG, where there are phosphodiester linkages between the DNA nucleosides and potentially a further phosphodiester at the 5′ or 3′ end of the trinucleotide.

In some embodiments, the linker nucleoside sequence is CA. In other words, in some embodiments, the sequence of the linker nucleoside sequence is CA. In some embodiments, the linker nucleotide sequence is 5′-CA-3′. In some embodiments, the linker nucleoside sequence is the dinucleotide CA, wherein the C nucleoside is linked to the conjugate moiety by a phosphodiester bond, the C nucleoside is linked to the A nucleoside by a phosphodiester internucleoside linkage, and the A nucleoside is linked to the 5′ nucleoside of the antisense oligonucleotide of the invention by a phosphodiester internucleoside linkage.

In some embodiments, the linker is a biocleavable linker. Biocleavable linkers comprises or consist of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195 (incorporated by reference herein). In a conjugate with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

### Pharmaceutically Acceptable Salts

In some embodiments, the oligonucleotide or the oligonucleotide conjugate of the invention is in the form of a pharmaceutically acceptable salt. In some embodiments, the oligonucleotide is in the form of a pharmaceutically acceptable salt. In some embodiments, the oligonucleotide conjugate is in the form of a pharmaceutically acceptable salt.

The term “salt” as used herein conforms to its generally known meaning, i.e. an ionic assembly of anions and cations.

The term “pharmaceutically acceptable salts” refers to those salts, which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compounds of the present invention can also be present in the form of zwitterions. Particularly preferred pharmaceutically acceptable salts of oligonucleotides or oligonucleotide conjugates of the invention are the salts of hydrochloric acid, hydrobromic acid, sulfuric acid, phosphoric acid and methanesulfonic acid.

In some embodiments, the pharmaceutically acceptable salt is a sodium salt or a potassium salt. In some embodiments, the oligonucleotide or the oligonucleotide conjugate of the invention is in the form of a sodium salt. In some embodiments, the oligonucleotide of the invention is in the form of a sodium salt. In some embodiments, the oligonucleotide conjugate of the invention is in the form of a sodium salt. In some embodiments, the oligonucleotide or the oligonucleotide conjugate of the invention is in the form of a potassium salt. In some embodiments, the oligonucleotide of the invention is in the form of a potassium salt. In some embodiments, the oligonucleotide conjugate of the invention is in the form of a potassium salt.

### Delivery of Antisense Oligonucleotides

In some embodiments, the oligonucleotide or the oligonucleotide conjugate of the invention is encapsulated in a lipid-based delivery vehicle, covalently linked to or encapsulated in a dendrimer, or conjugated to an aptamer. This may be for the purpose of delivering the oligonucleotides of the invention to the targeted cells and/or to improve the pharmacokinetics of the oligonucleotide. Examples of lipid-based delivery vehicles include oil-in-water emulsions, micelles, liposomes, and lipid nanoparticles.

In some embodiments, the oligonucleotide is encapsulated in a lipid-based delivery vehicle. In some embodiments, the oligonucleotide is covalently linked to a dendrimer. In some embodiments, the oligonucleotide is encapsulated in a dendrimer. In some embodiments, the oligonucleotide is conjugated to an aptamer.

In some embodiments, the oligonucleotide conjugate is encapsulated in a lipid-based delivery vehicle. In some embodiments, the oligonucleotide conjugate is covalently linked to a dendrimer. In some embodiments, the oligonucleotide conjugate is encapsulated in a dendrimer. In some embodiments, the oligonucleotide conjugate is conjugated to an aptamer.

### Pharmaceutical Compositions

The invention provides a pharmaceutical composition comprising the oligonucleotide or the oligonucleotide conjugate of the invention, and a pharmaceutically acceptable diluent, solvent, carrier, salt and/or adjuvant. In some embodiments, the pharmaceutical composition comprises an aqueous diluent or solvent. In some embodiments, the aqueous diluent or solvent is phosphate buffered saline. In some embodiments, the aqueous diluent or solvent is sterile.

The invention provides a pharmaceutical composition comprising the oligonucleotide of the invention, and a pharmaceutically acceptable salt. In some embodiments, the salt comprises a metal cation. In some embodiments, the pharmaceutically acceptable salt is selected from the group consisting of a sodium salt, a potassium salt and an ammonium salt.

The invention also provides a pharmaceutical solution of the oligonucleotide of the invention or the conjugate thereof, wherein the pharmaceutical solution comprises the oligonucleotide of the invention or the conjugate thereof and a pharmaceutically acceptable solvent, such as saline.

The invention also provides the oligonucleotide of the invention or the conjugate thereof in solid powdered form, such as in the form of a lyophilized powder.

### Method for Editing a Target Nucleic Acid

The invention provides an in vitro method for editing a target nucleic acid in a target cell, the method comprising administering an effective amount of the oligonucleotide or the oligonucleotide conjugate of the invention or the pharmaceutical composition of the invention to the target cell.

The invention also provides an in vivo method for editing a target nucleic acid in a target cell, the method comprising administering an effective amount of the oligonucleotide or the oligonucleotide conjugate of the invention or the pharmaceutical composition of the invention to the target cell.

The target nucleic acid comprises a target adenosine. The oligonucleotide or oligonucleotide conjugate binds to the target nucleic acid by complementary base pairing. The oligonucleotide or oligonucleotide conjugate recruits an ADAR to the target nucleic acid. In some embodiments, the ADAR is ADAR1 or ADAR2. In some embodiments of the methods of the invention, the target adenosine (A) is converted to inosine (1).

In some embodiments of the methods of the invention, the target nucleic acid is RNA. In some embodiments, the target nucleic acid is RNA. In some embodiments, the target nucleic acid encodes a protein. In some embodiments, the target nucleic acid is mRNA. In some embodiments, the target nucleic acid encodes alpha-1 antitrypsin (A1AT). In some embodiments, the target nucleic acid is a SERPINA1 mRNA.

In some embodiments of the methods of the invention, the oligonucleotide or oligonucleotide conjugate is capable of effecting conversion of an AAA codon encoding lysine to an IAA codon encoding glutamate on the target nucleic acid. In some embodiments of the methods of the invention, the oligonucleotide or oligonucleotide conjugate is capable of effecting conversion of an AAG codon encoding lysine to an IAG codon encoding glutamate on the target nucleic acid.

In some embodiments of the methods of the invention, the target adenosine corresponds to A1024 of SEQ ID NO 186. In some embodiments, the target nucleic acid comprises or consists of the sequence according to SEQ ID NO 186.

In some embodiments of the methods of the invention, the target cell is a mammalian cell. In some embodiments, the target cell is a human cell. In some embodiments of the methods, the target cell is a lung cell or a hepatocyte.

In some embodiments of the methods of the invention, the amount of edited target nucleic acid is increased compared to a control by at least 10%, at least 20%, at least 30%, at least 40%, at least 50%, at least 60%, at least 70%, at least 75%, at least 80%, at least 85%, at least 90%, at least 95% or 100%. In some embodiments, the control is a cell that has not been exposed to the oligonucleotide or the oligonucleotide conjugate. The amount of edited target nucleic acid may be determined by techniques known in the art. For example, the target nucleic acid may be amplified using PCR and then the proportion of edited target nucleic acid determined by sequencing. Examples of methods for determining the amount of edited target nucleic acid are described in Example 3 herein, in Merkle et al. 2019 Nature Biotechnology, Vol 37, 133-138 and in Qu et al. 2019 Nature Biotechnology, Vol 37, 1059-1069.

### Methods of Treatment, Methods of Prevention and Medical Uses

The invention provides a method for treating or preventing a disease comprising administering a therapeutically or prophylactically effective amount of the oligonucleotide or the oligonucleotide conjugate of the invention or the pharmaceutical composition of the invention to a subject suffering from or susceptible to a disease.

The invention provides an oligonucleotide or oligonucleotide conjugate of the invention or a pharmaceutical composition of the invention, for use in the treatment or prevention of a disease in a subject. The invention provides for an oligonucleotide of the invention, an oligonucleotide conjugate of the invention or a pharmaceutical composition of the invention, for use as a medicament. The invention provides an oligonucleotide of the invention, an oligonucleotide conjugate of the invention or a pharmaceutical composition of the invention for use in therapy.

The invention provides use of the oligonucleotide or the oligonucleotide conjugate of the invention or the pharmaceutical composition of the invention, for the preparation of a medicament for treatment or prevention of a disease in a subject. The invention provides for an oligonucleotide of the invention, an oligonucleotide conjugate of the invention or a pharmaceutical composition of the invention, for the preparation of a medicament.

The terms “treating”, “treatment” and “treats” as used herein refer to both treatment of an existing disease (e.g. a disease or disorder as herein referred to), or prevention of a disease, i.e. prophylaxis. It will therefore be recognized that treatment, as referred to herein may in some embodiments be prophylactic. Prophylactic can be understood as preventing A1AD.

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof.

The term “disease” as used herein refers to a state of dysfunction of the body. The term disease is herein synonymous with similar terms such as “condition” or “disorder”.

Many diseases are associated with mutations. A person skilled in the art will appreciate that the oligonucleotides provided herein, which facilitate correction of G-to-A mutations, may be used to treat many such diseases associated with mutations, particular G-to-A mutations. In some embodiments, the disease is associated with mutation of a G nucleotide (i.e. guanosine) to an A nucleotide (i.e. adenosine). In some embodiments, the disease is caused by mutation of a G nucleotide to an A nucleotide. In some embodiments, the disease is caused by mutation of a G nucleotide to an A nucleotide in the genome of the subject.

### Alpha 1 Antitrypsin Deficiency (A1AD)

In some embodiments of the method of the invention for treating or preventing a disease, the oligonucleotide for use, the oligonucleotide conjugate for use or the pharmaceutical composition for use of the invention, or the use of the invention, the disease is a disease associated with mutations in the SERPINA1 gene.

Alpha-1 antitrypsin (A1AT) deficiency (A1AD) is a disease associated with mutations in the SERPINA1 gene. SERPINA1 encodes A1AT, a serine protease inhibitor synthesized in the liver that is released to other tissues to protect them from endogenous inflammatory serine proteases, such as neutrophil elastase. Subjects suffering from A1AD express reduced levels of A1AT, which can lead to excessive breakdown of elastin in the lungs, and thereby reduced lung elasticity and associated health problems such as emphysema. Build-up of misfolded A1AT in the liver may also lead to liver-associated problems such as cirrhosis and jaundice.

In some embodiments of the method of the invention for treating or preventing a disease, the oligonucleotide for use, the oligonucleotide conjugate for use or the pharmaceutical composition for use of the invention, or the use of the invention, the disease is alpha 1 antitrypsin deficiency (A1AD).

Thus, the invention provides a method for treating or preventing A1AD comprising administering a therapeutically or prophylactically effective amount of the oligonucleotide or the oligonucleotide conjugate of the invention or the pharmaceutical composition of the invention to a subject suffering from or susceptible to A1AD.

The invention provides an oligonucleotide or oligonucleotide conjugate of the invention or a pharmaceutical composition of the invention, for use in the treatment or prevention of A1AD in a subject.

The invention provides use of the oligonucleotide or the oligonucleotide conjugate of the invention or the pharmaceutical composition of the invention, for the preparation of a medicament for treatment or prevention of A1AD in a subject.

Herein “preventing A1AD” includes preventing A1AD from occurring in a subject, and preventing the occurrence of symptoms of A1AD in a subject.

In some embodiments, the treatment or prevention comprises treating or preventing one or more symptoms of A1AD selected from liver damage, hepatic failure, cirrhosis, jaundice, breakdown of elastin in the lungs, emphysema and chronic obstructive pulmonary disease (COPD).

### Subject

For the purposes of the present invention, the “subject” or “patient” may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably, the subject is human. In some embodiments, the subject is suffering from a disease as referred to herein, such as A1AD. In some embodiments, the subject is susceptible to said disease.

### Sequence Listing

The sequence listing submitted with this application is hereby incorporated by reference. In the event of a discrepancy between the sequence listing and the specification or Figures, the information disclosed in the specification (including the Figures) shall be deemed to be correct.

Each reference herein to a SEQ ID NO (sequence identifier number) refers to the sequence represented by that SEQ ID NO. Each reference herein to CMP ID NO (compound identifier number) refers to the compound represented by that CMP ID NO. Each reference herein to CNJ ID NO (conjugate identifier number) refers to the conjugate represented by that CNJ ID NO.

### Helm Notation

Oligonucleotides (compounds) of the invention and oligonucleotide conjugates (conjugates) of the invention are depicted herein using Hierarchical Editing Language for Macromolecules (HELM) notation.

HELM is a notation format designed to depict the structure of macromolecules. Full details of HELM notation may be found at www.pistoiaalliance.org/helm-tools/, in Zhang et al. J. Chem. Inf. Model. 2012, 52, 2796-2806 (which initially described HELM notation) and in Milton et al. J. Chem Inf. Model. 2017, 57, 1233-1239 (which describes HELM version 2.0).

Briefly, a macromolecule is depicted as a “HELM string”, which is divided into sections. The first section of the HELM string lists the molecules comprised in the macromolecule. The second section lists the connections between molecules within the macromolecule. Third, fourth and fifth sections (which may be used in HELM strings for more complex macromolecules) are not used in the HELM strings herein. One or more dollar sign $ marks the end of a section of a HELM string.

Compounds of the invention are represented by a HELM string consisting of a single first section defining the oligonucleotide.

Conjugates of the invention are represented by a HELM string consisting of two sections: a first section, which defines the oligonucleotide (which may include a nucleotide linker sequence) and the conjugate moiety, and a second section, which defines the connection between the oligonucleotide and the conjugate moiety.

Each molecule listed in the first section of a HELM string is given an identifier (e.g. “RNA1” for a nucleic acid, “PEPTIDE1” for an amino acid sequence, “CHEM1” for a chemical structure) and the structure of the molecule is defined by notation in braces { } immediately following the identifier. “RNA1” is the identifier of the oligonucleotide part of the compound or conjugate; the oligonucleotide is defined in braces { } after the first instance of “RNA1”; the second instance of “RNA1” in each conjugate string is defining the location of the link between the oligonucleotide and the conjugate moiety. “CHEM1” is the identifier of the conjugate moiety part of the conjugate; the conjugate moiety is defined in braces { } after the first instance of “CHEM1”; the second instance of “CHEM1” in each conjugate string is defining the location of the link between the oligonucleotide and the conjugate moiety. “V2.0” indicates that HELM version 2.0 is used.

The HELM notations used to define the structure of each molecule in braces { } in the first section of HELM strings for the compounds and conjugates of the present invention are as follows:
demarcates nucleosides,
R(A) is an RNA adenine nucleoside,
R(C) is an RNA cytosine nucleoside,
R(G) is an RNA guanine nucleoside,
R([ln]) is an RNA hypoxanthine nucleoside,
R(U) is an RNA uracil nucleoside,
[dR] is a DNA nucleoside lacking a nucleobase (abasic),
[dR](A) is a DNA adenine nucleoside,
[dR](C) is a DNA cytosine nucleoside,
[dR](G) is a DNA guanine nucleoside,
[dR]([ln]) is a DNA hypoxanthine nucleoside,
[dR](T) is a DNA thymine nucleoside,
[dR]([2AP]) is a DNA 2-amino purine nucleoside,
[dR]([apC]) is a DNA aminoethyl phenoxazine nucleoside,
[dR]([5BrC]) is a DNA 5-bromo cytosine nucleoside,
[dR]([prpC]) is a DNA 5-propynyl cytosine nucleoside,
[dR]([PyrC]) is a DNA pyrrolo cytosine nucleoside,
[dR]([PPG]) is a DNA 7-deaza-8-aza guanine nucleoside,
[dR](Z) is a DNA nucleoside comprising Benner's base,
[mR](A) is a 2′-O-methyl RNA adenine nucleoside,
[mR](C) is a 2′-O-methyl RNA cytosine nucleoside,
[mR](G) is a 2′-O-methyl RNA guanine nucleoside,
[mR]([ln]) is a 2′-O-methyl RNA hypoxanthine nucleoside,
[mR](U) is a 2′-O-methyl RNA uracil nucleoside,
[fR](A) is a 2′-fluoro RNA adenine nucleoside,
[fR](C) is a 2′-fluoro RNA cytosine nucleoside,
[fR](G) is a 2′-fluoro RNA guanine nucleoside,
[fR]([ln]) is a 2′-fluoro RNA hypoxanthine nucleoside,
[fR](U) is a 2′-fluoro RNA uracil nucleoside,
[FANA](A) is a FANA adenine nucleoside,
[FANA](G) is a FANA guanine nucleoside,
[FANA](C) is a FANA cytosine nucleoside,
[LR](A) is a beta-D-oxy-LNA adenine nucleoside,
[LR](G) is a beta-D-oxy-LNA guanine nucleoside,
[LR]([5meC]) is a beta-D-oxy-LNA 5-methyl cytosine nucleoside,
[MOE] is a 2′-MOE RNA nucleoside lacking a nucleobase (abasic),
[MOE](A) is a 2′-MOE RNA adenine nucleoside,
[MOE](G) is a 2′-MOE RNA guanine nucleoside,
[MOE](T) is a 2′-MOE RNA thymine nucleoside,
[MOE]([5meC]) is a 2′-MOE RNA 5-methyl cytosine nucleoside,
[idR](A) is an iDNA adenine nucleoside,
[idR](C) is an iDNA cytosine nucleoside,
[idR](G) is an iDNA guanine nucleoside,
[ScEt](G) is an ScET guanine nucleoside,
[SNA](A) is an SNA adenine nucleoside,
[SNA](G) is an SNA guanine nucleoside,
[SNA]([5meC]) is an SNA 5-methyl cytosine nucleoside,
[TNA](A) is a TNA adenine nucleoside,
[TNA](G) is a TNA guanine nucleoside.
[TNA]([5meC]) is a TNA 5-methyl cytosine nucleoside,
[P] is a phosphodiester internucleoside linkage,
[sP] is a phosphorothioate internucleoside linkage,
[ssP] is a left-handed stereodefined isomer of a phosphorothioate internucleoside linkage (Sp).
[srP] is a right-handed stereodefined isomer of a phosphorothioate internucleoside linkage (Rp), and
[PS2] is a phosphorodithioate internucleoside linkage.

As noted above, in the context of the invention, a second section is used only in HELM strings representing conjugates of the invention. This second section lists the connections between the molecules listed in the first section. Each pair of molecules that are connected are defined by listing their identifiers, and then the attachment points between them (i.e. the point at which there is a covalent bond between the molecules) are defined.

In HELM strings representing the conjugates of the invention there is a single connection (between the conjugate moiety and the oligonucleotide). This single connection is represented in all HELM strings herein as follows:
CHEM1,RNA1,1:R2-1:R1.

This indicates that the conjugate moiety (CHEM1) is attached to the oligonucleotide (RNA1) by a covalent bond between the R2 attachment point of the first monomer of CHEM1 (which is the entire conjugate moiety) (indicated by “1:R2”) and the R1 attachment point of the first monomer of RNA1 (indicated by “1:R1”).

As noted above, “RNA1” indicates that the molecule is nucleic acid, braces { } define the part of the HELM string that refers to the nucleic acid molecule (oligonucleotide), “$$$$” indicates the end of the molecule and “V2.0” indicates that HELM version 2.0 is used. In a HELM string referring to an oligonucleotide (i.e. compound) of the invention, these characters are thus unnecessary as they do not provide any further information that is not evident from the molecule being an oligonucleotide. Therefore a simplified form of any HELM string herein can be formed by removing RNA1, { }, $$$$ and V2.0 from the HELM string. A HELM string simplified in this way is referred to herein as a “simplified HELM string”. A simplified HELM string refers to exactly the same molecule as the corresponding full HELM string. A simplified HELM string may be a more readable way to present the HELM string.

Thus, any HELM string herein referring to an oligonucleotide (i.e. compound) of the invention may be alternatively depicted using a simplified HELM string. In other words, a simplified HELM string for a given compound is interchangeable with the full HELM string for that compound.

### Example of HELM Notation

For example, CMP ID NO 32_1 is represented by the following HELM string (as depicted in Table 4):

RNA1{[mR](C)[sP].[mR](C)[sP].[mR](C)[sP].[mR](C)
[sP].[mR](A)[sP].[fR](G)[sP].[mR](C)[sP].[fR](A)
[sP].[mR](G)[sP].[fR](C)[sP].[mR](U)[sP].[fR](U)
[sP].[mR](C)[sP][fR].(A)[sP].[mR](G)[sP].[fR](U)
[sP].[mR](C)[sP].[fR](C)[sP].[mR](C)[sP].[fR](U)
[sP].[mR](U)[sP].[fR](U)[sP].[mR](C)[sP].[dR](T)
[sP].[dR](C)[sP].[dR]([In])[sP].[mR](U)[sP].[fR]
(C)[sP].[mR](G)[sP].[fR](A)[sP].[mR](U)[sP].[fR]
(G)[sP].[mR](G)[sP].[fR](U)[sP].[mR](C)[sP].[mR]
(A)[sP].[mR](G)[sP].[mR](C)[sP].[mR](A)[sP].[mR]
(C)}$$$$V2.0

This HELM string consists of a single section listing the oligonucleotide of CMP ID NO 32_1. The initial “RNA1” indicates the molecule is a nucleic acid (oligonucleotide). The structure of the oligonucleotide is presented using HELM notation in the braces { } following RNA1. “$$$$” marks the end of the section, and of the HELM string as a whole. “V2.0” indicates that HELM version 2.0 is used. A simplified HELM string referring to the same molecule, wherein RNA1, { }, $$$$ and V2.0 are removed, would be as follows:

[mR](C)[sP].[mR](C)[sP].[mR](C)[sP].[mR](C)[sP].
[mR](A)[sP].[fR](G)[sP].[mR](C)[sP].[fR](A)[sP].
[mR](G)[sP].[fR](C)[sP].[mR](U)[sP].[fR](U)[sP].
[mR](C)[sP].[fR](A)[sP].[mR](G)[sP].[fR](U)[sP].
[mR](C)[sP].[fR](C)[sP].[mR](C)[sP].[fR](U)[sP].
[mR](U)[sP].[fR](U)[sP].[mR](C)[sP].[dR](T)[sP].
[dR](C)[sP].[dR]([In])[sP].[mR](U)[sP].[fR](C)
[sP].[mR](G)[sP].[fR](A)[sP].[mR](U)[sP].[fR](G)
[sP].[mR](G)[sP].[fR](U)[sP].[mR](C)[sP].[mR](A)
[sP].[mR](G)[sP].[mR](C)[sP].[mR](A)[sP].[mR](C)

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, biochemistry, cell biology, virology or related fields are intended to be within the scope of the following claims.

### Numbered Paragraphs

Particular embodiments of the invention are as defined in the following numbered paragraphs.

1. An oligonucleotide comprising
an editing region that comprises an editing nucleoside,
a 5′ mixmer region positioned 5′ to the editing region, and
a 3′ mixmer region positioned 3′ to the editing region.

2. An oligonucleotide comprising an editing region that comprises an editing nucleoside.

3. The oligonucleotide of paragraph 1 or paragraph 2, wherein
the editing nucleoside is designated as position 0,
each nucleoside 5′ to the editing nucleoside is designated as position +x, wherein x is the number of nucleosides 5′ to the editing nucleoside at that position including the nucleoside at that position, and
each nucleoside 3′ to the editing nucleoside is designated as position −y, wherein y is the number of nucleosides 3′ to the editing nucleoside at that position including the nucleoside at that position.

4. The oligonucleotide of any one of the preceding paragraphs, wherein the oligonucleotide comprises the structure:

X⁺¹—X⁰—X⁻¹
wherein
X⁺¹ is the 5′ mixmer region,
X⁰ is the editing region, and
X⁻¹ is the 3′ mixmer region.

5. The oligonucleotide of any one of the preceding paragraphs, wherein the 5′ mixmer region comprises a first type of 5′ mixmer nucleoside and a second type of 5′ mixmer nucleoside, wherein the sugar moiety of the first type of 5′ mixmer nucleoside is different to the sugar moiety of the second type of 5′ mixmer nucleoside.

6. The oligonucleotide of any one of the preceding paragraphs, wherein the 3′ mixmer region comprises a first type of 3′ mixmer nucleoside and a second type of 3′ mixmer nucleoside, wherein the sugar moiety of the first type of 3′ mixmer nucleoside is different to the sugar moiety of the second type of 3′ mixmer nucleoside.

7. The oligonucleotide of any one of the preceding paragraphs, wherein the 5′ mixmer region comprises an alternating pattern of the first type of 5′ mixmer nucleosides and the second type of 5′ mixmer nucleosides.

8. The oligonucleotide of paragraph 7, wherein the 5′ mixmer region comprises an alternating pattern of single nucleosides of the first type of 5′ mixmer nucleosides and single nucleosides of the second type of 5′ mixmer nucleosides.

9. The oligonucleotide of any one of the preceding paragraphs, wherein the 3′ mixmer region comprises an alternating pattern of the first type of 3′ mixmer nucleoside and the second type of 3′ mixmer nucleoside.

10. The oligonucleotide of paragraph 9, wherein the 3′ mixmer region comprises an alternating pattern of single nucleosides of the first type of 3′ mixmer nucleosides and single nucleosides of the second type of 3′ mixmer nucleosides.

11. The oligonucleotide of any one of the preceding paragraphs, wherein the 5′ mixmer region comprises the structure:

(Y⁵—Z⁵—)ₘ
wherein
Y⁵ is one or more nucleosides of the first type of 5′ mixmer nucleoside,
Z⁵ is one or more nucleosides of the second type of 5′ mixmer nucleoside, and
m is a number from 1 to 20.

12. The oligonucleotide of any one of the preceding paragraphs, wherein the 3′ mixmer region comprises the structure:

(Y³—Z³—)ₙ
wherein
Y³ is one or more nucleosides of the first type of 3′ mixmer nucleoside,
Z³ is one or more nucleosides of the second type of 3′ mixmer nucleoside, and
n is a number from 1 to 20.

13. The oligonucleotide of paragraph 12, wherein each Y⁵ is independently 1, 2, 3 or 4 nucleosides, preferably 1 nucleoside;
each Z⁵ is independently 1, 2, 3 or 4 nucleosides, preferably 1 nucleoside;
each Y³ is independently 1, 2, 3 or 4 nucleosides, preferably 1 nucleoside; and/or
each Z³ is independently 1, 2, 3 or 4 nucleosides, preferably 1 nucleoside.

14. The oligonucleotide of paragraph 12 or paragraph 13, wherein
m is 1, 2, 3, 4, 5, 6, 7, 8, 9, 10, 11, 12, 13, 14, 15, 16, 17, 18, 19 or 20, preferably 6, 7, 8, 9, 10, 11, 12, 13, 14 or 15, more preferably 9; and/or
n is 1, 2, 3, 4, 5, 6, 7, 8, 9, 10, 11, 12, 13, 14, 15, 16, 17, 18, 19 or 20, preferably 3, 4, 5, 6, 7, 8 or 9, more preferably 4.

15. The oligonucleotide of any one of the preceding paragraphs, wherein the 5′ mixmer region is from 15 to 25 nucleosides long.

16. The oligonucleotide of any one of the preceding paragraphs, wherein the 5′ mixmer region is 15, 16, 17, 18, 19, 20, 21, 22, 23, 24 or 25 nucleosides long, preferably 18 nucleosides long.

17. The oligonucleotide of any one of the preceding paragraphs, wherein the 3′ mixmer region is from 4 to 12 nucleosides long.

18. The oligonucleotide of any one of the preceding paragraphs, wherein the 3′ mixmer region is 4, 5, 6, 7, 8, 9, 10, 11, or 12 nucleosides long, preferably 9 nucleosides long.

19. The oligonucleotide of any one of paragraphs 3 to 18, wherein the 5′ mixmer region comprises or consists of the nucleosides of from position +1 to position +10, +11, +12, +13, +14, +15, +16, +17, +18, +19, +20, +21, +22, +23, +24 or +25, preferably from position +2 to position +10, +11, +12, +13, +14, +15, +16, +17, +18, +19, +20, +21, +22, +23, +24 or +25, more preferably from position +2 to position +15, +16, +17, +18, +19, +20, +21, +22, +23 or +24, more preferably from position +2 to position +19, +20, +21, +22, +23 or +24.

20. The oligonucleotide of paragraph 19, wherein the 5′ mixmer region comprises or consists of the nucleosides of positions +2 to +19.

21. The oligonucleotide of any one of paragraphs 3 to 20, wherein the 3′ mixmer region comprises or consists of the nucleosides of from position −1 to position −3, −4, −5, −6, −7, −8, −9, −10, −11, −12, −13, −14, −15, −16 or −17, preferably from position −2 to position −3, −4, −5, −6, −7, −8, −9, −10, −11, −12, −13, −14, −15, −16 or −17, more preferably from position −2 to position −5, −6, −7, −8, −9, −10, −11, −12, −13, −14 or −15, more preferably from position −2 to position −5, −6, −7, −8, −9 or −10.

22. The oligonucleotide of paragraph 21, wherein the 3′ mixmer region comprises or consists of the nucleosides of positions −2 to −10.

23. The oligonucleotide of any of paragraphs 3 to 22, wherein the 5′ mixmer region consists of the nucleosides of from position +2 to position +15, +16, +17, +18, +19, +20, +21, +22, +23 or +24, preferably from position +2 to position +17, +18, +19, +20, +21, +22, +23 or +24; and the 3′ mixmer region consists of the nucleosides of from position −2 to position −5, −6, −7, −8, −9, −10, −11, −12, −13, −14 or −15, more preferably from position −2 to position −5, −6, −7, −8, −9 or −10.

24. The oligonucleotide of paragraph 23, wherein
the 5′ mixmer region consists of the nucleosides of positions +2 to +19; and
the 3′ mixmer region consists of the nucleosides of positions −2 to −10.

25. The oligonucleotide of any one of paragraphs 5 to 24, wherein
(a) the first type of 5′ mixmer nucleoside is selected from the group consisting of DNA, RNA, 2′-O-methyl-RNA, 2′-O-methoxyethyl-RNA (MOE-RNA), 2′-fluoro-RNA, linked nucleic acid (LNA), arabinonucleic acid (ANA) and 2′-fluoroarabinonucelic acid (FANA) nucleosides, preferably wherein the first type of 5′ mixmer nucleoside is a 2′-O-methyl-RNA nucleoside or a 2′-fluoro-RNA nucleoside, more preferably a 2′-fluoro-RNA nucleoside; and/or
(b) the second type of 5′ mixmer nucleoside is selected from the group consisting of DNA, RNA, 2′-O-methyl-RNA, MOE-RNA, 2′-fluoro-RNA, LNA, ANA and FANA nucleosides, preferably wherein the second type of 5′ mixmer nucleoside is a 2′-O-methyl-RNA nucleoside or a 2′-fluoro-RNA nucleoside, more preferably a 2′-O-methyl-RNA nucleoside; and/or
(c) the first type of 3′ mixmer nucleoside is selected from the group consisting of DNA, RNA, 2′-O-methyl-RNA, MOE-RNA, 2′-fluoro-RNA, LNA, ANA and FANA nucleosides, preferably wherein the first type of 3′ mixmer nucleoside is a 2′-O-methyl-RNA nucleoside or a 2′-fluoro-RNA nucleoside, more preferably a 2′-O-methyl-RNA nucleoside; and/or
(d) the second type of 3′ mixmer nucleoside is selected from the group consisting of DNA, RNA, 2′-O-methyl-RNA, MOE-RNA, 2′-fluoro-RNA, LNA, ANA and FANA nucleosides, preferably wherein the second type of 3′ mixmer nucleoside is a 2′-O-methyl-RNA nucleoside or a 2′-fluoro-RNA nucleoside, more preferably a 2′-fluoro-RNA nucleoside.

26. The oligonucleotide of paragraph 25, wherein
the first type of 5′ mixmer nucleoside is a 2′-fluoro-RNA nucleoside, and/or
the second type of 5′ mixmer nucleoside is a 2′-O-methyl-RNA nucleoside, and/or
the first type of 3′ mixmer nucleoside is a 2′-fluoro-RNA nucleoside, and/or
the second type of 3′ mixmer nucleoside is a 2′-O-methyl-RNA nucleoside.

27. The oligonucleotide of paragraph 26, wherein
the first type of 5′ mixmer nucleoside is a 2′-fluoro-RNA nucleoside,
the second type of 5′ mixmer nucleoside is a 2′-O-methyl-RNA nucleoside,
the first type of 3′ mixmer nucleoside is a 2′-fluoro-RNA nucleoside, and
the second type of 3′ mixmer nucleoside is a 2′-O-methyl-RNA nucleoside.

28. The oligonucleotide of any one of the preceding paragraphs, wherein the 5′ mixmer region comprises or consists of 2′-fluoro-RNA nucleosides and 2′-O-methyl-RNA nucleosides.

29. The oligonucleotide of any one of the preceding paragraphs, wherein the 5′ mixmer region comprises or consists of alternating 2′-fluoro-RNA nucleosides and 2′-O-methyl-RNA nucleosides, preferably alternating single 2′-fluoro-RNA nucleosides and single 2′-O-methyl-RNA nucleosides.

30. The oligonucleotide of any one of the preceding paragraphs, wherein the 3′ mixmer region comprises or consists of 2′-fluoro-RNA nucleosides and 2′-O-methyl-RNA nucleosides.

31. The oligonucleotide of any one of the preceding paragraphs, wherein the 3′ mixmer region comprises or consists of alternating 2′-fluoro-RNA nucleosides and 2′-O-methyl-RNA nucleosides, preferably alternating single 2′-fluoro-RNA nucleosides and single 2′-O-methyl-RNA nucleosides.

32. The oligonucleotide of any one paragraphs 3 to 31, wherein each nucleoside at positions +2 to +19 is a 2′-fluoro-RNA nucleoside or a 2′-O-methyl-RNA nucleoside.

33. The oligonucleotide of any one paragraphs 3 to 32, wherein each nucleoside at positions −2 to −10 is a 2′-fluoro-RNA nucleoside or a 2′-O-methyl-RNA nucleoside.

34. The oligonucleotide of any one paragraphs 3 to 33, wherein each nucleoside at positions +2 to +24 is a 2′-fluoro-RNA nucleoside or a 2′-O-methyl-RNA nucleoside.

35. The oligonucleotide of any one paragraphs 3 to 34, wherein each nucleoside at positions −2 to −15 is a 2′-fluoro-RNA nucleoside or a 2′-O-methyl-RNA nucleoside.

36. The oligonucleotide of any one of paragraphs 3 to 35, wherein the nucleoside at one or more of positions +3, +5, +7, +9, +11, +13, +15, +17 and +19 is a 2′-fluoro-RNA nucleoside, preferably wherein the nucleoside at each of positions +3, +5, +7, +9, +11, +13, +15, +17 and +19 is a 2′-fluoro-RNA nucleoside.

37. The oligonucleotide of any one of paragraphs 3 to 36, wherein the nucleoside at one or more of positions −3, −5, −7 and −9 is a 2′-fluoro-RNA nucleoside, preferably wherein the nucleoside at each of positions −3, −5, −7 and −9 is a 2′-fluoro-RNA nucleoside.

38. The oligonucleotide of any one of paragraphs 3 to 37, wherein the nucleoside at one or more of positions +2, +4, +6, +8, +10, +12, +14 and +18 is a 2′-O-methyl-RNA nucleoside, preferably wherein the nucleoside at each of positions +2, +4, +6, +8, +10, +12, +14 and +18 is a 2′-O-methyl-RNA nucleoside.

39. The oligonucleotide of any one of paragraphs 3 to 38, wherein the nucleoside at one or more of positions −2, −4 and −6 is a 2′-O-methyl-RNA nucleoside, preferably wherein the nucleoside at each of positions −2, −4 and −6 is a 2′-O-methyl-RNA nucleoside.

40. The oligonucleotide of any one of paragraphs 3 to 39, wherein the nucleoside at one or more of positions +3, +5, +7, +9, +11, +13, +15, +17, +19, +21 and +23 is a 2′-fluoro-RNA nucleoside, preferably wherein the nucleoside at each of positions +3, +5, +7, +9, +11, +13, +15, +17, +19, +21 and +23 is a 2′-fluoro-RNA nucleoside.

41. The oligonucleotide of any one of paragraphs 3 to 40, wherein the nucleoside at one or more of positions −3, −5, −7, −9, −11, −13 and −15 is a 2′-fluoro-RNA nucleoside, preferably wherein the nucleoside at each of positions −3, −5, −7, −9, −11, −13 and −15 is a 2′-fluoro-RNA nucleoside.

42. The oligonucleotide of any one of paragraphs 3 to 41, wherein the nucleoside at one or more of positions +2, +4, +6, +8, +10, +12, +14, +18, +20, +22 and +24 is a 2′-O-methyl-RNA nucleoside, preferably wherein the nucleoside at each of positions +2, +4, +6, +8, +10, +12, +14, +18, +20, +22 and +24 is a 2′-O-methyl-RNA nucleoside.

43. The oligonucleotide of any one of paragraphs 3 to 42, wherein the nucleoside at one or more of positions −2, −4, −6, −10, −12 and −14 is a 2′-O-methyl-RNA nucleoside, preferably wherein the nucleoside at each of positions −2, −4, −6, −10, −12 and −14 is a 2′-O-methyl-RNA nucleoside.

44. The oligonucleotide of any one paragraphs 3 to 43, wherein the nucleoside at position −2 is a 2′-O-methyl-RNA nucleoside.

45. The oligonucleotide of any one of paragraphs 3 to 44, wherein the nucleoside at position −3 is a 2′-fluoro-RNA nucleoside.

46. The oligonucleotide of any one of paragraphs 3 to 45, wherein the nucleoside at position −8 is a 2′-fluoro-RNA nucleoside.

47. The oligonucleotide of any one of paragraphs 3 to 46, wherein the nucleoside at position +16 is a 2′-fluoro-RNA nucleoside.

48. The oligonucleotide of any one of paragraphs 3 to 47, wherein the nucleoside at position +17 is a 2′-fluoro-RNA nucleoside.

49. The oligonucleotide of any one of paragraphs 3 to 45, 47 or 48, wherein the nucleoside at position −8 is a 2′-O-methyl-RNA nucleoside.

50. The oligonucleotide of any one of paragraphs 3 to 46, 48 or 49, wherein the nucleoside at position +16 is a 2′-O-methyl-RNA nucleoside.

51. The oligonucleotide of any one of the preceding paragraphs, wherein the oligonucleotide is for editing a target nucleic acid.

52. The oligonucleotide of paragraph 51, wherein the target nucleic acid comprises a target adenosine.

53. The oligonucleotide of paragraph 51 or paragraph 52, wherein the oligonucleotide is capable of binding to the target nucleic acid by complementary base pairing.

54. The oligonucleotide of any one of the preceding paragraphs, wherein the oligonucleotide is a guide oligonucleotide for an adenosine deaminase acting on RNA (ADAR).

55. The oligonucleotide of any one of the preceding paragraphs, wherein the oligonucleotide is capable of recruiting an ADAR to the target nucleic acid.

56. The oligonucleotide of paragraph 54 or paragraph 55, wherein the ADAR is ADAR1 or ADAR2.

57. The oligonucleotide of any one of paragraphs 52 to 56, wherein the oligonucleotide is capable of effecting conversion of the target adenosine (A) to inosine (1).

58. The oligonucleotide of any one of paragraphs 51 to 57, wherein the target nucleic acid is RNA.

59. The oligonucleotide of any one of paragraphs 51 to 58, wherein the target nucleic acid encodes a protein.

60. The oligonucleotide of any one of paragraphs 51 to 59, wherein the target nucleic acid is mRNA.

61. The oligonucleotide of any one of paragraphs 51 to 60, wherein the target nucleic acid encodes alpha-1 antitrypsin (A1AT).

62. The oligonucleotide of any one of paragraphs 51 to 61, wherein the target nucleic acid is a SERPINA1 mRNA.

63. The oligonucleotide of any one of paragraphs 51 to 62, wherein the oligonucleotide is capable of effecting conversion of an AAA codon encoding lysine to an IAA codon encoding glutamate on the target nucleic acid.

64. The oligonucleotide of any one of paragraphs 51 to 63, wherein the oligonucleotide is capable of effecting conversion of an AAG codon encoding lysine to an IAG codon encoding glutamate on the target nucleic acid.

65. The oligonucleotide of any one of paragraphs 52 to 64, wherein the target adenosine corresponds to A1024 of SEQ ID NO 186.

66. The oligonucleotide of any one of paragraphs 51 to 65, wherein the target nucleic acid comprises or consists of the sequence according to SEQ ID NO 186.

67. The oligonucleotide of any one of paragraphs 51 to 66, wherein the editing nucleoside is not complementary to the target adenosine.

68. The oligonucleotide of any one of the preceding paragraphs, wherein the nucleobase of the editing nucleoside is selected from the group consisting of cytosine, 5-methyl cytosine, guanine and hypoxanthine.

69. The oligonucleotide of any one of the preceding paragraphs, wherein the editing nucleoside is cytidine (C).

70. The oligonucleotide of any one of the preceding paragraphs, wherein the editing region consists of the editing nucleoside.

71. The oligonucleotide of any one of paragraphs 3 to 70, wherein the editing region comprises or consist of the nucleosides at positions +1, 0 and −1.

72. The oligonucleotide of any one of the preceding paragraphs, wherein the editing region comprises or consists of an editing triplet consisting of 3 nucleosides, wherein the editing nucleoside is the central nucleoside of the editing triplet.

73. The oligonucleotide of paragraph 72, wherein the editing triplet is 5′-thymidine-cytidine-inosine-3′ (TCI).

74. The oligonucleotide of any one of paragraphs 5 to 73, wherein the editing region comprises one or more nucleosides which comprise a different sugar moiety to the sugar moiety of the first type of 5′ mixmer nucleoside, the second type of 5′ mixmer nucleoside, the first type of 3′ mixmer nucleoside, and/or the second type of 3′ mixmer nucleoside.

75. The oligonucleotide of paragraph 74, wherein each nucleoside of the editing region comprises a different sugar moiety to the sugar moiety of the first type of 5′ mixmer nucleoside, the second type of 5′ mixmer nucleoside, the first type of 3′ mixmer nucleoside, and the second type of 3′ mixmer nucleoside.

76. The oligonucleotide of any one of the preceding paragraphs, wherein each nucleoside of the editing region comprises the same sugar moiety.

77. The oligonucleotide of any one of the preceding paragraphs, wherein each nucleoside of the editing region is independently selected from the group consisting of DNA, RNA, 2′-O-methyl-RNA, 2′-fluoro-RNA, MOE-RNA, LNA, ANA, and FANA nucleosides.

78. The oligonucleotide of any one of the preceding paragraphs, wherein the editing region comprises one or more DNA nucleosides.

79. The oligonucleotide of any one of the preceding paragraphs, wherein the editing nucleoside is a DNA nucleoside.

80. The oligonucleotide of any one of the preceding paragraphs, wherein all nucleosides of the editing region are DNA nucleosides.

81. The oligonucleotide of any one of paragraphs 3 to 80, wherein the nucleoside at one or more of positions +1, 0 and −1 is a DNA nucleoside, preferably wherein the nucleoside at each of positions +1, 0 and −1 is a DNA nucleoside.

82. The oligonucleotide of paragraph 81, wherein the editing region consists of the nucleosides at positions +1, 0 and −1 of the oligonucleotide, wherein the nucleoside at position +1 is a thymidine DNA nucleoside, the nucleoside at position 0 is a cytidine DNA nucleoside, and the nucleoside at position −1 is an inosine DNA nucleoside.

83. The oligonucleotide of any one paragraphs 1 to 78, wherein the editing nucleoside is a FANA nucleoside.

84. The oligonucleotide of paragraph 83, wherein the editing nucleoside is a FANA nucleoside and all other nucleosides in the editing region are DNA nucleosides.

85. The oligonucleotide of any one of paragraphs 3 to 78, wherein the nucleoside at position 0 is a FANA nucleoside.

86. The oligonucleotide of paragraph 85, wherein
(a) the nucleoside at position +1 is a DNA nucleoside, or
(b) the nucleoside at position −1 is a DNA nucleoside, or
(c) the nucleoside at each of positions +1 and −1 is a DNA nucleoside.

87. The oligonucleotide of paragraph 86, wherein the editing region consists of the nucleosides at positions +1, 0 and −1 of the oligonucleotide, wherein the nucleoside at position +1 is a thymidine DNA nucleoside, the nucleoside at position 0 is a cytidine FANA nucleoside, and the nucleoside at position −1 is an inosine DNA nucleoside.

88. The oligonucleotide of any one of the preceding paragraphs, wherein the oligonucleotide is from 30 to 61 nucleosides long, preferably from 30 to 50 nucleosides long, more preferably from 35 to 45 nucleosides long.

89. The oligonucleotide of any one of the preceding paragraphs, wherein the oligonucleotide is 30, 31, 32, 33, 34, 35, 36, 37, 38, 39, 40, 41, 42, 43, 44, 45, 46, 47, 48, 49 or 50 nucleosides long, preferably 35, 36, 37, 38, 39, 40, 41, 42, 43, 44 or 45 nucleosides long, more preferably 40 nucleosides long.

90. The oligonucleotide of any one of the preceding paragraphs, wherein the oligonucleotide comprises exactly 20, 21, 22, 23, 24, 25, 26, 27, 28, 29 or 30 nucleosides 5′ to the editing nucleoside, preferably 24, 25, 26 or 27 nucleosides 5′ to the editing nucleoside, more preferably exactly 24 nucleosides 5′ to the editing nucleoside.

91. The oligonucleotide of any one of the preceding paragraphs, wherein the oligonucleotide comprises exactly 4, 5, 6, 7, 8, 9, 10, 11, 12, 13, 14, 15, 16, 17, 18, 19 or 20 nucleotides 3′ to the editing nucleoside, preferably exactly 12, 13, 14 or 15 nucleotides 3′ to the editing nucleoside, more preferably exactly 15 nucleotides 3′ to the editing nucleoside.

92. The oligonucleotide of any one of the preceding paragraphs, wherein the oligonucleotide comprises exactly 24 nucleotides 5′ to the editing nucleoside and exactly 15 nucleotides 3′ to the editing nucleoside.

93. The oligonucleotide of any one of the preceding paragraphs, wherein the oligonucleotide comprises one or more phosphorothioate internucleoside linkages.

94. The oligonucleotide of any one of paragraphs 3 to 93, wherein the internucleoside linkage between nucleosides at positions +1 and 0 is a phosphodiester internucleoside linkage.

95. The oligonucleotide of any one of paragraphs 3 to 94, wherein the internucleoside linkage between nucleosides at positions 0 and −1 is a phosphodiester internucleoside linkage.

96. The oligonucleotide of any one of paragraphs 3 to 95, wherein the internucleoside linkage between nucleosides at positions −1 and −2 is a phosphorothioate internucleoside linkage.

97. The oligonucleotide of any one of paragraphs 3 to 96, wherein the internucleoside linkage between nucleosides at positions −2 and −3 is a phosphodiester internucleoside linkage.

98. The oligonucleotide of any one of paragraphs 94 to 97, wherein all other internucleoside linkages are phosphorothioate internucleoside linkages.

99. The oligonucleotide of any one of paragraphs 1 to 93, wherein all internucleoside linkages of the oligonucleotide are phosphorothioate internucleoside linkages.

100. The oligonucleotide of any one of the preceding paragraphs, wherein the oligonucleotide comprises a 5′ flank region positioned 5′ to the 5′ mixmer region.

101. The oligonucleotide of any one of the preceding paragraphs, wherein the oligonucleotide comprises a 3′ flank region positioned 3′ to the 3′ mixmer region.

102. The oligonucleotide of paragraph 102, wherein the oligonucleotide comprises the structure:

X⁺²—X⁺¹—X⁰—X⁻¹—X⁻²
wherein
X⁺² is the 5′ flank region,
X⁺¹ is the 5′ mixmer region,
X⁰ is the editing region,
X⁻¹ is the 3′ mixmer region, and
X⁻² is the 3′ flank region.

103. The oligonucleotide of any one of paragraphs 100 to 102, wherein the 5′ flank region is 1, 2, 3, 4 or 5 nucleosides long, preferably 5 nucleosides long.

104. The oligonucleotide of any one of paragraphs 101 to 103, wherein the 3′ flank region is 1, 2, 3, 4 or 5 nucleosides long, preferably 5 nucleosides long.

105. The oligonucleotide of any one of paragraphs 100 to 104, wherein the 5′ flank region consists of the 1, 2, 3, 4 or 5 nucleosides at the 5′ end of the oligonucleotide, preferably the 5 nucleosides at the 5′ end of the oligonucleotide.

106. The oligonucleotide of any one of paragraphs 101 to 105, wherein the 3′ flank region consists of the 1, 2, 3, 4 or 5 nucleosides at the 3′ end of the oligonucleotide, preferably the 5 nucleosides at the 3′ end of the oligonucleotide.

107. The oligonucleotide of any one of paragraphs 100 to 106, wherein the 5′ flank region comprises one or more sugar modified nucleosides.

108. The oligonucleotide of any one of paragraphs 101 to 107, wherein the 3′ flank region comprises one or more sugar modified nucleosides.

109. The oligonucleotide of paragraph 107 or paragraph 108, wherein the one or more sugar modified nucleosides are independently selected from the group consisting of 2′-O-methyl-RNA, 2′-fluoro-RNA, 2′-O-methoxyethyl-RNA (MOE-RNA) and LNA nucleosides.

110. The oligonucleotide of any one of paragraphs 100 to 109, wherein all nucleosides of the 5′ flank region are sugar modified nucleosides.

111. The oligonucleotide of any one of paragraphs 101 to 110, wherein all nucleosides of the 3′ flank region are sugar modified nucleosides.

112. The oligonucleotide of any one of paragraphs 100 to 111, wherein all nucleosides of the 5′ flank region are 2′-O-methyl-RNA.

113. The oligonucleotide of any one of paragraphs 101 to 112, wherein all nucleosides of the 3′ flank region are 2′-O-methyl-RNA.

114. The oligonucleotide of any one of the preceding paragraphs, wherein the oligonucleotide comprises or consists of a sequence that is complementary to a SERPINA1 mRNA.

115. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% complementarity to positions 986 to 1062 of SEQ ID NO 186, wherein any match or mismatch between the editing nucleoside and the target adenosine is not included when determining the percentage complementarity.

116. The oligonucleotide of any one of the preceding paragraphs, wherein the sequence of the oligonucleotide comprises or consists of from 30 to 61 contiguous nucleosides from the following sequence:

(SEQ ID NO 87)
CUCUAAAAACAUGGCCCCAGCAGCUUCAGUCCCUUUCTCIUCGAUGGU
CAGCACAGCCUUAUGCACGGCCUUGGUGU,

or from a variant of SEQ ID NO 87 comprising exactly 1, exactly 2 or exactly 3 single nucleoside substitutions,
wherein I is inosine, and
wherein the oligonucleotide comprises positions 38, 39 and 40 of SEQ ID NO 87.

117. The oligonucleotide of paragraph 116, wherein positions 38, 39 and 40 of SEQ ID NO 87 are the editing triplet.

118. The oligonucleotide of paragraph 116 or paragraph 117, wherein the sequence of the oligonucleotide comprises or consists of from 30 to 50 contiguous nucleosides from SEQ ID NO 87 or from the variant thereof.

119. The oligonucleotide of paragraph 118, wherein the sequence of the oligonucleotide comprises or consists of 30, 31, 32, 33, 34, 35, 36, 37, 38, 39, 40, 41, 42, 43, 44, 45, 46, 47, 48, 49 or 50 contiguous nucleosides, preferably 40 contiguous nucleosides, from SEQ ID NO 87 or from the variant thereof.

120. The oligonucleotide of any one of paragraphs 116 to 119, wherein the variant of SEQ ID NO 87 comprises exactly 1 single nucleoside substitution.

121. The oligonucleotide of any one of paragraphs 116 to 120, wherein the variant of SEQ ID NO 87 comprises a nucleoside substitution at position 38, 39 and/or 40 of SEQ ID NO 87.

122. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% identity to any one of SEQ ID NOs 1 to 86.

123. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 and 86.

124. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% identity to any one of SEQ ID NOs 2, 3, 7, 8, 9, 10, 13, 19, 20, 21, 22, 23, 26, 29, 30, 31, 32, 33, 34, 35, 36, 41, 42, 43, 45, 59, 60, 61, 62, 63, 64 and 65.

125. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% identity to any one of SEQ ID NOs 3, 7, 8, 9, 19, 20, 21, 22, 29, 30, 31, 32, 33, 34, 42, 43, 62 and 63.

126. The oligonucleotide of any one of the preceding paragraphs, wherein the sequence of the oligonucleotide comprises or consists of a sequence having at least 80%, at least 85%, at least 90%, at least 95%, at least 96%, at least 97%, at least 98%, at least 99% or 100% identity to SEQ ID NO 32.

127. The oligonucleotide of any one of the preceding paragraphs, wherein the oligonucleotide comprises or consists of any one of CMP ID NOs 1_1, 2_1, 3_1, 3_2, 3_3, 4_1, 5_1, 6_1, 7_1, 8_1, 9_1, 10_1, 11_1, 121, 131, 141, 151, 161, 171, 181, 191, 20_1, 21_1, 22_1, 23_1, 24_1, 25_1, 26_1, 26_2, 26_3, 26_4, 26_5, 26_6.26_7, 26_8, 26_9, 27_1, 28_1, 29_1, 30_1, 31_1, 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82, 32_83, 33_1, 34_1, 35_1, 36_1, 37_1, 38_1, 39_1, 40_1, 40_2, 40_3, 40_4, 40_5, 40_6, 40_7, 40_8, 40_9, 40_10, 41_1, 41_2, 41_3, 41_4, 41_5, 41_6, 41_7, 41_8, 41_9, 41_10, 42_1, 42_2, 42_3, 42_4, 42_5, 42_6, 43_1, 43_3, 44_1, 45_1, 46_1, 47_1, 47_2, 48_1, 49_1, 50_1, 51_1, 52_1, 53_1, 54_1, 55_1, 56_1, 57_1, 58_1, 59_1, 60_1, 61_1, 62_1, 63_1, 64_1, 65_1, 66_1, 67_1, 68_1.69_1, 70_1, 71_1, 72_1, 73_1, 74_1, 75_1, 76_1, 77_1, 78_1.79_1, 801, 81_1, 82_1, 83_1, 84_1, 85_1 and 86_1.

128. The oligonucleotide of any one of the preceding paragraphs, wherein the oligonucleotide comprises or consists of any one of CMP ID NOs 2_1, 3_1, 3_3, 7_1, 8_1, 9_1, 10_1, 13_1, 19_1, 20_1, 21_1, 22_1, 231, 268, 29_1, 30_1, 31_1, 32_1, 322, 324, 32_5, 32_6, 32_7, 32_8, 32_9, 32_10, 32_11, 32_12, 32_13, 32_14, 32_16, 32_17, 32_18, 32_19, 32_20, 32_21, 32_22, 32_23, 32_29, 32_33, 32_34, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 33_1, 34_1, 35_1, 36_1, 41_8, 42_6, 43_1, 43_3, 45_1, 59_1, 60_1, 61_1, 62_1, 63_1, 64_1 and 65_1.

129. The oligonucleotide of any one of the preceding paragraphs, wherein the oligonucleotide comprises or consists of any one of CMP ID NOs 3_1, 7_1, 8_1, 9_1, 19_1, 20_1, 21_1, 22_1, 29_1, 30_1, 31_1.19_1, 32_1, 32_4, 32_5, 32_6.32_7, 32_8, 32_9, 32_10, 32_11, 32_12, 32_13, 32_14, 32_16, 32_17, 32_18, 32_19, 32_20, 32_21, 32_22, 32_23, 32_33, 32_47, 32_48, 32_49, 32_50, 32_51, 32_52, 32_53, 32_54, 32_55, 32_56, 32_57, 3258, 3260, 3261, 3262, 3263, 3264, 3265, 33_1, 34_1, 42_6, 43_1, 62_1 and 63_1.

130. The oligonucleotide of any one of the preceding paragraphs, wherein the oligonucleotide comprises or consists of any one of CMP ID NOs 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82 and 32_83.

131. The oligonucleotide of any one of the preceding paragraphs, wherein the oligonucleotide comprises or consists of CMP ID NO 32_1.

132. An oligonucleotide as depicted in FIG. 29.

133. An oligonucleotide conjugate comprising the oligonucleotide of any one of the preceding paragraphs covalently attached to at least one conjugate moiety.

134. The oligonucleotide conjugate of paragraph 133, wherein the conjugate moiety is covalently attached to the oligonucleotide via a linker.

135. The oligonucleotide conjugate of paragraph 134 or paragraph 135, wherein the conjugate moiety is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

136. The oligonucleotide conjugate of paragraph 135, wherein the conjugate moiety is an N-acetylgalactosamine (GalNAc) conjugate moiety.

137. The oligonucleotide or the oligonucleotide conjugate of any one of the preceding paragraphs, wherein the oligonucleotide or the oligonucleotide conjugate is in the form of a pharmaceutically acceptable salt.

138. The oligonucleotide or the oligonucleotide conjugate of paragraph 137, wherein the salt is a sodium salt or a potassium salt.

139. The oligonucleotide or the oligonucleotide conjugate of any one of the preceding paragraphs, wherein the oligonucleotide or the oligonucleotide conjugate is encapsulated in a lipid-based delivery vehicle, covalently linked to or encapsulated in a dendrimer, or conjugated to an aptamer.

140. A pharmaceutical composition comprising the oligonucleotide or the oligonucleotide conjugate of any one of paragraphs 1 to 139, and a pharmaceutically acceptable diluent, solvent, carrier, salt and/or adjuvant.

141. The pharmaceutical composition of paragraph 140 wherein the pharmaceutical composition comprises an aqueous diluent or solvent, such as phosphate buffered saline.

142. An in vitro method for editing a target nucleic acid in a target cell, the method comprising administering an effective amount of the oligonucleotide or the oligonucleotide conjugate of any one of paragraphs 1 to 139 or the pharmaceutical composition of paragraph 140 or paragraph 141 to the target cell.

143. An in vivo method for editing a target nucleic acid in a target cell, the method comprising administering an effective amount of the oligonucleotide or the oligonucleotide conjugate of any one of paragraphs 1 to 139 or the pharmaceutical composition of paragraph 140 or paragraph 141 to the target cell.

144. The method of paragraph 142 or paragraph 143, wherein the target nucleic acid comprises a target adenosine.

145. The method of any one of paragraphs 142 to 144, wherein the target cell is a mammalian cell.

146. The method of paragraph 145, wherein the target cell is a human cell.

147. The method of any one of paragraphs 142 to 146, wherein the target cell is a lung cell or a hepatocyte.

148. The method of any one of paragraphs 142 to 147, wherein the amount of edited target nucleic acid is increased compared to a control by at least 10%, at least 20%, at least 30%, at least 40%, at least 50%, at least 60%, at least 70%, at least 75%, at least 80%, at least 85%, at least 90%, at least 95% or 100%.

149. The method of paragraph 148, wherein the control is a cell that has not been exposed to the oligonucleotide or the oligonucleotide conjugate.

150. The method of any one of paragraphs 142 to 149, wherein the oligonucleotide binds to the target nucleic acid by complementary base pairing.

151. The method of any one of paragraphs 142 to 150, wherein the oligonucleotide recruits an ADAR to the target nucleic acid.

152. The method of paragraph 151, wherein the ADAR is ADAR1 or ADAR2.

153. The method of any one of paragraphs 142 to 152, wherein the target adenosine (A) is converted to inosine (1).

154. The method of any one of paragraphs 142 to 153, wherein the target nucleic acid encodes a protein.

155. The method of any one of paragraphs 142 to 154, wherein the target nucleic acid is mRNA.

156. The method of any one of paragraphs 142 to 155, wherein the target nucleic acid encodes alpha-1 antitrypsin (A1AT).

157. The method of any one of paragraphs 142 to 156, wherein the target nucleic acid is a SERPINA1 mRNA.

158. The method of any one of paragraphs 142 to 157, wherein an AAA codon encoding lysine is converted to an IAA codon encoding glutamate on the target nucleic acid.

159. The method of any one of paragraphs 142 to 158, wherein an AAG codon encoding lysine is converted to an IAG codon encoding glutamate on the target nucleic acid.

160. The method of any one of paragraphs 142 to 159, wherein the target adenosine is A1024 on a target nucleic acid as defined by SEQ ID NO 186.

161. The method of any one of paragraphs 142 to 160, wherein the target nucleic acid comprises or consists of the sequence according to SEQ ID NO 186.

162. A method for treating or preventing a disease comprising administering a therapeutically or prophylactically effective amount of the oligonucleotide or the oligonucleotide conjugate of any one of paragraphs 1 to 139 or the pharmaceutical composition of paragraph 140 or paragraph 141 to a subject suffering from or susceptible to a disease.

163. The oligonucleotide or the oligonucleotide conjugate of any one of paragraphs 1 to 139 or the pharmaceutical composition of paragraph 140 or paragraph 141, for use in the treatment or prevention of a disease in a subject.

164. Use of the oligonucleotide or the oligonucleotide conjugate of any one of paragraphs 1 to 139 or the pharmaceutical composition of paragraph 140 or paragraph 141, for the preparation of a medicament for treatment or prevention of a disease in a subject.

165. The method of paragraph 162, the oligonucleotide for use, the oligonucleotide conjugate for use or the pharmaceutical composition for use according to paragraph 163, or the use according to paragraph 164, wherein the disease is alpha 1 antitrypsin deficiency (A1AD).

166. The method, the oligonucleotide for use, the oligonucleotide conjugate for use, the pharmaceutical composition for use, or the use according to paragraph 165, wherein the treatment or prevention comprises treating or preventing one or more symptoms of A1AD selected from liver damage, hepatic failure, cirrhosis, jaundice, breakdown of elastin in the lungs, emphysema and chronic obstructive pulmonary disease (COPD).

### EXAMPLES

### Example 1—Oligonucleotide Sequences and Compounds

Oligonucleotides (compounds) and conjugates for editing SERPINA1, GAPDH or mHprt mRNA were synthesised.

### Oligonucleotide Synthesis

Oligonucleotide synthesis is generally known in the art. The oligonucleotides and conjugates of the invention may be synthesized by any such method known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on a Mermade 192 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC), ion exchange chromatography or by solid phase extractions and characterized by UPLC, and the molecular mass is further confirmed by ESI-MS.

### Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC:

The crude compounds are purified by preparative RP-HPLC on a Phenomenex Jupiter® C18 10 μm 150×10 mm column. 0.1 M ammonium acetate pH 8 and acetonitrile is used as buffers at a flow rate of 5 mL/min. The collected fractions are lyophilized to give the purified compound typically as a white solid.

### Abbreviation

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Tables of Sequences, Compounds and Conjugates

Tables 3, 5 and 7 below show the sequences of the oligonucleotides for editing SERPINA1, GAPDH and mHprt respectively. The code used in Tables 3, 5 and 7 is as follows:
Code for nucleobases: A=adenine, ab=abasic (i.e. no nucleobase), C=cytosine, E=5-methyl cytosine, F=7-deaza-8-azaguanine, G=guanine, I=hypoxanthine (i.e. an inosine nucleotide), prpC=5-propynyl cytosine, pyrC=pyrrolo cytosine, T=thymine, U=uracil, Z=Benner's base, 2AP=2-amino purine, apC=aminoethyl phenoxazine, 5brC=5-bromo cytosine

Tables 4, 6 and 8 below show the sequences, pattern of sugar modifications and pattern of internucleoside linkages of the oligonucleotides for editing SERPINA1, GAPDH and mHprt respectively using HELM strings. Table 9 also describes a conjugate using a HELM string.

An explanation of HELM notation used in Tables 4, 6, 8 and 9 is provided in the detailed description herein.

TABLE 3
SERPINA1 editing sequences

| SEQ | | | CMP |
|---|---|---|---|
| ID | | | ID |
| NO | Sequence | Length | NOS |
| 1 | UAAAAACAUGGCCCCAGCAGCUUCAGUCCCUUUCUCIUCGAUGGUCAGC | 49 | 1_1 |
| 2 | ACAUGGCCCCAGCAGCUUCAGUCCCUUUCUCGUCGAUGGUCAGCACAGCCUUAUGCACGGC | 61 | 2_1 |
| 3 | ACAUGGCCCCAGCAGCUUCAGUCCCUUUCUCIUCGAUGGUCAGCACAGCCUUAUGCACGGC | 61 | 3_1 |
| | | | to |
| | | | 3_3 |
| 4 | UGGCCCCAGCAGCUUCAGUCCCUUUCTCIUCGAUGGUCAGCACAGCCUUAUGCAC | 55 | 4_1 |
| 5 | GCCCCAGCAGCUUCAGUCCCUUUCTCIUCGAUGGUCAGCACAGCCUUAUGC | 51 | 5_1 |
| 6 | GCCCCAGCAGCUUCAGUCCCUUUCTCIUCGAUGGUC | 36 | 6_1 |
| 7 | ACAUGGCCCCAGCAGCUUCAGUCCCUUUCTCIUCGAUGGUCAGCAC | 46 | 7_1 |
| 8 | ACAUGGCCCCAGCAGCUUCAGUCCCUUUCTCIUCGAUGGUCAGCA | 45 | 8_1 |
| 9 | ACAUGGCCCCAGCAGCUUCAGUCCCUUUCTCIUCGAUGGUCAGC | 44 | 9_1 |
| 10 | ACAUGGCCCCAGCAGCUUCAGUCCCUUUCTCIUCGAUGGUCAG | 43 | 10_1 |
| 11 | ACAUGGCCCCAGCAGCUUCAGUCCCUUUCTCIUCGAUGGUCA | 42 | 11_1 |
| 12 | ACAUGGCCCCAGCAGCUUCAGUCCCUUUCTCIUCGAUGGUC | 41 | 12_1 |
| 13 | ACAUGGCCCCAGCAGCUUCAGUCCCUUUCTCIUCGAUGGU | 40 | 13_1 |
| 14 | ACAUGGCCCCAGCAGCUUCAGUCCCUUUCTCIUCGAUGG | 39 | 14_1 |
| 15 | ACAUGGCCCCAGCAGCUUCAGUCCCUUUCTCIUCGAUG | 38 | 15_1 |
| 16 | ACAUGGCCCCAGCAGCUUCAGUCCCUUUCTCIUCGAU | 37 | 16_1 |
| 17 | ACAUGGCCCCAGCAGCUUCAGUCCCUUUCTCIUCGA | 36 | 17_1 |
| 18 | ACAUGGCCCCAGCAGCUUCAGUCCCUUUCTCIUCG | 35 | 18_1 |
| 19 | GCCCCAGCAGCUUCAGUCCCUUUCTCIUCGAUGGUCAGCAC | 41 | 19_1 |
| 20 | GCCCCAGCAGCUUCAGUCCCUUUCTCIUCGAUGGUCAGCA | 40 | 20_1 |
| 21 | GCCCCAGCAGCUUCAGUCCCUUUCTCIUCGAUGGUCAGC | 39 | 21_1 |
| 22 | GCCCCAGCAGCUUCAGUCCCUUUCTCIUCGAUGGUCAG | 38 | 22_1 |
| 23 | GCCCCAGCAGCUUCAGUCCCUUUCTCIUCGAUGGU | 35 | 23_1 |
| 24 | GCCCCAGCAGCUUCAGUCCCUUUCTCIUCGAUGG | 34 | 24_1 |
| 25 | GCCCCAGCAGCUUCAGUCCCUUUCTCIUCGAUG | 33 | 25_1 |
| 26 | GCCCCAGCAGCUUCAGUCCCUUUCTCIUCGAU | 32 | 26_1 |
| | | | to |
| | | | 26_9 |
| 27 | GCCCCAGCAGCUUCAGUCCCUUUCTCIUCGA | 31 | 27_1 |
| 28 | GCCCCAGCAGCUUCAGUCCCUUUCTCIUCG | 30 | 28_1 |
| 29 | CAUGGCCCCAGCAGCUUCAGUCCCUUUCTCIUCGAUGGUCAGCAC | 45 | 29_1 |
| 30 | AUGGCCCCAGCAGCUUCAGUCCCUUUCTCIUCGAUGGUCAGCAC | 44 | 30_1 |
| 31 | UGGCCCCAGCAGCUUCAGUCCCUUUCTCIUCGAUGGUCAGCAC | 43 | 31_1 |
| 32 | CCCCAGCAGCUUCAGUCCCUUUCTCIUCGAUGGUCAGCAC | 40 | 32_1 |
| | | | to |
| | | | 32_83 |
| 33 | CCCAGCAGCUUCAGUCCCUUUCTCIUCGAUGGUCAGCAC | 39 | 33_1 |
| 34 | CCAGCAGCUUCAGUCCCUUUCTCIUCGAUGGUCAGCAC | 38 | 34_1 |
| 35 | CAGCAGCUUCAGUCCCUUUCTCIUCGAUGGUCAGCAC | 37 | 35_1 |
| 36 | AGCAGCUUCAGUCCCUUUCTCIUCGAUGGUCAGCAC | 36 | 36_1 |
| 37 | CUUCAGUCCCUUUCTCIUCGAUGGUCAGCACAGCCUUAUGC | 41 | 37_1 |
| 38 | CUUCAGUCCCUUUCTCIUCGAUGGUCAGCACAGCCUUAUGCACGGC | 46 | 38_1 |
| 39 | GCCCCAGCAGCAUCACUCCCUUUCTCIUCGAU | 32 | 39_1 |
| 40 | CCCCAGCAGCUUCAGUCCCUUUCTAIUCGAUGGUCAGCAC | 40 | 40_1 |
| | | | to |
| | | | 40_10 |
| 41 | CCCCAGCAGCUUCAGUCCCUUUCTGIUCGAUGGUCAGCAC | 40 | 41_1 |
| | | | to |
| | | | 41_10 |
| 42 | CCCCAGCAGCUUCAGUCCCUUUCTEIUCGAUGGUCAGCAC | 40 | 42_1 |
| | | | to |
| | | | 42_6 |
| 43 | CCCCAGCAGCUUCAGUCCCUUUCTIIUCGAUGGUCAGCAC | 40 | 43_1 |
| | | | to |
| | | | 43_3 |
| 44 | CCCCAGCAGCUUCAGUCCCUUUCTZIUCGAUGGUCAGCAC | 40 | 44_1 |
| 45 | CCCCAGCAGCUUCAGUCCCUUUCTFIUCGAUGGUCAGCAC | 40 | 45_1 |
| 46 | CCCCAGCAGCUUCAGUCCCUUUCTpyrCIUCGAUGGUCAGCAC | 40 | 46_1 |
| 47 | CCCCAGCAGCUUCAGUCCCUUUCTabIUCGAUGGUCAGCAC | 40 | 47_1 |
| | | | and |
| | | | 47_2 |
| 48 | CCCCAGCAGCUUCAGUCCCUUUCTprpCIUCGAUGGUCAGCAC | 40 | 48_1 |
| 49 | CCCCAGCAGCUUCAGUCCCUUUCT2APIUCGAUGGUCAGCAC | 40 | 49_1 |
| 50 | CCCCAGCAGCUUCAGUCCCUUUCTapCIUCGAUGGUCAGCAC | 40 | 50_1 |
| 51 | CCCCAGCAGCUUCAGUCCCUUUCT5brCIUCGAUGGUCAGCAC | 40 | 51_1 |
| 52 | CUCUAAAAACAUGGCCCCAGCAGCUUCAGUCCCUUUCTCI | 40 | 52_1 |
| 53 | UCUAAAAACAUGGCCCCAGCAGCUUCAGUCCCUUUCTCIU | 40 | 53_1 |
| 54 | CUAAAAACAUGGCCCCAGCAGCUUCAGUCCCUUUCTCIUC | 40 | 54_1 |
| 55 | UAAAAACAUGGCCCCAGCAGCUUCAGUCCCUUUCTCIUCG | 40 | 55_1 |
| 56 | AAAAACAUGGCCCCAGCAGCUUCAGUCCCUUUCTCIUCGA | 40 | 56_1 |
| 57 | AAAACAUGGCCCCAGCAGCUUCAGUCCCUUUCTCIUCGAU | 40 | 57_1 |
| 58 | AAACAUGGCCCCAGCAGCUUCAGUCCCUUUCTCIUCGAUG | 40 | 58_1 |
| 59 | AACAUGGCCCCAGCAGCUUCAGUCCCUUUCTCIUCGAUGG | 40 | 59_1 |
| 60 | CAUGGCCCCAGCAGCUUCAGUCCCUUUCTCIUCGAUGGUC | 40 | 60_1 |
| 61 | AUGGCCCCAGCAGCUUCAGUCCCUUUCTCIUCGAUGGUCA | 40 | 61_1 |
| 62 | UGGCCCCAGCAGCUUCAGUCCCUUUCTCIUCGAUGGUCAG | 40 | 62_1 |
| 63 | GGCCCCAGCAGCUUCAGUCCCUUUCTCIUCGAUGGUCAGC | 40 | 63_1 |
| 64 | CCCAGCAGCUUCAGUCCCUUUCTCIUCGAUGGUCAGCACA | 40 | 64_1 |
| 65 | CCAGCAGCUUCAGUCCCUUUCTCIUCGAUGGUCAGCACAG | 40 | 65_1 |
| 66 | CAGCAGCUUCAGUCCCUUUCTCIUCGAUGGUCAGCACAGC | 40 | 66_1 |
| 67 | AGCAGCUUCAGUCCCUUUCTCIUCGAUGGUCAGCACAGCC | 40 | 67_1 |
| 68 | GCAGCUUCAGUCCCUUUCTCIUCGAUGGUCAGCACAGCCU | 40 | 68_1 |
| 69 | CAGCUUCAGUCCCUUUCTCIUCGAUGGUCAGCACAGCCUU | 40 | 69_1 |
| 70 | AGCUUCAGUCCCUUUCTCIUCGAUGGUCAGCACAGCCUUA | 40 | 70_1 |
| 71 | GCUUCAGUCCCUUUCTCIUCGAUGGUCAGCACAGCCUUAU | 40 | 71_1 |
| 72 | CUUCAGUCCCUUUCTCIUCGAUGGUCAGCACAGCCUUAUG | 40 | 72_1 |
| 73 | UUCAGUCCCUUUCTCIUCGAUGGUCAGCACAGCCUUAUGC | 40 | 73_1 |
| 74 | UCAGUCCCUUUCTCIUCGAUGGUCAGCACAGCCUUAUGCA | 40 | 74_1 |
| 75 | CAGUCCCUUUCTCIUCGAUGGUCAGCACAGCCUUAUGCAC | 40 | 75_1 |
| 76 | AGUCCCUUUCTCIUCGAUGGUCAGCACAGCCUUAUGCACG | 40 | 76_1 |
| 77 | GUCCCUUUCTCIUCGAUGGUCAGCACAGCCUUAUGCACGG | 40 | 77_1 |
| 78 | UCCCUUUCTCIUCGAUGGUCAGCACAGCCUUAUGCACGGC | 40 | 78_1 |
| 79 | CCCUUUCTCIUCGAUGGUCAGCACAGCCUUAUGCACGGC | 40 | 79_1 |
| | C | | |
| 80 | CCUUUCTCIUCGAUGGUCAGCACAGCCUUAUGCACGGC | 40 | 80_1 |
| | CU | | |
| 81 | CUUUCTCIUCGAUGGUCAGCACAGCCUUAUGCACGGC | 40 | 81_1 |
| | CUU | | |
| 82 | UUUCTCIUCGAUGGUCAGCACAGCCUUAUGCACGGC | 40 | 82_1 |
| | CUUG | | |
| 83 | UUCTCIUCGAUGGUCAGCACAGCCUUAUGCACGGC | 40 | 83_1 |
| | CUUGG | | |
| 84 | UCTCIUCGAUGGUCAGCACAGCCUUAUGCACGGC | 40 | 84_1 |
| | CUUGGU | | |
| 85 | CTCIUCGAUGGUCAGCACAGCCUUAUGCACGGC | 40 | 85_1 |
| | CUUGGUG | | |
| 86 | TCIUCGAUGGUCAGCACAGCCUUAUGCACGGC | 40 | 86_1 |
| | CUUGGUGU | | |
| 87 | CUCUAAAAACAUGGCCCCAGCAGCUUCAGUCCCUUUCTCIUCGAUGGUCAGCACAGCCUUAUGCACGGC | 77 | - |
| | CUUGGUGU | | |

TABLE 4
SERPINA1 editing compounds

| CMP | | |
|---|---|---|
| ID | | |
| NO | Name | HELM string |
| 1_1 | 35-1-13 | [symbol] |
| | SERP1 | |
| | 28 PSI | |
| 2_1 | 30-1-30 | [symbol] |
| | SERP1 | |
| | 20PS | |
| 3_1 | 30-1-30 | [symbol] |
| | SERP1 | |
| | 20 PSI | |
| 4_1 | 27-1-27 | [symbol] |
| | SERP1 | |
| | all PSI | |
| | 2′F/2′ | |
| | OMe alt | |
| 5_1 | 25-1-25 | [symbol] |
| | SERP1 | |
| | all PSI | |
| | 2′F/2′ | |
| | OMe alt | |
| 6_1 | 25-1-10 | |
| | SERP1 | |
| | all PSI | |
| | 2′F/2′ | |
| | OMe alt | |
| 3_2 | 30-1-30 | [symbol] |
| | SERP1 | |
| | all PSI | |
| 3_3 | 30-1-30 | [symbol] |
| | SERP1 | |
| | all PSI | |
| | 2′F/2′ | |
| | OMe alt | |
| 7_1 | 30-1-15 | [symbol] |
| | SERP1 | |
| | all PSI | |
| | 2′F/2′ | |
| | OMe alt | |
| 8_1 | 30-1-14 | [symbol] |
| | SERP1 | |
| | all PSI | |
| | 2′F/2′ | |
| | OMe alt | |
| 9_1 | 30-1-13 | [symbol] |
| | SERP1 | |
| | all PSI | |
| | 2′F/2′ | |
| | OMe alt | |
| 10_1 | 30-1-12 | [symbol] |
| | SERP1 | |
| | all PSI | |
| | 2′F/2′ | |
| | OMe alt | |
| 11_1 | 30-1-11 | [symbol] |
| | SERP1 | |
| | all PSI | |
| | 2′F/2′ | |
| | OMe alt | |
| 12_1 | 30-1-10 | [symbol] |
| | SERP1 | |
| | all PSI | |
| | 2′F/2′ | |
| | OMe alt | |
| 13_1 | 30-1-9 | [symbol] |
| | SERP1 | |
| | all PSI | |
| | 2′F/2′ | |
| | OMe alt | |
| 14_1 | 30-1-8 | [symbol] |
| | SERP1 | |
| | all PSI | |
| | 2′F/2′ | |
| | OMe alt | |
| 15_1 | 30-1-7 | [symbol] |
| | SERP1 | |
| | all PSI | |
| | 2′F/2′ | |
| | OMe alt | |
| 16_1 | 30-1-6 | [symbol] |
| | SERP1 | |
| | all PSI | |
| | 2′F/2′ | |
| | OMe alt | |
| 17_1 | 30-1-5 | [symbol] |
| | SERP1 | |
| | all PSI | |
| | 2′F/2′ | |
| | OMe alt | |
| 18_1 | 30-1-4 | [symbol] |
| | SERP1 | |
| | all PSI | |
| | 2′F/2′ | |
| | OMe alt | |
| 19_1 | 25-1-15 | [symbol] |
| | SERP1 | |
| | all PSI | |
| | 2′F/2′ | |
| | OMe alt | |
| 20_1 | 25-1-14 | [symbol] |
| | SERP1 | |
| | all PSI | |
| | 2′F/2′ | |
| | OMe alt | |
| 21_1 | 25-1-13 | [symbol] |
| | SERP1 | |
| | all PSI | |
| | 2′F/2′ | |
| | OMe alt | |
| 22_1 | 25-1-12 | [symbol] |
| | SERP1 | |
| | all PSI | |
| | 2′F/2′ | |
| | OMe alt | |
| 23_1 | 25-1-9 | [symbol] |
| | SERP1 | |
| | all PSI | |
| | 2′F/2′ | |
| | OMe alt | |
| 24_1 | 25-1-8 | [symbol] |
| | SERP1 | |
| | all PSI | |
| | 2′F/2′ | |
| | OMe alt | |
| 25_1 | 25-1-7 | [symbol] |
| | SERP1 | |
| | all PSI | |
| | 2′F/2′ | |
| | OMe alt | |
| 26_1 | 25-1-6 | [symbol] |
| | SERP1 | |
| | all PSI | |
| | 2′F/2′ | |
| | OMe alt | |
| 27_1 | 25-1-5 | [symbol] |
| | SERP1 | |
| | all PSI | |
| | 2′F/2′ | |
| | OMe alt | |
| 28_1 | 25-1-4 | [symbol] |
| | SERP1 | |
| | all PSI | |
| | 2′F/2′ | |
| | OMe alt | |
| 29_1 | 29-1-15 | [symbol] |
| | SERP1 | |
| | all PSI | |
| | 2′F/2′ | |
| | OMe alt | |
| 30_1 | 28-1-15 | [symbol] |
| | SERP1 | |
| | all PSI | |
| | 2′F/2′ | |
| | OMe alt | |
| 31_1 | 27-1-15 | [symbol] |
| | SERP1 | |
| | all PSI | |
| | 2′F/2′ | |
| | OMe alt | |
| 32_1 | 24-1-15 | [symbol] |
| | SERP1 | |
| | all PSI | |
| | 2′F/2′ | |
| | OMe alt | |
| 33_1 | 23-1-15 | [symbol] |
| | SERP1 | |
| | all PSI | |
| | 2′F/2′ | |
| | OMe alt | |
| 34_1 | 22-1-15 | [symbol] |
| | SERP1 | |
| | all PSI | |
| | 2′F/2′ | |
| | OMe alt | |
| 35_1 | 21-1-15 | [symbol] |
| | SERP1 | |
| | all PSI | |
| | 2′F/2′ | |
| | OMe alt | |
| 36_1 | 20-1-15 | [symbol] |
| | SERP1 | |
| | all PSI | |
| | 2′F/2′ | |
| | OMe alt | |
| 37_1 | 15-1-25 | [symbol] |
| | SERP1 | |
| | all PSI | |
| | 2′F/2′ | |
| | OMe alt | |
| 38_1 | 15-1-30 | [symbol] |
| | SERP1 | |
| | all PSI | |
| | 2′F/2′ | |
| | OMe alt | |
| 39_1 | WV-27820 | [symbol] |
| | SERPINA1 | |
| | 25-1-6 | |
| 26_2 | WV-27820 | [symbol] |
| | SERPINA1 | |
| | 25-1-6 | |
| | without | |
| | mismatch | |
| 26_3 | WV-27820 | [symbol] |
| | SERPINA1 | |
| | 25-1-6 | |
| | w/o | |
| | mism | |
| | endblock | |
| 26_4 | SERPINA1 | [symbol] |
| | 25-1-6 | |
| | 2′OMe | |
| | dd 2′F/ | |
| | OMe alt | |
| 26_5 | SERPINA1 | [symbol] |
| | 25-1-6 | |
| | 2′OMe | |
| | dd 2′F/ | |
| | OMe alt | |
| | 4nt | |
| | endblock | |
| 26_6 | SERPINA1 | [symbol] |
| | 25-1-6 | |
| | 2′OMe | |
| | dd 2′F/ | |
| | OMe alt2 | |
| 26_7 | SERPINA1 | [symbol] |
| | 25-1-6 | |
| | all OMe | |
| 26_8 | SERPINA1 | [symbol] |
| | 25-1-6 | |
| | 2′F/OMe | |
| | alt | |
| 26_9 | SERPINA1 | [symbol] |
| | 25-1-6 | |
| | 2′F/OMe | |
| | alt2 | |
| 40_1 | 24-1-15 | [symbol] |
| | SERPINA1- | |
| | central | |
| | “C” modi- | |
| | fication: | |
| | [LR](A) | |
| | [sP] | |
| 41_1 | 24-1-15 | [symbol] |
| | SERPINA1- | |
| | central | |
| | “C” modi- | |
| | fication: | |
| | [LR](G) | |
| | [sP] | |
| 42_1 | 24-1-15 | [symbol] |
| | SERPINA1- | |
| | central | |
| | “C” modi- | |
| | fication: | |
| | [LR] | |
| | ([5mec]) | |
| | [sP] | |
| 40_2 | 24-1-15 | [symbol] |
| | SERPINA1- | |
| | central | |
| | “C” modi- | |
| | fication: | |
| | [MOE](A) | |
| | [sP] | |
| 41_2 | 24-1-15 | [symbol] |
| | SERPINA1- | |
| | central | |
| | “C” modi- | |
| | fication: | |
| | [MOE](G) | |
| | [sP] | |
| 42_2 | 24-1-15 | [symbol] |
| | SERPINA1- | |
| | central | |
| | “C” modi- | |
| | fication: | |
| | [MOE] | |
| | ([5meC]) | |
| | [sP] | |
| 40_3 | 24-1-15 | [symbol] |
| | SERPINA1- | |
| | central | |
| | “C” modi- | |
| | fication: | |
| | [fR](A) | |
| | [sP] | |
| 41_3 | 24-1-15 | [symbol] |
| | SERPINA1- | |
| | central | |
| | “C” modi- | |
| | fication: | |
| | [fR](G) | |
| | [sP] | |
| 32_2 | 24-1-15 | [symbol] |
| | SERPINA1- | |
| | central | |
| | “C” modi- | |
| | fication: | |
| | [fR](C) | |
| | [sP] | |
| 43_1 | 24-1-15 | [symbol] |
| | SERPINA1- | |
| | central | |
| | “C” modi- | |
| | fication: | |
| | (R)(n) | |
| | [sP] | |
| 40_4 | 24-1-15 | [symbol] |
| | SERPINA1- | |
| | central | |
| | “C” modi- | |
| | fication: | |
| | [mR](A) | |
| | [sP] | |
| 41_4 | 24-1-15 | [symbol] |
| | SERPINA1- | |
| | central | |
| | “C” modi- | |
| | fication: | |
| | [mR](G) | |
| | [sP] | |
| 32_3 | 24-1-15 | [symbol] |
| | SERPINA1- | |
| | central | |
| | “C” modi- | |
| | fication: | |
| | [mR](C) | |
| | [sP] | |
| 43_2 | 24-1-15 | [symbol] |
| | SERPINA1- | |
| | central | |
| | “C” modi- | |
| | fication: | |
| | [mR]([In]) | |
| | (sP] | |
| 42_3 | 24-1-15 | [symbol] |
| | SERPINA1- | |
| | central | |
| | “C” modi- | |
| | fication: | |
| | [mR] | |
| | ([5meC]) | |
| | [sP] | |
| 43_3 | 24-1-15 | [symbol] |
| | SERPINA1- | |
| | central | |
| | “C” modi- | |
| | fication: | |
| | [dR]([In]) | |
| | [sP] | |
| 40_5 | 24-1-15 | [symbol] |
| | SERPINA1- | |
| | central | |
| | “C” modi- | |
| | fication: | |
| | [TNA](A) | |
| | [sP] | |
| 41_5 | 24-1-15 | [symbol] |
| | SERPINA1- | |
| | central | |
| | “C” modi- | |
| | fication: | |
| | [TNA](G) | |
| | [sP] | |
| 42_4 | 24-1-15 | [symbol] |
| | SERPINA1- | |
| | central | |
| | “C” modi- | |
| | fication: | |
| | [TNA] | |
| | ([5meC]) | |
| | [sP] | |
| 40_6 | 24-1-15 | [symbol] |
| | SERPINA1- | |
| | central | |
| | “C” modi- | |
| | fication: | |
| | [SNA](A) | |
| | [sP] | |
| 41_6 | 24-1-15 | [symbol] |
| | SERPINA1- | |
| | central | |
| | “C” modi- | |
| | fication: | |
| | [SNA](G) | |
| | [sP] | |
| 42_5 | 24-1-15 | [symbol] |
| | SERPINA1- | |
| | central | |
| | “C” modi- | |
| | fication: | |
| | [SNA] | |
| | ([5meC]) | |
| | [sP] | |
| 44_1 | 24-1-15 | [symbol] |
| | SERPINA1- | |
| | central | |
| | “C” modi- | |
| | fication: | |
| | [dR](Z) | |
| | [sP] | |
| 45_1 | 24-1-15 | [symbol] |
| | SERPINA1- | |
| | central | |
| | “C” modi- | |
| | fication: | |
| | [dR] | |
| | ([PPG]) | |
| | [sP] | |
| 41_7 | 24-1-15 | [symbol] |
| | SERPINA1- | |
| | central | |
| | “C” modi- | |
| | fication: | |
| | [ScEt](G) | |
| | [sP] | |
| 46_1 | 24-1-15 | [symbol] |
| | SERPINA1- | |
| | central | |
| | “C” modi- | |
| | fication: | |
| | [dR] | |
| | ([PyrC]) | |
| | [sP] | |
| 40_7 | 24-1-15 | [symbol] |
| | SERPINA1- | |
| | central | |
| | “C” modi- | |
| | fication: | |
| | [FANA](A) | |
| | [sP] | |
| 41_8 | 24-1-15 | [symbol] |
| | SERPINA1- | |
| | central | |
| | “C” modi- | |
| | fication: | |
| | [FANA](G) | |
| | [sP] | |
| 32_4 | 24-1-15 | [symbol] |
| | SERPINA1- | |
| | central | |
| | “C” modi- | |
| | fication: | |
| | [FANA](C) | |
| | [sP] | |
| 47_1 | 24-1-15 | [symbol] |
| | SERPINA1- | |
| | central | |
| | “C” modi- | |
| | fication: | |
| | [MOE][sP] | |
| 47_2 | 24-1-15 | [symbol] |
| | SERPINA1- | |
| | central | |
| | “C” modi- | |
| | fication: | |
| | [dR][sP] | |
| 32_5 | 24-1-15 | [symbol] |
| | SERP1 | |
| | all PSI | |
| | 2′F/2′ | |
| | OMe alt1 | |
| 32_6 | 24-1-15 | [symbol] |
| | SERP1 | |
| | all PSI | |
| | 2′F/2′ | |
| | OMe alt1 | |
| | Ome 23 | |
| 32_7 | 24-1-15 | [symbol] |
| | SERP1 | |
| | all PSI | |
| | 2′F/2′ | |
| | OMe alt1 | |
| | Ome 21 | |
| 32_8 | 24-1-15 | [symbol] |
| | SERP1 | |
| | all PSI | |
| | 2′F/2′ | |
| | OMe alt1 | |
| | Ome 19 | |
| 32_9 | 24-1-15 | [symbol] |
| | SERP1 | |
| | all PSI | |
| | 2′F/2′ | |
| | OMe alt1 | |
| | Ome 17 | |
| 32_10 | 24-1-15 | [symbol] |
| | SERP1 | |
| | all PSI | |
| | 2′F/2′ | |
| | OMe alt1 | |
| | Ome 15 | |
| 32_11 | 24-1-15 | [symbol] |
| | SERP1 | |
| | all PSI | |
| | 2′F/2′ | |
| | OMe alt1 | |
| | Ome 13 | |
| 32_12 | 24-1-15 | [symbol] |
| | SERP1 | |
| | all PSI | |
| | 2′F/2′ | |
| | OMe alt1 | |
| | Ome 11 | |
| 32_13 | 24-1-15 | [symbol] |
| | SERP1 | |
| | all PSI | |
| | 2′F/2′ | |
| | OMe alt1 | |
| | Ome 9 | |
| 32_14 | 24-1-15 | [symbol] |
| | SERP1 | |
| | all PSI | |
| | 2′F/2′ | |
| | OMe alt1 | |
| | Ome 7 | |
| 32_15 | 24-1-15 | [symbol] |
| | SERP1 | |
| | all PSI | |
| | 2′F/2′ | |
| | OMe alt1 | |
| | Ome 5 | |
| 32_16 | 24-1-15 | [symbol] |
| | SERP1 | |
| | all PSI | |
| | 2′F/2′ | |
| | OMe alt1 | |
| | Ome 3 | |
| 32_17 | 24-1-15 | [symbol] |
| | SERP1 | |
| | all PSI | |
| | 2′F/2′ | |
| | OMe alt1 | |
| | Ome -3 | |
| 32_18 | 24-1-15 | [symbol] |
| | SERP1 | |
| | all PSI | |
| | 2′F/2′ | |
| | OMe alt1 | |
| | Ome -5 | |
| 32_19 | 24-1-15 | [symbol] |
| | SERP1 | |
| | all PSI | |
| | 2′F/2′ | |
| | OMe alt1 | |
| | Ome -7 | |
| 32_20 | 24-1-15 | [symbol] |
| | SERP1 | |
| | all PSI | |
| | 2′F/2′ | |
| | OMe alt1 | |
| | Ome -9 | |
| 32_21 | 24-1-15 | [symbol] |
| | SERP1 | |
| | all PSI | |
| | 2′F/2′ | |
| | OMe alt1 | |
| | Ome -11 | |
| 32_22 | 24-1-15 | [symbol] |
| | SERP1 | |
| | all PSI | |
| | 2′F/2′ | |
| | OMe alt1 | |
| | Ome -13 | |
| 32_23 | 24-1-15 | [symbol] |
| | SERP1 | |
| | all PSI | |
| | 2′F/2′ | |
| | OMe alt1 | |
| | Ome -15 | |
| 32_24 | 24-1-15 | [symbol] |
| | SERP1 | |
| | all PSI | |
| | 2′F/2′ | |
| | OMe alt2 | |
| 32_25 | 24-1-15 | [symbol] |
| | SERP1 | |
| | all PSI | |
| | 2′F/2′ | |
| | OMe alt2 | |
| | Ome 8 | |
| 32_26 | 24-1-15 | [symbol] |
| | SERP1 | |
| | all PSI | |
| | 2′F/2′ | |
| | OMe alt2 | |
| | Ome 6 | |
| 32_27 | 24-1-15 | [symbol] |
| | SERP1 | |
| | all PSI | |
| | 2′F/2′ | |
| | OMe alt2 | |
| | Ome 4 | |
| 32_28 | 24-1-15 | [symbol] |
| | SERP1 | |
| | all PSI | |
| | 2′F/2′ | |
| | OMe alt2 | |
| | Ome 2 | |
| 32_29 | 24-1-15 | [symbol] |
| | SERP1 | |
| | all PSI | |
| | 2′F/2′ | |
| | OMe alt2 | |
| | Ome -2 | |
| 32_30 | 24-1-15 | [symbol] |
| | SERP1 | |
| | all PSI | |
| | 2′F/2′ | |
| | OMe alt2 | |
| | Ome -4 | |
| 32_31 | 24-1-15 | [symbol] |
| | SERP1 | |
| | all PSI | |
| | 2′F/2′ | |
| | OMe alt2 | |
| | Ome -6 | |
| 32_32 | 24-1-15 | [symbol] |
| | SERP1 | |
| | all PSI | |
| | 2′F/2′ | |
| | OMe alt2 | |
| | Ome -8 | |
| 40_8 | 24-1-15 | [symbol] |
| | SERPINA1- | |
| | central | |
| | “C” modi- | |
| | fication: | |
| | [dR](A) | |
| | [srP] | |
| 40_9 | 24-1-15 | [symbol] |
| | SERPINA1- | |
| | central | |
| | “C” modi- | |
| | fication: | |
| | [dR](A) | |
| | [ssP] | |
| 41_9 | 24-1-15 | [symbol] |
| | SERPINA1- | |
| | central | |
| | “C” modi- | |
| | fication: | |
| | [dR](G) | |
| | [ssP] | |
| 32_33 | 24-1-15 | [symbol] |
| | SERPINA1- | |
| | central | |
| | “C” modi- | |
| | fication: | |
| | [dR](C) | |
| | [srP] | |
| 32_34 | 24-1-15 | [symbol] |
| | SERPINA1- | |
| | central | |
| | “C” modi- | |
| | fication: | |
| | [dR](C) | |
| | [ssP] | |
| 40_10 | 24-1-15 | [symbol] |
| | SERPINA1- | |
| | central | |
| | “C” modi- | |
| | fication: | |
| | [idR](A) | |
| | [sP] | |
| 41_10 | 24-1-15 | [symbol] |
| | SERPINA1- | |
| | central | |
| | “C” modi- | |
| | fication: | |
| | [idR](G) | |
| | (sP] | |
| 32_35 | 24-1-15 | [symbol] |
| | SERPINA1- | |
| | central | |
| | “C” modi- | |
| | fication: | |
| | [idR](C) | |
| | [sP] | |
| 42_6 | 24-1-15 | [symbol] |
| | SERPINA1- | |
| | central | |
| | “C” modi- | |
| | fication: | |
| | [dR] | |
| | ([5meC]) | |
| | [sP] | |
| 48_1 | 24-1-15 | [symbol] |
| | SERPINA1- | |
| | central | |
| | “C” modi- | |
| | fication: | |
| | [dR] | |
| | ([prpC]) | |
| | [sP] | |
| 49_1 | 24-1-15 | [symbol] |
| | SERPINA1- | |
| | central | |
| | “C” modi- | |
| | fication: | |
| | [dR] | |
| | ([2AP]) | |
| | [sP] | |
| 50_1 | 24-1-15 | [symbol] |
| | SERPINA1- | |
| | central | |
| | “C” modi- | |
| | fication: | |
| | [dR] | |
| | ([apC]) | |
| | [sP] | |
| 51_1 | 24-1-15 | [symbol] |
| | SERPINA1- | |
| | central | |
| | “C” modi- | |
| | fication: | |
| | [dR] | |
| | ([5BrC]) | |
| | [sP] | |
| 32_36 | 24-1-15 | [symbol] |
| | SERP1 | |
| | all PSI | |
| | 2′F/2′ | |
| | OMe alt2 | |
| | Ome 24 | |
| 32_37 | 24-1-15 | [symbol] |
| | SERP1 | |
| | all PSI | |
| | 2′F/2′ | |
| | OMe alt | |
| | 2 Ome 22 | |
| 32_38 | 24-1-15 | [symbol] |
| | SERP1 | |
| | all PSI | |
| | 2′F/2′ | |
| | OMe alt2 | |
| | Ome 20 | |
| 32_39 | 24-1-15 | [symbol] |
| | SERP1 | |
| | all PSI | |
| | 2′F/2′ | |
| | OMe alt2 | |
| | Ome 18 | |
| 32_40 | 24-1-15 | [symbol] |
| | SERP1 | |
| | all PSI | |
| | 2′F/2′ | |
| | OMe alt2 | |
| | Ome 16 | |
| 32_41 | 24-1-15 | [symbol] |
| | SERP1 | |
| | all PSI | |
| | 2′F/2′ | |
| | OMe alt2 | |
| | Ome 14 | |
| 32_42 | 24-1-15 | [symbol] |
| | SERP1 | |
| | all PSI | |
| | 2′F/2′ | |
| | OMe alt2 | |
| | Ome 12 | |
| 32_43 | 24-1-15 | [symbol] |
| | SERP1 | |
| | all PSI | |
| | 2′F/2′ | |
| | OMe alt2 | |
| | Ome 10 | |
| 32_44 | 24-1-15 | [symbol] |
| | SERP1 | |
| | all PSI | |
| | 2′F/2′ | |
| | OMe alt2 | |
| | Ome -10 | |
| 32_45 | 24-1-15 | [symbol] |
| | SERP1 | |
| | all PSI | |
| | 2′F/2′ | |
| | OMe alt2 | |
| | Ome -12 | |
| 32_46 | 24-1-15 | [symbol] |
| | SERP1 | |
| | all PSI | |
| | 2′F/2′ | |
| | OMe alt2 | |
| | Ome -14 | |
| 32_47 | 24-1-15 | [symbol] |
| | SERP1 | |
| | all PSI | |
| | 2′F/2′ | |
| | OMe alt1 | |
| | F 24 | |
| 32_48 | 24-1-15 | [symbol] |
| | SERP1 | |
| | all PSI | |
| | 2′F/2′ | |
| | OMe alt1 | |
| | F 22 | |
| 32_49 | 24-1-15 | [symbol] |
| | SERP1 | |
| | all PSI | |
| | 2′F/2′ | |
| | OMe alt1 | |
| | F 20 | |
| 32_50 | 24-1-15 | [symbol] |
| | SERP1 | |
| | all PSI | |
| | 2′F/2′ | |
| | OMe alt1 | |
| | F 18 | |
| 32_51 | 24-1-15 | [symbol] |
| | SERP1 | |
| | all PSI | |
| | 2′F/2′ | |
| | OMe alt1 | |
| | F 16 | |
| 32_52 | 24-1-15 | [symbol] |
| | SERP1 | |
| | all PSI | |
| | 2′F/2′ | |
| | OMe alt1 | |
| | F 14 | |
| 32_53 | 24-1-15 | [symbol] |
| | SERP1 | |
| | all PSI | |
| | 2′F/2′ | |
| | OMe alt1 | |
| | F 12 | |
| 32_54 | 24-1-15 | [symbol] |
| | SERP1 | |
| | all PSI | |
| | 2′F/2′ | |
| | OMe alt1 | |
| | F 10 | |
| 32_55 | 24-1-15 | [symbol] |
| | SERP1 | |
| | all PSI | |
| | 2′F/2′ | |
| | OMe alt1 | |
| | F 8 | |
| 32_56 | 24-1-15 | [symbol] |
| | SERP1 | |
| | all PSI | |
| | 2′F/2′ | |
| | OMe alt1 | |
| | F 6 | |
| 32_57 | 24-1-15 | [symbol] |
| | SERP1 | |
| | all PSI | |
| | 2′F/2′ | |
| | OMe alt1 | |
| | F 4 | |
| 32_58 | 24-1-15 | [symbol] |
| | SERP1 | |
| | all PSI | |
| | 2′F/2′ | |
| | OMe alt1 | |
| | F 2 | |
| 32_59 | 24-1-15 | [symbol] |
| | SERP1 | |
| | all PSI | |
| | 2′F/2′ | |
| | OMe alt1 | |
| | F -2 | |
| 32_60 | 24-1-15 | [symbol] |
| | SERP1 | |
| | all PSI | |
| | 2′F/2′ | |
| | OMe alt1 | |
| | F -4 | |
| 32_61 | 24-1-15 | [symbol] |
| | SERP1 | |
| | all PSI | |
| | 2′F/2′ | |
| | OMe alt1 | |
| | F -6 | |
| 32_62 | 24-1-15 | [symbol] |
| | SERP1 | |
| | all PSI | |
| | 2′F/2′ | |
| | OMe alt1 | |
| | F -8 | |
| 32_63 | 24-1-15 | [symbol] |
| | SERP1 | |
| | all PSI | |
| | 2′F/2′ | |
| | OMe alt1 | |
| | F -10 | |
| 32_64 | 24-1-15 | [symbol] |
| | SERP1 | |
| | all PSI | |
| | 2′F/2′ | |
| | OMe alt1 | |
| | F -12 | |
| 32_65 | 24-1-15 | [symbol] |
| | SERP1 | |
| | all PSI | |
| | 2′F/2′ | |
| | OMe alt1 | |
| | F -14 | |
| 32_66 | 24-1-15 | [symbol] |
| | SERP1 | |
| | all PSI | |
| | 2′F/2′ | |
| | OMe alt2 | |
| | F 23 | |
| 32_67 | 24-1-15 | [symbol] |
| | SERP1 | |
| | all PSI | |
| | 2′F/2′ | |
| | OMe alt2 | |
| | F 21 | |
| 32_68 | 24-1-15 | [symbol] |
| | SERP1 | |
| | all PSI | |
| | 2′F/2′ | |
| | OMe alt2 | |
| | F 19 | |
| 32_69 | 24-1-15 | [symbol] |
| | SERP1 | |
| | all PSI | |
| | 2′F/2′ | |
| | OMe alt2 | |
| | F 17 | |
| 32_70 | 24-1-15 | [symbol] |
| | SERP1 | |
| | all PSI | |
| | 2′F/2′ | |
| | OMe alt2 | |
| | F 15 | |
| 32_71 | 24-1-15 | [symbol] |
| | SERP1 | |
| | all PSI | |
| | 2′F/2′ | |
| | OMe alt2 | |
| | F 13 | |
| 32_72 | 24-1-15 | [symbol] |
| | SERP1 | |
| | all PSI | |
| | 2′F/2′ | |
| | OMe alt2 | |
| | F 11 | |
| 32_73 | 24-1-15 | [symbol] |
| | SERP1 | |
| | all PSI | |
| | 2′F/2′ | |
| | OMe alt2 | |
| | F 9 | |
| 32_74 | 24-1-15 | [symbol] |
| | SERP1 | |
| | all PSI | |
| | 2′F/2′ | |
| | OMe alt2 | |
| | F 7 | |
| 32_75 | 24-1-15 | [symbol] |
| | SERP1 | |
| | all PSI | |
| | 2′F/2′ | |
| | OMe alt2 | |
| | F 5 | |
| 32_76 | 24-1-15 | [symbol] |
| | SERP1 | |
| | all PSI | |
| | 2′F/2′ | |
| | OMe alt2 | |
| | F 3 | |
| 32_77 | 24-1-15 | [symbol] |
| | SERP1 | |
| | all PSI | |
| | 2′F/2′ | |
| | OMe alt2 | |
| | F -3 | |
| 32_78 | 24-1-15 | [symbol] |
| | SERP1 | |
| | all PSI | |
| | 2′F/2′ | |
| | OMe alt2 | |
| | F -5 | |
| 32_79 | 24-1-15 | [symbol] |
| | SERP1 | |
| | all PSI | |
| | 2′F/2′ | |
| | OMe alt2 | |
| | F -7 | |
| 32_80 | 24-1-15 | [symbol] |
| | SERP1 | |
| | all PSI | |
| | 2′F/2′ | |
| | OMe alt2 | |
| | F -9 | |
| 32_81 | 24-1-15 | [symbol] |
| | SERP1 | |
| | all PSI | |
| | 2′F/2′ | |
| | OMe alt2 | |
| | F -11 | |
| 32_82 | 24-1-15 | [symbol] |
| | SERP1 | |
| | all PSI | |
| | 2′F/2′ | |
| | OMe alt2 | |
| | F -13 | |
| 32_83 | 24-1-15 | [symbol] |
| | SERP1 | |
| | all PSI | |
| | 2′F/2′ | |
| | OMe alt2 | |
| | F -15 | |
| 52_1 | 38-1-1 | [symbol] |
| | SERP1 | |
| | all PSI | |
| | 2′F/2′ | |
| | Ome alt | |
| 53_1 | 37-1-2 | [symbol] |
| | SERP1 | |
| | all PSI | |
| | 2′F/2′ | |
| | Ome alt | |
| 54_1 | 36-1-3 | [symbol] |
| | SERP1 | |
| | all PSI | |
| | 2′F/2′ | |
| | Ome alt | |
| 55_1 | 35-1-4 | [symbol] |
| | SERP1 | |
| | all PSI | |
| | 2′F/2′ | |
| | Ome alt | |
| 56_1 | 34-1-5 | [symbol] |
| | SERP1 | |
| | all PSI | |
| | 2′F/2′ | |
| | Ome alt | |
| 57_1 | 33-1-6 | [symbol] |
| | SERP1 | |
| | all PSI | |
| | 2′F/2′ | |
| | Ome alt | |
| 58_1 | 32-1-7 | [symbol] |
| | SERP1 | |
| | all PSI | |
| | 2′F/2′ | |
| | Ome alt | |
| 59_1 | 31-1-8 | [symbol] |
| | SERP1 | |
| | all PSI | |
| | 2′F/2′ | |
| | Ome alt | |
| 60_1 | 29-1-10 | [symbol] |
| | SERP1 | |
| | all PSI | |
| | 2′F/2′ | |
| | Ome alt | |
| 61_1 | 28-1-11 | [symbol] |
| | SERP1 | |
| | all PSI | |
| | 2′F/2′ | |
| | Ome alt | |
| 62_1 | 27-1-12 | [symbol] |
| | SERP1 | |
| | all PSI | |
| | 2′F/2′ | |
| | Ome alt | |
| 63_1 | 26-1-13 | [symbol] |
| | SERP1 | |
| | all PSI | |
| | 2′F/2′ | |
| | Ome alt | |
| 64_1 | 23-1-16 | [symbol] |
| | SERP1 | |
| | all PSI | |
| | 2′F/2′ | |
| | Ome alt | |
| 65_1 | 22-1-17 | [symbol] |
| | SERP1 | |
| | all PSI | |
| | 2′F/2′ | |
| | Ome alt | |
| 66_1 | 21-1-18 | [symbol] |
| | SERP1 | |
| | all PSI | |
| | 2′F/2′ | |
| | Ome alt | |
| 67_1 | 20-1-19 | [symbol] |
| | SERP1 | |
| | all PSI | |
| | 2′F/2′ | |
| | Ome alt | |
| 68_1 | 19-1-20 | [symbol] |
| | SERP1 | |
| | all PSI | |
| | 2′F/2′ | |
| | Ome alt | |
| 69_1 | 18-1-21 | [symbol] |
| | SERP1 | |
| | all PSI | |
| | 2′F/2′ | |
| | Ome alt | |
| 70_1 | 17-1-22 | [symbol] |
| | SERP1 | |
| | all PSI | |
| | 2′F/2′ | |
| | Ome alt | |
| 71_1 | 16-1-23 | [symbol] |
| | SERP1 | |
| | all PSI | |
| | 2′F/2′ | |
| | Ome alt | |
| 72_1 | 15-1-24 | [symbol] |
| | SERP1 | |
| | all PSI | |
| | 2′F/2′ | |
| | Ome alt | |
| 73_1 | 14-1-25 | [symbol] |
| | SERP1 | |
| | all PSI | |
| | 2′F/2′ | |
| | Ome alt | |
| 74_1 | 13-1-26 | [symbol] |
| | SERP1 | |
| | all PSI | |
| | 2′F/2′ | |
| | Ome alt | |
| 75_1 | 12-1-27 | [symbol] |
| | SERP1 | |
| | all PSI | |
| | 2′F/2′ | |
| | Ome alt | |
| 76_1 | 11-1-28 | [symbol] |
| | SERP1 | |
| | all PSI | |
| | 2′F/2′ | |
| | Ome alt | |
| 77_1 | 10-1-29 | [symbol] |
| | SERP1 | |
| | all PSI | |
| | 2′F/2′ | |
| | Ome alt | |
| 78_1 | 9-1-30 | [symbol] |
| | SERP1 | |
| | all PSI | |
| | 2′F/2′ | |
| | Ome alt | |
| 79_1 | 8-1-31 | [symbol] |
| | SERP1 | |
| | all PSI | |
| | 2′F/2′ | |
| | Ome alt | |
| 80_1 | 7-1-32 | [symbol] |
| | SERP1 | |
| | all PSI | |
| | 2′F/2′ | |
| | Ome alt | |
| 81_1 | 6-1-33 | [symbol] |
| | SERP1 | |
| | all PSI | |
| | 2′F/2′ | |
| | Ome alt | |
| 82_1 | 5-1-34 | [symbol] |
| | SERP1 | |
| | all PSI | |
| | 2′F/2′ | |
| | Ome alt | |
| 83_1 | 4-1-35 | [symbol] |
| | SERP1 | |
| | all PSI | |
| | 2′F/2′ | |
| | Ome alt | |
| 84_1 | 3-1-36 | [symbol] |
| | SERP1 | |
| | all PSI | |
| | 2′F/2′ | |
| | Ome alt | |
| 85_1 | 2-1-37 | [symbol] |
| | SERP1 | |
| | all PSI | |
| | 2′F/2′ | |
| | Ome alt | |
| 86_1 | 1-1-38 | [symbol] |
| | SERP1 | |
| | all PSI | |
| | 2′F/2′ | |
| | Ome alt | |

[symbol]

indicates data missing or illegible when filed

TABLE 5
GAPDH editing sequences

| SEQ ID | | | |
|---|---|---|---|
| NO | Sequence (clean) | Length | CMP ID NO |
| 88 | ACCUUCUUGAUGUCAUCAUAUUUGGCAGGUUUUUCCAGACGGCAGGUCAGGU | 52 | 88_1 to 88_14 |
| 89 | ACCUUCUTGAUGUCATCAUAUTUGGCAGGTUTUTCCAGACGGCAGGUCAGGU | 52 | 89_1 |
| 90 | ACCUUCUTGATGUCAUCAUAUUTGGCAGGUUTUUCCAGACGGCAGGUCAGGU | 52 | 90_1 |
| 91 | CUUGAUGUCAUCAUAUUUGGCAGGUUUUUCCAGACGGCAGGUCAGGUCCACC | 61 | 91_1 to 91_3 |
| | ACUGACACG | | |
| 92 | GUCAUCAUAUUUGGCAGGUUUUUCCAGACGGCAGGUCAGG | 40 | 92_1 to 92_62 |
| 93 | CUUGAUGUCAUCAUAUUUGGCAGGUUUUUCZAGACGGCAGGUCAGGUCCACC | 61 | 93_1 |
| | ACUGACACG | | |
| 94 | GUCAUCAUAUUUGGCAGGUUUUUCCAGACGGCAGGUC | 37 | 94_1 |
| 95 | GUCAUCAUAUUUGGCAGGUUUUUCCAGACGGCAGG | 35 | 95_1 |
| 96 | GUCAUCAUAUUUGGCAGGUUUUUCCAGACGGCA | 33 | 96_1 and 96_2 |
| 97 | GUCAUCAUAUUUGGCAGGUUUUUCCAGACGG | 31 | 97_1 |
| 98 | GUCAUCAUAUUUGGCAGGUUUUUCCAGAC | 29 | 98_1 and 98_2 |
| 99 | CAUCAUAUUUGGCAGGUUUUUCCAGACGGCAGGUC | 35 | 99_1 |
| 100 | CAUCAUAUUUGGCAGGUUUUUCCAGACGGCAGG | 33 | 100_1 |
| 101 | CAUCAUAUUUGGCAGGUUUUUCCAGACGGCA | 31 | 101_1 and 101_2 |
| 102 | CAUCAUAUUUGGCAGGUUUUUCCAGACGG | 29 | 102_1 |
| 103 | CAUCAUAUUUGGCAGGUUUUUCCAGAC | 27 | 103_1 |
| 104 | UCAUAUUUGGCAGGUUUUUCCAGACGGCAGGUC | 33 | 104_1 |
| 105 | UCAUAUUUGGCAGGUUUUUCCAGACGGCAGG | 31 | 105_1 |
| 106 | UCAUAUUUGGCAGGUUUUUCCAGACGGCA | 29 | 106_1 |
| 107 | UCAUAUUUGGCAGGUUUUUCCAGACGG | 27 | 107_1 |
| 108 | UCAUAUUUGGCAGGUUUUUCCAGAC | 25 | 108_1 |
| 109 | GUCAUCAUAUUUGGCAGGUUUUUCGAGACGGCAGGUCAGG | 40 | 109_1 and 109_2 |
| 110 | GUCAUCAUAUUUGGCAGGUUUUUCAAGACGGCAGGUCAGG | 40 | 110_1 to 110_11 |
| 111 | GUCAUCAUAUUUGGCAGGUUUUUCTAGACGGCAGGUCAGG | 40 | 111_1 |
| 112 | GUCAUCAUAUUUGGCAGGUUUUUCIAGACGGCAGGUCAGG | 40 | 112_1 to 112_3 |
| 113 | GUCAUCAUAUUUGGCAGGUUUUUGCAGACGGCAGGUCAGG | 40 | 113_1 |
| 114 | GUCAUCAUAUUUGGCAGGUUUUUACAGACGGCAGGUCAGG | 40 | 114_1 |
| 115 | GUCAUCAUAUUUGGCAGGUUUUUTCAGACGGCAGGUCAGG | 40 | 115_1 |
| 116 | GUCAUCAUAUUUGGCAGGUUUUUICAGACGGCAGGUCAGG | 40 | 116_1 |
| 117 | GUCAUCAUAUUUGGCAGGUUUUUCCGGACGGCAGGUCAGG | 40 | 117_1 |
| 118 | GUCAUCAUAUUUGGCAGGUUUUUCCCGACGGCAGGUCAGG | 40 | 118_1 |
| 119 | GUCAUCAUAUUUGGCAGGUUUUUCCTGACGGCAGGUCAGG | 40 | 119_1 |
| 120 | GUCAUCAUAUUUGGCAGGUUUUUCCIGACGGCAGGUCAGG | 40 | 120_1 |
| 121 | GUCAUCAUAUUUGGCAGGUUUUUCEAGACGGCAGGUCAGG | 40 | 121_1 to 121_5 |
| 122 | GUCAUCAUAUUUGGCAGGUUUUUCZAGACGGCAGGUCAGG | 40 | 122_1 |
| 123 | GUCAUCAUAUUUGGCAGGUUUUUCpyrCAGACGGCAGGUCAGG | 40 | 123_1 |
| 124 | GUCAUCAUAUUUGGCAGGUUUUUCabAGACGGCAGGUCAGG | 40 | 124_1 to 124_2 |
| 125 | GUCAUCAUAUUUGGCAGGUUUUUC5brCAGACGGCAGGUCAGG | 40 | 125_1 |

TABLE 6
GAPDH editing compounds

| CMP ID | | |
|---|---|---|
| NO | Name | HELMstring |
| 88_1 | 35-1-16 | RNA1{[mR](A)[sP].[mR](C)[sP].[mR](C)[sP].[mR](U)[P].[mR](U)[P].R(C)[sP].R(U)[sP].R(U)[sP].R(G)[sP].R(A)[sP].R(U)[sP].R(G)[sP].R(U)[sP]. |
| | GAPDH | R(C)[sP].R(A)[sP].R(U)[sP].R(C)[sP].R(A)[sP].R(U)[sP].R(A)[sP].R(U)[P].R(U)[sP].R(U)[sP].R(G)[sP].R(G)[sP].R(C)[sP].R(A)[sP].R(G)[sP]. |
| | all PS | (A)[sP].R(G)[sP].R(G)[sP].R(U)[sP].[mR](C)(sP].[mR](A)[sP].[mR)(G)sP].[mR](G)[sP].[mR](U)}$$$$V2.0 |
| 88 2 | 35-1-16 | RNA1{[mR](A)[sP].[mR](C)[sP].[mR](C)[sP].[mR](U)[sP].[mR](U)[sP].R(C)[sP].R(U)[sP].R(U)[sP].R(G)[sP].R(A)[sP].R(U)[sP].R(G)[sP].R(U) |
| | GAPDH | [sP].R(C)[sP].R(A)[sP].R(U)[sP].R(C)[sP].R(A)[sP].R(U)[sP].R(A)[sP].R(U)[sP].R(U)[sP].R(U)[sP].R(G)[sP].R(G)[P].R(C)[sP].R(A)[sP].R(G) |
| | 48 PS | [sP].R(G)[sP].R(U)[sP].R(U)[sP].R(U)[sP].R(U)[sP].R(U)[sP].R(C)P.R(C)P.R(A)P.R(G)[sP].R(A)[P].R(C)[sP].R(G)[sP].R(G)[sP].R(C)[sP].R(A) |
| | | [sP].R(G)[sP].R(G)[sP].R(U)[sP].[mR](C)[sP].[mR](A)[sP].[mR](G)[sP].[mR](G)[sP].[mR](U)}$$$$V2.0 |
| 88_3 | 35-1-16 | RNA1{[mR](A)[sP][mR](C)[sP].[mR](C)[P].[mR](U)[sP].[mR](U)[sP].[fR](C)[sP].[mR](U)[P][[R](U)[sP].[mR](G)[sP].[[R](A)[sP].[mR](U)[sP]. |
| | GAPDH | [fR)(G)(sP].[mR](U)[sP].[fR)(C)[sP].[mR](A)(sP].[fR](U)[sP].[mR](C)[sP}.[fR](A)(sP].[mR](U)[sP].[mR](U)[sP].[fR](U)[sP][mR}(U)P.[fR] |
| | 28 | (G)P.[mR](G)P.[fR](C)P.[mR](A)P.[fR](G)P.[mR](G)P.[{R}(U)P.[mR](U)P.[{R}(U)P.[mR](U)P.[[R](U)P.[dR](C)P.[dR](C)P.[dR](A)P.[fR](G)P.[mR] |
| | PS DNA | ([A)P.[fR](C)P.[mR](G)P.[fR](G)P.[mR](C)P.[[R](A)P.[mR](G)P.[fR](G)[sP].[mR](U)[sP].[mR](C)[sP][mR](A)[sP].[mR](G)[sP].[mR](G)[sP][mR] |
| | central | (U)}$$$$V2.0 |
| | triplet | |
| | 2′OMe/ | |
| | 2′F | |
| | alter- | |
| | nating | |
| 88_4 | 35-1-16 | RNA1{[mR](A)[sP][mR](C)[sP].[mR](C)[sP].[mR](U)[sP].[mR](U)[sP]R(C)[sP].R(U)[sP].R(U)[sP].R(G)[sP].R(A)[sP].R(U)[sP].R(G)[sP].R(U)[sP] |
| | GAPDH | R(G)[sP].R(U)[sP].R(U)[sP].R(U)[P].R(U)P.R(U)P.R(C)P.R(C)P.R(A)P.R(G)P.R(A)P.R(C)[sP].R(G)[sP].R(G)[sP].R(C)[sP].R(A)[sP].R(G)[sP]R(G) |
| | 44 PS | [sP].R(U)[sP].R(U)P.R(U)P.R(U)P.R(U)P.R(C)P.R(C)P.R(A)P.R(G)P.R(A)P.R(C)P.R(G)P.R(G)[sP].R(C)[sP].R(A)[sP].R(G)[sP].R(G)[sP].R(U)[sP]. |
| | | [mR](C)[sP].[mR](A)[sP].[mR](G)[sP].[mR](G)[sP].[mR](U)}$$$$V2.0 |
| 88_5 | 35-1-16 | RNA1{[mR](A)[sP].[mR](C)[sP].[mR](C)[sP].[mR](U)[sP].[mR](U)[sP].R(C)[sP].R(U)[sP].R(U)[sP].R(G)[sP].R(A)[sP].R(U)[sP].R(G)[sP].R(U)[sP]. |
| | GAPDH | R(C)[sP].R(A)[sP].R(U)[sP].R(C)[sP].R(A)[sP].R(U)[sP].R(A)[sP].R(U)[sP].R(U)[sP].R(G)[sP].R(G)[sP].R(C)[sP].R(A)[sP].R(G)[sP].R(G)[sP]. |
| | 40 PS | R(U)[sP].R(U)P.R(U)P.R(U)s.R(U)P.R(C)P.R(C)P.R(A)P.R(G)P.R(A)P.R(C)P.R(G)P.R(G)P.R(C)[sP].R(A)[sP].R(G)[sP].R(G)[sP].R(U)[sP].[mR](C) |
| | | [sP].[mR](A)[sP].[mR](G)[sP].[mR](G)[sP].[mR](U)$$$$V2.0 |
| 88_6 | 35-1-16 | RNA1{[mR](A)[sP].[mR](C)[sP].[mR](C)[sP].[mR](U)[sP].[mR](U)[sP].R(C)[sP].R(U)[sP].R(U)[sP].R(G)[sP].R(A)[sP].R(U)[sP].R(G)[sP].R(U)[sP]. |
| | GAPDH | R(C)[sP].R(A)[sP].R(U)[sP].R(C)[P].R(A)[sP].R(U)[sP].R(A)[sP].R(U)[sP].R(U)[sP].R(U)[sP].R(G)[sP].R(G)[sP].R(C)[sP].R(A)[sP].R(G)[sP]. |
| | 36 PS | R(G)P.R(U)P.R(U)P.R(U)P.R(U)P.R(U)P.R(C)P.R(C)P.R(A)P.R(G)P.R(A)P.R(C)P.R(G)P.R(G)P.R(C)P.R(A)[sP].R(G)[sP].R(G)[sP].R(U)[sP].[mR(C)[sP]. |
| | | [mR](A)[sP].[mR](G)[sP].[mR](G)[sP].[mR((U)}$$$$V2.0 |
| 88_7 | 35-1-16 | RNA1{[mR](A)(sP].[mR](C)[sP].[mR](C)[sP].[mR](U)[sP].[mR](U)[sP].[mR](C)[sP].R(U)[sP].R(U)[sP].R(G)[sP].R(A)[sP].R(U)[sP].R(G)[sP].R(U) |
| | GAPDH | [sP].R(C)[sP].R(A)[sP].R(U)[sP].R(C)[sP].R(A)[sP].R(U)[P].R(A)[sP].R(U)[sP].R(U)[sP].R(U)[sP].R(G)[sP].R(G)[sP].R(C)[sP].R(A)P.R(G)P.R(G) |
| | 32 PS | P.R(U)P.R(U)P.R(U)P.R(U)P.R(U)P.R(C)P.R(C)P.R(A)P.R(G)P.R(A)P.R(C)P.R(G)P.R(G)P.R(C)P.R(A)P.R(G)P.R(G)[sP].R(U)[P].[mR](C)[sP].[mR)(A) |
| | | [sP].[mR](G)(sP][mR](G)[sP].[mR](U)}$$$$V2.0 |
| 88_8 | 35-1-16 | RNA1[mR)(A)[sP].[mR](C)[P].[mR(C)[sP][mR](U)[sP].[mR](U)[sP].R(C)[sP].R(U)[sP].R(U)[sP].R(G)[P].R(A)[sP].R(U)[sP].R(G)[sP].R(U)[sP].R(C) |
| | GAPDH | [sP].R(A)[sP].R(U)[sP].R(C)[sP].R(A)[sP].R(U)[P].R(A)[sP].R(U)[sP].R(U)[sP].R(U)P.R(G)P.R(G)P.R(C)P.R(A)P.R(G)P.R(G)P.R(U)P.R(U)P.R(U)P. |
| | 28 PS | R(U)P.R(U)P.R(C)P.R(C)P.R(A)P.R(G)P.R(A)P.R(C)P.R(G)P.R(G)P.R(C)P.R(A)P.R(G)P.R(G)[sP].R(U)[sP].[mR](C)[sP][mR](A)[sP].[mR](G)[sP][mR] |
| | | (U)}$$$$V2.0 |
| 88_9 | 35-1-16 | RNA1{[mR](A)[P].[mR](C)[sP].[mR](C)[P][mR](U)[sP].[mR](U)[sP].R(C)[sP].R(U)[P].R(U)[sP].R(G)[sP].R(A)[sP].R(U)[sP].R(G)[sP].R(U)[sP].R(C) |
| | GAPDH | [sP].R(A)[sP].R(U)[sP].R(C)[sP].R(A)[sP].R(U)P.R(A)P.R(U)P.R(U)P.R(U)P.R(G)P.R(G)P.R(C)P.R(A)P.R(G)P.R(G)P.R(U)P.R(U)P.R(UP.R(U)P.R(U)P. |
| | 24 PS | R(C)P.R(C)P.R(A)P.R(G)P.R(A)P.R(C)P.R(G)P.R(G)P.R(C)P.R(A)P.R(G)P.R(G)[sP].R(U)[sP].[mR](C)[sP].[mR](A)[sP][mR](G)[sP].[mR](U)}$$$$V2.0 |
| 88_10 | 35-1-16 | RNA1{[mR](A)[sP].[mR](C)[sP].[mR](C)[sP].[mR](U)[sP].[mR](U)[sP].R(C)[sP].R(U)[sP].R(U)[sP].R(G)[P].R(A)[sP].R(U)[sP].R(G)[sP].R(U)[sP]. |
| | GAPDH | R(C)[sP].R(A)P.R(U)P.R(C)P.R(A)P.R(U)P.R(A)P.R(U)P.R(U)P.R(U)P.R(G)P.R(G)P.R(C)P.R(A)P.R(G)P.R(G)P.R(U)P.R(U)P.R(U)P.R(U)P.R(U)P.R(C)P. |
| | 20 PS | R(C)P.R(A)P.R(G)P.R(A)P.R(C)P.R(G)P.R(G)P.R(C)P.R(A)P.R(G)P.R(G)[sP].R(U)[sP].[mR](C)[sP].[mR](A)[sP][mR](G)[sP].[mR)(G)(sP).[mR] |
| | | (U)}$$$$V2.0 |
| 88_11 | 35-1-16 | RNA1{[mR](A)[sP][mR](C)[P].[mR](C)[sP].[mR](U)[sP].[mR](U)[sP].R(C)[sP].R(U)[sP].R(U)[sP].R(G)[P].R(A)[sP].R(U)P.R(G)P.R(U)P.R(C)P.R(A)P. |
| | GAPDH | R(U)P.R(C)P.R(A)P.R(U)P.R(A)P.R(U)P.R(U)P.R(U)P.R(G)P.R(G)P.R(C)P.R(A)P.R(G)P.R(G)P.R(U)P.R(U)P.R(U)P.R(U)P.R(U)P.R(C)P.R(C)P.R(A)P.R(G) |
| | 16 PS | P.R(A)P.R(C)P.R(G)P.R(G)P.R(C)P.R(A)P.R(G)P.R(G)[sP].R(U)[sP].[mR](C)[sP].[mR](A)[sP].[mR](G)[sP].[mR](G)[sP].[mR((U)}$$$$V2.0 |
| 88_12 | 35-1-16 | RNA1{[mR](A)[sP][mR](C)[sP].[mR](C)[sP].[mR](U)[sP].[mR](U)[sP].R(C)[sP].R(U)P.R(U)P.R(G)P.R(A)P.R(U)P.R(G)P.R(U)P.R(C)P.R(A)P.R(U)P.R(C) |
| | GAPDH | P.R(A)P.R(U)P.R(A)P.R(U)P.R(U)P.R(U)P.R(G)P.R(G)P.R(C)P.R(A)P.R(G)P.R(G)P.R(U)P.R(U)P.R(U)P.R(U)P.R(U)P.R(C)P.R(C)P.R(A)P.R(G)P.R(A)P.R |
| | 12 PS | (C)P.R(G)P.R(G)P.R(C)P.R(A)P.R(G)P.R(G)[sP].R(U)[sP].[mR](C)[sP].[mR](A)[sP].[mR](G)[sP].[mR](G)[sP].[mR](U)}$$$$V2.0 |
| 88_13 | 35-1-16 | RNA1{[mR(A)[sP].[mR](C)[sP].[mR](C)[sP].[mR](U)[sP].[mR](U)[sP].R(C)[sP].R(U)[P].R(U)[sP].R(G)[sP].R(A)[sP].R(U)[sP].R(G)[sP].R(U)[sP]. |
| | GAPDH | R(C)[sP].R(A)[sP].R(U)[P].R(C)[sP].R(A)[sP].R(U)[sP].R(A)[sP].R(U)[sP].R(U)[sP].R(U)[sP].R(G)[sP].R(G)[sP].R(C)[P].R(A)[sP].R(G)P.R(R(G) |
| | 28 | [sP].R(U)[sP].[mR](C)[sP].[mR](A)[sP].[mR](G)[sP].[mR](G)[sP].[mR](U)}$$$$V2.0 |
| | PS + | |
| | 16PS dd | |
| | struc- | |
| | ture | |
| | no PS | |
| 89_1 | 35-1-16 | RNA1{[mR](A)[P].[mR](C)[sP].[mR](C)[sP].[mR](U)[sP].[mR](U)[sP].[dR](C)[sP].[mR](U)[sP].[dR](T)[sP].[mR](G)[sP].[dR](A)[sP].[mR](U)[sP]. |
| | GAPDH | [(dR)(G)[sP).[mR](U)[sP].[dR](C)\|sP],[mR](A)\|sP].[dR](T)[sP].[mR](C)(SP)[dR](A)(sP].[mR](U)(SP).[dR](A)(sP].[mR(U)(sP][dR](T)[sP][mR(U)P |
| | 28 | [dR](G)P.[mR](G)P.[dR](C)P.[mR](A)P[dR](G)P.[mR](G)P.[dR](T)P.[mR](U)P.[dR](T)P.[mR](U)P.[dR](T)P.[dR](C)P.[dR](C)P[dR](A)P.[dR](G)P.[mR] |
| | PS DNA | (A)P.[dR](C)P.[mR](G)P.[dR](G)P.[mR](C)P.[dR](A)P.[mR](G)P.[dR](G)[sP][mR](U)[sP].[mR](C)[sP].[mR](A)[sP].[mR](G)[P][mR](G)[sP].[mR](U)} |
| | central | $$$$V2.0 |
| | triplet | |
| | 2′OMe/ | |
| | DNA | |
| | alter- | |
| | nating | |
| 88_14 | 35-1-16 | RNA1{[mR](A)[sP].[mR](C)[sP].[mR](C)[sP].[mR](U)[sP][mR](U)[sP][mR](C)[sP].[mR](U)[sP].[[R](U)[sP].[mR](G)[sP][mR](A)[sP][[R](U)[sP].[mR |
| | GAPDH | (G)[sP].[mR](U)[sP].[[R](C)[sP].[mR](A)[sP].[mR](U)[sP].[[R](C)[sP].[mR](A)[sP].[mR](U)[sP].[fR](A)[sP][mR](U)[sP].[mR](U)[sP].[R](U)P. |
| | 2′Ome/ | [mR(G)P.[mR](G)P.[fR](C)P.[mR](A)P.[mR](G)P.[fR](G)P.[mR](U)P.[mR](U)P.[fR](U)P.[mR](U)P.[mR](U)P.[dR](C)P[dR](C)P.[dR](A)P.[fR](G)P.[mR] |
| | 2′F | (A)P.[mR](C)P.[[R](G)P.[mR](G)P.[mR](C)P.[fR](A)P.[mR](G)P.[mR](G)[P][mR](U)[sP].[mR](C)[sP].[mR](A)[sP].[mR](G)[sP][mR](G)[sP].[mR] |
| | mixmer | (U)}$$$$V2.0 |
| 90_1 | 35-1-16 | RNA1{[mR](A)[sP].[mR](C)[sP].[mR](C)[sP].[mR](U)[sP].[mR](U)[sP].[mR](C)[sP].[mR](U)[sP].[dR](T)[sP].[mR](G)[sP].[mR](A)[sP].[dR](T)[sP]. |
| | GAPDH | [mR](G)[sP].[mR](U)[sP].[dR](C)[sP].[mR](A)[sP].[mR](U)[sP].[dR](C)[sP].[mR](A)[sP].[mR](U)[sP].[dR](A)[sP].[mR](U)[sP].[mR](U)[sP].[dR] |
| | 2′Ome/ | (T)P.[mR](G)P.[mR](G)P.[dR](C)P.[mR](A)P.[mR](G)P.[dr}(G)P].[mR](U)P.[mR(U)P.[dR(T)P.[mR](U)P.[mR](U)P.[dR](C)P.[dR](C)P.[dR](A)P.[dR(G) |
| | DNA | P.[mR](A)P.[mR](C)P.[dR](G)P.[mR](G)P.[mR](C)P.[dR](A)P.[mR](G)P.[mR](G)[P].[mR](U)[sP].[mR](C)[sP].[mR](A)[sP].[mR](G)[sP].[mR](G)[sP]. |
| | mixmer | [mR](U)}$$$$V2.0 |
| 91_1 | 30-1-30 | RNA1{[mR](C)[sP].[mR](U)[sP].[mR](U)[sP].[mR](G)[sP].[mR](A)[sP].[fR](U)[sP].[mR](G)[sP].[fR](U)[sP].[mR](C)[sP].[fR](A)[sP].[mR](U)[sP]. |
| | hGAPDH | [fR](C)[sP].[mR](A)[sP].[fR](U)[sP].[mR](A)[sP].[fR](U)[sP].[mR](U)[sP].[fR](U)[sP].[mR](G)[sP].[fR](G)[sP].[mR](C)[sP].[fR](A)[sP].[mR] |
| | ORF #2 | (G)[sP].[fR](G)[sP].[mR](U)[sP].[fR](U)[sP].[mR](U)(sP].[fR](U)[sP].[mR](U)(sP].[dR](C)[sP].[dR](C)[sP].[dR](A)[sP].[mR](G)[sP].[fR](A) |
| | UAG all | [sP].[mR](C)[sP].[fR(G)[sP].[mR](G)[sP].[fR](C)[sP].[mR](A)[sP].[fR](G)[sP].[mR](G)[sP].[fR](U)[sP].[mR](C)[sP].[fR](A)[sP].[mR](G)[sP]. |
| | PS 2′F/ | [fR](G)[sP].[mR](U)[sP].[fR](C)[sP].[mR](C)[sP].[fR](A)[sP].[mR](C)[sP].[fR](C)[sP].[mR](A)[sP].[fR](C)[sP].[mR](U)[sP].[fR](G)[sP].[mR] |
| | 2′Ome | (A)[sP].[fR](C)[sP].[mR](A)[sP].[mR](C)[sP].[mR](G)}$$$$V2.0 |
| | alt | |
| 91_2 | 30-1-30 | RNA1{[mR](C)[sP].[mR](U)[sP].[mR](U)[P].[mR](G)[sP].[mR](A)[sP].R(U)[sP].R(G)[sP].R(U)[sP].R(C)[sP].R(A)[sP].R(U)[sP].R(C)[P].R(A)[sP]. |
| | hGAPDH | R(U)[sP].R(A)[sP].R(U)[sP].R(U)[sP].R(U)[sP].R(G)[sP].R(G)[sP].R(C)[sP].R(A)[sP].R(G)[sP].R(G)[P].R(U)[sP].R(U)[sP].R(U)[sP].R(U)[sP]. |
| | ORF | R(U)[sP].R(C)[sP].R(C)[sP].R(A)[sP].R(G)[sP].R(A)[P].R(C)[P].R(G)[sP].R(G)[sP].R(C)[sP].R(A)[sP].R(G)[sP].R(G)[sP].R(U)[sP].R(C)[sP].R |
| | #2 UAG | (A)[sP].R(G)[sP].R(G)[sP].R(U)[sP].R(C)[sP].R(C)[sP].R(A)[sP].R(C)[sP].R(C)[sP].R(A)[sP].R(C)[P].R(U)[sP].R(G)[sP].[mR](A)[sP].[mR](C) |
| | all PS | [sP].[mR](A)[sP].[mR](C)[sP].[mR)(G)$$$$V2.0 |
| 91_3 | 30-1-30 | RNA1{[mR](C)[sP].[mR](U)[sP].[mR](U)[P].[mR](G)[sP].[mR](A)[sP].R(U)[sP].R(G)[sP].R(U)[sP].R(C)[sP].R(A)[sP].R(U)[sP].R(C)[sP].R(A)[sP]. |
| | hGAPDH | R(U)[sP].R(A)[sP].R(U)[sP].R(U)[sP].R(U)[sP].R(G)[P].R(G)[sP].R(C)[sP].R(A)[sP].R(G)[sP].R(G)[sP].R(U)[sP].R(U)[sP].R(U)[sP].R(U)[sP]. |
| | ORF | R(U)[sP].[dR](C)[sP].[dR](C)[sP].[R](A)[sP].R(G)[sP].R(A)[sP].R(C)[sP].R(G)[sP].R(G)[P].R(C)[sP].R(A)[sP].R(G)[sP].R(G)[sP].R(U)[sP].R(C] |
| | #2 UAG | [sP].[mR](A)[sP].[mR](C)[sP].[mR](G)}$$$$V2.0 |
| | all PS | |
| | DNA | |
| | center | |
| 92_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sp].[mR](G)[sP].[mR](U)[sP].[mR](C)[sP].[mR)(A)[sP].[mR](G)(sP).[mR](G)}$$$$V2.0 |
| | I 2′F/ | |
| | 2′Ome | |
| | alt | |
| 93_1 | 30-1-30 | RNA1{[mR](C)[sP].[mR](U)[sP].[mR](U)[sP].[mR](G)[sP].[mR](A)[sP].R(U)[Sp].R(G)[Sp].R(U)[Sp].R(C)[Sp].R(A)[Sp].R(U)[Sp].R(C)[Sp].R(A)[Sp]. |
| | hGAPDH | R(U)[sP].R(A)[sP].R(U)[sP].R(U)[sP].R(U)[Sp].R(G)[Sp].R(G)[Sp].R(C)[Sp].R(A)[Sp].R(G)[Sp].R(G)[Sp].R(U)[Sp].R(U)[Sp].R(U)[Sp].R(U)[Sp]. |
| | ORF #2 | R(U)[sP].[dR](C)[sP].[dR](Z)[sP].[dR](A)[sP].R(G)[sP].R(A)[sP].R(C)[sP].R(G)[sP].R(G)[sP].R(C)[sP].R(A)[sP].R(G)[sP].R(G)[sP].R(U)[sP]. |
| | UAG all | R(C)[SP].R(A)[sP].R(G)[sP].R(G)[sP].R(U)[sP].R(C)[sP].R(C)[sP].R(A)[sP].R(C)[sP].R(C)[sP].R(A)[sP].R(C)[sP].R(U)[P].R(G)[sP].[mR](A)[sP]. |
| | PS DNA | [mR](C)[sP].[mR](A)[sP].[mR](C)[sP].[mR](G)}$$$$$V2.0 |
| | center | |
| 94_1 | 24-1-12 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].mR(U)[sP]. |
| | GAPDH | [fR](U)[sP].[mR](G)[sP].[fR](G)[sP].[mR](C)[P].[[R](A)[P][mR](G)[sP].[fR](G)[sP].[mR](U)[sP][[R](U)(sP].[mR](U)[sP][[R](U)[sP].[mR](U) |
| | ORF #2 | [sP].[dR](C)[P][dR(C)[sP].[dR](A)[sP][mR](G)[sP].[fR](A)[sP][mR](C)[sP].[fR](G)[sP].[mR](G)[sP].[fR](C)[P].[mR](A)[sP].[mR](G)[P][mR](G) |
| | all PS | [sP].[mR(U)[sP][mR](C)}$$$$V2.0 |
| | I 2′F/ | |
| | 2′Ome | |
| | alt | |
| 95_1 | 24-1-10 | RNA1{[mR](G)[sP].[mR](U)[sP].[mR](C)[sP][mR](A)[sP][mR(U)[sP].[fR](C)[sP].[mR](A)[sP].[R](U)[sP][mR](A)[sP][IR](U)[sP].[mR](U)[sP]. |
| | GAPDH | [fR](U)[P].[mR](G)[sP].[fR](G)[P].[mR](C)[sP][fR](A)[sP].[mR](G)[sP][fR](G)[sP][mR](U)[sP].[[R](U)[sP][mR](U)[sP].[[R](U)[sP][mR](U)[P] |
| | ORF #2 | [dR](C)[P].[dR](C)[P].[dR](A)[sP].[mR](G)[sP][fR](A)[P].[mR](C)[sP].[fR](G)[sP].[mR](G)[sP].[mR](C)[sP].[mR](A)[sP].[mR](G)[sP][mR](G)}$ |
| | all PS | $$$V2.0 |
| | I 2′F/ | |
| | 2′Ome | |
| | alt | |
| 96_1 | 24-1-8 | RNA1{[mR)(G)[sP].[mR](U)[sP].[mR](C)[sP].[mR](A)[sP].[mR(U)[sP].[[R](C)[sP].[mR](A)[sP].[IR](U)[P].[mR](A)[sP].[[R](U)[sP].[mR](U)[sP]. |
| | GAPDH | [fR(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR)(C)[sP].[dR](C)[sP].[dR](A)[sP].[mR](G)[sP].[fR](A)[sP].[mR)(C)[sP].[mR](G)[sP].[mR](G)[sP].[mR](C)[sP].[mR](U)}$$$$V2.0 |
| | all PS | |
| | I 2′F/ | |
| | 2′Ome | |
| | alt | |
| 97_1 | 24-1-6 | RNA1{[mR](G)[sP].[mR](U)[sP].[mR](C)[sP].[mR](A)[sP].[mR](U)[sP].[fR](C)[sP].[mR](A)[sP].[[R](U)[P].[mR](A)[P].[[R](U)[sP].[mR](U)[sP]. |
| | GAPDH | [fR](U)[sP].[mR](G)[sP].[fR](G)[sP].[mR](C)[sP].[fR](A)[sP].[mR](G)[sP].[[R](G)[sP].[mR](U)[sP].[fR](U)[sP].[mR](U)[sP].[[R](U)[sP].[mR] |
| | ORF #2 | (U)[sP].dR](C)[sP].[dR](C)[sP].[dR](A)[sP].[mR](G)[sP].[mR](A)[sP].[mR](C)[sP].[mR](G)[sP].[mR](G)}$$$$V2.0 |
| | all PS | |
| | I 2′F/ | |
| | 2′Ome | |
| | alt | |
| 98_1 | 24-1-4 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP] |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF alt | (U)[sP].[dR](C)[sP].[dR](C)[sP].[dR](A)[sP].[mR](G)[sP].[mR](A)[sP].[mR](C)}$$$$V2.0 |
| 99_1 | 22-1-12 | RNA1{[mR](C)[sP].[mR](A)[sP].[mR](U)[sP].[mR](C)[sP].[mR](A)[sP].[fR](U)[P].[mR](A)[P].[[R](U)[P].[mR](U)[sP].[fR](U)[sP].[mR](G)[sP]. |
| | GAPDH | [fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR](U)[sP].[dR](C)[sP].[dR] |
| | ORF #2 | (C)[sP].[dR](A)[sP].[mR](G)[sP].[fR](A)[sP].[mR](C)[sP].[fR](G)[sP].[mR](G)[sP].[fR](C)[sP].[mR](A)[sP].[mR](G)[sP][mR](G)[sP].[mR](U) |
| | all PS | [sP].[mR](C)}$$$$V2.0 |
| | I 2′F/ | |
| | 2′Ome | |
| | alt | |
| 100_1 | 22-1-10 | RNA1{[mR](C)[sP].[mR](A)[P].[mR](U)[P].[mR](C)[sP].[mR](A)[sP].[fR](U)[sP].[mR](A)[sP].[[R](U)[sP].[mR](U)[sP].[fR](U)[sP].[mR](G)[sP]. |
| | GAPDH | [fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR](U)(sP].[R](C)[sP].[dR] |
| | ORF #2 | (C)[sP].[dR(A)[sP].[mR](G)[sP].[fR](A)[sP].[mR](C)[sP].[fR](G)[sP].[mR](G)(sP].[mR(C)(sP][mR(A)[sP].[mR((G)[sP].[mR)(G)}$$$$V2.0 |
| | all PS | |
| | I 2′F/ | |
| | 2′Ome | |
| | alt | |
| 101_1 | 22-1-8 | RNA1{[mR](C)[sP].[mR](A)[sP].[mR](U)[sP].[mR](C)[sP].[mR](A)[sP].[fR](U)[sP].[mR](A)[sP].[fR](U)[sP].[mR](U)[sP].[fR](U)[sP].[mR](G)[sP]. |
| | GAPDH | [fR](G)[sP].[mR](C)[sP].[fR](A)[sP].[mR](G)[sP].[fR](G)[sP].[mR](U)[sP].[fR](U)[sP].[mR](U)[sP].[fR](U)[sP][mR](U)[sP][dR](C)[sP].[dR](C) |
| | ORF #2 | [sP].[dR](A)[sP].[mR)(G)[sP].[fR](A)[sP].[mR(C)[sP].[mR](G)[sP].[mR](G)[sP].[mR](C)[sP].[mR(U)}$$$$V2.0 |
| | all PS | |
| | I 2′F/ | |
| | 2′Ome | |
| | alt | |
| 102_1 | 22-1-6 | RNA1{[mR](C)[sP].[mR](A)[sP].[mR](U)[sP].[mR](C)[sP].[mR](A)[sP].[fR](U)[sP].[mR](A)[sP].[fR](U)[sP].[mR](U)[sP].[fR](U)[sP].[mR](G)[sP]. |
| | GAPDH | [fR](G)[sP].[mR](C)[sP].[fR](A)[sP].[mR](G)[sP].[fR](G)[sP].[mR](U)[sP].[fR](U)[sP].[mR](U)[sP][R](U)[sP][mR](U)[sP].[dR](C)[sP].[dR](C) |
| | ORF #2 | [sP].[dR](A)[sP].[mR](G)[sP].[mR](A)[sP].[mR](C)[sP].[mR](G)[sP].[mR](G)}$$$$V2.0 |
| | all PS | |
| | I 2′F/ | |
| | 2′Ome | |
| | alt | |
| 103_1 | 22-1-4 | RNA1{[mR)(C)[sP].[mR](A)[sP].[mR](U)[sP].[mR](C)[sP].[mR](A)[sP].[fR](U)[sP].[mR](A)[sP][[R](U)[sP].[mR](U)[P][fR](U)[sP][mR](G)[sP]. |
| | GAPDH | [fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR](U)[sP].[dR](C)[sP]. |
| | ORF #2 | [dR](C)[sP].[dR](A)[sP].[mR)(G)(sP].[mR](A)(sP].[mR](C)}$$$$V2.0 |
| | all PS | |
| | I 2′F/ | |
| | 2′Ome | |
| | alt | |
| 104_1 | 20-1-12 | RNA1{[mR)(U)[sP].[mR](C)[sP][mR](A)[sP][mR](U)[sP].[mR](A)[sP].[fR](U)[sP].[mR](U)[sP].[fR](U)[sP][mR](G)[sP].[fR](G)[sP].[mR](C)[sP]. |
| | GAPDH | [fR](A)[sP].[mR](G)[sP].[fR](G)[sP][mR](U)[sP].[fR](U)[P][mR(U)[sP].[fR](U)[sP][mR](U)[P].[dR](C)[P].[dR](C)[P].[dR](A)[sP].[mR](G)(sP]. |
| | ORF #2 | [fR](A)[sP].[mR](C)[sP].[fR](G)[sP].[mR](G)[sP].[fR](C)[sP].[mR](A)[sP].[mR](G)[sP}.[mR](G)[sP].[mR](U)[sP].[mR](C)}$$$$V2.0 |
| | all PS | |
| | I 2′F/ | |
| | 2′Ome | |
| | alt | |
| 105_1 | 20-1-10 | RNA1{[mR](U)[sP].[mR](C)[sP].[mR](A)[sP].[mR](U)[sP].[mR](A)[sP].[fR](U)[sP].[mR](U)[sP].[fR](U)[sP].[mR](G)[sP].[fR](G)[sP].[mR](C)[sP]. |
| | GAPDH | [fR](A)[sP].[mR](G)[sP].[fR](G)[sP].[mR](U)[sP].[fR](U)[sP].[mR](U)[sP].[fR](U)[P].[mR](U)[sP].[dR](C)[sP].[dR](C)[sP].[dR](A)[sP].[mR](G) |
| | ORF #2 | [sP].[fR](A)[sP].[mR](C)[sP].[fR](G)[sP].[mR](G)[sP].[mR](C)(sP].[mR](A)(sP][mR](G)\|sP].[mR](G)}$$$$V2.0 |
| | all PS | |
| | I 2′F/ | |
| | 2′Ome | |
| | alt | |
| 106_1 | 20-1-8 | RNA1{[mR](U)[sP].[mR](C)[sP].[mR](A)[sP].[mR](U)[sP].[mR](A)[sP].[fR](U)[sP].[mR](U)[sP].[fR](U)[sP].[mR](G)[sP].[fR](G)[sP].[mR](C)[sP]. |
| | GAPDH | [fR](A)[sP].[mR](G)[sP].[fR](G)[sP].[mR](U)[sP].[fR](U)[sP].[mR](U)[sP].[fR](U)[sP].[mR](U)[sP].[dR](C)[sP][dR](C)[sP].[dR](A)[sP].[mR] |
| | ORF #2 | (G)[sP].[fR](A)[sP].[mR](C)[sP].[mR(G)[sP].[mR](G)[sP].[mR](C)[sP].[mR)(U)}$$$$V2.0 |
| | all PS | |
| | I 2′F/ | |
| | 2′Ome | |
| | alt | |
| 107_1 | 20-1-6 | RNA1{[mR](U)[sP].[mR](C)[sP].[mR](A)[sP].[fR](U)[sP].[mR](U)[sP].[fR](U)[sP].[mR](G)[sP].[fR](G)[sP].[mR](C)[sP].[fR](A)[sP].[mR](G)[sP]. |
| | GAPDH | [fR](G)[sP].[mR](U)[sP].[fR](U)[sP].[mR](U)[sP].[fR](U)[sP].[mR](U)[sP].[dR](C)[P][dR](C)[sP][dR](A)[sP][mR](G)[sP].[mR](A)[sP].[mR](C) |
| | ORF #2 | [sP].[mR](G)[sP].[mR](G)}$$$$V2.0 |
| | all PS | |
| | I 2′F/ | |
| | 2′Ome | |
| | alt | |
| 108_1 | 20-1-4 | RNA1{[mR(U)[P].[mR](C)[sP].[mR](A)[sP].[mR](U)[sP].[mR](A)[sP][fR](U)[sP].[mR](U)[sP][fR](U)[sP].[mR](G)[P][[R](G)[P].[mR](C)[sP]. |
| | GAPDH | [fR](A)[sP].[mR](G)[sP].[fR](G)[sP].[mR](U)[sP].[fR](U)[sP].[mR](U)[sP].[fR](U)[P].[mR](U)[sP][dR](C)[sP][dR](C)[sP].[dR](A)[sP].[mR](G) |
| | ORF #2 | [sp].[mR](A)[sP].[mR](C)}$$$$V2.0 |
| | all PS | |
| | I 2′F/ | |
| | 2′Ome | |
| | alt | |
| 92_2 | 24-1-15 | RNA1{[mR](G)P.[mR](U)[sP].[mR](C)[sP][mR](A)[sP].[mR](U)[sP].[fR](C)[sP].[mR](A)[sP].[fR](U)[sP].[mR](A)[sP].[fR](U)[sP].[mR](U)[P]. |
| | GAPDH | [fR](U)[sP].[mR](G)[sP].[fR](G)[sP].[mR](C)[sP].[fR](A)[sP].[mR](G)[sP].[fR](G)[sP].[mR](U)[P][fR](U)[sP].[mR](U)[sP].[fR](U)[sP].[mR](U) |
| | ORF #2 | [sP].[dR](C)[sP].[dR](C)[P].[dR](A)[sP].[mR](G)[sP].[fR](A)[sP].[mR](C)[sP].[fR](G)[sP].[mR](G)[sP].[fR](C)[P].[mR](A)[sP].[fR](G)[sP]. |
| | 2′F/ | [mR](G)[sP].[mR](U)[sP][mR](C)[sP].[mR](A)[sP].[mR](G)[sP].[mR](G)}$$$$V2.0 |
| | 2′Ome | |
| | alt | |
| | PO 23 | |
| 92_3 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR].(U)[sP].[mR](G)[sP].[fR](G)[sP].[mR](C)[sP].[fR](A)[sP].[mR](G)[sP].[fR](G)[sP].[mR](U)[P].[fR](U)[sP].[mR](U)[sP].[fR](U)[P].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP][fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 22 | |
| 92_4 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP][mR](U)[sP].[fR](U)[sP][mR](U)[sP].[fR](U)[sP][mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 21 | |
| 92_5 | 24-1-15 | RNA1{[mR](G)[sP].[mR](U)[sP].[mR](C)[sP].[mR](U)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[P].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 20 | |
| 92_6 | 24-1-15 | RNA1{[mR](G)[sP].[mR](U)[sP].[mR](C)[sP].[mR](A)[sP].[mR](U)P.[{R}(C)[sP].[mR](A)[sP].[fR](U)[sP].[mR](A)[sP].[fR](U)[sP].[mR(U)[sP]. |
| | GAPDH | [fR](U)[sP].[mR](G)[sP].[fR](G)[sP].[mR](C)[sP].[fR](A)[sP].[mR](G)(sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 19 | |
| 92_7 | 24-1-15 | RNA1{[mR](G)[sP].[mR](U)[sP].[mR](C)[sP].[mR](A)[sP].[mR](U)P.[fR](C)[sP].[mR](A)[sP}.[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[P].[dR](C)[sP].[dR](A)[P].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 18 | |
| 92_8 | 24-1-15 | RNA1{[mR](G)[sP].[mR](U)[sP].[mR](C)[sP].[mR](A)[sP].[mR](U)[sP].[fR](C)[sP].[mR](U)P.[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[P].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[P].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 17 | |
| 92_9 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP}.[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sp].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 16 | |
| 92_10 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](U)P.[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[P].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR]9C)[sP].[mR](A)[sP].[mR](G)[sP].[mR](G)}$$$$V2.0 |
| | 2′Ome | |
| | alt | |
| | PO 15 | |
| 92_11 | 24-1-15 | RNA1{[mR](G)[sP].[mR](U)[sP].[mR](C)[sP].[mR](A)[sP].[mR](U)[sP][[R](C)[sP].[mR](A)[sP].[fR](U)(sP].[mR](A)[sP].[fR](U)P.[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP][mR](U)[sP].[[R](U)[sP].[mR] |
| | ORF #2 | [(U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[P].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 14 | |
| 92_12 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)P. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[P].[mR] |
| | ORF #2 | [U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)[sP].[mR](A)(sP].[mR](G)[sP].[mR)(G)}$$$$V2.0 |
| | 2′Ome | |
| | alt | |
| | PO 13 | |
| 92_13 | 24-1-15 | RNA1{[mR](G)[sP].[mR](U)[sP].[mR](C)[sP].[mR](A)[sP].[mR](U)[P].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)(sP].[mR](C)[sP].[mR](A)[sP].[mR](G)[sP].[mR](G)}$$$$V2.0 |
| | 2′Ome | |
| | alt | |
| | PO 12 | |
| 92_14 | 24-1-15 | RNA1{[mR](G)[sP].[mR](U)[sP][mR](C)[sP].[mR](A)[sP].[mR](U)[sP].[fR](C)[sP].[mR](A)[sP].[fR](U)[sP].[mR](A)[sP].[R](U)[sP][mR](U)[sP]. |
| | GAPDH | [fR](U)[sP].[mR](G)P.[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)[P].[fR](G)[sP].[mR](G)[sP].[fR](C)[P].[mR](A)[sP].[fR](G)[sP]. |
| | 2′F/ | [mR](G)[sP].[mR](U)[sP].[mR](C)[sP].[mR](A)[sP].[mR](G)[sP]0.[mR](G)}$$$$V2.0 |
| | 2′Ome | |
| | alt | |
| | PO 11 | |
| 92_15 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)(sP].[mR](A)[sP].[fR](U)[P].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | [U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)(sP].[mR](C)[sP].[mR](A)[sP].[mR](G)[sP].[mR](G)}$$$$V2.0 |
| | 2′Ome | |
| | alt | |
| | PO 10 | |
| 92_16 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)[sP].[fR](G)[sP].[mR](C)P.[fR](A)[sP].[mR](G)[sP].[fR](G)[sP].[mR](U)[sP].[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[P].[mR](A)[sP].[fR](G)[sP]. |
| | 2′F/ | [mR](G)[sP].[mR](U)[sP].[mR)(C)[sP].[mR](A)[sP].[mR](G)[sP].[mR](G)}$$$$V2.0 |
| | 2′Ome | |
| | alt | |
| | PO 9 | |
| 92_17 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | [U)[sP].[dR](C)[sP].[dR](C)[sP].[dR](A)[sP].[mR](G)[sP].[fR](A)[sP].[mR](C)[sP].[fR](G)(sP].[mR](G)[sP].[fR](C)[sP].[mR](A)[sP].[fR](G) |
| | 2′F/ | [sP].[mr](G)[sP].[mR](U)(sP].[mR](C)[sP].[mR](A)[sP].[mR](G)[sP].[mR)(G)}$$$$V2.0 |
| | 2′Ome | |
| | alt | |
| | PO 8 | |
| 92_18 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)P.[fR](G)[sP].[mR](U)[sP].[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)(sP].[fR](G)[sP].[mR](G)[P].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 7 | |
| 92_19 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)[sP].[fR](G)[sP].[mR](C)[sP].[fR](A)[sP].[mR](G)[P].[fR](G)P.[mR](U)[sP].[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[P].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 6 | |
| 92_20 | 24-1-15 | RNA1{[mR](G)[sP].[mR](U)[sP].[mR](C)[sP].[mR](A)[sP].[mR](U)[P].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)P.[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR)(C)[sP].[mR](A)[sP].[mR](G)[sP].[mR](G)}$$$$V2.0 |
| | 2′Ome | |
| | alt | |
| | PO 5 | |
| 92_21 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[P].[fR](G)[P].[mR](G)[sP].[fR](C)[sP].[mR](A)[sP].[fR](G) |
| | 2′F/ | [sP].[mR](G)[sP].[mR](U)[sP].[mR](C)[sP].[mR](A)[sP].[mR](G)sP].[mR](G)}$$$$V2.0 |
| | 2′Ome | |
| | alt | |
| | PO 4 | |
| 92_22 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)P.[fR](U)[sP].[mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)(sP].[mR](C)[sP].[mR](A)[sP].[mR](G)[sP].[mR](G)}$$$$V2.0 |
| | 2′Ome | |
| | alt | |
| | PO 3 | |
| 92_23 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)P.[dR](C)[P].[dR](C)[sP].[dR](A)[sP].[mR](G)[sP].[fR](A)[sP].[mR](C)[sP].[fR](G)[sP].[mR](G)[sP].[fR](C)[sP].[mR](A)[P].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 2 | |
| 92_24 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)P.[dR](C)[sP].[dR](C)[sP}.[dR](A)[sP].[mR](G)[sP].[fR](A)[sP].[mR](C)[sP].[fR](G)[sP].[mR](G)[sP}.[fR](C)[sP].[mR](A)[sP].[fR](G)[sP]. |
| | 2′F/ | [mR](G)[sP].[mR](U)(sP].[mR](C)[sP].[mR](A)[sP].[mR](G)[sP].[mR](G)}$$$$V2.0 |
| | 2′Ome | |
| | alt | |
| | PO 1 | |
| 92_25 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)(sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)P.[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO 0 | |
| 92_26 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mA](A)[sP].[fR](U)[sP].[mR](U)[sP]. |
| | GAPDH | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)P.[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)(SP).[mR](C)[sP].[mR](A)[sP].[mR](G)\|sP].[mR](G)}$$$$V2.0 |
| | 2′Ome | |
| | alt | |
| | PO −1 | |
| 92_27 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](C)[sP].[fR](G)[sP].[mR](G)[sP].[fR](C)[sP].[mR](A)[sP].[fR](G)[sP].[mR] |
| | ORF #2 | (G)[sP].[dR](C)[sP].[dR](C)[sP].[dR](A)P.[mR](G)[sP].[fR](A)[sP].[mR](C)[sP].[fR](G)[sP].[mR](G)[sP].[fR](C)(sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −2 | |
| 92_28 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)P.[mR](C)[sP].[fR](G)[sP].[mR](G)[sP].[fR](C)[sP].[mR](A)[sP].[fR](G)[sP]. |
| | 2′F/ | [mR](G)[sP].[mR)(U)[sP].[mR](C)\|sP].[mR](A)[sP].[mR](G)[sP].[mR](G)}$$$$V2.0 |
| | 2′Ome | |
| | alt | |
| | PO −3 | |
| 92_29 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)(sP].[mR](G)[sP].[fR](A)P.[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −4 | |
| 92_30 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)P.[fR](G)[sP].[mR](G)[sP].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −5 | |
| 92_31 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)P.[mR](G)[sP].[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −6 | |
| 92_32 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR(C)[sP].[dR](C)[sP].[dR](A)[sP].[mR](G)[sP].[fR](A)[sP].[mR](C)[sP].[fR](G)[sP].[mR](G)P.[fR](C)[sP].[mR](A)[sP].[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −7 | |
| 92_33 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](U)P.[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −8 | |
| 92_34 | 24-1-15 | RNA1{[mR](G)[sP].[mR](U)[sP].[mR](C)[sP].[mR](A)[sP].[mR](U)[sP].[fR](C)[sP].[mR](A)[$P].[fR](U)[sP].[mR](A)(SP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR(G)[sP].[mR](G)[sP].[fR](C)[sP].[mR](U)P.[fR](G)[sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −9 | |
| 92_35 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)P. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −10 | |
| 92_36 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP}.[mR](A)[sP].[fR](G) |
| | 2′F/ | [sp].[mR](G)P.[mR](U)[sP].[mR](C)[sP].[mR](A)[sP].[mR](G)[sP].[mR](G)}$$$$V2.0 |
| | 2′Ome | |
| | alt | |
| | PO −11 | |
| 92_37 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)P.[mR](C)[sP].[mR](A)[sP].[mR](G)[sP].[mR](G)}$$$$V2.0 |
| | 2′Ome | |
| | alt | |
| | PO −12 | |
| 92_38 | 24-1-15 | RNA1{[mR](G)[sP].[mR](U)[sP].[mR](C)[sP].[mR](A)(sP].[mR](U)[sP].[fR](C)[sP].[mR](A)[sP].[fR](U)(sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)(sP].[mR](U)[sP].[mR(C)P.[mR](A)[sP].[mR](G)[sP].[mR](G)}$$$$V2.0 |
| | 2′Ome | |
| | alt | |
| | PO −13 | |
| 92_39 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[P].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)(sP].[mR](U)P.[mR](G)[sP].[mR](G)}$$$$V2.0 |
| | 2′Ome | |
| | alt | |
| | PO −14 | |
| 92_40 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR]G)(sP].[mR(U)[sP].[mR(C)[sP].[mR](A)[sP].[mR)(G)P[mR](G)}$$$$V2.0 |
| | 2′Ome | |
| | alt | |
| | PO −15 | |
| 92_41 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)P.[fR](G)P.[mR](U)[sP].[fR](U)[sP].[mR](U)[sP].[fR](U)[sP].[mR](U) |
| | ORF #2 | [sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR](C)[sP].[mR](A)(sP].[mR(G)[sP].[mR](G)}$$$$V2.0 |
| | 2′Ome | |
| | alt | |
| | PO 6, | |
| | 7, 11 | |
| 92_42 | 24-1-15 | RNA1{[mR](G)[sP].[mR](U)[P].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)P.[dR](A)P.[mR](G)[sP].[fR](A)[P].[mR](C)[sP].[fR](G)[sP].[mR](G)[sP].[fR](C)[sP].[mR](A)[sP].[fR](G)(sP]. |
| | 2′F/ | [mR](G)[sP].[mR](U)[sP].[mR](C)[sP].[mR](A)[sP].[mR](G)[sP].[mR](G)}$$$$V2.0 |
| | 2′Ome | |
| | alt | |
| | PO −1, | |
| | −2 | |
| 92_43 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)P.[fR](A)P.[mR](C)P.[fR](G)[sP].[mR](G)[sP].[fR](C)[sP].[mR](A)[sP].[fR](G)[sP].[mR] |
| | 2′F/ | (G)[sP].[mR](U)[sP].[mR](C)[sP].[mR](A)[sP].[mR)(G)(sP].[mR](G)}$$$$V2.0 |
| | 2′Ome | |
| | alt | |
| | PO −3, | |
| | −4, −5 | |
| 92_44 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | PS | [sP].[mR](G)[Sp].[mR](U)[sP].[mR](C)[sP].[mR](A)[sP].[mR)(G)[sP].[mR](G)}$$$$V2.0 |
| | 2′F/ | |
| | 2′Ome | |
| | alt | |
| | PO −1, | |
| | −2, −3, | |
| | −4, −5 | |
| 92_45 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR}(U)[sP].[mR](G)P.[fR](G)[sP].[mR](C)[sP].[fR](A)[sP].[mR](G)P.[fR](G)[sP].[mR](U)[sP].[fR](U)[sP].[mR](U)[sP].[fR](U)[sP].[mR](U) |
| | ORF #2 | [sP].[dR](C)[sP].[dR](C)P.[dR](A)[sP].[mR](G)P.[fR](A)P.[mR](C)P.[fR](G)[sP].[mR](G)[sP].[fR](C)[sP].[mR](A)[sP].[fR](G)[sP].[mR](G)[sP]. |
| | 2′F/ | [mR](U)[sP].[mR](C)(sP].[mR](A)(SP).[mR](G)[sP].[mR)(G)}$$$$V2.0 |
| | 2′Ome | |
| | alt PO | |
| | −1, −3, | |
| | −4, −5, | |
| | 7, 11 | |
| 92_46 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)(sP].[mR](A)[sP].[fR](U)[sP].[mR](U)[sP]. |
| | GAPDH | [fR](U)[sP].[mR](G)P.[fR](G)[sP].[mR](C)[sP].[fR](A)[sP].[mR](G)P.[fR](G)P.[mR](U)[sP].[fR](U)[sP].[mR](U)[sP].[fR](U)[sP].[mR](U)[sP]. |
| | ORF #2 | [dR](C)[sP].[dR](C)P.[dR](A)P.[mR](G)P.[fR](A)P.[mR](C)P.[fR](G)[sP].[mR](G)[sP].[fR](C)[sP].[mR](A)[sP].[fR](G)[sP].[mR](G)[sP].[mR](U) |
| | 2′F/ | [sP].[mR](C)[sP].[mR](A)[sP].[mR](G)[sP].[mR](G)}$$$$V2.0 |
| | 2′Ome | |
| | alt PO | |
| | −1, −2, | |
| | −3, −4, | |
| | -5, 6, | |
| | 7, 11 | |
| 92_47 | 24-1-15 | RNA1{[mR](G)[sP].[mR](U)[sP].[mR](C)[sP].[mR](A)[sP].[mR](U)[sP].[fR](C)[sP].[mR]A)[sP].[fR](U)[sP].[mR](A)[sP].[fR](U)[sP].[mR](U)[sP]. |
| | GAPDH | [fR}(U)[sP].[mR](G)P.[fR](G)[sP].[mR](C)[sP].[fR](A)[sP].[mR](G)P.[fR](G)P.[mR](U)[sP].[fR](U)[sP].[mR](U)[sP].[fR](U)[sP].[mR](U)[sP]. |
| | ORF #2 | [dR](C)[sP].[dR](C)[sP].[dR](A)[sP].[mR](G)P.[fR](A)P.[mR](C)P.[fR](G)[sP].[mR](G)[sP].[fR](C)[sP].[mR](A)[sP].[fR](G)[sP].[mR](G)[sP]. |
| | 2′F/ | [mR](U)[sP].[mR](C)[sP].[mR](A)[sP].[mR](G)[sP].[mR](G)}$$$$V2.0 |
| | 2′Ome | |
| | alt PO | |
| | −3, −4, | |
| | −5, 6, | |
| | 7, 11 | |
| 92_48 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U) |
| | GAPDH | [sP].[fR](U)[sP].[mR](G)P.[fR](G)[sP].[mR](C)[sP].[fR](A)[sP].[mR](G)P.[fR](G)[sP].[mR](U)[sP].[fR](U)[sP].[mR](U)[sP].[fR](U)[sP].[mR] |
| | ORF #2 | (U)[sP].[dR(C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | 2′F/ | [sP].[mR](G)[sP].[mR](U)[sP].[mR](C)[sP].[mR](A)[sP].[mR](G)[sP].[mR](G)}$$$$V2.0 |
| | 2′Ome | |
| | alt PO | |
| | 8, 12 | |
| 92_49 | 24-1-15 | RNA1{[mR](G)[sP].[mR](U)P.[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)(sP].[fR](A)[sP].[mR](G)[sP].[fR](G)[sP].[mR](U)[sP].[fR](U)[sP].[mR](U)[sP].[fR](U)[sP]. |
| | ORF #2 | [mR](U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP]. |
| | 2′F/ | [fR](G)[sP].[mR](G)[sP].[mR](U)[sP].[mR](C)[sP].[mR)(U)P.[mR(G)[sP].[mR)(G)}$$$$V2.0 |
| | 2′Ome | |
| | alt | |
| | PO 22, | |
| | −14 | |
| 92_50 | 24-1-15 | RNA1{[mR](G)[sP].[mR](U)P.[mR](C)P.[mR](A)[sP].[mR](U)[sP].[fR](C)[sP].[mR](A)[sP].[fR](U)[sP].[mR](A)[sP].[fR](U)[sP].[mR](U)[sP].[fR] |
| | GAPDH | (U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR](U) |
| | ORF #2 | [sP].[dR](C)[sP].[dR](C)[sP].[dR](A)(sP].[mR](G)[sP].[fR](A)[sP].[mR](C)[sP].[fR](G)[sP].[mR](G)[sP].[fR](C)[sP].[mR](A)[sP].[fR](G)[sP]. |
| | 2′F/ | [mR](G)[sP].[mR](U)[sP].[mR](C)P.[mR(U)P.[mR](G)[sP].[mR](G)}$$$$V2.0 |
| | 2′Ome | |
| | alt | |
| | PO 22, | |
| | 21, | |
| | −13, | |
| | −14 | |
| 92_51 | 24-1-15 | RNA1{[mR](G)[sP].[mR](U)P.[mR](C)P.[mR](U)P.[mR](U)[sP].[fR](C)[sP].[mR](A)[sP].[fR](U)[sP].[mR](A)[sP].[fR](U)[sP].[mR](U)[sP].[fR](U) |
| | GAPDH | [sP].[mR](G)[sP].[fR](G)[sP].[mR](C)[sP].[fR](A)[sP].[mR](G)[sP].[fR](G)(sP].[mR](U)[sP].[fR](U)[sP].[mR](U)[sP].[fR](U)[sP].[mR(U)[sP]. |
| | ORF #2 | [dR](C)[sP].[dR](A)[sP].[mR](G)P.[fR](A)P.[mR](C)P.[fR](G)[sP].[mR](G)[sP].[fR](C)[sP].[mR](A)[sP].[fR](G)[sP].[mR](G)[sP].[mR](U)[sP]. |
| | 2′F/ | [mR](C)[sP].[mR](U)[sP].[mR](G)[sP].[mR](G)}$$$$V2.0 |
| | 2′Ome | |
| | alt | |
| | PO 20 | |
| | to 22, | |
| | −12 to | |
| | −14 | |
| 92_52 | 24-1-15 | RNA1{[mR](G)[sP].[mR](U)P.[mR](C)P.[mR](U)P.[mR](U)[sP].[fR](C)[sP].[mR](A)[sP].[fR](U)[sP].[mR](A)[sP].[fR](U)[sP].[mR](U)[sP].[fR](U) |
| | GAPDH | [sP].[mR](G)P.[fR](G)[sP].[mR](C)[sP].[fR](A)[sP].[mR](G)P.[fR](G)[sP].[mR](U)[sP].[fR](U)[sP].[mR](U)[sP].[fR](U)[sP].[mR](U)[sP].[dR] |
| | ORF #2 | (C)[sP].[dR](C)P.[dR](A)[sP].[mR](G)P.[fR](A)P.[mR](C)P.[fR](G)[sP].[mR](G)[sP].[fR](C)[sP].[mR](A)[sP].[fR](G)[sP].[mR](G)[sP][mR](U)P. |
| | 2′F/ | [mR](C)P.[mR](U)P.[mR](G)[sP].[mR](G)}$$$$V2.0 |
| | 2′Ome | |
| | alt PO | |
| | 20 to | |
| | 22, | |
| | −12 to | |
| | −14 | |
| | and PO | |
| | −1, −3, | |
| | −4, −5, | |
| | 7, 11 | |
| 92_53 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)P.[fR](G)P.[mR](U)[sP].[fR](U)[sP].[mR](U)[sP].[fR](U)[sP].[mR](U) |
| | ORF #2 | [sP].[dR](C)[sP].[dR](C)[sP].[mR](U)P.[mR](G)P.[fR](A)P.[mR](C)P.[fR](G)[sP].[mR](G)[sP].[fR](C)[sP].[mR](A)[sP].[fR](G)[sP].[mR](G)[sP]. |
| | 2′F/ | [mR](U)[sP].[mR](C)[sP].[mR](A)[sP].[mR](G)[sP].[mR](G)}$$$$V2.0 |
| | 2′Ome | |
| | alt | |
| | PO −2, | |
| | −3, −4, | |
| | −5, 6, | |
| | 7, 11 | |
| | Ome −1 | |
| 92_54 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[mR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR)(U)[sP].[mR](C)[sP].[mR](A)(sP].[mR(G)[sP].[mR](G)}$$$$V2.0 |
| | 2′F/ | |
| | 2′Ome | |
| | alt | |
| | 2′Ome 0 | |
| 92_55 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[mR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP}.[mR](C)[sP].[mR](A)[sP].[mR](G)[sP].[mR](G)}$$$$V2.0 |
| | 2′F/ | |
| | 2′Ome | |
| | alt | |
| | 2′Ome | |
| | −1 | |
| 92_56 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[mR](C)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP].[mR](C)[sP].[mR](A)[sP].\|mR](G)[sP].[mR](G)}$$$$V2.0 |
| | 2′F/ | |
| | 2′Ome | |
| | alt | |
| | 2′Ome 1 | |
| 92_57 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)(sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[mR](C)[sP].[dR](C)[sP].[mR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)(sP].[mR](U)(sP].[mR](C)[sP].[mR](A)[sP].[mR](G)[sP].[mR](G)}$$$$V2.0 |
| | 2′F/ | |
| | 2′Ome | |
| | alt | |
| | 2′Ome | |
| | 1, −1 | |
| 109_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](G)[sP].[dR](A)[sP].[mR](G)[sP].[fR](A)[sP].[mR](C)[sP].[fR](G)[sP].[mR](G)[sP].[fR](C)(sP].[mR](A)[sP].[fR](G) |
| | all PS | [sP].[mR](G)[sP].[mR](U)[sP].[mR](C)[sP].[mR](A)[sP].[mR](G)[sP].[mR](G)}$$$$V2.0 |
| | 2′F/ | |
| | 2′Ome | |
| | alt | |
| | G 0 | |
| 110_1 | 24-1-15 | RNA1{[mR](G)[sP].[mR](U)[sP].[mR](C)[sP].[mR](A)[sP].[mR(U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)(sP].[mR](U)(sP].[mR](C)[sP].[mR](A)[sP].[mR](G)[sP].[mR](G)}$$$$V2.0 |
| | 2′F/ | |
| | 2′Ome | |
| | alt | |
| | A 0 | |
| 111_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](T)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP].[mR](C)[sP].[mR](A)[sP].[mR](G)[sP].[mR](G)}$$$$V2.0 |
| | 2′F/ | |
| | 2′Ome | |
| | alt | |
| | T 0 | |
| 112_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR]([In])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | all PS | (G)[sP].[mR](G)[sP].[mR](U)[sP].[mR](C)[sP].[mR](A)[sP].[mR](G)\|sP].[mR](G)}$$$$V2.0 |
| | 2′F/ | |
| | 2′Ome | |
| | alt | |
| | I 0 | |
| 113_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](G)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP].[mR](C)[sP].[mR](A)[sP].[mR](G)[sP].[mR](G)}$$$$V2.0 |
| | 2′F/ | |
| | 2′Ome | |
| | alt | |
| | G 1 | |
| 114_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](A)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)(sP].[mR](U)[sP].[mR(C)[sP].[mR](A)[sP].[mR](G)[sP].[mR](G)}$$$$V2.0 |
| | 2′F/ | |
| | 2′Ome | |
| | alt | |
| | A 1 | |
| 115_1 | 24-1-15 | RNA1{[mR](G)[sP].[mR](U)[P].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[aP].[fR](G)[sP].[mR](U)[sP].[fR](U)[sP].[mR](U)[sP].[fR](U)[sP].[mR] |
| | ORF #2 | (U)[sP].[dR](T)[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mr}(G)(sP].[mR](U)[sP].[mR](C)[sP].[mR](A)[sP].[mR](G)(sP].[mR](G)}$$$$V2.0 |
| | 2′F/ | |
| | 2′Ome | |
| | alt | |
| | T 1 | |
| 116_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR]([In])[sP].[dR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | all PS | (G)[sP].[mR](G)[sP].[mR](U)[sP].[mR(C)[sP].[mR](A)\|sP].[mR](G)\|sP].[mR](G)}$$$$V2.0 |
| | 2′F/ | |
| | 2′Ome | |
| | alt | |
| | I 1 | |
| 117_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](G)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP].[mR](C)[sP].[mR](A)[sP].[mR](G)[sP].[mR](G)}$$$$V2.0 |
| | 2′F/ | |
| | 2′Ome | |
| | alt | |
| | G −1 | |
| 118_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](C)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)[sP].[mR](U)[sP].[mR](C)(sP].[mR](A)(sP].[mR](G)\|sP].[mR](G)}$$$$V2.0 |
| | 2′F/ | |
| | 2′Ome | |
| | alt | |
| | C −1 | |
| 119_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR](T)[sP].[mR](G)[sP].[fR](A)[sP].[mR](C)[sP].[fR](G)[sP].[mR](G)[sP].[fR](C)[sP].[mR](A)[sP].[fR](G) |
| | all PS | [sP].[mR](G)(sP).[mR](U)[sP].[mR](C)\|sP].[mR)(A)\|sP].[mR)(G)(sP].[mR](G)}$$$$V2.0 |
| | 2′F/ | |
| | 2′Ome | |
| | alt | |
| | T −1 | |
| 120_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | ORF #2 | (U)[sP].[dR](C)[sP].[dR](C)[sP].[dR]([In])[sP].[mR](G)[sP].[fR](A)[sP].[mR](C)[sP].[fR](G)[sP].[mR](G)[sP].[fR](C)[sP].[mR](A)(sP].[fR] |
| | all PS | (G)[sP].[mR](U)[sP].[mR](C)[sP].[mR](A)[sP].[mR](G)[sP].[mR](G)}$$$$V2.0 |
| | 2′F/ | |
| | 2′Ome | |
| | alt | |
| | I −1 | |
| 92_58 | 24-1-15 | RNA1{[MOE}(G)[sP].[MOE](T)[sP].[MOE]([5meC])[sP].[MOE](A)[sP].[MOE](T)[sP].[fR](C)[sP].[mR](A)[sP].[fR](U)[sP].[mR](A)[sP].[fR](U)[sP]. |
| | GAPDH | [mR](U)[sP].[fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP]. |
| | ORF #2 | [fR](U)[sP].[mR](U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP]. |
| | all PS | [mR](A)[sP].[fR](G)(SP).[MOE](G)[sP].[MOE](T)[sP].[MOE]([5meC])[sP].[MOE](A)[sP].[MOE](G)[sP].[MOE](G)}$$$$V2.0 |
| | 2′F/ | |
| | 2′Ome | |
| | alt MOE | |
| | flanks | |
| 96_2 | 24-1-8 | RNA1{[MOE](G)[sP].[MOE](T)[sP].[MOE]([5meC])[sP].[MOE](A)[sP].[MOE](T)[sP].[fR](C)[sP].[mR](A)[sP].[fR](U)[sP].[mR](A)[sP].[fR](U)[sP]. |
| | GAPDH | [mR](U)[sP].[fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP]. |
| | ORF #2 | [fR](U)[sP].[mR](U)[sP].[dR](C)[sP].[dR](C)[sP].[dR](A)[sP].[mR](G)[sP].[fR](A)[P].[MOE]([5meC])[sP].[MOE](G)[sP].[MOE](G)[sP].[MOE] |
| | all PS | ([5meC])[sP].[MOE](A)}$$$$V2.0 |
| | I 2′F/ | |
| | 2′Ome | |
| | alt MOE | |
| | flanks | |
| 99_2 | 22-1-12 | RNA1{[MOE]([5meC])[sP].[MOE](A)[sP].[MOE](T)[sP].[MOE]([5meC])[sP].[MOE](A)[sP].[fR](U)[sP].[mR](A)[sP].[fR](U)[sP].[mR](U)[sP].[fR] |
| | GAPDH | (U)[sP].[mR)(G)[sP].[fR](G)[sP].[mR](C)[sP].[fR](A)[sP].[mR](G)[sP].[fR](G)[sP].[mR](U)[sP].[fR](U)[sP].[mR](U)(sP].[fR](U)[sP].[mR] |
| | ORF #2 | (U)[sP].[dR](C)[sP].[dR](C)[sP].[dR](A)[sP].[mR](G)[sP].[fR](A)[sP].[mR](C)[sP].[fR](G)[sP].[mR](G)[sP].[fR](C)[sP].[MOE](A)[sP].[MOE] |
| | all PS | (G)[sP].[MOE](G)[sP].[MOE)(T)[sP].[MOE]([6meC])}$$$$V2.0 |
| | I 2′F/ | |
| | 2′Ome | |
| | alt MOE | |
| | flanks | |
| 101_2 | 22-1-8 | RNA1{[MOE]([5meC])[sP].[MOE](A)[sP].[MOE](T)[sP].[MOE]([5meC])[sP].[MOE](A)[sP].[fR](U)[sP].[mR](A)[sP].[fR](U)[sP].[mR](U)[sP].[fR] |
| | GAPDH | (U)[sP].[mR](G)[sP].[fR](G)[sP].[mR](C)[sP].[fR](A)[sP].[mR](G)[sP].[fR](G)[sP].[mR](U)[sP].[fR](U)[sP].[mR(U)[sP].[fR](U)[sP].[mR](U) |
| | ORF #2 | [sP].[dR](C)[sP].[dR](C)[sP].[dR](A)[sP].[mR](G)[sP].[fR](A)[sP].[MOE]([5meC])[sP].[MOE)(G)[sP].[MOE](G)[sP].[MOE]([5meC])[sP].[MOE] |
| | all PS | (A)}$$$$V2.0 |
| | I 2′F/ | |
| | 2′ Ome | |
| | alt MOE | |
| | flanks | |
| 110_2 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)(SP).[mR](G)[sP].[fR](G)[sP].[mR](C)[sP].[fR](A)[sP].[mR](G)[sP].[fR)(G)[sP].[mR(U)\|sP].[fR](U)[sP].[mR](U)[sP].[fR](U)[sP].[mR](U) |
| | central | [sP].[dR](C)[sP].[LR](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | “C” | [mR](G)[sP].[mR](U)[sP].[mR](C)[sP].[mR](A)[sP].[mR(G)[sP].[mR](G)}$$$$V2.0 |
| | modifi- | |
| | cation: | |
| | [LR](A) | |
| | [sP] | |
| 109_2 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$v2.0 |
| | modifi- | |
| | cation: | |
| | [LR](G) | |
| | [sP] | |
| 121_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[LR]([5meC])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](U)[sP].[mR](C)[sP].[mR](A)[sP].[mR](G)[sP].[mR](G)}$$$$V2.0 |
| | modifi- | |
| | cation: | |
| | [LR] | |
| | ([5me- | |
| | C]) | |
| | [sP] | |
| 110_3 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[MOE](A)[sP].[dR](A)[sP].[mR](G)[sP].[fR](A)[sP].[mR](C)[sP].[fR](G)[sP].[mR](G)[sP].[fR](C)[sP].[mR](A)(sP].[fR](G) |
| | “C” | [sP].[mR](G)[sP].[mR](U)[sP].[mR](C)\|sP].[mR](A)[sP].[mR](G)[sP].[mR](G)}$$$$V2.0 |
| | modifi- | |
| | cation: | |
| | [MOE] | |
| | (A) | |
| | [sP] | |
| 109_3 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR[(G)[sP].[fR](G)[sP].[mR](U)[sP].[fR](U)[sP].[mR](U)[sP].[fR](U)[sP].[mR] |
| | central | (U)[sP].[dR](C)[sP].[MOE](G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [MOE] | |
| | (G) | |
| | [sP] | |
| 121_2 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[MOE]([5meC])[sP].[dR](A)[sP].[mR](G)[sP].[fR](A)[sP].[mR](C)[sP].[fR](G)[sP].[mR](G)[sP].[fR](C)[sP].[mR](A)[sP]. |
| | “C” | [fR](G)[sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [MOE] | |
| | ([5me- | |
| | C]) | |
| | [sP] | |
| 110_4 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[fR](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [fR](A) | |
| | [sP] | |
| 109_4 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[fR](G)[sP].[dR](A)[sP].[mR](G)(sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [fR](G) | |
| | [sP] | |
| 92_59 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[fR](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)sP].[mR)(C)(sP].[mR](A)(sP].[mR](G)[sP].[mR](G)}$$$$V2.0 |
| | modifi- | |
| | cation: | |
| | [fR]@ | |
| | [SP] | |
| 112_2 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[fR]([In])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](G)[sP].[mR](U)[sP].[mR)(C)(sP].[mR](A)(sP].[mR](G)\|sP].[mR](G)}$$$$V2.0 |
| | modifi- | |
| | cation: | |
| | [fR] | |
| | (In) | |
| | [sP] | |
| 110_5 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[mR](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [mR](A) | |
| | [sP] | |
| 109_5 | 24-1-15 | RNA1{[mR](G)[sP].[mR](U)[sP].[mR](C)[sP].[mR](A)[sP].[mR](U)[sP].[fR](C)[sP}.[mR](A)[sP].[fR](U)[sP].[mR](A)[sP}.[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[mR}(G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)(sP].[mR](U)[sP].[mR](C)[sP].[mR](A)[sP].[mR)(G)(sP].[mR](G)}$$$$V2.0 |
| | modifi- | |
| | cation: | |
| | [mR](G) | |
| | [sP] | |
| 112_3 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[mR]([In])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](G)sP].[mR](U)[sP].[mR(C)[sP].[mR](A)[sP].[mR](G)[sP].[mR](G)}$$$$V2.0 |
| | modifi- | |
| | cation: | |
| | [mR] | |
| | ([In]) | |
| | [sP] | |
| 121_3 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[P].[fR](U)[sP].[mR] |
| | central | (U)[sP].[dR](C)[sP].[mR]([5meC])[sP].[dR](A)[sP].[mR](G)[sP].[fR](A)[P].[mR](C)[sP].[fR](G)[sP].[mR](G)[sP].[fR](C)[sP].[mR](A)[sP].[fR] |
| | “C” | (G)[sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [mR] | |
| | ((5me- | |
| | C]) | |
| | [sP] | |
| 110_6 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[TNA](A)[sP].[dR](A)[sP].[mR](G)[sP].[fR](A)[sP].[mR](C)[sP].[fR](G)[sP].[mR](G)[sP].[fR](C)[P][mR](A)[sP].[fR](G) |
| | “C” | [sP].[mR](G)[sP].[mR)(U)[sP].[mR](C)\|sP].[mR](A)\|sP].[mR](G)(sP].[mR](G)}$$$$V2.0 |
| | modifi- | |
| | cation: | |
| | [TNA] | |
| | (A) | |
| | [sP] | |
| 109_6 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[TNA)(G)[sP].[dR](A)(sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [TNA] | |
| | (G) | |
| | [sP] | |
| 121_4 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[TNA]([5meC])[sP].[dR](A)[sP].[mR](G)[sP].[fR](A)[sP].[mR](C)[sP].[fR](G)[sP].[mR](G)[sP].[fR](C)[sP].[mR](A)[sP]. |
| | “C” | [fR](G)[sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [TNA] | |
| | ([5me- | |
| | C]) | |
| | [sP] | |
| 110_7 | 24-1-15 | RNA1{[mR](G)[sP].[mR](U)[sP].[mR](C)[sP].[mR](A)[sP].[mR](U)[sP].[fR](C)[sP}.[mR](A)[sP}.[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR}(G)[sP].[mR](U)[sP].[fR](U)[sP].[mR](U)[sP].[fR](U)[sP].[mR] |
| | central | (U)[sP].[dR](C)[sP].[SNA](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [SNA] | |
| | (A) | |
| | [sP] | |
| 109_7 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[SNA](G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)sP).[mR](U)[sP].[mR](C)[sP][mR](A)[sP].[mR](G)[sP].[mR](G)}$$$$V2.0 |
| | modifi- | |
| | cation: | |
| | [SNA] | |
| | (G) | |
| | [sP] | |
| 121_5 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[SNA]([5meC])[sP].[dR](A)(sP].[mR](G)[sP].[fR](A)[sP].[mR](C)[sP].[fR](G)[sP].[mR](G)[sP].[fR](C)[sP].[mR](A)[sP]. |
| | “C” | [fR](G)[sP].[mR](G)(sP].[mR](U)[sP].[mR(C)[sP].[mR](A)[sP].[mR](G)(sP].[mR](G)}$$$$V2.0 |
| | modifi- | |
| | cation: | |
| | [SNA] | |
| | ([5me- | |
| | C]) | |
| | [sP] | |
| 122_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](Z)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR)(U)[sP].[mR](C)[sP].[mR(A)(sP].[mR(G)[sP].[mR](G)}$$$$V2.0 |
| | modifi- | |
| | cation: | |
| | [dR](Z) | |
| | [sP] | |
| 109_8 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR]((U)[sP].[fR](U)[sP].[mR](U)[sP].[fR](U)[sP].[mR] |
| | central | (U)[sP].[dR](C)[sP].[ScEt)(G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [ScEt] | |
| | (G)[sP] | |
| 123_1 | 24-1-15 | RNA1{[mR](G)[sP].[mR](U)[sP].[mR(C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR]([PyrC])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [dR] | |
| | ([Pyr- | |
| | C]) | |
| | [sP] | |
| 110_8 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [dR](C)[sP].[FANA](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G)[sP]. |
| | central | [mR](G)[sP].[mR](U)[sP].[mR](C)[sP].[mR](A)[sP].[mR](G)[sP].[mR](G)}$$$$V2.0 |
| | “C” | |
| | modifi- | |
| | cation: | |
| | [FANA] | |
| | (A)[sP] | |
| 109_9 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[FANA](G)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [FANA] | |
| | (G)[sP] | |
| 92_60 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[P].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[FANA](C)[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](G)sP].[mR](U)(SP).[mR](C)[sP].[mR](A)[sP].[mR](G)[sP].[mR](G)}$$$$V2.0 |
| | modifi- | |
| | cation: | |
| | [FANA] | |
| | (C)[P] | |
| 124_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR(U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[1dMOE][sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | (G)[sP].[mR](G)[sP].[mR](U)[sP].[mR](C)[sP].[mR](A)[sP].[mR](G)(sP].[mR](G)}$$$$V2.0 |
| | modifi- | |
| | cation: | |
| | [MOE] | |
| | [sP] | |
| 124_2 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[12ddR][sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [dR] | |
| | [sP] | |
| 110_9 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](A)[srP].[dR](A)[sP].[mR](G)[sP].[fR](A)[sP].[mR](C)[sP].[fR](G)[sP].[mR](G)[sP].[fR](C)(sP].[mR](A)(sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [dR](A) | |
| | [srP] | |
| 110_10 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)(sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](A)[ssP].[dR](A)[sP].[mR](G)[sP].[fR](A)[sP].[mR](C)[sP].[fR](G)[sP].[mR](G)[sP].[fR](C)[sP].[mR](A)[sP].[fR}(G) |
| | “C” | [sP].[mR](G)[sP].[mR(U)[sP].[mR](C)\|sP].[mR)(A)[sP].[mR](G)(sP].[mR](G)}$$$$V2.0 |
| | modifi- | |
| | cation: | |
| | [dR](A) | |
| | [ssP] | |
| 109_10 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[P]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](G)[ssP].[dR](A)[sP].[mR](G)[sP].[fR](A)[sP].[mR](C)[sP].[fR](G)[sP].[mR](G)[sP].[fR](C)[sP].[mR](A)[sP].[fR}(G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [dR](G) | |
| | [ssP] | |
| 92_61 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](C)[srP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)(sP].[mR(U)[sP].[mR](C)[sP].[mR)(A)\|sP].[mR](G)(sP].[mR](G)}$$$$V2.0 |
| | modifi- | |
| | cation: | |
| | [dR](C) | |
| | [srP] | |
| 92_62 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR](C)[ssP].[dR](A)[sP].[mR}(G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)[sP].[mR](A)[sP].[mR](G)[sP].[mR](G)}$$$$V2.0 |
| | modifi- | |
| | cation: | |
| | [dR](C) | |
| | [ssP] | |
| 110_11 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR](U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[idR](A)[sP].[dR](A)[sP].[mR](G)[sP].[fR](A)[sP].[mR](C)[sP].[fR](G)[sP].[mR](G)[sP].[fR](C)[sP].[mR](A)[sP].[fR](G) |
| | “C” | [sP].[mR](G)[sP].[mR](U)[sP].[mR](C)(sP].[mR](A)[sP].[mR](G)[sP].[mR](G)}$$$$V2.0 |
| | modifi- | |
| | cation: | |
| | [dR](A) | |
| | [sP] | |
| 125_1 | 24-1-15 | RNA1{[mR](G)[sP].[mR](U)[sP].[mR](C)[sP].[mR](A)[sP].[mR](U)[sP].[fR](C)[sP].[mR](A)[sP].[fR](U)[sP].[mR](A)[sP].[fR](U)[sP].[mR](U)[sP]. |
| | GAPDH - | [fR}(U)[sP].[mR](G)[sP].[fR](G)[sP].[mR](C)[sP].[fR](A)[sP].[mR](G)[sP].[fR](G)[sP].[mR](U)[sP].[fR](U)[sP].[mR](U)[sP].[fR](U)[sP].[mR] |
| | central | (U)[sP].[dR](C)[sP].[dR]([5BrC])[sP].[dR](A)[sP].[mR](G)[sP].[fR](A)[sP].[mR](C)[sP].[fR](G)[sP].[mR](G)[sP].[fR](C)[sP].[mR](A)[sP].[fR] |
| | “C” | (G)[sP].[mR](G)[sP].[mR](U)[sP].[mR](C)[sP].[mR](A)[sP].[mR](G)[sP].[mR)(G)}$$$$V2.0 |
| | modifi- | |
| | cation: | |
| | [dR] | |
| | ([5Br- | |
| | C]) | |
| | [sP] | |

TABLE 7
mHprt editing sequences

| SEQ | | | |
|---|---|---|---|
| ID NO | Sequence (clean) | Length | CMP ID NOS |
| 126 | GGCAGAUGGCCACACGACUAGAACACCUGCCAAUUUUACUGGCAACAUAAA | 51 | 126_1_and_ |
| | CAGCACUCCU | | 126_2 |
| 127 | AUGGCCACACGACUAGAACACCUGCCAAUUUUACUGGCAACAUAAACAGCA | 51 | 127_1 |
| 128 | AUGGCCACACGACUAGAACACCUGCCAAUUUUACUGGCAACAUAAACAGC | 50 | 128 |
| 129 | AUGGCCACACGACUAGAACACCUGCCAAUUUUACUGGCAACAUAAACAG | 49 | 129 |
| 130 | AUGGCCACACGACUAGAACACCUGCCAAUUUUACUGGCAACAUAAACA | 48 | 130 |
| 131 | AUGGCCACACGACUAGAACACCUGCCAAUUUUACUGGCAACAUAAAC | 47 | 131 |
| 132 | AUGGCCACACGACUAGAACACCUGCCAAUUUUACUGGCAACAUAAA | 46 | 132 |
| 133 | AUGGCCACACGACUAGAACACCUGCCAAUUUUACUGGCAACAUAA | 45 | 133 |
| 134 | AUGGCCACACGACUAGAACACCUGCCAAUUUUACUGGCAACAUA | 44 | 134 |
| 135 | AUGGCCACACGACUAGAACACCUGCCAAUUUUACUGGCAACAU | 43 | 135 |
| 136 | AUGGCCACACGACUAGAACACCUGCCAAUUUUACUGGCAACA | 42 | 136 |
| 137 | AUGGCCACACGACUAGAACACCUGCCAAUUUUACUGGCAAC | 41 | 137 |
| 138 | AUGGCCACACGACUAGAACACCUGCCAAUUUUACUGGCAA | 40 | 138 |
| 139 | AUGGCCACACGACUAGAACACCUGCCAAUUUUACUGGCA | 39 | 139_1 |
| 140 | AUGGCCACACGACUAGAACACCUGCCAAUUUUACUGGC | 38 | 140 |
| 141 | AUGGCCACACGACUAGAACACCUGCCAAUUUUACUGG | 37 | 141 |
| 142 | AUGGCCACACGACUAGAACACCUGCCAAUUUUACUG | 36 | 142_1 |
| 143 | AUGGCCACACGACUAGAACACCUGCCAAUUUUACU | 35 | 143_1 |
| 144 | AUGGCCACACGACUAGAACACCUGCCAAUUUUAC | 34 | 144 |
| 145 | AUGGCCACACGACUAGAACACCUGCCAAUUUUA | 33 | 145_1 |
| 146 | AUGGCCACACGACUAGAACACCUGCCAAUUUU | 32 | 146_1 |
| 147 | AUGGCCACACGACUAGAACACCUGCCAAUUU | 31 | 147 |
| 148 | AUGGCCACACGACUAGAACACCUGCCAAUU | 30 | 148 |
| 149 | GGCAGAUGGCCACACGACUAGAACACCUGCCAAUUUUACUGGCAAC | 46 | 149 |
| 150 | GCAGAUGGCCACACGACUAGAACACCUGCCAAUUUUACUGGCAAC | 45 | 150_1 |
| 151 | CAGAUGGCCACACGACUAGAACACCUGCCAAUUUUACUGGCAAC | 44 | 151 |
| 152 | AGAUGGCCACACGACUAGAACACCUGCCAAUUUUACUGGCAAC | 43 | 152_1 |
| 153 | GAUGGCCACACGACUAGAACACCUGCCAAUUUUACUGGCAAC | 42 | 153_1 |
| 154 | UGGCCACACGACUAGAACACCUGCCAAUUUUACUGGCAAC | 40 | 154_1 |
| 155 | GGCCACACGACUAGAACACCUGCCAAUUUUACUGGCAAC | 39 | 155_1 |
| 156 | GCCACACGACUAGAACACCUGCCAAUUUUACUGGCAAC | 38 | 156 |
| 157 | CCACACGACUAGAACACCUGCCAAUUUUACUGGCAAC | 37 | 157 |
| 158 | CACACGACUAGAACACCUGCCAAUUUUACUGGCAAC | 36 | 158_1 |
| 159 | UGGCCACACGACUAGAACACCUGCCAAUUUUACUGGC | 37 | 159_1 |
| 160 | UGGCCACACGACUAGAACACCUGCCAAUUUUACUG | 35 | 160_1 |
| 161 | UGGCCACACGACUAGAACACCUGCCAAUUUUAC | 33 | 161_1 |
| 162 | UGGCCACACGACUAGAACACCUGCCAAUUUU | 31 | 162_1 |
| 163 | UGGCCACACGACUAGAACACCUGCCAAUU | 29 | 163 |
| 164 | GCCACACGACUAGAACACCUGCCAAUUUUACUGGC | 35 | 164 |
| 165 | GCCACACGACUAGAACACCUGCCAAUUUUACUG | 33 | 165_1 |
| 166 | GCCACACGACUAGAACACCUGCCAAUUUUAC | 31 | 166_1 |
| 167 | GCCACACGACUAGAACACCUGCCAAUUUU | 29 | 167_1 |
| 168 | GCCACACGACUAGAACACCUGCCAAUU | 27 | 168_1 |
| 169 | CACACGACUAGAACACCUGCCAAUUUUACUGGC | 33 | 169_1 |
| 170 | CACACGACUAGAACACCUGCCAAUUUUACUG | 31 | 170_1 |
| 171 | CACACGACUAGAACACCUGCCAAUUUUAC | 29 | 171 |
| 172 | CACACGACUAGAACACCUGCCAAUUUU | 27 | 172_1 |
| 173 | CACACGACUAGAACACCUGCCAAUU | 25 | 173_1 |
| 174 | UGGCCACACGACUAGAACACCUGCGAAUUUUACUGGCAAC | 40 | 174 |
| 175 | UGGCCACACGACUAGAACACCUGCAAAUUUUACUGGCAAC | 40 | 175_1 |
| 176 | UGGCCACACGACUAGAACACCUGCUAAUUUUACUGGCAAC | 40 | 176_1 |
| 177 | UGGCCACACGACUAGAACACCUGCIAAUUUUACUGGCAAC | 40 | 177_1 |
| 17 | UGGCCACACGACUAGAACACCUGGCAAUUUUACUGGCAAC | 40 | 178_1 |
| 179 | UGGCCACACGACUAGAACACCUGACAAUUUUACUGGCAAC | 40 | 179_1 |
| 180 | UGGCCACACGACUAGAACACCUGUCAAUUUUACUGGCAAC | 40 | 180_1 |
| 181 | UGGCCACACGACUAGAACACCUGICAAUUUUACUGGCAAC | 40 | 181_1 |
| 182 | UGGCCACACGACUAGAACACCUGCCGAUUUUACUGGCAAC | 40 | 182_1 |
| 183 | UGGCCACACGACUAGAACACCUGCCCAUUUUACUGGCAAC | 40 | 183_1 |
| 184 | UGGCCACACGACUAGAACACCUGCCUAUUUUACUGGCAAC | 40 | 184_1 |
| 185 | UGGCCACACGACUAGAACACCUGCCIAUUUUACUGGCAAC | 40 | 185_1 |

TABLE 8
mHprt editing compounds

| CMP | | |
|---|---|---|
| ID | | |
| NO | Name | HELM string |
| 126_1 | 30-1-30 mHprt | RNA1{[mR](G)[sP].[mR](G)[sP].[mR](C)[sP].[mR](A)[sP].[mR](G)[sP] |
| | 3UTR UAG1 all | .R(A)[sP].R(U)[sP].R(G)[sP].R(G)[sP].R(C)[sP].R(C)[sP].R(A)[sP].R |
| | PS | (C)[sP].R(A)[sP].R(G)[sP].R(G)[sP].R(A)[sP].R(C)[sP].R(U)[sP].R(A |
| | | )[sP].R(G)[sP].R(A)[sP].R(A)[sP].R(C)[sP].R(A)[sP].R(C)[sP].R(C)[ |
| | | sP].R(U)[sP].[fR](G)[sP].R(C)[sP].R(C)[sP].R(A)[sP].R(A)[sP].R(U) |
| | | [sP].R(U)[sP].R(U)[sP].R(U)[sP].R(A)[sP].R(C)[sP].R(U)[sP].R(G)[s |
| | | P].R(G)[sP].R(C)[sP].R(A)[sP].R(A)[sP].R(C)[sP].R(A)[sP].R(U)[sP] |
| | | .R(C)[sP].R(A)[sP].R(A)[sP].R(C)[sP].R(A)[sP].R(G)[sP].R(G)[sP].R |
| | | (A)[sP].[mR](C)[sP].[mR](U)[sP].[mR](C)[sP].[mR](C)[sP].[mR](U)}$ |
| | | $$$V2.0 |
| 126_2 | 30-1-30 mHprt | RNA1{[mR](G)[sP].[mR](G)[sP].[mR](C)[sP].[mR](A)[sP].[mR](G)[sP]. |
| | 3UTR UAG1 all | [fR](A)[sP].[mR](U)[sP].[fR](G)[sP].[mR](G)[sP].[fR](C)[sP].[mR]( |
| | PS 2′F/2′Ome alt | C)[sP].[fR](A)[sP].[mR](C)[sP].[fR](A)[sP].[mR](C)[sP].[fR](G)[sP |
| | | ].[mR](A)[sP].[fR](C)[sP].[mR](U)[sP].[fR](A)[sP].[mR](G)[sP].[fR |
| | | ](A)[sP].[mR](A)[sP].[fR](C)[sP].[mR](A)[sP].[fR](C)[sP].[mR](C)[ |
| | | sP].[fR](U)[sP].[mR](G)[sP].[dR](C)[sP].[dR](C)[sP].[dR](A)[sP].[ |
| | | mR](A)[sP].[IR](U)[sP].[mR](U)[SP].[fR](U)[sP].[mR](U)[sP].[fR](A |
| | | )[sP].[mR](C)[sP].[fR](U)[sP].[mR](G)[sP].[fR](G)[sP].[mR](C)[sP] |
| | | .[fR](A)[sP].[mR](A)[SP].[fR](C)[sP].[mR](A)[SP].[fR](U)[sP].[mR] |
| | | (A)[sP].[fR](A)[sP].[mR](A)[sP].[fR](C)[sP].[mR](A)[sP].[fR](G)[s |
| | | P].[mR](C)[sP].[fR](A)[sP].[mR](C)[sP].[mR](U)[sP].[mR](C)[sP].[m |
| | | R](C)[sP].[mR](U)}$$$$V2.0 |
| 127_1 | 25-1-25 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | | G)[sP].[mR](G)[sP].[fR](C)[sP].[fR](C)[sP].[fR](A)[sP].[mR](A)[sP |
| | | ].[fR](U)[sP].[mR](U)[sP].[fR](U)[sP].[mR](U)[sP].[fR](A)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[fR](C)[sP].[mR](A)[sP].[fR](U)[sP] |
| | | .[mR](A)[sP].[fR](A)[sP].[mR](A)[sP].[mR](C)[sP].[mR](A)[sP].[mR] |
| | | (G)[sP].[mR](C)[sP].[mR](U)}$$$$V2.0 |
| 128_1 | 25-1-24 mHprt | RNA1{[mR+(A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[fR](C)[sP].[mR](A)[sP].[fR](U)[sP] |
| | | .[mR](A)[sP].[mR](A)[sP].[mR](A)[sP].[mR](C)[sP].[mR](A)[sP].[mR] |
| | | (G)[sP].[mR](C)}$$$$V2.0 |
| 129_1 | 25-1-23 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[fR](C)[sP].[mR](A)[sP].[fR](U)[sP] |
| | | .[mR](A)[sP].[mR](A)[sP].[mR](A)[sP].[mR](C)[sP].[mR](A)[sP].[mR] |
| | | (G)}$$$$V2.0 |
| 130_1 | 25-1-22 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[fR](C)[sP].[mR](A)[sP].[fR](U)[sP] |
| | | .[mR](A)[sP].[mR](A)[sP].[mR](A)[sP].[mR](C)[sP].[mR](U)}$$$$V2.0 |
| 131_1 | 25-1-21 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[mR](C)[sP].[mR](A)[sP].[mR](U)[sP] |
| | | .[mR](A)[sP].[mR](A)[sP].[mR](A)[sP].[mR](C)}$$$$V2.0 |
| 132_1 | 25-1-20 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[mR](C)[sP].[mR](A)[sP].[fR](U)[sP] |
| | | .[mR](A)[sP].[mR](A)[sP].[mR](U)}$$$$V2.0 |
| 133_1 | 25-1-19 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[mR](C)[sP].[mR](A)[sP].[mR](U)[sP] |
| | | .[mR](A)[sP].[mR](U)}$$$$V2.0 |
| 134_1 | 25-1-18 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[fR](A)[sP].[mR](A)[sP].[mR](C)[sP].[mR](A)[sP].[mR](U)[sP] |
| | | .[mR](U)}$$$$V2.0 |
| 135_1 | 25-1-17 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[mR](A)[sP].[mR](A)[sP].[mR](C)[sP].[mR](A)[sP].[mR](U)}$$$ |
| | | $V2.0 |
| 136_1 | 25-1-16 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[fR](G)[sP].[mR](C |
| | | )[sP].[mR](A)[sP].[mR](A)[sP].[mR](C)[sP].[mR](U)}$$$$V2.0 |
| 137_1 | 25-1-15 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[mR](G)[sP].[mR](C |
| | | )[sP].[mR](A)[sP].[mR](A)[sP].[mR](C)}$$$$V2.0 |
| 138_1 | 25-1-14 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[fR](U)[sP].[mR](G)[sP].[mR](G)[sP].[mR](C |
| | | )[sP].[mR](A)[sP].[mR](U)}$$$$V2.0 |
| 139_1 | 25-1-13 mHprt | RNA1{[mR](A)[sP].[mR](U)[sP].[mR](G)[sP].[mR](G)[sP].[mR](C)[sP]. |
| | 3′UTR all | [mR](C)[sP].[fR](A)[sP].[mR](C)[sP].[fR](A)[sP].[mR](C)[sP].[fR]( |
| | PS 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | fR](A)[sP].[mR](C)[sP].[mR](U)[sP].[mR](G)[sP].[mR](G)[sP].[mR](C |
| | | )[sP].[mR](U)}$$$$V2.0 |
| 140_1 | 25-1-12 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | mR](A)[sP].[mR](C)[sP].[mR](U)[sP].[mR](G)[sP].[mR](G)[sP].[mR](C |
| | | )}$$$$V2.0 |
| 141_1 | 25-1-11 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | mR](A)[sP].[mR](C)[sP].[mR](U)[sP].[mR](G)[sP].[mR](G)}$$$$V2.0 |
| 142_1 | 25-1-10 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[fR](U)[sP].[mR](U)[sP].[ |
| | | mR](A)[sP].[mR](C)[sP].[mR](U)[sP].[mR](G)}$$$$V2.0 |
| 143_1 | 25-1-9 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[mR](U)[sP].[mR](U)[sP].[ |
| | | mR](U)[sP].[mR](A)[sP].[mR](C)[mR](U)}$$$$V2.0 |
| 144_1 | 25-1-8 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[fR](U)[sP].[mR](U)[sP].[mR](U)[sP].[mR](U)[sP].[ |
| | | mR](A)[sP].[mR](C)}$$$$V2.0 |
| 145_1 | 25-1-7 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[mR](U)[sP].[mR](U)[sP].[mR](U)[sP].[mR](U)[sP].[ |
| | | mR](U)}$$$$V2.0 |
| 146_1 | 25-1-6 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[mR](U)[sP].[mR](U)[sP].[mR](U)[sP].[mR](U)}$$$$V |
| | | 2.0 |
| 147_1 | 25-1-5 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[mR](U)[sP].[mR](U)[sP].[mR](U)}$$$$V2.0 |
| 148_1 | 25-1-4 mHprt | RNA1{[mR](A)[sP].[mR](U)[sP].[mR](G)[sP].[mR](G)[sP].[mR](C)[sP]. |
| | 3′UTR all PS | [mR](C)[sP].[fR](A)[sP].[mR](C)[sP].[fR](A)[sP].[mR](C)[sP].[fR]( |
| | 2′F/2′OMe alt | G)[sP].[mR](A)[sP].[fR](C)[sP].[mR](U)[sP].[fR](A)[sP].[mR](G)[sP |
| | | ].[fR](A)[sP].[mR](A)[sP].[fR](C)[sP].[mR](A)[sP].[fR](C)[sP].[mR |
| | | ](C)[sP].[fR](U)[sP].[mR](G)[sP].[dR](C)[sP].[dR](C)[sP].[dR](A)[ |
| | | sP].[mR](A)[sP].[mR](U)[sP].[mR](U)}$$$$V2.0 |
| 149_1 | 30-1-15 mHprt | RNA1{[mR](G)[sP].[mR](G)[sP].[mR](C)[sP].[mR](A)[sP].[mR](G)[sP]. |
| | 3′UTR all PS | [fR](A)[sP].[mR](U)[sP].[fR](G)[sP].[mR](G)[sP].[fR](C)[sP].[mR]( |
| | 2′F/2′OMe alt | C)[sP].[fR](A)[sP].[mR](C)[sP].[fR](A)[sP].[mR](C)[sP].[fR](G)[sP |
| | 3′UTR all PS | ].[mR](A)[sP].[fR](C)[sP].[mR](U)[sP].[fR](A)[sP].[mR](G)[sP].[fR |
| | | ](A)[sP].[mR](A)[sP].[fR](C)[sP].[mR](A)[sP].[fR](C)[sP].[mR](C)[ |
| | | sP].[fR](U)[sP].[mR](G)[sP].[dR](C)[sP].[dR](C)[sP].[dR](A)[sP].[ |
| | | mR](A)[sP].[fR](U)[sP].[mR](u)[sP].[fR](U)[sP].[mR](U)[sP].[fR](A |
| | | )[sP].[fR](U)[sP].[mR](G)[sP][mR](G)[sP].[mR](C)[sP].[mR](A)[sP]. |
| | | [mR](A)[sP].[mR](C)}$$$$V2.0 |
| 150_1 | 29-1-15 mHprt | RNA1{[mR](G)[sP].[mR](C)[sP].[mR](A)[sP].[mR](G)[sP].[mR](A)[sP]. |
| | 3′UTR all PS | [fR](A)[sP].[mR](U)[sP].[fR](G)[sP].[mR](G)[sP].[fR](C)[sP].[mR]( |
| | 2′F/2′OMe alt | C)[sP].[fR](A)[sP].[mR](C)[sP].[fR](A)[sP].[mR](C)[sP].[fR](G)[sP |
| | | ].[mR](A)[sP].[fR](C)[sP].[mR](U)[sP].[fR](A)[sP].[mR](G)[sP].[fR |
| | | ](A)[sP].[mR](A)[sP].[fR](C)[sP].[mR](A)[sP].[fR](C)[sP].[mR](C)[ |
| | | sP].[fR](U)[sP].[mR](G)[sP].[dR](C)[sP].[dR](C)[sP].[dR](A)[sP].[ |
| | | mR](A)[sP].[fR](U)[sP].[mR](u)[sP].[fR](U)[sP].[mR](U)[sP].[fR](A |
| | | )[sP].[mR](C)[sP].[fR](U)[sP].[mR](G)[sP][mR](G)[sP].[mR](C)[sP]. |
| | | [mR](A)[sP].[mR](A)[sP].[mR](C)}$$$$V2.0 |
| 151_1 | 28-1-15 mHprt | RNA1{[mR](C)[sP].[mR](A)[sP].[mR](GA)[sP].[mR](U)[sP].[fR](G |
| | 3′UTR all | )[sP].[mR](G)[sP].[fR](C)[sP].[fR](C)[sP].[mR](C)[sP].[fR](A |
| | PS 2′F/2′OMe alt | )[sP].[mR](C)[sP].[fR](A)[sP].[mR](C)[sP].[fR](G)[sP].[mR](A |
| | | )[sP].[fR](C)[sP].[mR](U)[sP].[fR](A)[sP].[mR](G)[sP].[fR](A |
| | | )[sP].[mR](A)[sP].[fR](C)[sP].[mR](A)[sP].[fR](C)[sP].[mR](C |
| | | )[sP].[fR](U)[sP].[mR](G)[sP].[dR](C)[sP].[dR](C)[sP].[dR](A |
| | | )[sP].[mR](A)[sP].[fR](U)[sP].[mR](U)[sP].[fR](U)[sP].[mR](U |
| | | )[sP].[fR](A)[sP].[mR](C)[sP].[fR](U)[sP].[mR](G)[sP].[mR](G |
| | | )[sP].[mR](C)[sP].[mR](A)[sP].[mR](A)[sP].[mR](C)}$$$$V2.0 |
| 152_1 | 27-1-15 mHprt | RNA1{[mR](A)[sP].[mR](G)[sP].[mR](A)[sP].[mR](U)[sP].[mR](G) |
| | 3′UTR all | [sP].[mR](G)[sP].[fR](C)[sP].[mR](C)[sP].[fR](A)[sP].[mR](C) |
| | PS 2′F/2′OMe alt | [sP].[fR](A)[sP].[mR](C)[sP].[fR](G)[sP].[mR](A)[sP].[fR](C) |
| | | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[fR](U)[sP].[mR](U)[sP].[fR](U)[sP].[mR](U)[sP].[fR](A) |
| | | [sP].[mR](C)[sP].[fR](U)[sP].[mR](G)[sP].[mR](G)[sP].[mR](C) |
| | | [sP].[mR](A)[sP].[mR](A)[sP].[mR](C)}$$$$V2.0 |
| 153_1 | 26-1-15 mHprt | RNA1{[mR](G)[sP].[mR](A)[sP].[mR](U)[sP].[mR](G)[sP].[mR](G) |
| | 3′UTR all | [sP].[fR](C)[sP].[mR](C)[sP].[fR](A)[sP].[mR](C)[sP].[fR](A) |
| | PS 2′F/2′OMe alt | [sP].[mR](C)[sP].[fR](G)[sP].[mR](A)[sP].[fR](C)[sP].[mR](U) |
| | | [sP].[fR](A)[sP].[mR](G)[sP].[fR](A)[sP].[mR](A)[sP].[fR](C) |
| | | [sP].[mR](A)[sP].[fR](C)[sP].[mR](C)[sP].[fR](U)[sP].[mR](G) |
| | | [sP].[dR](C)[sP].[dR](C)[sP].[dR](A)[sP].[mR](A)[sP].[fR](U) |
| | | [sP].[mR](U)[sP].[fR](U)[sP].[mR](U)[sP].[fR](A)[sP].[mR](C) |
| | | [sP].[fR](U)[sP].[mR](G)[sP].[mR](G)[sP].[mR](C)[sP].[mR](A) |
| | | [sP].[mR](A)[sP].[mR](C)}$$$$V2.0 |
| 154_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 155_1 | 23-1-15 mHprt | RNA1{[mR](G)[sP].[mR](G)[sP].[mR](C)[sP].[mR](C)[sP].[mR](A) |
| | 3′UTR all | [sP].[mR](C)[sP].[fR](A)[sP].[mR](C)[sP].[fR](G)[sP].[mR](A) |
| | PS 2′F/2′OMe alt | [sP].[fR](C)[sP].[mR](U)[sP].[fR](A)[sP].[mR](G)[sP].[fR](A) |
| | | [sP].[mR](A)[sP].[fR](C)[sP].[mR](A)[sP].[fR](C)[sP].[mR](C) |
| | | [sP].[fR](U)[sP].[mR](G)[sP].[dR](C)[sP].[dR](C)[sP].[dR](A) |
| | | [sP].[mR](A)[sP].[fR](U)[sP].[mR](U)[sP].[fR](U)[sP].[mR](U) |
| | | [sP].[fR](A)[sP].[mR](C)[sP].[fR](U)[sP].[mR](G)[sP].[mR](G) |
| | | [sP].[mR](C)[sP].[mR](A)[sP].[mR](A)[sP].[mR](C)}$$$$V2.0 |
| 156_1 | 22-1-15 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[fR](U)[sP].[mR](U)[sP].[fR](U)[sP].[mR](U)[sP].[fR](A) |
| | | [sP].[mR](C)[sP].[fR](U)[sP].[mR](G)[sP].[mR](G)[sP].[mR](C) |
| | | [sP].[mR](A)[sP].[mR](A)[sP].[mR](C)}$$$$V2.0 |
| 157_1 | 21-1-15 mHprt | RNA1{[mR](C)[sP].[mR](C)[sP].[mR](A)[sP].[mR](C)[sP].[mR](A) |
| | 3′UTR all | [sP].[mR](C)[sP].[fR](G)[sP].[mR](A)[sP].[fR](C)[sP].[mR](U) |
| | PS 2′F/2′OMe alt | [sP].[fR](A)[sP].[mR](G)[sP].[fR](A)[sP].[mR](A)[sP].[fR](C) |
| | | [sP].[mR](A)[sP].[fR](C)[sP].[mR](C)[sP].[fR](U)[sP].[mR](G) |
| | | [sP].[dR](C)[sP].[dR](C)[sP].[dR](A)[sP].[mR](A)[sP].[fR](U) |
| | | [sP].[mR](U)[sP].[fR](U)[sP].[mR](U)[sP].[fR](A)[sP].[mR](C) |
| | | [sP].[fR](U)[sP].[mR](G)[sP].[mR](G)[sP].[mR](C)[sP].[mR](A) |
| | | [sP].[mR](A)[sP].[mR](C)}$$$$V2.0 |
| 158_1 | 20-1-15 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[fR](U)[sP].[mR](U) |
| | | [sP].[fR](U)[sP].[mR](U)[sP].[fR](A)[sP].[mR](C)[sP].[fR](U) |
| | | [sP].[mR](G)[sP].[mR](G)[sP].[mR](C)[sP].[mR](A)[sP].[mR](A) |
| | | [sP].[mR](C)}$$$$V2.0 |
| 159_1 | 24-1-12 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[mR](U)[sP].[mR](G) |
| | | [sP].[mR](G)[sP].[mR](C)}$$$$V2.0 |
| 160_1 | 24-1-10 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[fR](U) |
| | | [sP].[mR](U)[sP].[mR](A)[sP].[mR](C)[sP].[mR](U)[sP].[mR](G) |
| | | }$$$$V2.0 |
| 161_1 | 24-1-8 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[mR](U) |
| | | [sP].[mR](U)[sP].[mR](A)[sP].[mR](C)}$$$$V2.0 |
| 162_1 | 24-1-6 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[mR](U)[sP].[mR](U)[sP].[mR](U) |
| | | [sP].[mR](U)}$$$$V2.0 |
| 163 1 | 24-1-4 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[mR](U)[sP].[mR](U)}$$$$V2.0 |
| 164_1 | 22-1-12 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[fR](U)[sP].[mR](U)[sP].[fR](U)[sP].[mR](U)[sP].[fR](A) |
| | | [sP].[mR](C)[sP].[mR](U)[sP].[mR](G)[sP].[mR](G)[sP].[mR](C) |
| | | }$$$$V2.0 |
| 165_1 | 22-1-10 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[fR](U)[sP].[mR](U)[sP].[fR](U)[sP].[mR](U)[sP].[mR](A) |
| | | [sP].[mR](C)[sP].[mR](U)[sP].[mR](G)}$$$$V2.0 |
| 166_1 | 22-1-8 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[fR](U)[sP].[mR](U)[sP].[mR](U)[sP].[mR](A)[sP].[mR](C) |
| | | }$$$$V2.0 |
| 167_1 | 22-1-6 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[mR](U)[sP].[mR](U)[sP].[mR](U)[sP].[mR](U)}$$$$V2.0 |
| 168_1 | 22-1-4 mHprt | RNA1{[mR](G)[sP].[mR](C)[sP].[mR](C)[sP].[mR](A)[sP].[mR](C) |
| | 3′UTR all | [sP].[fR](A)[sP].[mR](C)[sP].[fR](G)[sP].[mR](A)[sP].[fR](C) |
| | PS 2′F/2′OMe alt | [sP].[mR](U)[sP].[fR](A)[sP].[mR](G)[sP].[fR](A)[sP].[mR](A) |
| | | [sP].[fR](C)[sP].[mR](A)[sP].[fR](C)[sP].[mR](C)[sP].[fR](U) |
| | | [sP].[mR](G)[sP].[dR](C)[sP].[dR](C)[sP].[dR](A)[sP].[mR](A) |
| | | [sP].[mR](U)[sP].[mR](U)}$$$$V2.0 |
| 169_1 | 20-1-12 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[fR](U)[sP].[mR](U) |
| | | [sP].[fR](U)[sP].[mR](U)[sP].[fR](A)[sP].[mR](C)[sP].[mR](U) |
| | | [sP].[mR](G)[sP].[mR](G)[sP].[mR](C)}$$$$V2.0 |
| 170 1 | 20-1-10 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[fR](U)[sP].[mR](U) |
| | | [sP].[fR](U)[sP].[mR](U)[sP].[mR](A)[sP].[mR](C)[sP].[mR](U) |
| | | [sP].[mR](G)}$$$$V2.0 |
| 171_1 | 20-1-8 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[fR](U)[sP].[mR](U) |
| | | [sP].[fR](U)[sP].[mR](U)[sP].[mR](A)[sP].[mR](C)}$$$$V2.0 |
| 172 1 | 20-1-6 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[mR](U)[sP].[mR](U) |
| | | [sP].[mR](U)[sP].[mR](U)}$$$$V2.0 |
| 173_1 | 20-1-4 mHprt | RNA1{[mR](C)[sP].[mR](A)[sP].[mR](C)[sP].[mR](A)[sP].[mR](C) |
| | 3′UTR all | [sP].[fR](G)[sP].[mR](A)[sP].[fR](C)[sP].[mR](U)[sP].[fR](A) |
| | PS 2′F/2′OMe alt | [sP].[mR](G)[sP].[fR](A)[sP].[mR](A)[sP].[fR](C)[sP].[mR](A) |
| | | [sP].[fR](C)[sP].[mR](C)[sP].[fR](U)[sP].[mR](G)[sP].[dR](C) |
| | | [sP].[dR](C)[sP].[dR](A)[sP].[mR](A)[sP].[mR](U)[sP].[mR](U) |
| | | [sP].[mR](U)}$$$$V2.0 |
| 174_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt G 0 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](G) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 175_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt A 0 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](A) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 176_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt U 0 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](T) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 177_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt 1 0 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](ln |
| | | )[sP].[dR](A)[sP].[mR](A)[sP].[fR](U)[sP].[mR](U)[sP].[fR](U |
| | | )[sP].[mR](U)[sP].[fR](A)[sP].[mR](C)[sP].[fR](U)[sP].[mR](G |
| | | )[sP].[mR](G)[sP].[mR](C)[sP].[mR](A)[sP].[mR](A)[sP].[mR](C |
| | | )}$$$$V2.0 |
| 178 1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt G 1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](G)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 179_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt A 1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](A)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 180_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt U 1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](T)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 181_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt I 1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](ln)[sP].[dR](C |
| | | )[sP].[dR](A)[sP].[mR](A)[sP].[fR](U)[sP].[mR](U)[sP].[fR](U |
| | | )[sP].[mR](U)[sP].[fR](A)[sP].[mR](C)[sP].[fR](U)[sP].[mR](G |
| | | )[sP].[mR](G)[sP].[mR](C)[sP].[mR](A)[sP].[mR](A)[sP].[mR](C |
| | | )}$$$$V2.0 |
| 182_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt G −1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](G)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 183_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt C −1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](C)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 184_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt U −1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](T)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 185_1 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt I −1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](ln)[sP].[mR](A)[sP].[fR](U)[sP].[mR](U)[sP].[fR](U |
| | | )[sP].[mR](U)[sP].[fR](A)[sP].[mR](C)[sP].[fR](U)[sP].[mR](G |
| | | )[sP].[mR](G)[sP].[mR](C)[sP].[mR](A)[sP].[mR](A)[sP].[mR](C |
| | | )}$$$$V2.0 |
| 154_2 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′Ome | [sP].[mR](A)[sP].[fR](C)[sP].[mR](U)[sP].[fR](A)[sP].[mR](G) |
| | alt PO 1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 154_3 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO 0 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)P.[dR](C)[sP |
| | | ].[dR](A)[sP].[mR](A)[sP].[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_4 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO −1 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | P.[dR](A)[sP].[mR](A)[sP].[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_5 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO −2 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)P.[mR](A)[sP].[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_6 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′Ome | [sP].[mR](A)[sP].[fR](C)[sP].[mR](U)[sP].[fR](A)[sP].[mR](G) |
| | alt PO −3 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](U)P.[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_7 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′Ome | [sP].[mR](A)[sP].[fR](C)[sP].[mR](U)[sP].[fR](A)[sP].[mR](G) |
| | alt PO −4 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)P.[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_8 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO −5 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)P.[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_9 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO 6 | [sP].[fR](A)[sP].[mR](A)[sP].[fR](C)P.[mR](A)[sP].[fR](C)[sP |
| | | ].[mR](C)[sP].[fR](U)[sP].[mR](G)[sP].[dR](C)[sP].[dR](C)[sP |
| | | ].[dR](A)[sP].[mR](A)[sP].[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_10 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO 7 | [sP].[fR](A)[sP].[mR](U)P.[fR](C)[sP].[mR](A)[sP].[fR](C)[sP |
| | | ].[mR](C)[sP].[fR](U)[sP].[mR](G)[sP].[dR](C)[sP].[dR](C)[sP |
| | | ].[dR](A)[sP].[mR](A)[sP].[fR](U)[sP].[mR](U)[sP].[fR](U)[sP |
| | | ].[mR](U)[sP].[fR](A)[sP].[mR](C)[sP].[fR](U)[sP].[mR](G)[sP |
| | | ].[mR](G)[sP].[mR](C)[sP].[mR](A)[sP].[mR](A)[sP].[mR](C)}$$ |
| | | $$V2.0 |
| 154_11 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO 11 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](A)[sP].[fR](U)[sP].[mR](U)[sP].[fR](U) |
| | | [sP].[mR](U)[sP].[fR](A)[sP].[mR](C)[sP].[fR](U)[sP].[mR](G) |
| | | [sP].[mR](G)[sP].[mR](C)[sP].[mR](A)[sP].[mR](A)[sP].[mR](C) |
| | | }$$$$V2.0 |
| 154_12 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | PO 6, 7, 11 | [sP].[fR](A)[sP].[mR](U)P.[fR](C)P.[mR](A)[sP].[fR](C)[sP].[ |
| | | mR](C)[sP].[fR](U)[sP].[mR](G)[sP].[dR](C)[sP].[dR](C)[sP].[ |
| | | dR](A)[sP].[mR](A)[sP].[fR](U)[sP].[mR](U)[sP].[fR](U)[sP].[ |
| | | mR](U)[sP].[fR](A)[sP].[mR](C)[sP].[fR](U)[sP].[mR](G)[sP].[ |
| | | mR](G)[sP].[mR](C)[sP].[mR](A)[sP].[mR](A)[sP].[mR](C)}$$$$V |
| | | 2.0 |
| 154_13 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′Ome | [sP].[mR](A)[sP].[fR](C)[sP].[mR](U)[sP].[fR](A)[sP].[mR](G) |
| | alt PO −1, −2 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | P.[dR](A)P.[mR](A)[sP].[fR](U)[sP].[mR](U)[sP].[fR](U)[sP].[ |
| | | mR](U)[sP].[fR](A)[sP].[mR](C)[sP].[fR](U)[sP].[mR](G)[sP].[ |
| | | mR](G)[sP].[mR](C)[sP].[mR](A)[sP].[mR](A)[sP].[mR](C)}$$$$V |
| | | 2.0 |
| 154_14 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt PO | [sP].[mR](A)[sP].[fR](C)[sP].[mR](U)[sP].[fR](A)[sP].[mR](G) |
| | −3, −4, −5 | [sP].[fR](A)[sP].[mR](A)[sP].[fR](C)[sP].[mR](A)[sP].[fR](C) |
| | | [sP].[mR](C)[sP].[fR](U)[sP].[mR](G)[sP].[dR](C)[sP].[dR](C) |
| | | [sP].[dR](A)[sP].[mR](U)P.[fR](U)P.[mR](U)P.[fR](U)P.[mR](U) |
| | | [sP].[fR](A)[sP].[mR](C)[sP].[fR](U)[sP].[mR](G)[sP].[mR](G) |
| | | [sP].[mR](C)[sP].[mR](A)[sP].[mR](A)[sP].[mR](C)}$$$$V2.0 |
| 154_15 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe alt | [sP].[mR](A)[sP].[fR](C)[sP].[mR](U)[sP].[fR](A)[sP].[mR](G) |
| | PO −1, −2, −3, | [sP].[fR](A)[sP].[mR](A)[sP].[fR](C)[sP].[mR](A)[sP].[fR](C) |
| | −4, −5 | [sP].[mR](C)[sP].[fR](U)[sP].[mR](G)[sP].[dR](C)[sP].[dR](C) |
| | | P.[dR](A)P.[mR](U)P.[fR](U)P.[mR](U)P.[fR](U)[sP].[mR](U)[sP |
| | | ].[fR](A)[sP].[mR](C)[sP].[fR](U)[sP].[mR](G)[sP].[mR](G)[sP |
| | | ].[mR](C)[sP].[mR](A)[sP].[mR](A)[sP].[mR](C)}$$$$V2.0 |
| 154_16 | 24-1-15 mHprt | RNA1{[mR](U)[sP].[mR](G)[sP].[mR](G)[sP].[mR](C)[sP].[mR](C) |
| | 3′UTR all PS 2′F/ | [sP].[fR](A)[sP].[mR](C)[sP].[fR](A)[sP].[mR](C)[sP].[fR](G) |
| | 2′OMe alt PO −1, | [sP].[mR](A)[sP].[fR](C)[sP].[mR](U)[sP].[fR](A)[sP].[mR](G) |
| | −2, −3, −4, | [sP].[fR](A)[sP].[mR](U)P.[fR](C)P.[mR](A)[sP].[fR](C)[sP].[ |
| | −5, 6, 7, 11 | mR](C)[sP].[fR](U)[sP].[mR](G)[sP].[dR](C)[sP].[dR](C)P.[dR] |
| | | (A)P.[mR](U)P.[fR](U)P.[mR](U)P.[fR](U)[sP].[mR](U)[sP].[fR] |
| | | (A)[sP].[mR](C)[sP].[fR](U)[sP].[mR](G)[sP].[mR](G)[sP].[mR] |
| | | (C)[sP].[mR](A)[sP].[mR](A)[sP].[mR](C)}$$$$V2.0 |
| 154_17 | 24-1-15 mHprt | RNA1{[mR](U)[sP].[mR](G)[sP].[mR](G)[sP].[mR](C)[sP].[mR](C) |
| | 3′UTR all | [sP].[fR](A)[sP].[mR](C)[sP].[fR](A)[sP].[mR](C)[sP].[fR](G) |
| | PS 2′F/2′OMe | [sP].[mR](A)[sP].[fR](C)[sP].[mR](U)[sP].[fR](A)[sP].[mR](G) |
| | alt PO −3, | [sP].[fR](A)[sP].[mR](U)P.[fR](C)P.[mR](A)[sP].[fR](C)[sP].[ |
| | −4 −5, 6, | mR](C)[sP].[fR](U)[sP].[mR](G)[sP].[dR](C)[sP].[dR](C)[sP].[ |
| | 7, 11 | dR](A)[sP].[mR](U)P.[fR](U)P.[mR](U)P.[fR](U)[sP].[mR](U)[sP |
| | | ].[fR](A)[sP].[mR](C)[sP].[fR](U)[sP].[mR](G)[sP].[mR](G)[sP |
| | | ].[mR](C)[sP].[mR](A)[sP].[mR](A)[sP].[mR](C)}$$$$V2.0 |
| 154_18 | 24-1-15 mHprt | RNA1{[mR](U)[sP].[mR](G)P.[mR](G)P.[mR](C)P.[mR](C)[sP].[fR] |
| | 3′UTR all | (A)[sP].[mR](C)[sP].[fR](A)[sP].[mR](C)[sP].[fR](G)[sP].[mR] |
| | PS 2′F/2′OMe | (A)[sP].[fR](C)[sP].[mR](U)[sP].[fR](A)[sP].[mR](G)[sP].[fR] |
| | alt PO 20 to | (A)[sP].[mR](A)[sP].[fR](C)[sP].[mR](A)[sP].[fR](C)[sP].[mR] |
| | 22 and −12 to | (C)[sP].[fR](U)[sP].[mR](G)[sP].[dR](C)[sP].[dR](C)[sP].[dR] |
| | −14 | (A)[sP].[mR](U)[sP].[fR](U)[sP].[mR](U)[sP].[fR](U)[sP].[mR] |
| | | (U)[sP].[fR](A)[sP].[mR](C)[sP].[fR](U)[sP].[mR](G)[sP].[mR] |
| | | (G)[sP].[mR](C)[sP].[mR](A)[sP].[mR](A)[sP].[mR](C)}$$$$V2.0 |

TABLE 9
mHprt editing conjugate

| CNJ | | |
|---|---|---|
| ID | | |
| NO | Name | HELM string |
| 126_ | 30-1-30 | CHEM1{[5gn2c6]}\|RNA1{P.[mR](G) |
| 2_1 | mHprt | [sP].[mR](G)[sP].[mR](C)[sP].[ |
| | 3UTR | mR](A)[sP].[mR](G)[sP].[fR](A) |
| | UAG1 all | [sP].[mR](U)[sP][fR](G)[sP][mR |
| | PS 2′F/ | ](G)[sP].[fR](C)[sP].[mR](C)[s |
| | 2′OMe alt | P].[fR](A)[sP].[mR](C)[sP][fR] |
| | | (A)[sP].[mR](C)[sP].[fR](G)[sP |
| | | ].[mR](A)[sP].[fR](C)[sP].[mR] |
| | | (U)[sP][fR](A)[sP].[mR](G)[sP]. |
| | | [fR](A)[sP].[mR](A)[sP].[fR](C |
| | | )[sP].[mR](A)[sP].[fR](C)[sP][ |
| | | mR](C)[sP].[fR](U)[sP][mR](G)[P |
| | | ].[dR](C)[sP].[dR](C)[sP].[dR] |
| | | (A)[sP].[mR](A)[sP][fR](U)[sP] |
| | | .[mR](U)[sP][fR](U)sP].[mR(U)] |
| | | [sP][fR](A)[sP].[mR](C)[sP][fR |
| | | ](U)[sP].[mR](G)[sP].[fR](G)[sP |
| | | ].[mR](C)[sP].[fR](A)[sP][mR]( |
| | | A)[sP].[fR](C)[sP].[mR](A)[sP] |
| | | .[fR](U)[sP].[mR](A)[sP].[fR]( |
| | | A)[sP][mR](A)[sP][fR](C)[sP].[ |
| | | mR](A)[sP].[fR](G)[sP][mR](C)[ |
| | | sP][fR](A)[sP].[mR](C)[sP].[m |
| | | R](U)[sP].[mR](C)[sP].[mR](C)[ |
| | | P].[mR](U)}$CHEM1, |
| | | RNA1,1: R2-1: R1$$$V2.0 |

### Example 2—Effect of Mixmer on Stability

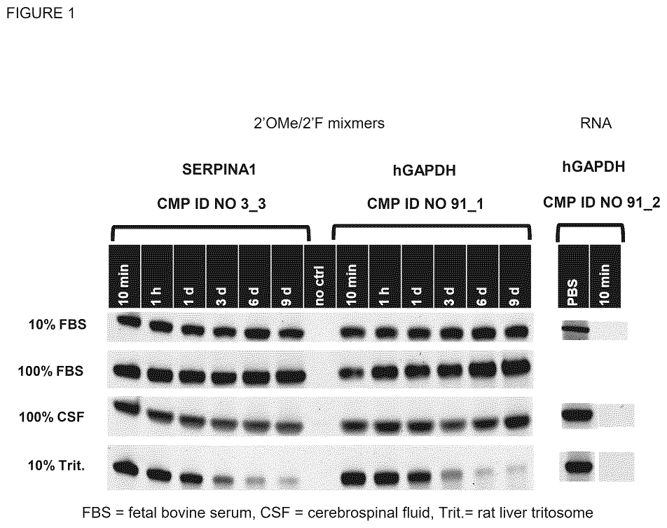

The effect of changing an editing oligonucleotide comprising predominantly RNA nucleosides to an editing oligonucleotide comprising a mixmer of alternating 2′-O-methyl-RNA and 2′-fluoro-RNA nucleosides on the stability of the editing oligonucleotide was investigated (FIG. 1).

Oligonucleoties were diluted to a concentration of 5 μM in PBS, FBS (Sigma Aldrich), human cerebrospinal fluid (CSF, Zen-Bio) or a 10% solution of rat liver tritosomes in catabolism buffer (SEKISUI XenoTech) and incubated at 37° C. After different time intervals as indicated, samples were diluted in sample loading buffer to 0.5 μM and treated with Proteinase K (Thermo Fisher Scientific) for 30 mins according to the manufacturer's protocol. After Proteinase K inactivation, samples were loaded and run on a 15% TBE-Urea Gel (Thermo Fisher Scientific) and bands were visualized by SYBR Gold (Thermo Fisher Scientific) staining.

These data show that an oligonucleotide comprising a mixmer structure of alternating 2′-O-methyl-RNA and 2′-fluoro-RNA nucleosides (CMP ID NOs 3_3 and 91_1 in FIG. 1) is much more resistant to degradation than an oligonucleotide predominantly comprised of RNA nucleosides (CMP ID NO 91_2 in FIG. 1).

### Example 3—Method for Determining Editing Efficiency

The efficiency of editing particular target mRNA (SERPINA1, GAPDH or mHprt) when using different guide oligonucleotides was determined using the following methodology.

In general, cells were transfected with editing oligonucleotides, then RNA was extracted, the target sequence amplified by PCR and the percentage of edited RNA determined by sequencing. The particular cell type and editing target is indicated in each Example or Figure.

### HuH-7/3T3 Cell Experiments

HuH-7-SERPINA1-PiZ cells were custom-CRISPR-engineered to introduce the SERPINA1-PiZ E342K mutation in a wild-type HuH-7 cell line by Horizon Discovery.

7500 cells per well of HuH-7 wt (JCRB), HuH-7-SERPINA1-PiZ or 3T3-L1 (ATCC) were seeded in 96-well plates in the respective media recommended by the vendor. 24 hours after seeding, medium was changed and cells were transfected with 20 μL of a mix of ASO (50 nM final concentration in well if not stated otherwise) and 0.3 μl Lipofectamine RNAiMAX (Thermo Fisher Scientific) per well in OptiMEM (Thermo Fisher Scientific).

Flp-In-T-REx 293-hADAR Cells

Stably expressing Flp-In-T-REx 293-hADAR1 p110/hADAR1 p150/hADAR2 were generated according to the manufacturer's protocol (Thermo Fisher) by co-transfecting the cDNAs of hADAR1 p110/hADAR1 p150/hADAR2 on pcDNA5 vectors obtained from commercially available cDNA vectors (ADAR1 (NM_001111) and RED1 (ADARB1) (NM_001112) Origene) with a Flp recombinase. This was followed by a selection with Hygromycin (100 μg/mL) and Blasticidin S (15 μg/mL) for three weeks. Cells were maintained in selection medium, but were seeded (15000 cells per well in a 96-well plate) in medium containing 1 μg/mL tetracycline and no selection antibiotics 24 h prior to transfection.

### Primary Mouse Hepatocytes

Freshly isolated primary mouse hepatocytes were reverse transfected by adding 25000 cells in Williams E medium supplemented with 10% FBS and 2 mM Glutamine (Sigma Aldrich) to a collagen coated plate (Corning) containing the transfection mix of ASO (50 nM final) and 0.3 μL Lipofectamine RNAiMAX (Thermo Fisher Scientific) in OptiMEM (Thermo Fisher Scientific). For reverse gymnosis, 25000 cells in medium were added to a collagen coated plate containing ASO diluted in PBS.

### RNA Extraction, PCR and Sequencing

Cells were harvested after 24 hours (if not stated otherwise) with RLT buffer and RNA was isolated using the RNeasy 96 kit (Qiagen) with on-column Dnase digest according to the manufacturer's protocol. Before a one-step RT-PCR (QuantaBio qScript XLT One Step Mix) was performed, an oligonucleotide complementary to editing oligonucleotide (“sense”) and the transcript specific forward and reverse primers (Table 10) were added to the isolated RNA and incubated at 90° C. for 2 min. Subsequently the PCR product was diluted 1:10000 in water and a second PCR (Phusion High Fidelity PCR Master Mix with GC Buffer, Thermo Fisher Scientific) was performed using primers with individual barcodes and Illumina adapters for NGS sequencing (Table 11). After the second PCR, the PCR products of the individual wells were pooled and purified with the Monarch PCR&DNA Cleanup KIT (NEB).

The indexed NGS library was sequenced on an Illumina Mini Seq system according to manufacturer's instructions. The generated fastq files were analyzed using the CLC Genomics Workbench Version 20.0.4 software (Qiagen). The part of the reads originated from the primers was trimmed off, the sequences were mapped to the target transcript and variant calling was performed on the mapped reads from each sample. A cut-off of minimum 1% was used as filter in the variant detection and % G at the editing target site was plotted as editing efficiency.

TABLE 10
Target specific primers (1. One-step PCR)

| | | SEQ |
|---|---|---|
| Name | sequence | ID NO |
| hSERPINA1 | CCACTTCTAGACCGAACG GCACCCCTGAAGCTCTCCA | 188 |
| fw | | |
| hSERPINA1 | CCATAACCGTTAGCCAGC GATAGACATGGGTATGGCCTC | 189 |
| rev | | |
| hSERPINA1 | GCCGTGCATAAGGCTGTGCTGACCATCGAC T | 190 |
| sense | CGAAAGGGACTGAAGCTGCTGGAAACATGTA /3InvdT/ | |
| mHprt | CCACTTCTAGACCGAACG GAAAAGCCAAATACAAAGCCTAAG | 191 |
| fw | | |
| mHprt | CCATAACCGTTAGCCAGC GTAACATTCATAGAAGGTTCATGC | 192 |
| rev | | |
| mHprt | ATACGAGGAGTCCTGTTGATGTTGCCAGTAAAATT C | 193 |
| sense | GCAGGTGTTCTAGTCCTGTGGCCATCTGCCTAGTC /3InvdT/ | |
| hGAPDH | CCACTTCTAGACCGAACG CGTGTCCCCACTGCCAAC | 194 |
| fw | | |
| hGAPDH | CCATAACCGTTAGCCAGC GCCTGCTTCACCACCTTC | 195 |
| rev | | |
| hGAPDH | CAGTGGTGGACCTGACCTGCCGTCT C | 196 |
| sense | GAAAAACCTGCCAAATATGATGACA /3InvdT/ | |

“/3InvdT/” stands for “3′ inverted dT”, referring to a DNA thymine nucleotide that has been “inverted” to form a 3′-3′ linkage, as per the following structure:

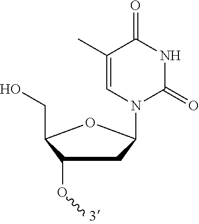

TABLE 11
Primers for sequencing (2. PCR)

| | | SEQ |
|---|---|---|
| Name | Sequence | ID NO |
| sq1_A | AATGATACGGCGACCACCGAGATCTACACAGGCTATAACACTCTTTCCC | 197 |
| | TACACGACGCTCTTCCGATCTNNNNCCATAACCGTTAGCCAGC | |
| sq1_B | AATGATACGGCGACCACCGAGATCTACACGCCTCTATACACTCTTTCCC | 198 |
| | TACACGACGCTCTTCCGATCTNNNNCCATAACCGTTAGCCAGC | |
| sq1_C | AATGATACGGCGACCACCGAGATCTACACAGGATAGGACACTCTTTCCC | 199 |
| | TACACGACGCTCTTCCGATCTNNNNCCATAACCGTTAGCCAGC | |
| sq1_D | AATGATACGGCGACCACCGAGATCTACACTCAGAGCCACACTCTTTCCC | 200 |
| | TACACGACGCTCTTCCGATCTNNNNCCATAACCGTTAGCCAGC | |
| sq1_E | AATGATACGGCGACCACCGAGATCTACACCTTCGCCTACACTCTTTCCC | 201 |
| | TACACGACGCTCTTCCGATCTNNNNCCATAACCGTTAGCCAGC | |
| sq1_F | AATGATACGGCGACCACCGAGATCTACACTAAGATTAACACTCTTTCCCT | 202 |
| | ACACGACGCTCTTCCGATCTNNNNCCATAACCGTTAGCCAGC | |
| sq1_G | AATGATACGGCGACCACCGAGATCTACACACGTCCTGACACTCTTTCCC | 203 |
| | TACACGACGCTCTTCCGATCTNNNNCCATAACCGTTAGCCAGC | |
| sq1_H | AATGATACGGCGACCACCGAGATCTACACGTCAGTACACACTCTTTCCC | 204 |
| | TACACGACGCTCTTCCGATCTNNNNCCATAACCGTTAGCCAGC | |
| sq1_1 | CAAGCAGAAGACGGCATACGAGATCGAGTAATGTGACTGGAGTTCAGA | 205 |
| | CGTGTGCTCTTCCGATCTNNNNCCACTTCTAGACCGAACG | |
| sq1_2 | CAAGCAGAAGACGGCATACGAGATTCTCCGGAGTGACTGGAGTTCAGA | 206 |
| | CGTGTGCTCTTCCGATCTNNNNCCACTTCTAGACCGAACG | |
| sq1_3 | CAAGCAGAAGACGGCATACGAGATCTATCGCTGTGACTGGAGTTCAGAC | 207 |
| | GTGTGCTCTTCCGATCTNNNNCCACTTCTAGACCGAACG | |
| sq1_4 | CAAGCAGAAGACGGCATACGAGATATGCGACGGTGACTGGAGTTCAGA | 208 |
| | CGTGTGCTCTTCCGATCTNNNNCCACTTCTAGACCGAACG | |
| sq1_5 | CAAGCAGAAGACGGCATACGAGATTTCGCGGAGTGACTGGAGTTCAGA | 209 |
| | CGTGTGCTCTTCCGATCTNNNNCCACTTCTAGACCGAACG | |
| sq1_6 | CAAGCAGAAGACGGCATACGAGATCATAGCCGGTGACTGGAGTTCAGA | 210 |
| | CGTGTGCTCTTCCGATCTNNNNCCACTTCTAGACCGAACG | |
| sq1_7 | CAAGCAGAAGACGGCATACGAGATGCGCATTAGTGACTGGAGTTCAGA | 211 |
| | CGTGTGCTCTTCCGATCTNNNNCCACTTCTAGACCGAACG | |
| sq1_8 | CAAGCAGAAGACGGCATACGAGATAGCTTCAGGTGACTGGAGTTCAGA | 212 |
| | CGTGTGCTCTTCCGATCTNNNNCCACTTCTAGACCGAACG | |
| sq1_9 | CAAGCAGAAGACGGCATACGAGATACGAATTCGTGACTGGAGTTCAGAC | 213 |
| | GTGTGCTCTTCCGATCTNNNNCCACTTCTAGACCGAACG | |
| sq1_10 | CAAGCAGAAGACGGCATACGAGATTTCTGAATGTGACTGGAGTTCAGAC | 214 |
| | GTGTGCTCTTCCGATCTNNNNCCACTTCTAGACCGAACG | |
| sq1_11 | CAAGCAGAAGACGGCATACGAGATGGAATCTCGTGACTGGAGTTCAGA | 215 |
| | CGTGTGCTCTTCCGATCTNNNNCCACTTCTAGACCGAACG | |
| sq1_12 | CAAGCAGAAGACGGCATACGAGATAATGAGCGGTGACTGGAGTTCAGA | 216 |
| | CGTGTGCTCTTCCGATCTNNNNCCACTTCTAGACCGAACG | |
| sq2_A | AATGATACGGCGACCACCGAGATCTACACGCTTTATAACACTCTTTCCCT | 217 |
| | ACACGACGCTCTTCCGATCTNNNNCCATAACCGTTAGCCAGC | |
| sq2_B | AATGATACGGCGACCACCGAGATCTACACCGTCCATAACACTCTTTCCC | 218 |
| | TACACGACGCTCTTCCGATCTNNNNCCATAACCGTTAGCCAGC | |
| sq2_C | AATGATACGGCGACCACCGAGATCTACACACATGATAACACTCTTTCCC | 219 |
| | TACACGACGCTCTTCCGATCTNNNNCCATAACCGTTAGCCAGC | |
| sq2_D | AATGATACGGCGACCACCGAGATCTACACACATTCTAACACTCTTTCCCT | 220 |
| | ACACGACGCTCTTCCGATCTNNNNCCATAACCGTTAGCCAGC | |
| sq2_E | AATGATACGGCGACCACCGAGATCTACACTACCTATAACACTCTTTCCCT | 221 |
| | ACACGACGCTCTTCCGATCTNNNNCCATAACCGTTAGCCAGC | |
| sq2_F | AATGATACGGCGACCACCGAGATCTACACAACAGTTAACACTCTTTCCC | 222 |
| | TACACGACGCTCTTCCGATCTNNNNCCATAACCGTTAGCCAGC | |
| sq2_G | AATGATACGGCGACCACCGAGATCTACACCGTAGGTAACACTCTTTCCC | 223 |
| | TACACGACGCTCTTCCGATCTNNNNCCATAACCGTTAGCCAGC | |
| sq2_H | AATGATACGGCGACCACCGAGATCTACACAGGCGATAACACTCTTTCCC | 224 |
| | TACACGACGCTCTTCCGATCTNNNNCCATAACCGTTAGCCAGC | |
| sq2_1 | CAAGCAGAAGACGGCATACGAGATTATGGAGCGTGACTGGAGTTCAGA | 225 |
| | CGTGTGCTCTTCCGATCTNNNNCCACTTCTAGACCGAACG | |
| sq2_2 | CAAGCAGAAGACGGCATACGAGATTATAAGACGTGACTGGAGTTCAGAC | 226 |
| | GTGTGCTCTTCCGATCTNNNNCCACTTCTAGACCGAACG | |
| sq2_3 | CAAGCAGAAGACGGCATACGAGATTAGGGCTTGTGACTGGAGTTCAGA | 227 |
| | CGTGTGCTCTTCCGATCTNNNNCCACTTCTAGACCGAACG | |
| sq2_4 | CAAGCAGAAGACGGCATACGAGATTAAGCGCTGTGACTGGAGTTCAGA | 228 |
| | CGTGTGCTCTTCCGATCTNNNNCCACTTCTAGACCGAACG | |
| sq2_5 | CAAGCAGAAGACGGCATACGAGATTACAGATAGTGACTGGAGTTCAGAC | 229 |
| | GTGTGCTCTTCCGATCTNNNNCCACTTCTAGACCGAACG | |
| sq2_6 | CAAGCAGAAGACGGCATACGAGATTATCACTAGTGACTGGAGTTCAGAC | 230 |
| | GTGTGCTCTTCCGATCTNNNNCCACTTCTAGACCGAACG | |
| sq2_7 | CAAGCAGAAGACGGCATACGAGATTAAGGCTGGTGACTGGAGTTCAGA | 23 |
| | CGTGTGCTCTTCCGATCTNNNNCCACTTCTAGACCGAACG | |
| sq2_8 | CAAGCAGAAGACGGCATACGAGATTAAATCCTGTGACTGGAGTTCAGAC | 232 |
| | GTGTGCTCTTCCGATCTNNNNCCACTTCTAGACCGAACG | |
| sq2_9 | CAAGCAGAAGACGGCATACGAGATTACCCTGAGTGACTGGAGTTCAGA | 233 |
| | CGTGTGCTCTTCCGATCTNNNNCCACTTCTAGACCGAACG | |
| sq2_10 | CAAGCAGAAGACGGCATACGAGATTAGTCAGGGTGACTGGAGTTCAGA | 234 |
| | CGTGTGCTCTTCCGATCTNNNNCCACTTCTAGACCGAACG | |
| sq2_11 | CAAGCAGAAGACGGCATACGAGATTAGATCGGGTGACTGGAGTTCAGA | 235 |
| | CGTGTGCTCTTCCGATCTNNNNCCACTTCTAGACCGAACG | |
| sq2_12 | CAAGCAGAAGACGGCATACGAGATTAGTGATAGTGACTGGAGTTCAGAC | 236 |
| | GTGTGCTCTTCCGATCTNNNNCCACTTCTAGACCGAACG | |
| sq3_A | AATGATACGGCGACCACCGAGATCTACACCCACGTTAACACTCTTTCCC | 237 |
| | TACACGACGCTCTTCCGATCTNNNNCCATAACCGTTAGCCAGC | |
| sq3_B | AATGATACGGCGACCACCGAGATCTACACATGAACTAACACTCTTTCCC | 238 |
| | TACACGACGCTCTTCCGATCTNNNNCCATAACCGTTAGCCAGC | |
| sq3_C | AATGATACGGCGACCACCGAGATCTACACGTTCCGTAACACTCTTTCCC | 239 |
| | TACACGACGCTCTTCCGATCTNNNNCCATAACCGTTAGCCAGC | |
| sq3_D | AATGATACGGCGACCACCGAGATCTACACTATGTCTAACACTCTTTCCCT | 240 |
| | ACACGACGCTCTTCCGATCTNNNNCCATAACCGTTAGCCAGC | |
| sq3_E | AATGATACGGCGACCACCGAGATCTACACCGGATGTAACACTCTTTCCC | 241 |
| | TACACGACGCTCTTCCGATCTNNNNCCATAACCGTTAGCCAGC | |
| sq3_F | AATGATACGGCGACCACCGAGATCTACACCCAGAGTAACACTCTTTCCC | 242 |
| | TACACGACGCTCTTCCGATCTNNNNCCATAACCGTTAGCCAGC | |
| sq3_G | AATGATACGGCGACCACCGAGATCTACACTAGGTATAACACTCTTTCCC | 243 |
| | TACACGACGCTCTTCCGATCTNNNNCCATAACCGTTAGCCAGC | |
| sq3_H | AATGATACGGCGACCACCGAGATCTACACCGGCTATAACACTCTTTCCC | 244 |
| | TACACGACGCTCTTCCGATCTNNNNCCATAACCGTTAGCCAGC | |
| sq3_1 | CAAGCAGAAGACGGCATACGAGATTAGCTTGCGTGACTGGAGTTCAGA | 245 |
| | CGTGTGCTCTTCCGATCTNNNNCCACTTCTAGACCGAACG | |
| sq3_2 | CAAGCAGAAGACGGCATACGAGATTAAGAATCGTGACTGGAGTTCAGAC | 246 |
| | GTGTGCTCTTCCGATCTNNNNCCACTTCTAGACCGAACG | |
| sq3_3 | CAAGCAGAAGACGGCATACGAGATTAAGAGCCGTGACTGGAGTTCAGA | 247 |
| | CGTGTGCTCTTCCGATCTNNNNCCACTTCTAGACCGAACG | |
| sq3_4 | CAAGCAGAAGACGGCATACGAGATTATGCTATGTGACTGGAGTTCAGAC | 248 |
| | GTGTGCTCTTCCGATCTNNNNCCACTTCTAGACCGAACG | |
| sq3_5 | CAAGCAGAAGACGGCATACGAGATTATCATCAGTGACTGGAGTTCAGAC | 249 |
| | GTGTGCTCTTCCGATCTNNNNCCACTTCTAGACCGAACG | |
| sq3_6 | CAAGCAGAAGACGGCATACGAGATTAGCCAAGGTGACTGGAGTTCAGA | 250 |
| | CGTGTGCTCTTCCGATCTNNNNCCACTTCTAGACCGAACG | |
| sq3_7 | CAAGCAGAAGACGGCATACGAGATTACCGTCAGTGACTGGAGTTCAGA | 25 |
| | CGTGTGCTCTTCCGATCTNNNNCCACTTCTAGACCGAACG | |
| sq3_8 | CAAGCAGAAGACGGCATACGAGATTAGTATTCGTGACTGGAGTTCAGAC | 252 |
| | GTGTGCTCTTCCGATCTNNNNCCACTTCTAGACCGAACG | |
| sq3_9 | CAAGCAGAAGACGGCATACGAGATTATGCTGCGTGACTGGAGTTCAGA | 253 |
| | CGTGTGCTCTTCCGATCTNNNNCCACTTCTAGACCGAACG | |
| sq3_10 | CAAGCAGAAGACGGCATACGAGATTAACTATCGTGACTGGAGTTCAGAC | 254 |
| | GTGTGCTCTTCCGATCTNNNNCCACTTCTAGACCGAACG | |
| sq3_11 | CAAGCAGAAGACGGCATACGAGATTAAGGCCTGTGACTGGAGTTCAGA | 255 |
| | CGTGTGCTCTTCCGATCTNNNNCCACTTCTAGACCGAACG | |
| sq3_12 | CAAGCAGAAGACGGCATACGAGATTACCCGAAGTGACTGGAGTTCAGA | 256 |
| | CGTGTGCTCTTCCGATCTNNNNCCACTTCTAGACCGAACG | |
| sq4_A | AATGATACGGCGACCACCGAGATCTACACATCTACTAACACTCTTTCCCT | 257 |
| | ACACGACGCTCTTCCGATCTNNNNCCATAACCGTTAGCCAGC | |
| sq4_B | AATGATACGGCGACCACCGAGATCTACACTAGTTCTAACACTCTTTCCCT | 258 |
| | ACACGACGCTCTTCCGATCTNNNNCCATAACCGTTAGCCAGC | |
| sq4_C | AATGATACGGCGACCACCGAGATCTACACATGTGATAACACTCTTTCCC | 259 |
| | TACACGACGCTCTTCCGATCTNNNNCCATAACCGTTAGCCAGC | |
| sq4_D | AATGATACGGCGACCACCGAGATCTACACTGCGTCTAACACTCTTTCCC | 260 |
| | TACACGACGCTCTTCCGATCTNNNNCCATAACCGTTAGCCAGC | |
| sq4_E | AATGATACGGCGACCACCGAGATCTACACGTACAATAACACTCTTTCCC | 261 |
| | TACACGACGCTCTTCCGATCTNNNNCCATAACCGTTAGCCAGC | |
| sq4_F | AATGATACGGCGACCACCGAGATCTACACTGGTCCTAACACTCTTTCCC | 262 |
| | TACACGACGCTCTTCCGATCTNNNNCCATAACCGTTAGCCAGC | |
| sq4_G | AATGATACGGCGACCACCGAGATCTACACTTAGCTTAACACTCTTTCCCT | 263 |
| | ACACGACGCTCTTCCGATCTNNNNCCATAACCGTTAGCCAGC | |
| sq4_H | AATGATACGGCGACCACCGAGATCTACACACTGAATAACACTCTTTCCC | 264 |
| | TACACGACGCTCTTCCGATCTNNNNCCATAACCGTTAGCCAGC | |
| sq4_1 | CAAGCAGAAGACGGCATACGAGATTACCGCTTGTGACTGGAGTTCAGAC | 265 |
| | GTGTGCTCTTCCGATCTNNNNCCACTTCTAGACCGAACG | |
| sq4_2 | CAAGCAGAAGACGGCATACGAGATTAAGCAGCGTGACTGGAGTTCAGA | 266 |
| | CGTGTGCTCTTCCGATCTNNNNCCACTTCTAGACCGAACG | |
| sq4_3 | CAAGCAGAAGACGGCATACGAGATTACGTGCTGTGACTGGAGTTCAGA | 267 |
| | CGTGTGCTCTTCCGATCTNNNNCCACTTCTAGACCGAACG | |
| sq4_4 | CAAGCAGAAGACGGCATACGAGATTAAACTTCGTGACTGGAGTTCAGAC | 268 |
| | GTGTGCTCTTCCGATCTNNNNCCACTTCTAGACCGAACG | |
| sq4_5 | CAAGCAGAAGACGGCATACGAGATTAAATGCAGTGACTGGAGTTCAGAC | 269 |
| | GTGTGCTCTTCCGATCTNNNNCCACTTCTAGACCGAACG | |
| sq4_6 | CAAGCAGAAGACGGCATACGAGATTATAGCTTGTGACTGGAGTTCAGAC | 270 |
| | GTGTGCTCTTCCGATCTNNNNCCACTTCTAGACCGAACG | |
| sq4_7 | CAAGCAGAAGACGGCATACGAGATTAGCTATTGTGACTGGAGTTCAGAC | 271 |
| | GTGTGCTCTTCCGATCTNNNNCCACTTCTAGACCGAACG | |
| sq4_8 | CAAGCAGAAGACGGCATACGAGATTAGCCATCGTGACTGGAGTTCAGA | 272 |
| | CGTGTGCTCTTCCGATCTNNNNCCACTTCTAGACCGAACG | |
| sq4_9 | CAAGCAGAAGACGGCATACGAGATTATAACAGGTGACTGGAGTTCAGAC | 273 |
| | GTGTGCTCTTCCGATCTNNNNCCACTTCTAGACCGAACG | |
| sq4_10 | CAAGCAGAAGACGGCATACGAGATTAAGACCGGTGACTGGAGTTCAGA | 274 |
| | CGTGTGCTCTTCCGATCTNNNNCCACTTCTAGACCGAACG | |
| sq4_11 | CAAGCAGAAGACGGCATACGAGATTACGCTGTGTGACTGGAGTTCAGA | 275 |
| | CGTGTGCTCTTCCGATCTNNNNCCACTTCTAGACCGAACG | |
| sq4_12 | CAAGCAGAAGACGGCATACGAGATTACTTTAGGTGACTGGAGTTCAGAC | 276 |
| | GTGTGCTCTTCCGATCTNNNNCCACTTCTAGACCGAACG | |
| sq5_A | AATGATACGGCGACCACCGAGATCTACACCCGTTGTAACACTCTTTCCC | 277 |
| | TACACGACGCTCTTCCGATCTNNNNCCATAACCGTTAGCCAGC | |
| sq5_B | AATGATACGGCGACCACCGAGATCTACACTATACCTAACACTCTTTCCCT | 278 |
| | ACACGACGCTCTTCCGATCTNNNNCCATAACCGTTAGCCAGC | |
| sq5_C | AATGATACGGCGACCACCGAGATCTACACACAAGTTAACACTCTTTCCC | 279 |
| | TACACGACGCTCTTCCGATCTNNNNCCATAACCGTTAGCCAGC | |
| sq5_D | AATGATACGGCGACCACCGAGATCTACACTTGGAATAACACTCTTTCCC | 280 |
| | TACACGACGCTCTTCCGATCTNNNNCCATAACCGTTAGCCAGC | |
| sq5_E | AATGATACGGCGACCACCGAGATCTACACGCCCTTTAACACTCTTTCCC | 281 |
| | TACACGACGCTCTTCCGATCTNNNNCCATAACCGTTAGCCAGC | |
| sq5_F | AATGATACGGCGACCACCGAGATCTACACTGATCTTAACACTCTTTCCCT | 282 |
| | ACACGACGCTCTTCCGATCTNNNNCCATAACCGTTAGCCAGC | |
| sq5_G | AATGATACGGCGACCACCGAGATCTACACGGAATTTAACACTCTTTCCC | 283 |
| | TACACGACGCTCTTCCGATCTNNNNCCATAACCGTTAGCCAGC | |
| sq5_H | AATGATACGGCGACCACCGAGATCTACACTGCCGATAACACTCTTTCCC | 284 |
| | TACACGACGCTCTTCCGATCTNNNNCCATAACCGTTAGCCAGC | |
| sq5_1 | CAAGCAGAAGACGGCATACGAGATTATTGTCAGTGACTGGAGTTCAGAC | 285 |
| | GTGTGCTCTTCCGATCTNNNNCCACTTCTAGACCGAACG | |
| sq5_2 | CAAGCAGAAGACGGCATACGAGATTAATTTGCGTGACTGGAGTTCAGAC | 286 |
| | GTGTGCTCTTCCGATCTNNNNCCACTTCTAGACCGAACG | |
| sq5_3 | CAAGCAGAAGACGGCATACGAGATTACGAATAGTGACTGGAGTTCAGAC | 287 |
| | GTGTGCTCTTCCGATCTNNNNCCACTTCTAGACCGAACG | |
| sq5_4 | CAAGCAGAAGACGGCATACGAGATTAACGCGTGTGACTGGAGTTCAGA | 288 |
| | CGTGTGCTCTTCCGATCTNNNNCCACTTCTAGACCGAACG | |
| sq5_5 | CAAGCAGAAGACGGCATACGAGATTATCTTAGGTGACTGGAGTTCAGAC | 289 |
| | GTGTGCTCTTCCGATCTNNNNCCACTTCTAGACCGAACG | |
| sq5_6 | CAAGCAGAAGACGGCATACGAGATTACTGCGTGTGACTGGAGTTCAGA | 290 |
| | CGTGTGCTCTTCCGATCTNNNNCCACTTCTAGACCGAACG | |
| sq5_7 | CAAGCAGAAGACGGCATACGAGATTAGGTGCAGTGACTGGAGTTCAGA | 291 |
| | CGTGTGCTCTTCCGATCTNNNNCCACTTCTAGACCGAACG | |
| sq5_8 | CAAGCAGAAGACGGCATACGAGATTACAGTTTGTGACTGGAGTTCAGAC | 292 |
| | GTGTGCTCTTCCGATCTNNNNCCACTTCTAGACCGAACG | |
| sq5_9 | CAAGCAGAAGACGGCATACGAGATTAGTGCGAGTGACTGGAGTTCAGA | 293 |
| | CGTGTGCTCTTCCGATCTNNNNCCACTTCTAGACCGAACG | |
| sq5_10 | CAAGCAGAAGACGGCATACGAGATTAGTTAGAGTGACTGGAGTTCAGAC | 294 |
| | GTGTGCTCTTCCGATCTNNNNCCACTTCTAGACCGAACG | |
| sq5_11 | CAAGCAGAAGACGGCATACGAGATTAACCCGAGTGACTGGAGTTCAGA | 295 |
| | CGTGTGCTCTTCCGATCTNNNNCCACTTCTAGACCGAACG | |
| sq5_12 | CAAGCAGAAGACGGCATACGAGATTACCAGTGTGACTGGAGTTCAGA | 296 |
| | CGTGTGCTCTTCCGATCTNNNNCCACTTCTAGACCGAACG | |
| sq6_A | AATGATACGGCGACCACCGAGATCTACACGTGAATTAACACTCTTTCCC | 297 |
| | TACACGACGCTCTTCCGATCTNNNNCCATAACCGTTAGCCAGC | |
| sq6_B | AATGATACGGCGACCACCGAGATCTACACTCACATTAACACTCTTTCCCT | 298 |
| | ACACGACGCTCTTCCGATCTNNNNCCATAACCGTTAGCCAGC | |
| sq6_C | AATGATACGGCGACCACCGAGATCTACACGACACGTAACACTCTTTCCC | 299 |
| | TACACGACGCTCTTCCGATCTNNNNCCATAACCGTTAGCCAGC | |
| sq6_D | AATGATACGGCGACCACCGAGATCTACACGTAAGTTAACACTCTTTCCC | 300 |
| | TACACGACGCTCTTCCGATCTNNNNCCATAACCGTTAGCCAGC | |
| sq6_E | AATGATACGGCGACCACCGAGATCTACACGGCTGATAACACTCTTTCCC | 301 |
| | TACACGACGCTCTTCCGATCTNNNNCCATAACCGTTAGCCAGC | |
| sq6_F | AATGATACGGCGACCACCGAGATCTACACAATTCCTAACACTCTTTCCCT | 302 |
| | ACACGACGCTCTTCCGATCTNNNNCCATAACCGTTAGCCAGC | |
| sq6_G | AATGATACGGCGACCACCGAGATCTACACGCAGGTTAACACTCTTTCCC | 303 |
| | TACACGACGCTCTTCCGATCTNNNNCCATAACCGTTAGCCAGC | |
| sq6_H | AATGATACGGCGACCACCGAGATCTACACTTGCTATAACACTCTTTCCCT | 304 |
| | ACACGACGCTCTTCCGATCTNNNNCCATAACCGTTAGCCAGC | |
| sq6_1 | CAAGCAGAAGACGGCATACGAGATTACAGCCTGTGACTGGAGTTCAGA | 305 |
| | CGTGTGCTCTTCCGATCTNNNNCCACTTCTAGACCGAACG | |
| sq6_2 | CAAGCAGAAGACGGCATACGAGATTACCGTTGGTGACTGGAGTTCAGA | 306 |
| | CGTGTGCTCTTCCGATCTNNNNCCACTTCTAGACCGAACG | |
| sq6_3 | CAAGCAGAAGACGGCATACGAGATTAGTGAATGTGACTGGAGTTCAGAC | 307 |
| | GTGTGCTCTTCCGATCTNNNNCCACTTCTAGACCGAACG | |
| sq6_4 | CAAGCAGAAGACGGCATACGAGATTAAGTGGCGTGACTGGAGTTCAGA | 308 |
| | CGTGTGCTCTTCCGATCTNNNNCCACTTCTAGACCGAACG | |
| sq6_5 | CAAGCAGAAGACGGCATACGAGATTACTACTAGTGACTGGAGTTCAGAC | 309 |
| | GTGTGCTCTTCCGATCTNNNNCCACTTCTAGACCGAACG | |
| sq6_6 | CAAGCAGAAGACGGCATACGAGATTAAGACATGTGACTGGAGTTCAGAC | 310 |
| | GTGTGCTCTTCCGATCTNNNNCCACTTCTAGACCGAACG | |
| sq6_7 | CAAGCAGAAGACGGCATACGAGATTACAATAGGTGACTGGAGTTCAGAC | 311 |
| | GTGTGCTCTTCCGATCTNNNNCCACTTCTAGACCGAACG | |
| sq6_8 | CAAGCAGAAGACGGCATACGAGATTATGGATAGTGACTGGAGTTCAGAC | 312 |
| | GTGTGCTCTTCCGATCTNNNNCCACTTCTAGACCGAACG | |
| sq6_9 | CAAGCAGAAGACGGCATACGAGATTACGCACTGTGACTGGAGTTCAGA | 313 |
| | CGTGTGCTCTTCCGATCTNNNNCCACTTCTAGACCGAACG | |
| sq6_10 | CAAGCAGAAGACGGCATACGAGATTAGGTAGCGTGACTGGAGTTCAGA | 314 |
| | CGTGTGCTCTTCCGATCTNNNNCCACTTCTAGACCGAACG | |
| sq6_11 | CAAGCAGAAGACGGCATACGAGATTAGCCTTGGTGACTGGAGTTCAGA | 315 |
| | CGTGTGCTCTTCCGATCTNNNNCCACTTCTAGACCGAACG | |
| sq6_12 | CAAGCAGAAGACGGCATACGAGATTAAGTCTTGTGACTGGAGTTCAGAC | 316 |
| | GTGTGCTCTTCCGATCTNNNNCCACTTCTAGACCGAACG | |
| sq7_A | AATGATACGGCGACCACCGAGATCTACACCGCATCTAACACTCTTTCCC | 317 |
| | TACACGACGCTCTTCCGATCTNNNNCCATAACCGTTAGCCAGC | |
| sq7_B | AATGATACGGCGACCACCGAGATCTACACGTCCCATAACACTCTTTCCC | 318 |
| | TACACGACGCTCTTCCGATCTNNNNCCATAACCGTTAGCCAGC | |
| sq7_C | AATGATACGGCGACCACCGAGATCTACACTGTGCCTAACACTCTTTCCC | 319 |
| | TACACGACGCTCTTCCGATCTNNNNCCATAACCGTTAGCCAGC | |
| sq7_D | AATGATACGGCGACCACCGAGATCTACACTAACAGTAACACTCTTTCCC | 320 |
| | TACACGACGCTCTTCCGATCTNNNNCCATAACCGTTAGCCAGC | |
| sq7_E | AATGATACGGCGACCACCGAGATCTACACCGAGCATAACACTCTTTCCC | 321 |
| | TACACGACGCTCTTCCGATCTNNNNCCATAACCGTTAGCCAGC | |
| sq7_F | AATGATACGGCGACCACCGAGATCTACACCCTAGCTAACACTCTTTCCC | 322 |
| | TACACGACGCTCTTCCGATCTNNNNCCATAACCGTTAGCCAGC | |
| sq7_G | AATGATACGGCGACCACCGAGATCTACACTACGTTTAACACTCTTTCCCT | 323 |
| | ACACGACGCTCTTCCGATCTNNNNCCATAACCGTTAGCCAGC | |
| sq7_H | AATGATACGGCGACCACCGAGATCTACACCGACTCTAACACTCTTTCCC | 324 |
| | TACACGACGCTCTTCCGATCTNNNNCCATAACCGTTAGCCAGC | |
| sq7_1 | CAAGCAGAAGACGGCATACGAGATTAGCTAAAGTGACTGGAGTTCAGAC | 325 |
| | GTGTGCTCTTCCGATCTNNNNCCACTTCTAGACCGAACG | |
| sq7_2 | CAAGCAGAAGACGGCATACGAGATTAGGCCGCGTGACTGGAGTTCAGA | 326 |
| | CGTGTGCTCTTCCGATCTNNNNCCACTTCTAGACCGAACG | |
| sq7_3 | CAAGCAGAAGACGGCATACGAGATTACTGCACGTGACTGGAGTTCAGA | 327 |
| | CGTGTGCTCTTCCGATCTNNNNCCACTTCTAGACCGAACG | |
| sq7_4 | CAAGCAGAAGACGGCATACGAGATTACATCTAGTGACTGGAGTTCAGAC | 328 |
| | GTGTGCTCTTCCGATCTNNNNCCACTTCTAGACCGAACG | |
| sq7_5 | CAAGCAGAAGACGGCATACGAGATTAGGACTGGTGACTGGAGTTCAGA | 329 |
| | CGTGTGCTCTTCCGATCTNNNNCCACTTCTAGACCGAACG | |
| sq7_6 | CAAGCAGAAGACGGCATACGAGATTACTATCAGTGACTGGAGTTCAGAC | 330 |
| | GTGTGCTCTTCCGATCTNNNNCCACTTCTAGACCGAACG | |
| sq7_7 | CAAGCAGAAGACGGCATACGAGATTAACGGTGGTGACTGGAGTTCAGA | 331 |
| | CGTGTGCTCTTCCGATCTNNNNCCACTTCTAGACCGAACG | |
| sq7_8 | CAAGCAGAAGACGGCATACGAGATTATCAAAGGTGACTGGAGTTCAGAC | 332 |
| | GTGTGCTCTTCCGATCTNNNNCCACTTCTAGACCGAACG | |
| sq7_9 | CAAGCAGAAGACGGCATACGAGATTACTGACCGTGACTGGAGTTCAGA | 333 |
| | CGTGTGCTCTTCCGATCTNNNNCCACTTCTAGACCGAACG | |
| sq7_10 | CAAGCAGAAGACGGCATACGAGATTACAAGTAGTGACTGGAGTTCAGAC | 334 |
| | GTGTGCTCTTCCGATCTNNNNCCACTTCTAGACCGAACG | |
| sq7_11 | CAAGCAGAAGACGGCATACGAGATTACTACATGTGACTGGAGTTCAGAC | 335 |
| | GTGTGCTCTTCCGATCTNNNNCCACTTCTAGACCGAACG | |
| sq7_12 | CAAGCAGAAGACGGCATACGAGATTAATCTTGGTGACTGGAGTTCAGAC | 336 |
| | GTGTGCTCTTCCGATCTNNNNCCACTTCTAGACCGAACG | |
| sq8_A | AATGATACGGCGACCACCGAGATCTACACGAACATTAACACTCTTTCCC | 337 |
| | TACACGACGCTCTTCCGATCTNNNNCCATAACCGTTAGCCAGC | |
| sq8_B | AATGATACGGCGACCACCGAGATCTACACAACGCCTAACACTCTTTCCC | 338 |
| | TACACGACGCTCTTCCGATCTNNNNCCATAACCGTTAGCCAGC | |
| sq8_C | AATGATACGGCGACCACCGAGATCTACACTGCGGTTAACACTCTTTCCC | 339 |
| | TACACGACGCTCTTCCGATCTNNNNCCATAACCGTTAGCCAGC | |
| sq8_D | AATGATACGGCGACCACCGAGATCTACACGTCATTTAACACTCTTTCCCT | 340 |
| | ACACGACGCTCTTCCGATCTNNNNCCATAACCGTTAGCCAGC | |
| sq8_E | AATGATACGGCGACCACCGAGATCTACACGGGACTTAACACTCTTTCCC | 341 |
| | TACACGACGCTCTTCCGATCTNNNNCCATAACCGTTAGCCAGC | |
| sq8_F | AATGATACGGCGACCACCGAGATCTACACGTAGATTAACACTCTTTCCC | 342 |
| | TACACGACGCTCTTCCGATCTNNNNCCATAACCGTTAGCCAGC | |
| sq8_G | AATGATACGGCGACCACCGAGATCTACACCATGGGTAACACTCTTTCCC | 343 |
| | TACACGACGCTCTTCCGATCTNNNNCCATAACCGTTAGCCAGC | |
| sq8_H | AATGATACGGCGACCACCGAGATCTACACCAGTTTTAACACTCTTTCCCT | 344 |
| | ACACGACGCTCTTCCGATCTNNNNCCATAACCGTTAGCCAGC | |
| sq8_1 | CAAGCAGAAGACGGCATACGAGATTATGAGTAGTGACTGGAGTTCAGAC | 345 |
| | GTGTGCTCTTCCGATCTNNNNCCACTTCTAGACCGAACG | |
| sq8_2 | CAAGCAGAAGACGGCATACGAGATTACGAGTGGTGACTGGAGTTCAGA | 346 |
| | CGTGTGCTCTTCCGATCTNNNNCCACTTCTAGACCGAACG | |
| sq8_3 | CAAGCAGAAGACGGCATACGAGATTACAAACCGTGACTGGAGTTCAGAC | 347 |
| | GTGTGCTCTTCCGATCTNNNNCCACTTCTAGACCGAACG | |
| sq8_4 | CAAGCAGAAGACGGCATACGAGATTATGACTTGTGACTGGAGTTCAGAC | 348 |
| | GTGTGCTCTTCCGATCTNNNNCCACTTCTAGACCGAACG | |
| sq8_5 | CAAGCAGAAGACGGCATACGAGATTAATTCCAGTGACTGGAGTTCAGAC | 349 |
| | GTGTGCTCTTCCGATCTNNNNCCACTTCTAGACCGAACG | |
| sq8_6 | CAAGCAGAAGACGGCATACGAGATTACGACTCGTGACTGGAGTTCAGA | 350 |
| | CGTGTGCTCTTCCGATCTNNNNCCACTTCTAGACCGAACG | |
| sq8_7 | CAAGCAGAAGACGGCATACGAGATTAGTCCGTGTGACTGGAGTTCAGA | 351 |
| | CGTGTGCTCTTCCGATCTNNNNCCACTTCTAGACCGAACG | |
| sq8_8 | CAAGCAGAAGACGGCATACGAGATTATTGCATGTGACTGGAGTTCAGAC | 352 |
| | GTGTGCTCTTCCGATCTNNNNCCACTTCTAGACCGAACG | |
| sq8_9 | CAAGCAGAAGACGGCATACGAGATTATGTTCAGTGACTGGAGTTCAGAC | 353 |
| | GTGTGCTCTTCCGATCTNNNNCCACTTCTAGACCGAACG | |
| sq8_10 | CAAGCAGAAGACGGCATACGAGATTACGCCAAGTGACTGGAGTTCAGA | 354 |
| | CGTGTGCTCTTCCGATCTNNNNCCACTTCTAGACCGAACG | |
| sq8_11 | CAAGCAGAAGACGGCATACGAGATTATCTACAGTGACTGGAGTTCAGAC | 355 |
| | GTGTGCTCTTCCGATCTNNNNCCACTTCTAGACCGAACG | |
| sq8_12 | CAAGCAGAAGACGGCATACGAGATTACGGCTAGTGACTGGAGTTCAGA | 356 |
| | CGTGTGCTCTTCCGATCTNNNNCCACTTCTAGACCGAACG | |

### Example 4—Effect of Mixmer on Editing Efficiency

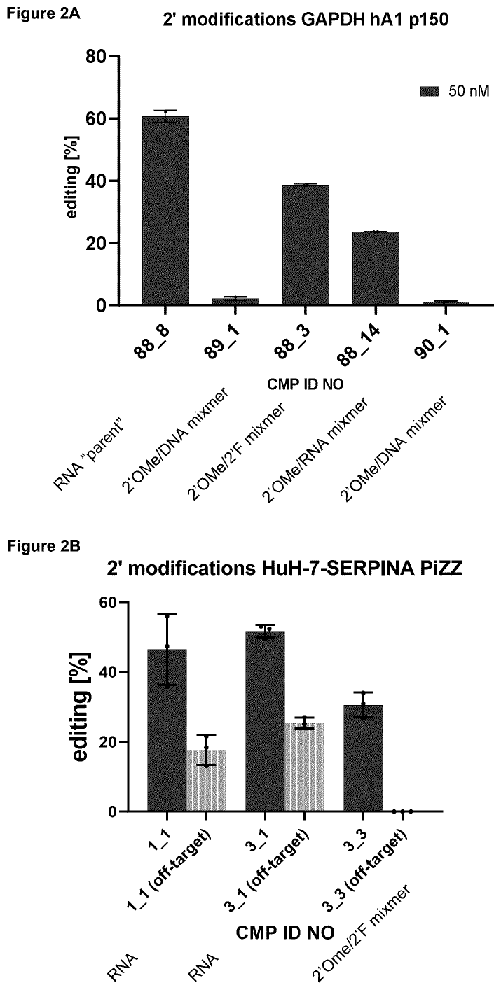

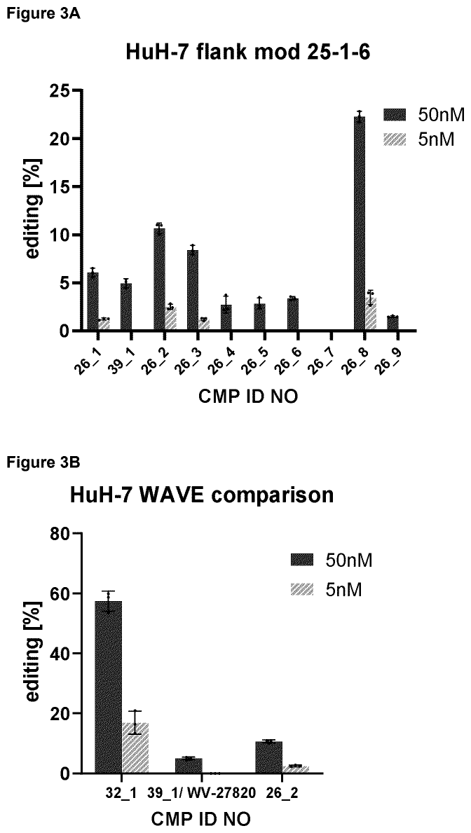

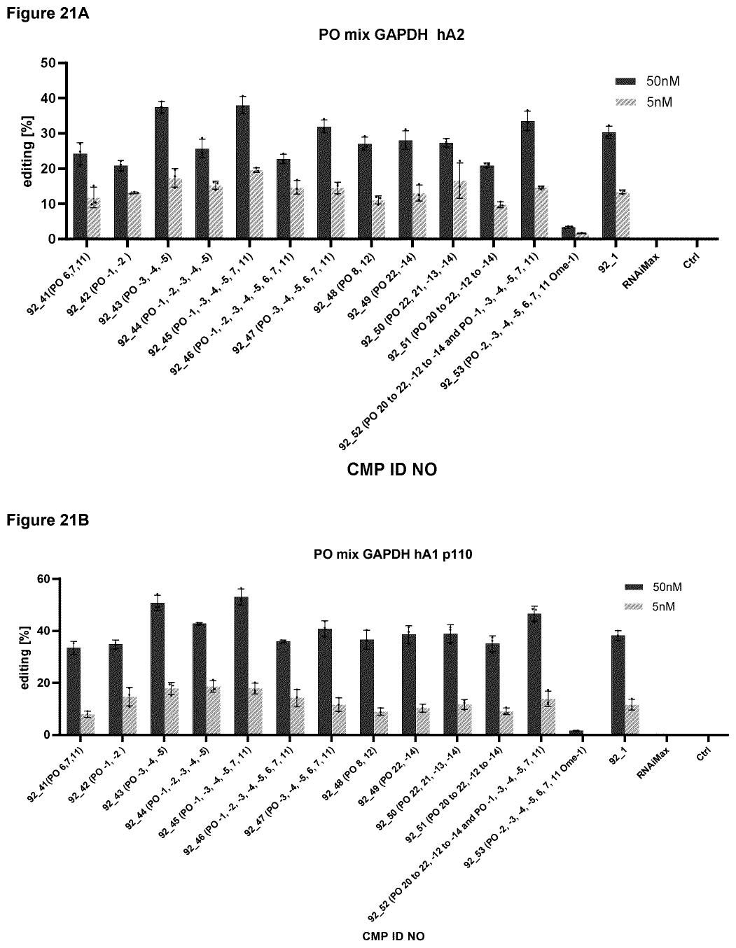

The effect of including a mixmer sequence in an editing oligonucleotide on editing of GAPDH in HEK-293 ADAR1 p150 cells (FIG. 2A) and on editing of SERPINA1 in HuH7 cells (FIG. 21B, FIG. 3A and FIG. 3B) mRNA was investigated.

Highest editing efficiency was achieved with a non-mixmer RNA sequence, but such an oligonucleotide would have low stability as indicated by Example 2 herein. Good editing efficiencies were also achieved with 2′-O-methyl-RNA/2′-fluoro-RNA mixmers and RNA/DNA mixmers.

### Example 5—Effect of Editing Region Inosine on Editing Efficiency

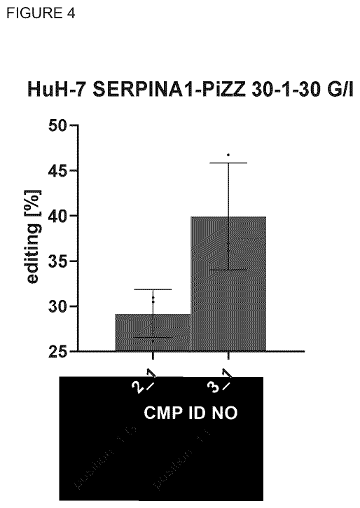

Replacement of guanosine with inosine at the −1 position of an editing oligonucleotide greatly enhanced editing of SERPINA1 mRNA (FIG. 4).

### Example 6—Effect of Benner's Base at Editing Nucleoside on Editing Efficiency

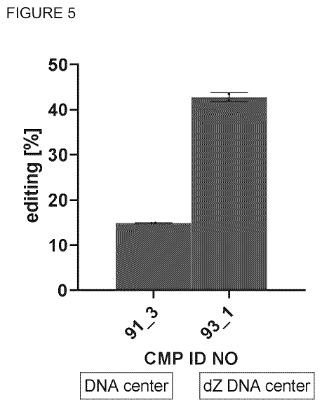

Replacement of cytosine with Benner's base (Z) at the 0 position (the editing nucleoside) of an editing oligonucleotide greatly enhanced editing of GAPDH mRNA (FIG. 5).

### Example 7—Effect of Length and Symmetry on Editing Efficiency

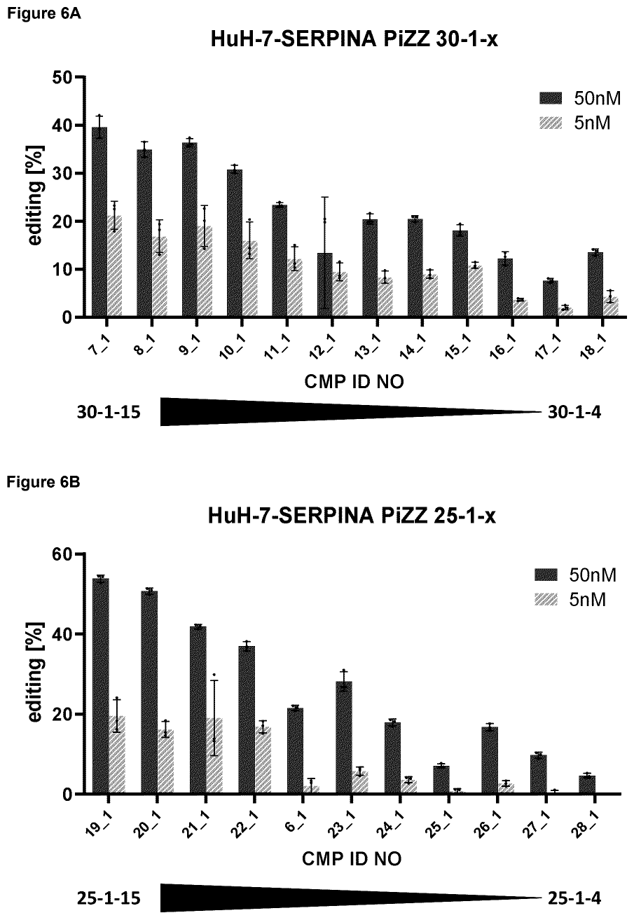

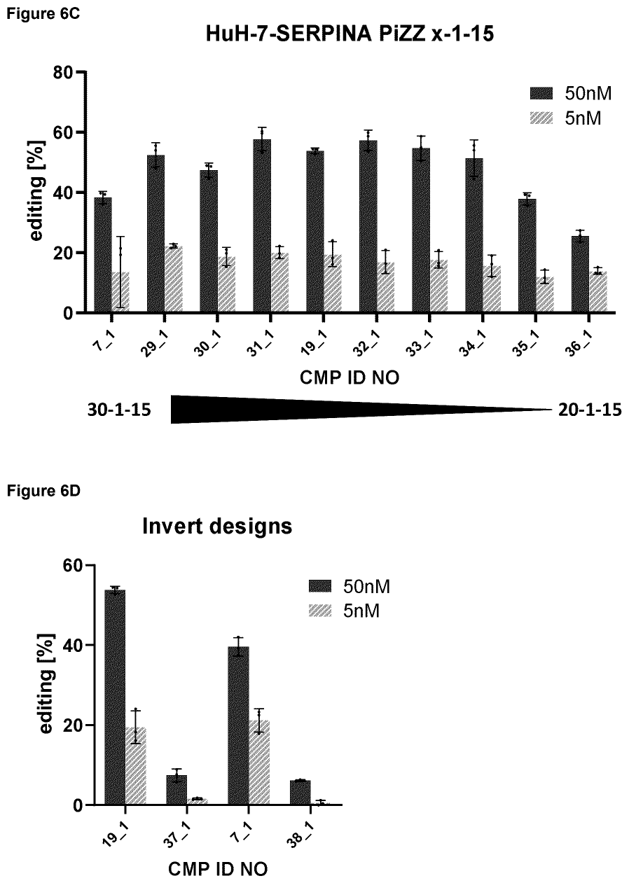

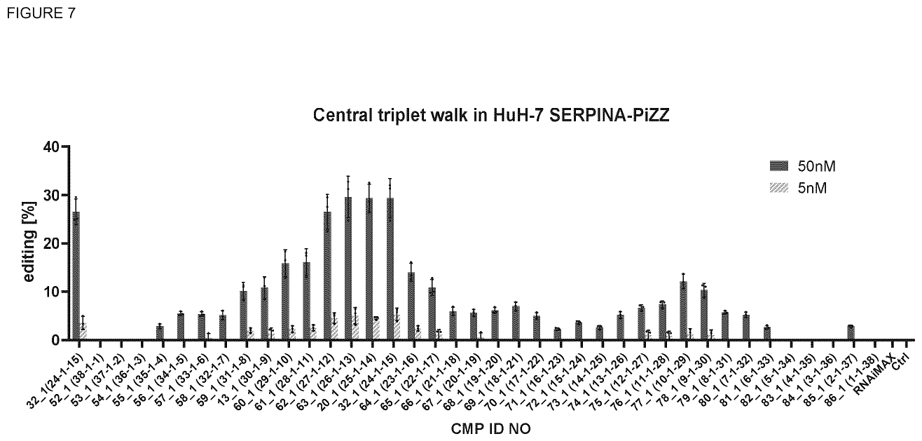

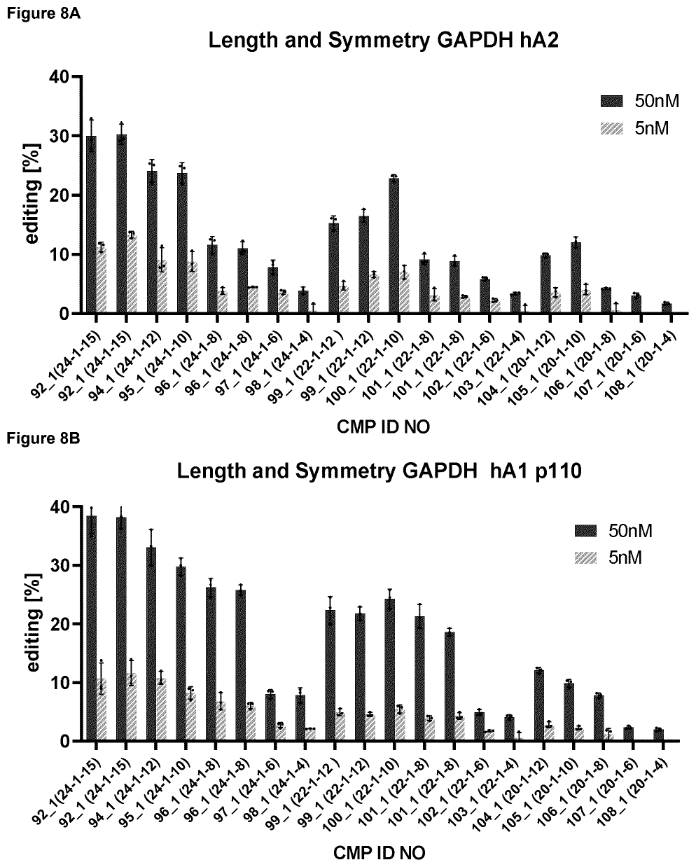

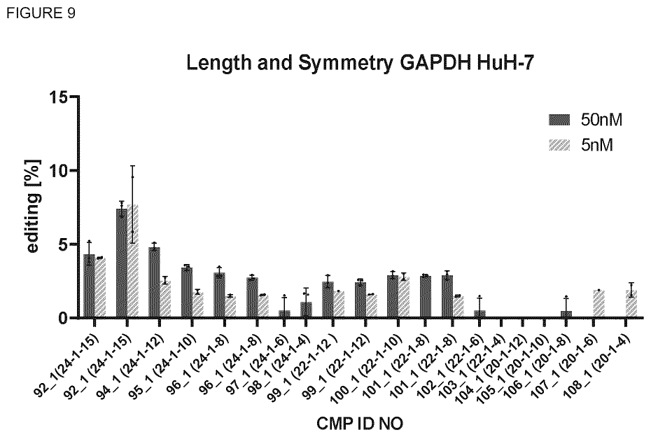

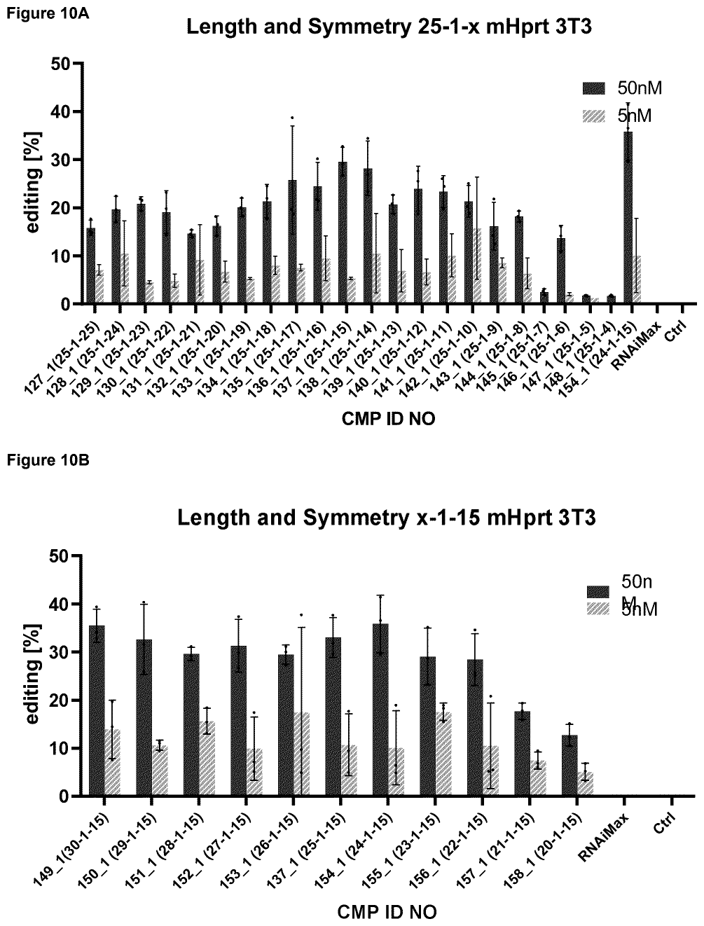

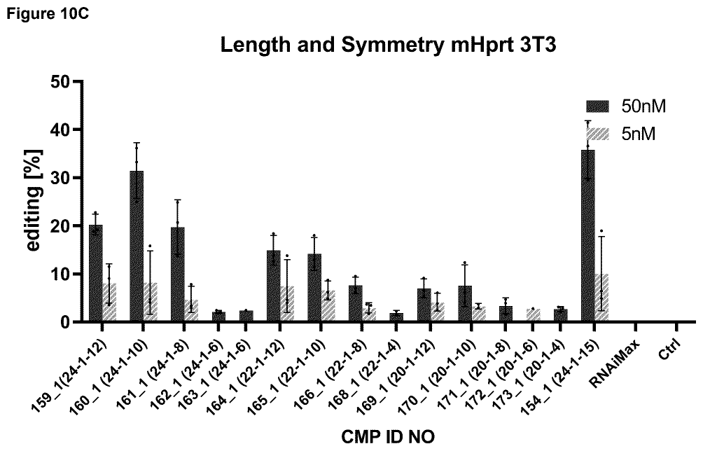

The effect of varying the length and symmetry of editing oligonucleotides on editing of SERPINA1 (FIGS. 6A, 6B and 6C and FIG. 7), GAPDH (FIGS. 8A and 8B and FIG. 9) and mHprt (FIGS. 10A, 10B and 10C) mRNA was investigated.

FIGS. 6A, 6B and 6C, FIGS. 8A and 8B, and FIG. 9 indicate that increasing the length of oligonucleotide 3′ to the editing nucleoside from 4 to 15 nucleosides improves editing efficiency. FIG. 7 shows that good editing efficiency is achieved in the range of symmetries from about 30-1-9 to about 22-1-17. FIG. 10A shows that good editing efficiency can be achieved with about 8 to 25 nucleosides 3′ to the editing nucleoside. FIG. 10B indicates that good editing efficiency can be achieved with about 22 to 30 nucleosides 5′ to the editing nucleoside

### Example 8—Effect of Editing Region Modifications on Editing Efficiency

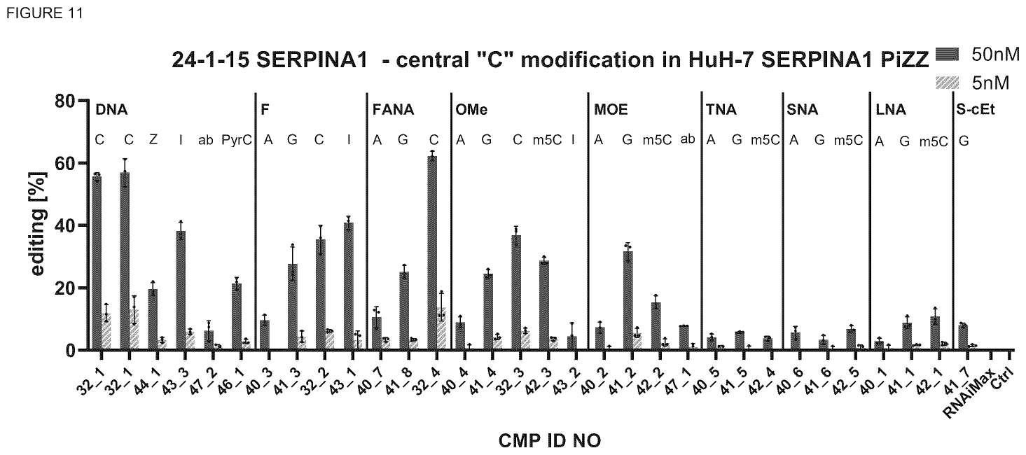

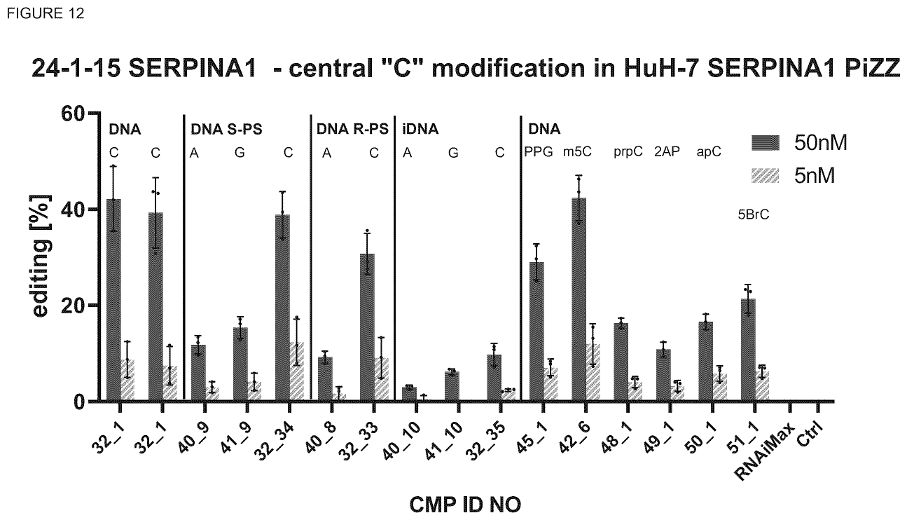

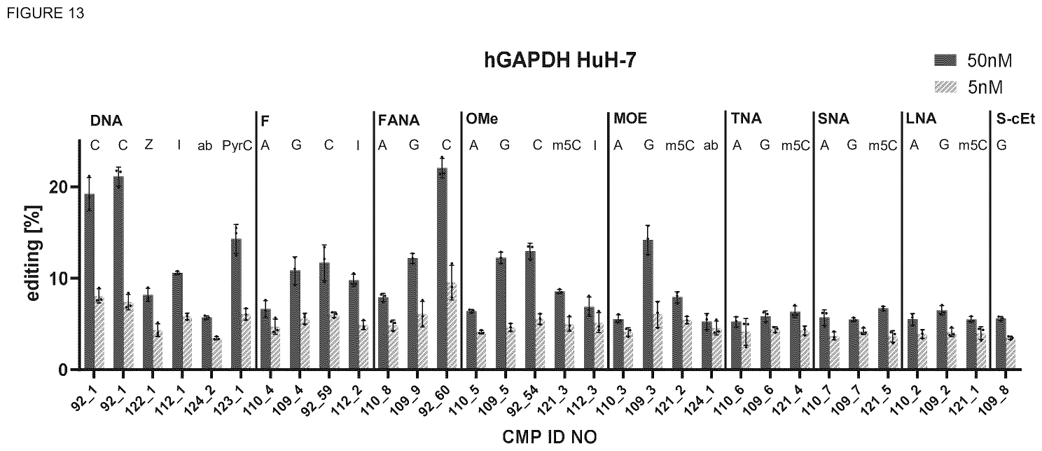

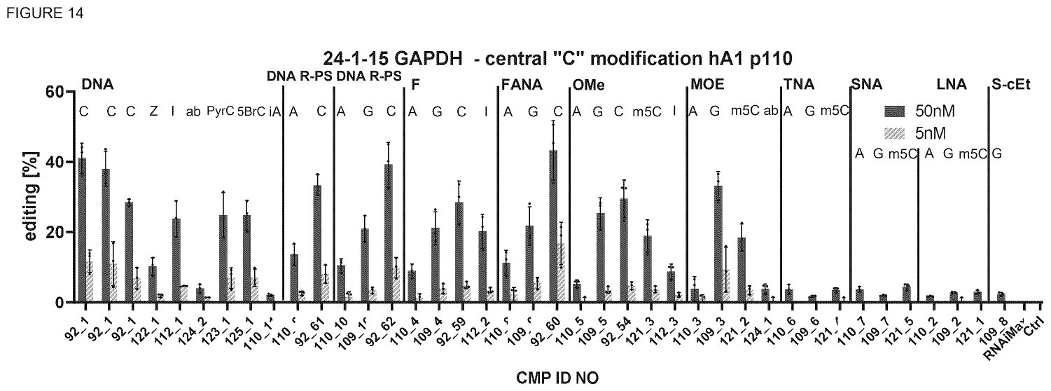

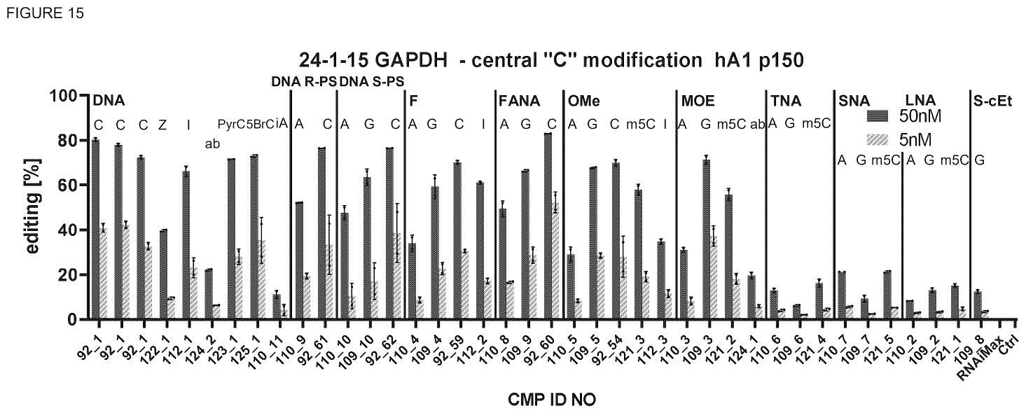

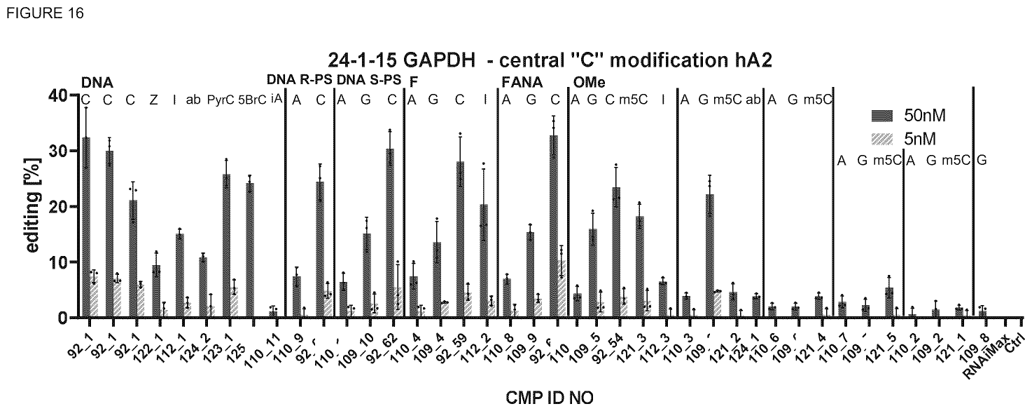

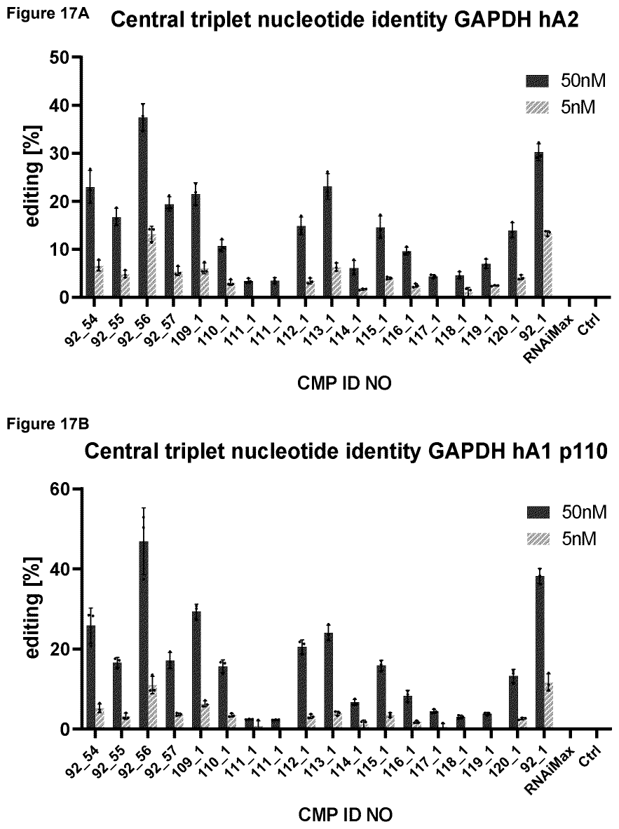

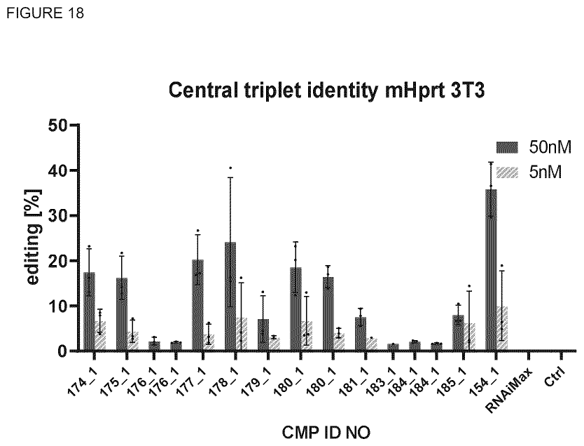

The effect of modifying nucleosides in the editing region of editing oligonucleotides on editing of SERPINA1 (FIGS. 11 and 12), GAPDH (FIG. 13 to FIGS. 17A and 17B) and mHprt (FIG. 18) mRNA was investigated. An editing nucleoside that is a DNA nucleoside or FANA nucleoside particularly enhances editing efficiency (see FIGS. 11, 12, 13, 14 and 16).

### Example 9—Effect of Internucleoside Linkages on Editing Efficiency

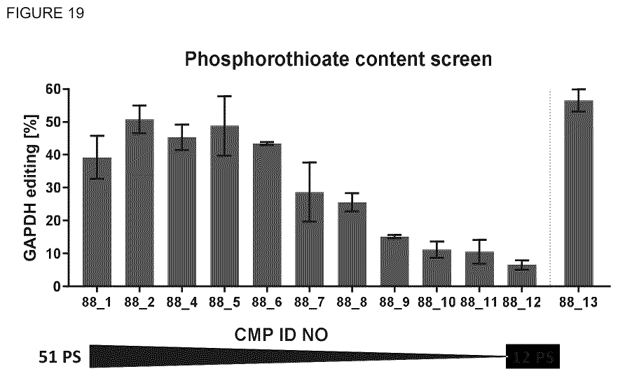

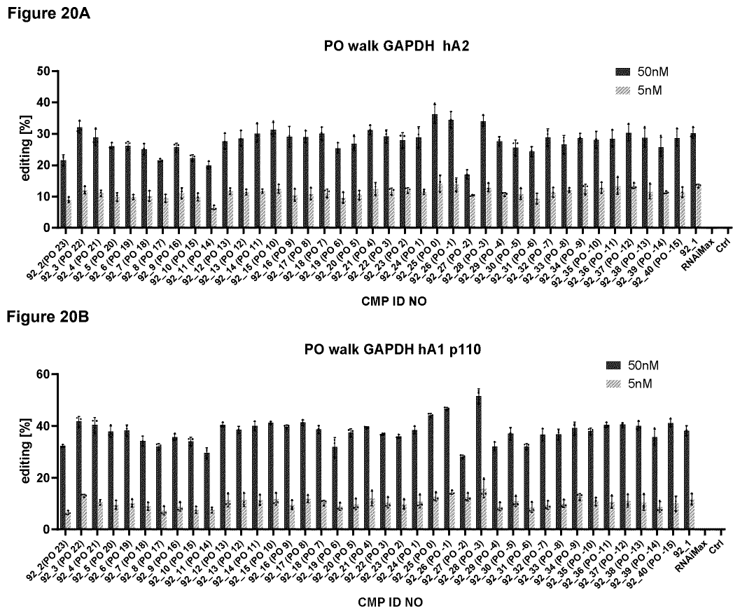

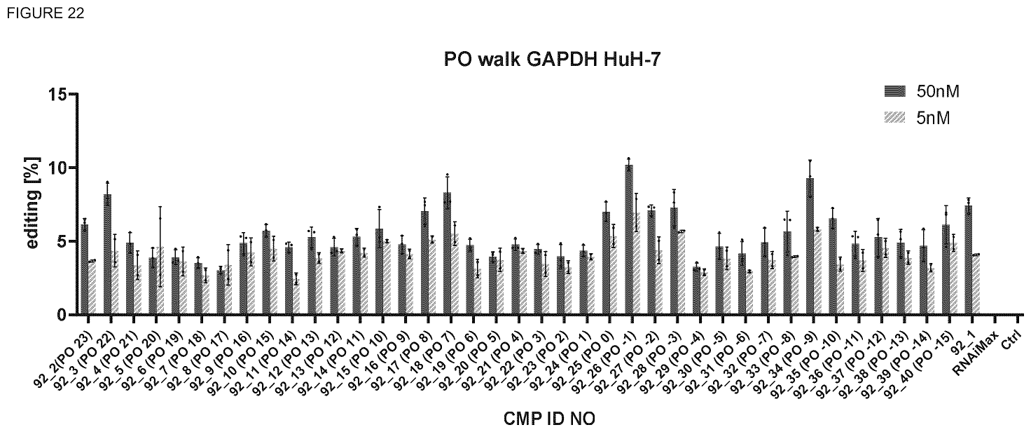

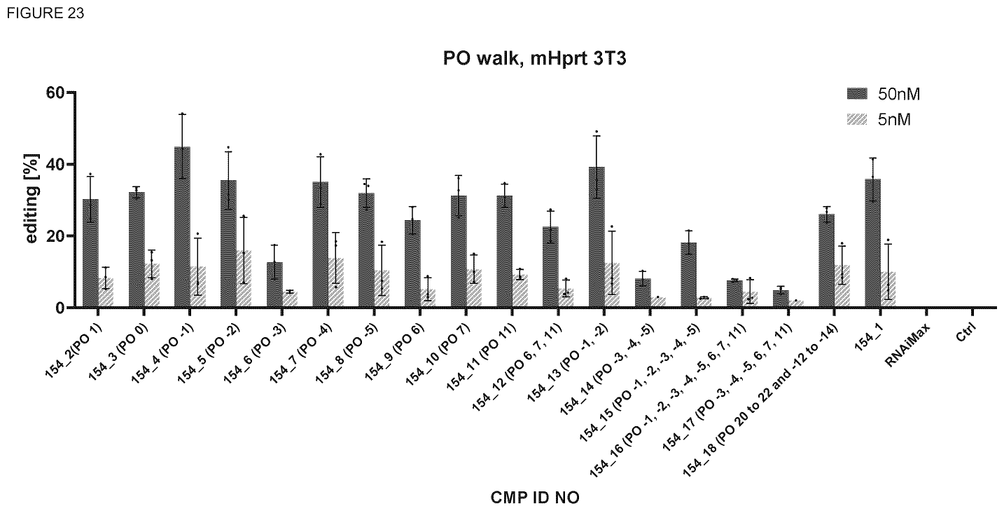

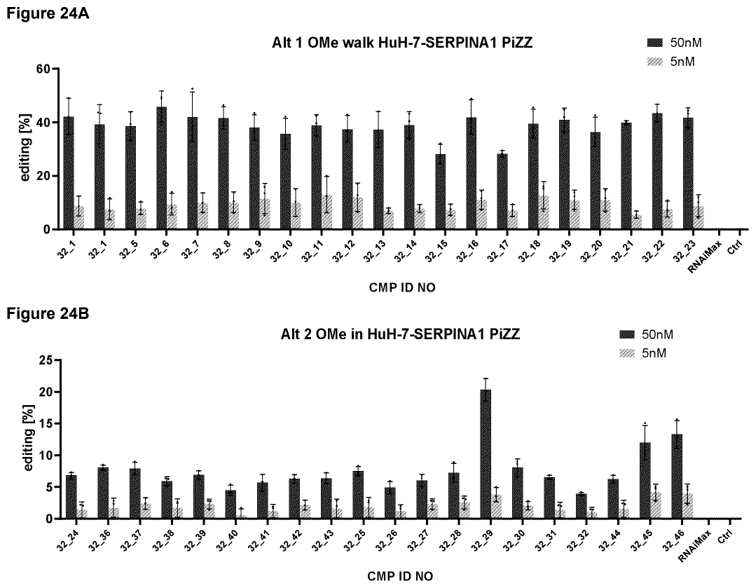

The effect of modifying internucleoside linkages of editing oligonucleotides on editing of GAPDH (FIGS. 19 to 22) and mHprt (FIG. 23) mRNA was investigated. FIG. 19 shows that high phosphorothioate (PS) content is tolerated, and even preferred, in an editing oligonucleotide.

### Example 10—Effect of Modified Sugar Moieties on Editing Efficiency

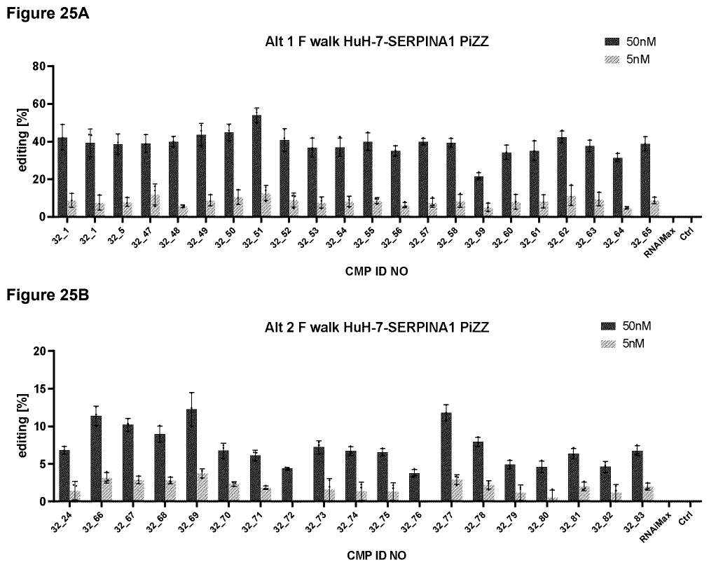

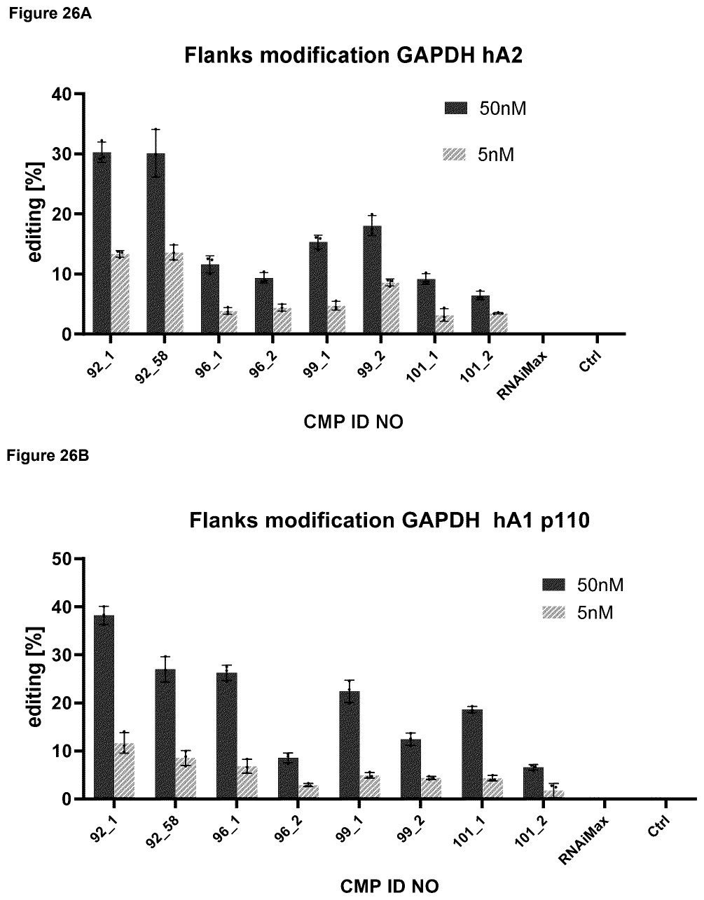

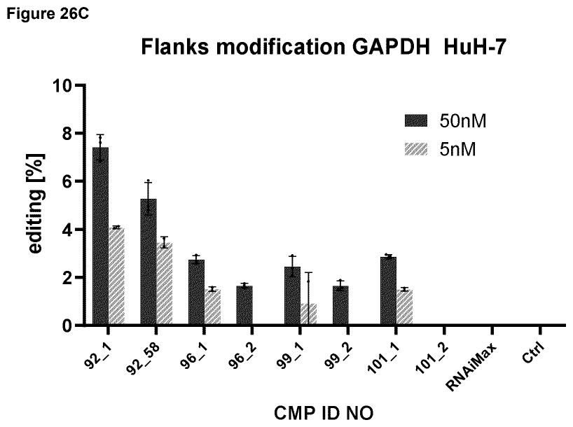

The effect of 2′-O-methyl and 2′-fluoro sugar modifications at particular positions in editing oligonucleotides on editing of SERPINA1 mRNA was investigated (FIGS. 24A and 24B and 25A and 25B).

2′-O-methyl-RNA at position −2 enhances editing (see FIG. 24B, CMP ID NO 32_29). 2′-fluoro-RNA at position −3 enhances editing (see FIG. 25B, CMP ID NO 32_77).

### Example 11—Effect of Flank Regions on Editing Efficiency

The effect of the presence of flank regions comprising 2′-O-methyl or 2′-MOE sugar modifications in editing oligonucleotides on editing of GAPDH mRNA was investigated (FIGS. 26A, 26B and 26C).

### Example 12—Effect of Conjugation on Editing Efficiency

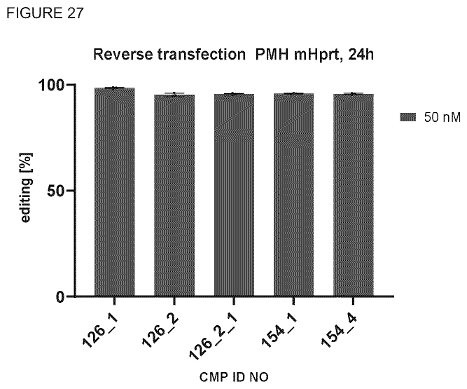

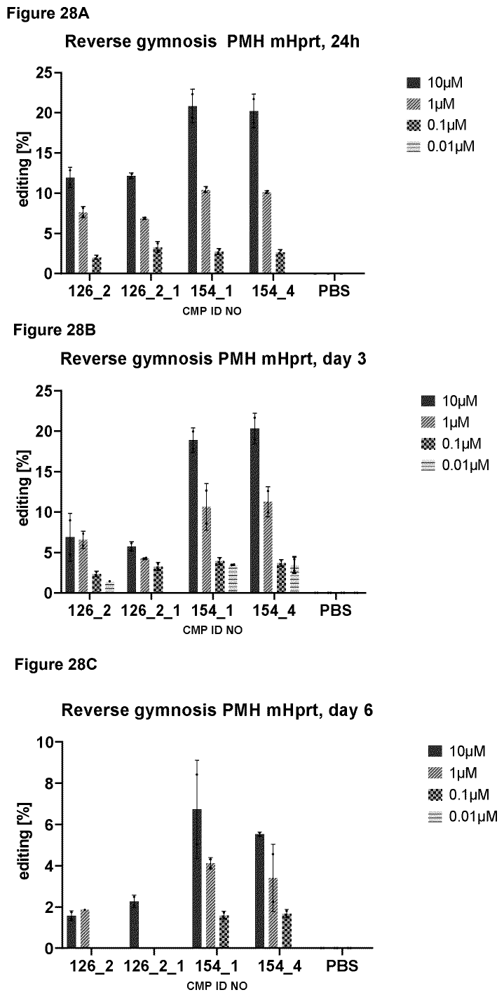

Mouse primary hepatocytes were exposed to editing oligonucleotides targeting mHprt, including an oligonucleotide conjugated to a GalNAc conjugate moiety. The conjugate (CNJ ID NO 126_21) was as effective at editing mHprt mRNA as a non-conjugated compound having the same oligonucleotide sequence and modification pattern (CMP ID NO 126_2) (FIG. 27 and FIGS. 28A, 28 B, and 28C).

1. An oligonucleotide comprising
an editing region that comprises an editing nucleoside,
a 5′ mixmer region positioned 5′ to the editing region, and
a 3′ mixmer region positioned 3′ to the editing region, wherein
the editing nucleoside is designated as position 0,
each nucleoside 5′ to the editing nucleoside is designated as position +x, wherein x is the number of nucleosides 5′ to the editing nucleoside at that position including the nucleoside at that position, and
each nucleoside 3′ to the editing nucleoside is designated as position −y, wherein y is the number of nucleosides 3′ to the editing nucleoside at that position including the nucleoside at that position.

2. The oligonucleotide of claim 1, wherein
(a) the 5′ mixmer region comprises a first type of 5′ mixmer nucleoside and a second type of 5′ mixmer nucleoside, wherein the sugar moiety of the first type of 5′ mixmer nucleoside is different to the sugar moiety of the second type of 5′ mixmer nucleoside; and/or
(b) the 3′ mixmer region comprises a first type of 3′ mixmer nucleoside and a second type of 3′ mixmer nucleoside, wherein the sugar moiety of the first type of 3′ mixmer nucleoside is different to the sugar moiety of the second type of 3′ mixmer nucleoside.

3. The oligonucleotide of claim 1, wherein
(a) the 5′ mixmer region comprises an alternating pattern of single nucleosides of the first type of 5′ mixmer nucleosides and single nucleosides of the second type of 5′ mixmer nucleosides; and/or
(b) the 3′ mixmer region comprises an alternating pattern of single nucleosides of the first type of 3′ mixmer nucleosides and single nucleosides of the second type of 3′ mixmer nucleosides.

4. The oligonucleotide of claim 1, wherein
(a) the 5′ mixmer region comprises the nucleosides from position +1 to position +10, +11, +12, +13, +14, +15, +16, +17, +18, +19, +20, +21, +22, +23, +24 or +25; and/or
(b) the 3′ mixmer region comprises the nucleosides from position −1 to position −3, −4, −5, −6, −7, −8, −9, −10, −11, −12, −13, −14, −15, −16 or −17.

5. The oligonucleotide of claim 2, wherein
(a) the first type of 5′ mixmer nucleoside is selected from the group consisting of DNA, RNA, 2′-O-methyl-RNA, 2′-O-methoxyethyl-RNA (MOE-RNA), 2′-fluoro-RNA, linked nucleic acid (LNA), arabinonucleic acid (ANA) and 2′-fluoroarabinonucelic acid (FANA) nucleosides; and/or
(b) the second type of 5′ mixmer nucleoside is selected from the group consisting of DNA, RNA, 2′-O-methyl-RNA, MOE-RNA, 2′-fluoro-RNA, LNA, ANA and FANA nucleosides; and/or
(c) the first type of 3′ mixmer nucleoside is selected from the group consisting of DNA, RNA, 2′-O-methyl-RNA, MOE-RNA, 2′-fluoro-RNA, LNA, ANA and FANA nucleosides; and/or
(d) the second type of 3′ mixmer nucleoside is selected from the group consisting of DNA, RNA, 2′-O-methyl-RNA, MOE-RNA, 2′-fluoro-RNA, LNA, ANA and FANA nucleosides.

6. (canceled)

7. The oligonucleotide of claim 1, wherein
(a) the nucleoside at one or more of positions +3, +5, +7, +9, +11, +13, +15, +17 and +19 is a 2′-fluoro-RNA nucleoside; and/or
(b) the nucleoside at one or more of positions −3, −5, −7 and −9 is a 2′-fluoro-RNA nucleoside; and/or
(c) the nucleoside at one or more of positions +2, +4, +6, +8, +10, +12, +14 and +18 is a 2′-O-methyl-RNA nucleoside; and/or
(d) the nucleoside at one or more of positions −2, −4 and −6 is a 2′-O-methyl-RNA nucleoside.

8. The oligonucleotide claim 1, wherein
(a) the nucleoside at position −2 is a 2′-O-methyl-RNA nucleoside; and/or
(b) the nucleoside at position −3 is a 2′-fluoro-RNA nucleoside; and/or
(c) the nucleoside at position −8 is a 2′-fluoro-RNA nucleoside; and/or
(d) the nucleoside at position +16 is a 2′-fluoro-RNA nucleoside; and/or
(e) the nucleoside at position +17 is a 2′-fluoro-RNA nucleoside.

9. The oligonucleotide of claim 1, wherein
(a) the nucleoside at position −8 is a 2′-O-methyl-RNA nucleoside; and/or
(b) the nucleoside at position +16 is a 2′-O-methyl-RNA nucleoside.

10. The oligonucleotide of claim 1, wherein the oligonucleotide is for editing a target nucleic acid comprising a target adenosine, wherein the oligonucleotide is capable of effecting conversion of the target adenosine (A) to inosine (I).

11. The oligonucleotide of claim 10, wherein the target nucleic acid is a SERPINA1 mRNA.

12. (canceled)

13. (canceled)

14. (canceled)

15. The oligonucleotide of claim 1, wherein
(a) the oligonucleotide comprises exactly 20, 21, 22, 23, 24, 25, 26, 27, 28, 29 or 30 nucleosides 5′ to the editing nucleoside; and/or
(b) the oligonucleotide comprises exactly 4, 5, 6, 7, 8, 9, 10, 11, 12, 13, 14, 15, 16, 17, 18, 19 or 20 nucleosides 3′ to the editing nucleoside.

16. The oligonucleotide of claim 1, wherein the sequence of the oligonucleotide comprises from 30 to 61 contiguous nucleosides from the following sequence:
(SEQ_ID_NO_87)
CUCUAAAAACAUGGCCCCAGCAGCUUCAGUCCCUUUCTC
IUCGAUGGUCAGCACAGCCUUAUGCACGGCCUUGGUGU,
or from a variant of SEQ ID NO 87 comprising exactly 1, exactly 2 or exactly 3 single nucleoside substitutions,
wherein I is inosine, and
wherein the oligonucleotide comprises positions 38, 39 and 40 of SEQ ID NO 87.

17. The oligonucleotide of claim 1, wherein the sequence of the oligonucleotide comprises a sequence having at least 95% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 or 86.

18. (canceled)

19. The oligonucleotide of claim 1, wherein the oligonucleotide comprises any one of CMP ID NOs 1_1, 2_1, 3_1, 3_2, 3_3, 4_1, 5_1, 6_1, 7_1, 8_1, 9_1, 10_1, 11_1, 12_1, 13_1, 14_1, 15_1, 16_1, 17_1, 18_1, 19_1, 20_1, 21_1, 22_1, 23_1, 24_1, 25_1, 26_1, 26_2, 26_3, 26_4, 26_5, 26_6, 26_7, 26_8, 26_9, 27_1, 28_1, 29_1, 30_1, 31_1, 32_1, 32_2, 32_3, 32_4, 32_5, 32_6, 32_7, 32_8, 32_9, 32_10, 32_11, 32_12, 32_13, 32_14, 32_15, 32_16, 32_17, 32_18, 32_19, 32_20, 32_21, 32_22, 32_23, 32_24, 32_25, 32_26, 32_27, 32_28, 32_29, 32_30, 32_31, 32_32, 32_33, 32_34, 32_35, 32_36, 32_37, 32_38, 32_39, 32_40, 32_41, 32_42, 32_43, 32_44, 32_45, 32_46, 32_47, 32_48, 32_49, 32_50, 32_51, 32_52, 32_53, 32_54, 32_55, 32_56, 32_57, 32_58, 32_59, 32_60, 32_61, 32_62, 32_63, 32_64, 32_65, 32_66, 32_67, 32_68, 32_69, 32_70, 32_71, 32_72, 32_73, 32_74, 32_75, 32_76, 32_77, 32_78, 32_79, 32_80, 32_81, 32_82, 32_83, 33_1, 34_1, 35_1, 36_1, 37_1, 38_1, 39_1, 40_1, 40_2, 40_3, 40_4, 40_5, 40_6, 40_7, 40_8, 40_9, 40_10, 41_1, 41_2, 413, 414, 415, 416, 417, 418, 419, 4110, 42_1, 422, 423, 42_4, 42_5, 42_6, 43_1, 43_2, 43_3, 44_1, 45_1, 46_1, 47_1, 47_2, 48_1, 49_1, 50_1, 51_1, 52_1, 53_1, 54_1, 55_1, 56_1, 57_1, 58_1, 59_1, 60_1, 61_1, 62_1, 63_1, 64_1, 65_1, 66_1, 67_1, 68_1, 69_1, 70_1, 71_1, 72_1, 73_1, 74_1, 75_1, 76_1, 77_1, 78_1, 79_1, 80_1, 81_1, 82_1, 83_1, 84_1, 85_1 or 86_1.

20. (canceled)

21. (canceled)

22. An oligonucleotide conjugate comprising the oligonucleotide of claim 1 covalently attached to at least one conjugate moiety.

23. (canceled)

24. A pharmaceutical composition comprising the oligonucleotide claim 1, and a pharmaceutically acceptable diluent, solvent, carrier, salt and/or adjuvant.

25. A method for editing a target nucleic acid in a target cell, the method comprising administering an effective amount of the oligonucleotide of claim 1 to the target cell.

26. A method of treating a subject having a disease comprising administering a therapeutically effective amount of the oligonucleotide of claim 1 to the subject.

27. The method of claim 26, wherein the disease is alpha 1 antitrypsin deficiency (A1AD).
